(19) United States
(12) Patent Application Publication
(10) Pub. No.: US 2023/0285662 A1
(43) Pub. Date: Sep. 14, 2023

(54) Medical Pump

(71) Applicant: DEKA Products Limited Partnership, Manchester, NH (US)

(72) Inventors: Michael J. Baier, Dunbarton, NH (US); David Blumberg, JR., Deerfield, NH (US); Hugh J. Boddington, Concord, NH (US); Thomas A. Friedrich, Loudon, NH (US); Paul G. Girouard, Allenstown, NH (US); Larry B. Gray, Merrimack, NH (US); David C. Nivens, Hollis, NH (US)

(21) Appl. No.: 18/198,905

(22) Filed: May 18, 2023

Related U.S. Application Data
(63) Continuation-in-part of application No. 16/542,789, filed on Aug. 16, 2019, now Pat. No. 11,707,615.
(60) Provisional application No. 62/765,100, filed on Aug. 16, 2018.

(51) Int. Cl. A61M 5/142; A61M 5/145

(52) U.S. Cl. CPC A61M 5/14228; A61M 5/1452; A61M 2005/14533

(57) ABSTRACT

A pump for treating a patient is disclosed that includes a plunger transitionable between a first position and a second position, wherein when a tube is loaded within the pump, the plunger applying a force against the tube when in the second position; a cam shaft configured to actuate the plunger to transition the plunger from the first position to the second position; a motor coupled to the cam shaft; a heatsink to dissipate heat from the motor, the heatsink including a first part and a second part; and a housing containing at least a portion of the plunger, the cam shaft, the motor and the heatsink, the first part of the heatsink extending through a surface of the housing, the second part is thermally in contact with the motor, and the first and second parts of the heatsink are movable relative to one another to define an adjustable length.

### CROSS-REFERENCE TO RELATED APPLICATIONS

The present disclosure is a continuation-in-part of application Ser. No. 16/542,789 filed Aug. 16, 2019 (Attorney Docket No. AA032), which claims priority from U.S. Prov. App. Ser. No. 62/765,100, filed Aug. 16, 2018, which is hereby incorporated herein, in its entirety, by reference.

### BACKGROUND

### Relevant Field

Peristaltic pumps are used in a variety of applications including medical applications. In medical applications, peristaltic pumps are used to infuse a patient with a fluid and typically have the benefit of isolating the medical fluid being infused into a patient while maintaining sterility. Some peristaltic pumps work by compressing or squeezing a length of flexible tubing thereby preventing the fluid being infused from coming into contact with the pump or internal pump mechanism. A mechanical mechanism of the peristaltic pump pinches a portion of the tubing to push fluid trapped in the tubing in the direction of the patient. Examples of peristaltic pumps include rotary peristaltic pumps and finger peristaltic pumps.

Rotary peristaltic pumps typically move liquids through flexible tubing placed in an arc-shaped platen. Rotary peristaltic pumps are generally made of two to four rollers placed on a roller carrier driven rotationally by a motor. A typical rotary peristaltic pump has a rotor assembly with pinch rollers that apply pressure to the flexible tubing at spaced locations to provide a squeezing action on the tubing against a counter surface. The occlusion of the tubing creates increased pressure ahead of the squeezed area and reduced pressure behind that area, thereby forcing a liquid through the tubing as the rotor assembly moves the pinch rollers along the tubing. In order to operate, there must always be an occlusion zone; in other words, at least one of the rollers is always pressing on the tube.

Finger peristaltic pumps are made of a series of fingers moving in cyclical fashion to flatten a flexible tube against a counter surface. The fingers move essentially vertically to form a zone of occlusion that moves fluid from upstream to downstream. The most commonly used finger pumps are linear, meaning that the counter surface is flat and the fingers are parallel. In this case, the fingers are controlled by a series of cams arranged one behind another, each cam cooperating with a finger. These cams may be placed in a helically offset manner on a shared shaft driven rotationally by a motor. There are also rotary-finger peristaltic pumps, which attempt to combine the advantages of roller pumps with those of finger pumps. In this type of pump, the counter surface is not flat, but arc-shaped, and the fingers are arranged radially inside the counter surface. In this case, a shared cam with multiple knobs placed in the center of the arc is used to activate the fingers.

### SUMMARY

In an embodiment of the present disclosure, a medical pump may include: a plunger that is transitionable between a first position and a second position, wherein when a tube is loaded within the pump and the plunger is in the second position, the plunger applies a force against the tube; a cam shaft configured to actuate the plunger to transition the plunger from the first position to the second position; a motor coupled to the cam shaft; a heatsink to dissipate heat from the motor, the heatsink including a first part and a second part; and a housing, the housing containing at least a portion of each of the plunger, the cam shaft, the motor and the heatsink. The first part of the heatsink may extend through a surface of the housing, the second part is thermally in contact with the motor, and the first and second parts of the heatsink are movable relative to one another along a longitudinally extending axis such that the first and second parts define an overall length that is adjustable. The first and second parts of the heatsink may be slidable relative to one another while frictionally fit together.

In an embodiment, the heatsink may further include: a thermally-conductive, braided wire having a first end and a second end, where the first end is coupled to the motor to dissipate heat from the motor, and/or the second end may be coupled to the heatsink. For example, the second end may be soldered onto the heatsink. The second end may be clipped onto the heatsink via a fastener such as a clip. The first end may be soldered onto the motor. The first end may be clipped onto the heatsink using the clip. The thermally-conductive, braided wire may include a metal. The metal may be copper, aluminum, and/or alloys thereof. The first end may be soldered onto the motor.

The first end of thermally-conductive, braided wire may be thermally coupled to the heatsink and the second end of the thermally-conductive, braided wire may be thermally coupled to the powerbar heat dissipater. The pump may include a heat pipe coupled to the powerbar heat dissipater and the thermally-conductive, braided wire to dissipate heat therebetween. The pump may include a heat pipe coupled to the powerbar heat dissipater and the heatsink to dissipate heat therebetween. The heatsink may be disposed on a back of the pump, and/or may be positioned to dissipate heat into a surrounding ambient air. The pump may include a heat pipe coupled to the thermally-conductive, braided wire and the heatsink to dissipate heat therebetween. In an embodiment, the thermally conductive, braided wire may comprises any suitable thermally conductive material including, for example, a non-metallic thermally conductive material.

The pump may also include a plunger configured for actuation against a tube; a cam shaft configured to actuate the plunger; a motor coupled to the cam shaft; a heatsink; and a planar-thermal connector having a first end and a second end, where the planar-thermal connector may be configured to thermally conduct heat from the motor. The planar-thermal connector may have at least one arm extending to thermally contact the motor. The planar-thermal connector may have two arms extending to contact two metal connectors, and/or the two metal connectors may be in thermal contact with the motor. The first end of the planar-thermal connector may be thermally coupled to the heatsink and the second end of the planar-thermal connector may be thermally coupled to the powerbar heat dissipater.

The pump may include a heat pipe coupled to the powerbar heat dissipater and the planar-thermal connector to dissipate heat therebetween. The pump may include a heat pipe coupled to the powerbar heat dissipater and the heatsink to dissipate heat therebetween. The planar-thermal connector may include two arms extending therefrom, and/or the two arms may be in thermal contact with the motor. The heatsink may be disposed on a back of the pump.

The pump the planar-thermal connector may include: a powerbar heat dissipater; two arms extending to contact two metal connectors, the two metal connectors being in thermal contact with the motor; and a heat-strap bracket configured to be disposed between the powerbar heat dissipater and the heatsink, the heat-strap bracket being configured to provide compliance between the powerbar heat dissipater and the heatsink. The heat-strap bracket may be electrically insulating. The heat-strap bracket includes a bottom side disposed away from the heatsink and toward a powerbar heat dissipater, where the first end of the planar-thermal connector may be disposed adjacent to the bottom side of the heat-strap bracket. The pump may include a thermal pad disposed between the first end of the planar-thermal connector and the powerbar heat dissipater. The at least one protrusion may be configured to limit a compression of the thermal pad. The heat-strap bracket may include at least one latch receiver configure to receive at least one latch, where each of the at least one latch may be configured to secure the planar-thermal connector to the heatsink, and/or the planar-thermal connector may include at least one hole to receive a respective one of the at least one latch. The heatsink may be positioned to dissipate heat into a surrounding ambient air. The pump may include a heat pipe that is coupled to the planar-thermal connector and the heatsink to dissipate heat therebetween. Additionally or alternatively, the pump may include a heat pipe coupled to the planar-thermal connector and the motor to dissipate heat therebetween.

In an embodiment, the pump may also include a platen configured to retain a tube; a plunger having an end effector configured for action against the tube; a platen disposed within the platen and adjacent to the end effector of the plunger, where the platen has a first end and a second end; a first adjuster disposed adjacent to the platen at the first end; and a second adjuster disposed adjacent to the platen at the second end. The first adjuster may be configured to actuate the platen toward or away from the plunger. The second adjuster may be configured to actuate the platen toward or away from the plunger. The first adjuster and the second adjuster are configured to actuate opposite ends of the platen.

In an embodiment, the pump may include: a first mount configured to secure the first end of the platen to the pump; and a second mount configured to secure the second end of the platen to the pump. The first end may be disposed adjacent to the first mount, where the central axis of the fastener may be parallel to the platen. The fastener may be a screw. The first adjuster defines a ramp having a first side positioned away from the first mount and a second side engaging with the first mount at an angle thereby defining the ramp. The first adjuster may be configured to actuate the first end of the platen toward the end effector when the first adjuster may be actuated away from the second adjuster. The first adjuster may include a planar portion that disposed between the platen and the pump. The first adjuster may include a thermal insulator, which may be, for example, a plastic.

In an embodiment, the pump may include a platen that may be configured to retain a tube; a shaft having a first end and second end. The pump also includes a plunger having an end effector configured for action against the tube, where the plunger may be pivotably attached to the shaft; a platen disposed within the platen and adjacent to the end effector of the plunger, where the platen has a first end and a second end; and a first shaft adjuster operatively engaged with the first end of the shaft.

In an embodiment, the pump may include a second shaft adjuster operatively engaged with the second end of the shaft. The first and second shaft adjusters are configured to cooperate to actuate the first end of the shaft. The pump may include an adjustment screw configured to actuate the first shaft adjuster. The pump may include a ramp disposed adjacent to the first shaft adjuster and configured to actuate shaft as the first shaft adjuster is actuated. The pump may include a plunger having an end effector configure to actuate toward and away from a tube, where the end effector may be formed from a thermally insulating material; and a cam shaft configured to actuate the plunger. The pump where the end effector may include and/or be formed from an insulating material. For example, the end effector may include and/or be formed from a plastic material.

In an embodiment, the pump may include a plunger having an end effector configure to actuate toward and away from a tube; a multi-stage spring configured to vary a bias of the end effector against the tube, and a cam shaft configured to actuate the plunger. The multi-stage spring may be a torsion spring. The multi-stage spring may include two springs. The multi-stage spring may be a conical torsional spring. The multi-stage spring may be a variable diameter spring. The multi-stage spring may be a variable-force torsional spring.

In an embodiment, the multi-stage spring may be a variable-stiffness torsional spring. The multi-stage spring includes a torsional spring and a leaf spring. A leaf spring may be coupled to a cam follower to provide a torsional force between a first positon and a second position. When the plunger applies a force to the tube and the leaf spring may be between a first stop and a second stop, a first force may be applied to the tube. When the leaf spring may be stopped by one of the first stop and the second stop and a cam follower of the plunger leaves the cam shaft, a second force may be applied to the tube. The pump may include: a position sensor operatively coupled to the end effector to estimate a position of the end effector; and a processor configured estimate a first position of the end effector when the first force may be applied to the tube and a second position of the end effector when the second force may be applied to the tube. The processor may be configured to estimate an amount of air within the tube using the first and second positions of the end effector. The processor estimates a first pressure within the tube when the first force may be applied to the tube. The processor estimates a second pressure within the tube when the second force may be applied to the tube. The processor estimates an amount of air within the tube using the first and second pressures within the tube. The processor estimates an amount of air within the tube using the first and second pressures within the tube by utilizing an ideal gas law. Embodiments of the described techniques may include hardware, a method or process, or computer software on a computer-accessible medium.

In an embodiment, a method for actuating a peristaltic pump may include: actuating a plunger toward a tube; transitioning a first spring within the plunger when a predetermined force may be applied to the plunger; determining a first position of the plunger when the first spring transitions, actuating the plunger toward the tube after the first spring transitions and until a cam follower of the plunger disengages from a cam; determining a second position of the plunger after the cam follower of the plunger disengages from the cam; determining a difference between the first and second positions; and calculating a volume of fluid in accordance with the difference.

In an embodiment, the method where the first spring may be configured to transition after sufficient force may be applied to the tube to substantially compress any gas within the tube. A second spring may be configured to actuate the plunger toward the tube. The cam follower may be configured to actuate the plunger away from the tube. The second spring may be configured to actuate the plunger against the tube when the cam follower of the plunger disengages from the cam. The first spring may be a leaf spring configured to couple the cam follower to the plunger. Embodiments of the described techniques may include hardware, a method or process, or computer software on a computer-accessible medium.

In an embodiment, the pump may include a platen; a plunger having an end effector configure to actuate toward and away from the platen; a piezoelectric transmitter disposed on a first position adjacent to the platen, the piezoelectric transmitter configured to generate an ultrasonic wave from the piezoelectric transmitter; a piezoelectric receiver disposed on a second position adjacent to the platen and at an opposite side of the platen relative to the first piezoelectric transmitter, the piezoelectric receiver configured to receive the ultrasonic wave from the piezoelectric transmitter; and a processor operatively coupled to the piezoelectric transmitter to generate the ultrasonic wave, the processor operatively coupled to the piezoelectric receiver go receive an ultrasonic signal corresponding to the received ultrasonic wave, where the processor may be configured to: set a trigger threshold to a first bubble threshold, and set the trigger threshold to a second bubble threshold when the ultrasonic signal may be below the first threshold for a first predetermined amount of time.

In an embodiment, the processor may be further configured to set the trigger threshold to the second bubble threshold when the ultrasonic signal may be below the first threshold and above a second threshold for a predetermined amount of time, the second threshold being lower than the first threshold. The processor may be further configured to set the trigger threshold to a third bubble threshold when the ultrasonic signal may be below the second threshold and below the third threshold for a second predetermined amount of time, the second threshold being lower than the first threshold and the second bubble threshold. The processor may be further configured to detect a bubble condition when ultrasonic signal may be below the trigger threshold. The processor may be further configured estimate a size of a bubble when in the bubble condition by integrating a volume of fluid delivered by the pump over time until the ultrasonic signal rises to above the trigger threshold. The processor may be configured to set the trigger threshold to the first bubble threshold when the ultrasonic signal rises from below the first threshold to above the first threshold. Embodiments of the described techniques may include hardware, a method or process, or computer software on a computer-accessible medium.

In an embodiment a pump for pumping fluid may include a platen; a plunger having an end effector configure to actuate toward and away from the platen; an ultrasonic air detector disposed in spaced relation to the platen, the ultrasonic air detector may include: a first piezoelectric transmitter disposed on a first position adjacent to the platen; a first piezoelectric receiver disposed on a second position adjacent to the platen and at an opposite side of the platen relative to the first piezoelectric transmitter; a second piezoelectric transmitter disposed on a third position adjacent to the platen; and a second piezoelectric receiver disposed on a fourth position adjacent to the platen and at an opposite side of the platen relative to the first piezoelectric transmitter, where the first piezoelectric transmitter may be upstream from the second piezoelectric transmitter. The pump also includes a processor operatively coupled to the ultrasonic air detector to receive a first ultrasonic signal corresponding to the first piezoelectric receiver and to receive a second ultrasonic signal corresponding to the second piezoelectric receiver, where the processor may be configured to: monitor the first ultrasonic signal for an air bubble, monitor the second ultrasonic signal for the air bubble only after a detection of the air bubble from the first ultrasonic signal, determine the air bubble has passed downstream only after the air bubble may be detected by the second ultrasonic signal, and estimate a size of the air bubble.

In an embodiment, the processor may be further configured to estimate the size of the air bubble by monitoring an amount of air that passes through the ultrasonic air detector. The size of the air bubble may be estimated when the first ultrasonic signal and the second ultrasonic signal do not detect the air bubble simultaneously. To update the estimate of the size of the air bubble, the processor may be further configured to: estimate a raw upstream estimate corresponding to a first volume of fluid pumped when the first ultrasonic signal corresponds to the air bubble, estimate a raw downstream estimate corresponding to a second volume of fluid pumped when the second ultrasonic signal corresponds to the air bubble, and update the estimate of the size of the air bubble in accordance with the first and second ultrasonic signals. To update the estimate of the size of the air bubble in accordance with the first and second ultrasonic signals, the processor may be configured to estimate the size of the air bubble as being zero if at least one of the raw upstream estimate and the raw downstream estimate may be zero. To update the estimate of the size of the air bubble in accordance with the first and second ultrasonic signals, the processor may be configured to determine a ratio of the raw upstream estimate over the raw downstream estimate. To update the estimate of the size of the air bubble in accordance with the first and second ultrasonic signals, the processor may be configured to estimate the size of the air bubble as being the raw downstream estimate if the ratio may be greater than 2. To update the estimate of the size of the air bubble in accordance with the first and second ultrasonic signals, the processor may be configured to estimate the size of the bubble as being the raw upstream estimate if the ratio may be less than 0.5. To update the estimate of the size of the air bubble in accordance with the first and second ultrasonic signals, the processor may be configured to estimate the size of the bubble as being an average of the raw upstream estimate and the raw downstream estimate. To update the estimate of the size of the air bubble in accordance with the first and second ultrasonic signals, the processor may be configured to estimate the size of the bubble as being: the raw downstream estimate if the ratio may be greater than 2, the raw upstream estimate if the ratio may be less than 0.5, and an average of the raw upstream estimate and the raw downstream estimate if the ratio may be less than or equal to 2 or may be greater than or equal 0.5.

The processor may be configured to discard any bubbles from a total of air volume when the processor determines a respective air bubble may be traveling upstream using the ultrasonic air detector. To update the estimate of the size of the air bubble in accordance with the first and second ultrasonic signals, the processor may be configured to ignore the air bubble if the estimate of the size of the air bubble may be less than a hold-up volume. The hold-up volume may be a cross-sectional area of a tube disposed within the ultrasonic air detector multiplied by a distance along the platen between the first and second piezoelectric transmitters. The pump may include a rib protruding from the ultrasonic air detector in a direction orthogonal to a length of the platen, the rib being disposed between one of the first piezoelectric transmitter and the first piezoelectric receiver, and one of the second piezoelectric transmitter and the first piezoelectric receiver. The first piezoelectric transmitter and the second piezoelectric transmitter are on a same side of the platen. The first piezoelectric transmitter and the second piezoelectric receiver are on a same side of the platen. Embodiments of the described techniques may include hardware, a method or process, or computer software on a computer-accessible medium.

An embodiment may include a pump that includes a platen; a plunger having an end effector configure to actuate toward and away from the platen; an ultrasonic air detector disposed in spaced relation to the platen, the ultrasonic air detector may include: a first piezoelectric transmitter disposed on a first position adjacent to the platen; a first piezoelectric receiver disposed on a second position adjacent to the platen and at an opposite side of the platen relative to the first piezoelectric transmitter; a second piezoelectric transmitter disposed on a third position adjacent to the platen; and a second piezoelectric receiver disposed on a fourth position adjacent to the platen and at an opposite side of the platen relative to the first piezoelectric transmitter, where the first piezoelectric transmitter may be upstream from the second piezoelectric transmitter. The pump may also include a processor that is operatively coupled to the ultrasonic air detector to receive a first ultrasonic signal corresponding to the first piezoelectric receiver and to receive a second ultrasonic signal corresponding to the second piezoelectric receiver, where the processor may be configured to have a plurality of states corresponding to the ultrasonic air detector, the plurality of states including a monitor-for-upstream-air state, a monitor-for-downstream-air state, and a monitor-for-fluid state, where: the monitor-for-upstream-air state transitions to the monitor-for-downstream-air state when the first ultrasonic signal indicates air, the monitor-for-downstream-air state transitions to the monitor-for-fluid state when the second ultrasonic signal indicates air, and the monitor-for-fluid state estimates a volume of an air bubble when the first and second ultrasonic signals indicate the volume of the air bubble may be greater than a fluid clear volume or one of the first and second ultrasonic signals indicate fluid has been detected that may be more than a single-sensor clear volume.

Embodiments may include one or more of the following features. The pump where the single-sensor clear volume may be a volume of consecutive fluid detected by a single one of the first ultrasonic signal or the second ultrasonic signal used to determine an end of a bubble. The single-sensor clear volume may be greater than the fluid clear volume. The fluid clear volume may be a volume of consecutive fluid detected by at least one of the first ultrasonic signal or the second ultrasonic signal used to determine an end of a bubble.

To update the estimate of a size of the air bubble in accordance with the first and second ultrasonic signals, the processor may be configured to ignore a bubble if the estimate of the size of the air bubble may be less than a hold-up volume. The hold-up volume may be a cross-sectional area of a tube disposed within the ultrasonic air detector multiplied by a distance along the platen between the first and second piezoelectric transmitters. To update the estimate of a size of the air bubble in accordance with the first and second ultrasonic signals, the processor may be configured to estimate the size of the air bubble as being zero if at least one of a raw upstream estimate and a raw downstream estimate may be zero. To update the estimate of a size of the air bubble in accordance with the first and second ultrasonic signals, the processor may be configured to determine a ratio of a raw upstream estimate over a raw downstream estimate. To update the estimate of the size of the air bubble in accordance with the first and second ultrasonic signals, the processor may be configured to estimate the size of the bubble as being the raw downstream estimate if the ratio may be greater than 2. To update the estimate of the size of the air bubble in accordance with the first and second ultrasonic signals, the processor may be configured to estimate the size of the air bubble as being the raw upstream estimate if the ratio may be less than 0.5. To update the estimate of the size of the air bubble in accordance with the first and second ultrasonic signals, the processor may be configured to estimate the size of the air bubble as being an average of the raw upstream estimate and the raw downstream estimate. To update the estimate of the size of the air bubble in accordance with the first and second ultrasonic signals, the processor may be configured to estimate the size of the air bubble as being: the raw downstream estimate if the ratio may be greater than 2, the raw upstream estimate if the ratio may be less than 0.5, and an average of the raw upstream estimate and the raw downstream estimate if the ratio may be less than or equal to 2 or may be greater than or equal 0.5. Embodiments of the described techniques may include hardware, a method or process, or computer software on a computer-accessible medium.

An embodiment may include a pump for treating a patient. The pump also includes an inlet valve; an outlet valve; a spring-biased plunger biased for actuation toward a tube; and a processor configured to: deliver fluid downstream for a predetermined amount of time, and reverse fluid flow for a predetermined amount, and repeat the deliver and reverse actions.

An embodiment may include one or more of the following features. The pump where the predetermined amount may be a predetermined amount of volume. The predetermined amount of volume may be a function of a fluid delivery rate. The predetermined amount may be a volume of fluid adjacent to the spring-biased plunger when the inlet and outlet valves are closed. The predetermined amount may be another predetermined amount of time, which may be a function of a fluid delivery rate. The reverse and repeat actions only occur when the deliver fluid downstream act may be within 0.1 ml/hr to 500 ml/hr. The predetermined amount may be 30 minutes. Prior to the reverse fluid flow act, the processor may be configured to: close the outlet valve, open the inlet valve, and actuate the spring-biased plunger away from the tube, and where in the reverse fluid flow act, the processor may be configured to: actuate the spring-biased plunger toward the tube to thereby reverse fluid flow for the predetermine amount, the predetermined amount being a volume of fluid adjacent to the spring-biased plunger when the inlet and outlet valves are closed.

The processor may be configured to actuate the spring-biased plunger away from the tube at 400 degrees per second prior to the reverse fluid flow act. The processor may be configured to actuate the spring-biased plunger toward the tube at 800 degrees per second when in the reverse fluid flow act. The processor may be configured to actuate the spring-biased plunger away from the tube at a first rotation rate prior to the reverse fluid flow act, the processor may be configured to actuate the spring-biased plunger toward the tube at a second rotation rate when in the reverse fluid flow act, and the absolute ratio value of the second rotation rate to the first rotation rate may be 2. The first rotation rate may be less than 266.6 degrees per second.

The processor may be configured to actuate the spring-biased plunger away from the tube at 200 degrees per second prior to the reverse fluid flow act. The processor may be configured to actuate the spring-biased plunger toward the tube at 800 degrees per second when in the reverse fluid flow act. The processor may be configured to actuate the spring-biased plunger toward the tube at 200 degrees per second when in the reverse fluid flow act.

Prior to the reverse fluid flow act, the processor may be configured to: close the outlet valve, open the inlet valve, and actuate the spring-biased plunger away from the tube, and where in the reverse fluid flow act, the processor may be configured to: actuate the spring-biased plunger toward the tube to thereby reverse fluid flow for a predetermine amount, where the predetermined amount may be less than a full pumping cycle.

Prior to the reverse fluid flow act, the processor may be configured to: close the outlet valve, open the inlet valve, and actuate the spring-biased plunger away from the tube, and where in the reverse fluid flow act, the processor may be configured to: actuate the spring-biased plunger toward the tube to thereby reverse fluid flow for a predetermine amount, where the predetermined amount may be more than a full pumping cycle. An upstream fluid pressure may be increased by the check valve when the pump reverses fluid flow.

Prior to the reverse fluid flow act, the processor may be configured to: close the outlet valve, open the inlet valve, and actuate the spring-biased plunger away from the tube, and where in the reverse fluid flow act, the processor may be configured to: actuate the spring-biased plunger toward the tube to thereby reverse fluid flow for a predetermine amount, where the predetermined amount corresponds to having an upstream fluid pressure being less than a predetermined threshold. Embodiments of the described techniques may include hardware, a method or process, or computer software on a computer-accessible medium.

An embodiment may include a pump for treating a patient may include an inlet valve; an outlet valve; a spring-biased plunger biased for actuation toward a tube; an actuator configured to actuate the spring-biased plunger; and a processor configured to: deliver fluid downstream for a predetermined amount of a first parameter, close a downstream valve, actuate the spring-biased plunger toward the tube, disengage the actuator from the spring-biased plunger, interrupt actuation of the actuator, reverse actuation of the actuator for a predetermined amount of a second parameter, and repeat the deliver and reverse actions.

Embodiments may include one or more of the following features. The pump where the first parameter may be time. The first parameter may be fluid volume. The first parameter may be a number of peristaltic pumping cycles. The first parameter may be an amount of air pumped passed a downstream air sensor. The second parameter may be time. The second parameter may be fluid volume. The second parameter may be a number of peristaltic pumping cycles. The second parameter may be a movement of the actuator. The actuator may be a cam shaft. Embodiments of the described techniques may include hardware, a method or process, or computer software on a computer-accessible medium.

An embodiment may include an infusion set that may include a tube having a first end configured to couple to a bag of fluid; a length of tubing configured for being operated on by a peristaltic pump, the length defining part of the tube; a nucleation site within the tube configured to cause a formation of bubbles within the tube; and an air trap coupled to the tube and configured to capture any bubbles upstream of the nucleation site.

In an embodiment, the infusion set where the nucleation site may be a restricted portion of the tube. The nucleation site may be an hour-glass shaped portion of the tube. The air trap may be configured to trap about 50 microliters of air. The air trap includes a dome to trap the bubbles. The air trap may be downstream of the length of tube. The air trap may be upstream of the length of tube. The nucleation site may be formed at a junction of the length of tubing and another portion of the tube, in which one of the lengths of tubing and the other portion of tube may be rolled inward during bonding. The rolled inward portion forms a toroid. An underside of the toroid defines the air trap. The nucleation site may be formed by roughing a portion of the toroid. The nucleation site may be formed by scoring a portion of the toroid. The nucleation site may be formed by etching a portion of the toroid. The nucleation site may be formed by roughing an inside wall of the tube. The nucleation site may be formed by scoring an inside wall of the tube. The nucleation site may be formed by etching an inside wall of the tube. The system according to one may include prongs disposed within the tube to create the nucleation site. Embodiments of the described techniques may include hardware, a method or process, and/or computer software on a computer-accessible medium.

An embodiment may include a system for infusion fluid that includes a peristaltic pump having a plunger. The system also includes an infusion set may include: a tube having a first end configured to couple to a bag of fluid; a length of tubing configured for being operated on by the plunger of the peristaltic pump, the length defining part of the tube; a nucleation site within the tube configured to cause a formation of bubbles within the tube; and an air trap coupled to the tube and configured to capture any bubbles upstream of the nucleation site; and a heater disposed upstream of the nucleation site, the heater configured to heat the tube to thereby facilitate bubble formation at the nucleation site.

Embodiments may include one or more of the following features. The system where the heater may be active when a flow rate of the peristaltic pump may be less than a threshold. The heater may be upstream of the peristaltic pump and the nucleation site may be downstream of the peristaltic pump, where a temperature of a fluid flowing through the tube decreases when traversing through the peristaltic pump. A temperature of a fluid flowing through the tube reaches a peak temperature at the nucleation site. The nucleation site may be a restricted portion of the tube. The nucleation site may be an hour-glass shaped portion of the tube. The air trap may be configured to trap about 50 microliters of air. The air trap includes a done to trap the bubbles. The air trap may be downstream of the length of tube. The air trap may be upstream of the length of tube. The nucleation site may be formed at a junction of the length of tubing and another portion of the tube, there one of the length of tubing and the other portion of tube may be rolled inward during bonding. The rolled inward portion forms a toroid. An underside of the toroid defines the air trap. The nucleation site may be formed by roughing a portion of the toroid. The nucleation site may be formed by scoring a portion of the toroid. The nucleation site may be formed by etching a portion of the toroid. The nucleation site may be formed by roughing an inside wall of the tube. The nucleation site may be formed by scoring an inside wall of the tube. The nucleation site may be formed by etching an inside wall of the tube. Embodiments of the described techniques may include hardware, a method or process, or computer software on a computer-accessible medium.

An embodiment may include a pressure sensor to measure fluid pressure. The pressure sensor also includes a first port configured to expand a first tube along a first direction; and a second port configured to attach to a second tube, the second port being in fluid communication with the second port where the first tube may be in fluid communication with the second tube.

Embodiments may include one or more of the following features. The pressure sensor where the first port may include: a first arm disposed adjacent to and parallel to a central axis of the first tube; and a second arm disposed adjacent and parallel to the central axis of the first tube, where the first arm may be disposed at an opposite side of the central axis. A distal end of the first arm arcs toward the central axis of the first tube. A distal end of the second arm arcs toward the central axis of the first tube. The first port includes a raised flange disposed along an inner wall of the first tube. The first port expands the first tube thereby defining a first flat side and a second flat side. The first elongated member may be pivotally coupled to the second elongated member. Distal ends of the first and second elongated members are attached to the first and second flat sides, respectively. The pressure sensor may include an actuation sensor configured to sense an amount of pivot of the first elongated member and the second elongated member of the clip. The pressure sensor may include a processor operatively coupled to the actuation sensor to receive the amount of pivot to calculate a fluid pressure inside of the first tube. Embodiments of the described techniques may include hardware, a method or process, or computer software on a computer-accessible medium.

An embodiment may include a system for pumping fluid an infusion set, may include: a tube having a first end configured to couple to a bag of fluid, a length of tubing configured for being operated on by a peristaltic pump, and a fluid bladder downstream of the length of tubing. The system also includes a peristaltic pump may include: a platen configured to retain the tube; a plunger having an end effector configured for action against the tube; and a platen disposed within the platen and adjacent to the end effector of the plunger. The fluid bladder may be configured to smooth a periodic pumping of fluid from the peristaltic pump.

Embodiments may include one or more of the following features. The system where the bladder may be shaped as a rectangular prism. The bladder has a portion with a reduced diameter configured to dislodge air bubbles within the tube. Embodiments of the described techniques may include hardware, a method or process, or computer software on a computer-accessible medium.

An embodiment may include a system for pumping fluid. The system also includes an infusion set may include a tube having a first end configured to couple to a bag of fluid, a length of tubing configured for being operated on by a peristaltic pump, and a length of compliant tubing downstream of the length of tubing. The system also includes a peristaltic pump may include: a platen configured to retain the tube; a plunger having an end effector configured for action against the tube; and a platen disposed within the platen and adjacent to the end effector of the plunger. The system also includes where the length of compliant tubing may be configured to smooth a periodic pumping of fluid from the peristaltic pump.

Embodiments may include one or more of the following features. The system where the compliant tubing has a low plasticity and high stretchability. The tube has a second end, where the infusion set includes a luer fitting disposed on the second end of the tube, where the luer includes a restriction configured to restrict fluid flow to thereby further smooth the periodic pump of fluid from the peristaltic pump. The restriction may be 17 thousandths of an inch. The tube may be at least 60 inches. The restriction may be configured to absorb 50 microliters of volume per periodic pumping.

In an embodiment, a captive screw system may include a screw having a head end, an shaft, and a threaded end, where the shaft may be disposed between the head and the threaded end and has a diameter less than a diameter of the threaded end; and a receiver hole having a threaded opening, a captive well, and a threaded hole configured to complementary engagement with the threaded end of the screw, where the captive well may be between the threaded opening and the threaded hole.

In an embodiment, a clamp for clamping to a pole may include a body having a first end and a second end; a first jaw member disposed on the first end of the body, the first jaw member configured to grasp onto a pole, the first jaw member fixable attached to the body; a second jaw member disposed may be spaced relation to the first jaw member; and a shaft disposed within a hole defined by the second end of the body, the shaft having a distal end and a proximal end, the distal end coupled to the second jaw member, the shaft having a collar and a threaded length, the threaded length extending from the proximal end to the collar, where the collar includes two horizontally-opposed cam follows opposed from a central axis of the shaft, where the hole of the second end of the body defines a plurality of partial-turn cam trenches configured to receive the two horizontally-opposed cam followers. The plurality of partial-turn cam trenches may be a plurality of quarter-turn cam trenches. The clamp may include a knob disposed on the proximal end of the shaft. Embodiments of the described techniques may include hardware, a method or process, or computer software on a computer-accessible medium.

In an embodiment, the pump also includes a lever that may be actuatable between a closed position and an open position. The pump also includes a first linkage coupled to the lever. The pump also includes a second linkage coupled to a pivot. The pump also includes a first rigid member pivotably coupled to the first linkage. The pump also includes a second rigid member pivotably coupled to the second linkage, the second rigid member pivotably coupled to the first rigid member. The pump also includes a hold. The pump also includes a first spring coupled to the hold and the first rigid member. The pump may also include a second spring coupled to the hold and the second rigid member. The first rigid member may include a protrusion, and the protrusion may extend away from a pivot of the first rigid member, where the first spring may be coupled to a distal end of the protrusion. The first rigid member, the second rigid member, the hold, the first spring, and the second spring are configured to provide a buckling action. The hold may be disposed at an opposite side of the first and second rigid members relative to the first and second linkages. When the first and second springs are in a relaxed state when the first and second rigid members are rotated on a respective pivot at a maximal distance between the respective pivots. When the first and second springs are in a charged state when the first and second rigid members are rotated on a respective pivot at a minimal distance between the respective pivots. When the first and second springs are in a relaxed state when the first and second rigid members are rotated on a respective pivot at a maximal distance between the respective pivots, where the second rigid member includes a stop, where the first and second rigid members cooperate to lock together at the maximal distance. The first rigid member, the second rigid member, the hold, the first spring, and the second spring are configured to provide a buckling action configured to reset the buckling action by actuation of the lever to the open position.

In an embodiment, a peristaltic pump for pumping fluid may include a platen configured for receiving a tube. The pump also includes a plunger configured for actuation toward and away from the platen. The pump also includes a position sensor configured to provide a position sensor signal of the plunger. The pump also includes a spring configured to bias the plunger toward the platen. The pump also includes an actuator configured to actuate the plunger. The pump also includes an inlet valve disposed adjacent to the platen and upstream of the plunger. The pump also includes an outlet valve disposed adjacent to the platen and downstream of the plunger. The pump also includes a processor configured to control actuation of the actuator, the processor configured to receive the position sensor signal.

The peristaltic pump may be configured to pump in a plurality of cycles, where each cycle includes a first stage, a second stage, a third stage, and a fourth stage; where: in the first stage, the inlet valve is opened and the plunger is actuated away from the tube, in the second stage, the inlet valve is closed, the plunger is moved toward the tube by the spring, and the actuator is mechanically disengaged from the plunger, the pump is configured to determine a first position of the plunger, in the third stage, the outlet valve is opened and the actuator allows the plunger to move toward the tube via a force from the spring bias to discharge fluid downstream passed the outlet valve, in the fourth stage, the outlet valve is closed prior to a full discharge of fluid by the plunger, the actuator is mechanically disengaged from the plunger, the pump may be configured to determine a second positon of the plunger, and a processor may be configured to determine an amount of fluid discharged downstream thereby adjusting for air adjacent to the plunger in the second stage. The first stage may be a fluid-fill stage, and/or the pump may be configured to prevent the plunger from engaging a mechanical stop in the fourth stage.

In an embodiment, a system for pumping fluid that may include a first tube configured to couple to a secondary source of fluid. The system also includes a second tube configured to couple to a primary source of fluid. The system also includes a check valve fluidly coupled to the second tube, the check valve configured to allow fluid flow away from the primary source of fluid. The system also includes a three-port connector having first, second, and third connections, where the first connection may be fluidly coupled to the first tube and the second connection may be fluidly coupled to the second tube with the check valve being between the three-port connector and the primary source of fluid. The system also includes a third tube fluidly coupled to the third connection, the third tube having a section of tubing configured for being actuated upon; and a peristaltic pump having a plunger, a processor, and a graphic user interface, the peristaltic pump configured to actuate the plunger against the section of tube of the third tube, the peristaltic pump having an inlet valve, where during a fluid fill stage, the inlet valve is open and the plunger is actuated away from a the tube, where the graphical user interface is configured to set the peristaltic pump to a secondary-infusion mode, where when in the secondary-infusion mode, the processor may be configured to apply an actuation limit to the plunger in the fluid fill stage to thereby mitigate sympathetic flow from the check valve.

In an embodiment, the actuation limit may be configured to keep an upstream fluid pressure above a predetermined threshold, which may be above a crack pressure of the check value. For example, the crack pressure may be between 1.5 to 5 pounds-per-square inch. The actuation limit is a plunger speed limit of the plunger. The actuation limit is a rotation limit of the cam shaft. The processor may be configured to activate the actuation limit when a flow rate of the peristaltic pump is greater than 250 milliliters per hour. The processor may be configured to limit a flow rate of the peristaltic pump to 500 milliliters or less when the peristaltic pump is set to the secondary-infusion mode. The actuation limit is configured to limit the sympathetic flow to less than 5% of a volume to be infusion. The volume to be infused may be between 50 milliliters to 1000 milliliters. A flow rate of the peristaltic pump may be configured to be 500 milliliters per hour or less when the peristaltic pump is in the secondary-infusion mode. Embodiments of the described techniques may include hardware, a method or process, or computer software on a computer-accessible medium.

An embodiment may include a system for pumping fluid. The system also includes a peristaltic pump having a pump body, a plunger, an inlet valve, an outlet valve, an ultrasonic air sensor, and a platen, where the plunger is configured to actuate toward and away from the platen, where the peristaltic pump includes a door, where the door and the pump body form a retainer well upstream from the ultrasonic air sensor. The system also includes a first administration tubing section. The system also includes a second administration tubing section. The system also includes a tube section configured for being actuated upon, the tube section being fluidly coupled to the first administration tubing section at a first end and the tube section being fluidly coupled to the second administration tubing section at a second end thereby forming a junction. The system may also include a retainer disposed on the junction and configured for being secured within the retainer well when the door is closed against the pump body. The junction may be solvent bonded. The retainer well may be disposed downstream from the outlet valve. The retainer well may be disposed downstream from the plunger. The retainer well may be disposed downstream from the inlet valve. The retainer well may be disposed downstream from the plunger and upstream from the outlet valve. The retainer may include a key and the retainer well includes a key well configured to receive the key.

In an embodiment, a system for pumping fluid may include a tube configured to move fluid; a peristaltic pump having a plunger, an inlet valve, an outlet valve, a platen, an actuator, and a spring, the actuator configured to actuate the plunger, the plunger configured for actuation toward and away from the platen, the spring configured to bias the plunger toward the platen, the tube configured for being positioned within the platen, the inlet valve disposed adjacent to the platen and upstream of the plunger, and an outlet valve disposed adjacent to the platen and downstream of the plunger, where the peristaltic pump is configured to pump in a plurality of cycles, where each cycle includes a first stage, a second stage, and a third stage. The system also includes a check valve operatively coupled to the tube, the check valve having a crack pressure, where the check valve is disposed in spaced relation to the peristaltic pump to be downstream of the peristaltic pump when the tube is positioned within the platen. The system also includes where: in the first stage, the inlet valve is opened and the plunger is actuated away from the tube thereby providing a nadir pressure within the tube, in the second stage, the inlet valve is closed, the plunger is moved toward the tube by the spring, and the actuator is mechanically disengaged from the plunger, in the third stage, the outlet valve is opened and the actuator allows the plunger to move toward the tube under the bias of the spring to discharge fluid downstream passed the outlet valve, and the crack pressure is configured to raise the nadir pressure above a predetermined threshold, where said predetermined pressure is above an outgassing pressure.

The outgassing pressure may be a function of an expected temperature range of the fluid. In the first stage, the peristaltic pump is configured to control the plunger actuation away from the tube to thereby keep the nadir pressure above the outgas pressure. The crack pressure may be adjustable. The pump is configured to control the crack pressure to adjust the predetermined pressure to be above a calculated outgas sing pressure that corresponds to the outgassing pressure. The calculated outgassing pressure is based on a measured temperature of the fluid. The calculated outgassing pressure is based on a measured pressure of the fluid. The calculated outgassing pressure is based on a measured ambient pressure. The calculated outgassing pressure is based on an expected amount of dissolved gas. The calculated outgassing pressure is calculated using an ideal gas law.

The foregoing are merely illustrative embodiments of the present disclosure. These and other aspects of the present disclosure are more fully described herein with reference to the accompanying drawings. Other embodiments may include corresponding computer systems, apparatus, and computer programs recorded on one or more computer storage devices, each configured to perform the actions of the methods.

### BRIEF DESCRIPTION OF THE DRAWINGS

These and other aspects will become more apparent from the following detailed description of the various embodiments of the present disclosure with reference to the drawings wherein:

FIG. 1 shows a front perspective view of a peristaltic pump in accordance with an embodiment of the present disclosure;

FIG. 2 shows the peristaltic pump of FIG. 1 with the door open and the lever in the open position in accordance with an embodiment of the present disclosure;

FIG. 2A shows the peristaltic pump of FIG. 1A with the door open and the lever in the open position in accordance with an embodiment of the present disclosure;

FIG. 3 shows a close up view of the opened door of the peristaltic pump of FIG. 1 in accordance with an embodiment of the present disclosure;

FIG. 4 shows the peristaltic pump of FIG. 1 with the door open and a flow stop loaded into the carriage of the peristaltic pump in accordance with an embodiment of the present disclosure;

FIG. 5 shows the peristaltic pump of FIG. 1 after the flow stop has been loaded into the carriage and the door has been shut, but prior to closing the lever, in accordance with an embodiment of the present disclosure;

FIG. 6 shows the back of the pump of FIG. 1 with the back housing, cabling and electronic circuit boards, removed in accordance with an embodiment of the present disclosure;

FIG. 7 shows the pump as shown in FIG. 6, but with the motor removed in accordance with an embodiment of the present disclosure;

FIG. 8 shows the pump as shown in FIG. 7 but at another angle in accordance with an embodiment of the present disclosure;

FIG. 9 shows the pump as shown in FIG. 7, but at a bottom-up angle from the back of the pump, in accordance with an embodiment of the present disclosure;

FIG. 10 shows a front view of a mechanical assembly including the shaft coupled to the lever of the pump of FIG. 1 with the lever in the open position in accordance with an embodiment of the present disclosure;

FIG. 11 shows the mechanical assembly of FIG. 10 with the lever in the closed position in accordance with an embodiment of the present disclosure;

FIG. 12 shows the back view of the mechanical assembly of FIG. 10 with the lever in the open position in accordance with an embodiment of the present disclosure;

FIG. 13 shows the back view of the mechanical assembly of FIG. 10 with the lever in the closed position in accordance with an embodiment of the present disclosure;

FIG. 14 is a cross-sectional view of the peristaltic pump of FIG. 1 showing the lift cam when the lever is in the closed position in accordance with an embodiment of the present disclosure;

FIG. 15 is a cross-sectional view of the peristaltic pump of FIG. 1 showing the lift cam when the lever is in between the closed position and the open position in accordance with an embodiment of the present disclosure;

FIG. 16 is a cross-sectional view of the peristaltic pump of FIG. 1 showing the lift cam when the lever is in the open position in accordance with an embodiment of the present disclosure;

FIG. 17 shows a close-up view of the latching sled of the mechanical assembly of the peristaltic pump of FIG. 1 when the lever is in the closed position in accordance with an embodiment of the present disclosure;

FIG. 18 shows a close-up view of the latching sled of the mechanical assembly of the peristaltic pump of FIG. 1 when the lever is between the closed position and the open position in accordance with an embodiment of the present disclosure;

FIG. 19 shows a close-up view of the latching sled of the mechanical assembly of the peristaltic pump of FIG. 1 when the lever is in the open position in accordance with an embodiment of the present disclosure;

FIG. 20 shows the door catch and latching sled of the peristaltic pump of FIG. 1 from the front side of the pump in accordance with an embodiment of the present disclosure;

FIG. 21 shows the latching sled of the peristaltic pump in accordance with an embodiment of the present disclosure;

FIG. 22 shows the door catch and latching sled of the peristaltic pump of FIG. 1 from the back side of the pump, the claw of the latching sled is in a locking position in accordance with an embodiment of the present disclosure;

FIG. 23 shows the door catch and latching sled of the peristaltic pump of FIG. 1 from the back side of the pump, the claw of the latching sled is in a retracted position in accordance with an embodiment of the present disclosure;

FIG. 24 shows the door catch and a portion of the block that seats the latching sled for the peristaltic pump of FIG. 1 in accordance with an embodiment of the present disclosure;

FIG. 25 shows the door catch for the peristaltic pump of FIG. 1 in accordance with an embodiment of the present disclosure;

FIG. 26 shows a cross-sectional view of the peristaltic pump of FIG. 1 with a hook cam in a non-hooking position in accordance with an embodiment of the present disclosure;

FIG. 27 shows the cross-sectional view of FIG. 26A, but with the hook cam partially actuated toward the cam follower of the latching sled in accordance with an embodiment of the present disclosure;

FIG. 28 shows the cross-sectional view of FIG. 26A, but with the hook cam fully actuated such that the hook has coupled to the cam follower of the latching sled and has fully retracted the latching sled in accordance with an embodiment of the present disclosure;

FIG. 29 shows the hook cam of the peristaltic pump of FIG. 1 in accordance with an embodiment of the present disclosure;

FIG. 30 shows an exploded view of a coupling for coupling together the main shaft to the upper shaft of the peristaltic pump of FIG. 1 in accordance with an embodiment of the present disclosure;

FIG. 31 shows an exploded view of the coupling of FIG. 30 but from another viewing angle in accordance with an embodiment of the present disclosure;

FIG. 32 shows a cross-sectional view of the peristaltic pump of FIG. 1 to illustrate the gears to actuate a carriage by actuation of the main shaft with the door open and the lifter pin actuated toward the open door in accordance with an embodiment of the present disclosure;

FIG. 33 shows the same cross-sectional view as in FIG. 32 but with the door closed which thereby actuates the lifter pin away from the door to compress the spring which actuates the lift in accordance with an embodiment of the present disclosure;

FIG. 34 shows a cross-sectional view of the peristaltic pump of FIG. 1 to show a cross-sectional view of the carriage assembly with the door open and the lever open in accordance with an embodiment of the present disclosure;

FIG. 35 shows the same cross-sectional view as in FIG. 34 but the door is closed which actuates the pawl in accordance with an embodiment of the present disclosure;

FIG. 36 shows the same cross-sectional view as in FIG. 35 but with the carriage in a rotated position which is caused by closure of the lever in accordance with an embodiment of the present disclosure;

FIG. 37 shows the carriage assembly of the peristaltic pump of FIG. 1 from a bottom side of the carriage in accordance with an embodiment of the present disclosure;

FIG. 38 shows the carriage assembly of the peristaltic pump of FIG. 1 from a top side of the carriage in accordance with an embodiment of the present disclosure;

FIG. 39 shows the carriage assembly of the peristaltic pump of FIG. 1 from a bottom side of the carriage assembly with the bottom portion of the carriage housing removed for clarity in accordance with an embodiment of the present disclosure;

FIGS. 40 and 41 show views of the carriage of the peristaltic pump of FIG. 1 in accordance with an embodiment of the present disclosure;

FIG. 42 shows the carriage of the peristaltic pump of FIG. 1 with the top portion removed in accordance with an embodiment of the present disclosure;

FIGS. 43-48 show several views of the flow stop that can be inserted into the carriage of the peristaltic pump of FIG. 1 in accordance with an embodiment of the present disclosure;

FIGS. 49-53 show a sequence of event to illustrate the flow stop of FIGS. 43-48 being inserted in the carriage assembly of the peristaltic pump of FIG. 1 in accordance with an embodiment of the present disclosure;

FIG. 54 shows the carriage assembly from the top side with a sensor board coupled thereto of the peristaltic pump of FIG. 1 in accordance with an embodiment of the present disclosure;

FIG. 55 shows the same view as FIG. 54 but with the sensor board shown as being transparent to show LEDs and the corresponding flow-stop ID sensor in accordance with an embodiment of the present disclosure;

FIG. 56 shows the carriage assembly from an angled bottom view to more clearly see the LEDs of the flow-stop ID sensor and a light pipe for the LEDs in accordance with an embodiment to the present disclosure;

FIG. 57 shows the light pipe used in the carriage assembly of the peristaltic pump of FIG. 1 in accordance with an embodiment of the present disclosure;

FIG. 58 shows a flow-chart diagram to illustrate a method of using the peristaltic pump of FIG. 1 in accordance with an embodiment of the present disclosure;

FIG. 59 shows a circuit of the peristaltic pump of FIG. 1 for driving the LEDs of the flow-stop ID sensor in accordance with an embodiment of the present disclosure;

FIG. 60 shows a circuit of the peristaltic pump of FIG. 1 showing the arrangement of the LEDs of the flow-stop ID sensor in accordance with an embodiment of the present disclosure;

FIG. 61 shows a circuit of the peristaltic pump of FIG. 1 for sensing light received after light from the LEDs has passed through the flow-stop ID holes of the extension of the flow stop in accordance with an embodiment of the present disclosure;

FIG. 62 shows a flow chart diagram illustrating a method of using data from the light sensor shown in FIG. 61 to identify a flow stop in accordance with an embodiment of the present disclosure;

FIG. 63 shows an alternative embodiment of the peristaltic pump of FIG. 1 where an alternative lift cam, an alternative mechanical linkage between the shaft and carriage, and an alternative door catch are used in accordance with an embodiment of the present disclosure;

FIG. 64 shows another view of the peristaltic pump of FIG. 63 to illustrate the operation of the lift cam in accordance with an embodiment of the present disclosure;

FIG. 65 shows a cross-sectional view of the lift cam of the peristaltic pump of FIG. 63 when the lever is in the open position accordance with an embodiment of the present disclosure;

FIGS. 66-72 show the lift cam of the peristaltic pump of FIG. 63 from various viewing angles in accordance with an embodiment of the present disclosure;

FIG. 73 shows the peristaltic pump of FIG. 63 from a back view to show a linkage bar between the door catch and a linear ratchet in accordance with an embodiment of the present disclosure;

FIG. 74 shows the peristaltic pump of FIG. 63 to provide another view of the linkage bar between the door catch and a linear ratchet in accordance with an embodiment of the present disclosure;

FIG. 75 shows a close-up view of the interface of the over-center spring and the door catch with the linkage bar of the peristaltic pump of FIG. 63 with door-catch in the door open position and the lever open;

FIG. 76 shows the same close-up view of FIG. 75 but with the door catch in the door shut position in accordance with an embodiment of the present disclosure;

FIG. 77 shows the same close-up view of FIG. 75 but with the door catch in the door shut position and the lever in the closed position in accordance with an embodiment of the present disclosure;

FIG. 78-84 show several views of the door catch of the peristaltic pump of FIG. 63 in accordance with an embodiment of the present disclosure;

FIG. 85 shows a close-up view of the linear ratchet when the door is open and the lever is open in accordance with an embodiment of the present disclosure;

FIG. 86 shows a close-up view of the linear ratchet when the door is closed and the lever is open in accordance with an embodiment of the present disclosure;

FIG. 87 shows a close-up view of the linear ratchet when the door is closed and the lever is closed in accordance with an embodiment of the present disclosure;

FIGS. 88-89 show the peristaltic pump of FIG. 63 with some parts removed to illustrate the mechanical linkage between the shaft and the carriage where the door-catch, the door, and the lever are in the open position in accordance with an embodiment of the present disclosure;

FIGS. 90-91 show the peristaltic pump of FIG. 63 with some parts removed to illustrate the mechanical linkage between the shaft and the carriage where the door and the door catch are in the closed position and the lever is in open position in accordance with an embodiment of the present disclosure;

FIG. 92 shows the peristaltic pump of FIG. 63 with some parts removed to illustrate the mechanical linkage between the shaft and the carriage where the door and the door catch are in the closed position while the lever is between the open and closed position in accordance with an embodiment of the present disclosure;

FIG. 93 shows the peristaltic pump of FIG. 63 with some parts removed to illustrate the mechanical linkage between the shaft and the carriage where the door, the door catch, and the lever are in the closed position in accordance with an embodiment of the present disclosure;

FIGS. 94-96 show the pawl of the peristaltic pump of FIG. 63 from several views in accordance with an embodiment of the present disclosure;

FIGS. 97-98 show the peristaltic pump of FIG. 63 with some parts removed to illustrate the mechanical linkage between the shaft and the carriage where the door and the door catch are in the closed position and the lever is in open position in accordance with an embodiment of the present disclosure;

FIGS. 99-101 show portions of the alternative mechanical assembly of the peristaltic pump of FIGS. 97-98 in accordance with an embodiment of the present disclosure;

FIGS. 102-105 show several views a flow-stop assembly in accordance with an embodiment of the present disclosure;

FIG. 106 shows a cross-sectional view of the flow-stop assembly of FIGS. 102-105 in accordance with an embodiment of the present disclosure;

FIGS. 107, 108A, and 108B show several views of the flow-stop assembly of FIGS. 102-105 with the top housing removed in accordance with an embodiment of the present disclosure;

FIGS. 109A-109B show an alternative embodiment of the flow-stop assembly of FIGS. 108A-108B with knife-edge pivots in accordance with an embodiment of the present disclosure;

FIGS. 110-114 show several views of the bottom housing of the flow-stop assembly of FIGS. 102-105 in accordance with an embodiment of the present disclosure;

FIGS. 115-119 show several views of the top housing of the flow-stop assembly of FIGS. 102-105 in accordance with an embodiment of the present disclosure;

FIGS. 120-124 show several views of a first link of the flow-stop assembly of FIGS. 102-105 having a plunger in according with an embodiment of the present disclosure;

FIGS. 125-129 show several views of a second link of the flow-stop assembly of FIGS. 102-105 in accordance with an embodiment of the present disclosure;

FIGS. 130-133 show several views of a tube coupling of the flow-stop assembly of FIGS. 102-105 in accordance with an embodiment of the present disclosure;

FIGS. 134-138 show the flow-stop assembly of FIGS. 102-105 being inserted into a carriage, in accordance with an embodiment of the present disclosure;

FIG. 139 shows a perspective view of the internal mechanism of the carriage when the end effector is engaged with a flange of the flow-stop assembly of FIGS. 102-105 in accordance with an embodiment of the present disclosure;

FIG. 140 shows a perspective view of the internal mechanism of the carriage when the end effector is engaged with a flange of the flow-stop assembly of FIGS. 102-105 in accordance with an embodiment of the present disclosure;

FIG. 141 shows the front of the carriage orifice with a cooperating surface in accordance with an embodiment of the present disclosure;

FIG. 142 shows the front of the carriage orifice with a cooperating surface when the flow-stop assembly has been inserted and a tube shutter retracted in accordance with an embodiment of the present disclosure;

FIGS. 143-146 show several views a flow-stop assembly in accordance with an embodiment of the present disclosure;

FIG. 147 shows a cross-sectional view of the flow-stop assembly of FIGS. 143-146 in accordance with an embodiment of the present disclosure;

FIGS. 148-150 show several views of the flow-stop assembly of FIGS. 143-146 with the top housing removed in accordance with an embodiment of the present disclosure;

FIGS. 151-155 show several views of the top housing of the flow-stop assembly of FIGS. 143-146 in accordance with an embodiment of the present disclosure;

FIGS. 156-160 show several views of the bottom housing of the flow-stop assembly of FIGS. 143-146 in accordance with an embodiment of the present disclosure;

FIGS. 161-165 show several views of a first link of the flow-stop assembly of FIGS. 143-146 having a plunger in according with an embodiment of the present disclosure;

FIGS. 166-170 show several views of a second link of the flow-stop assembly of FIGS. 143-146 in accordance with an embodiment of the present disclosure;

FIGS. 171-174 show several views of a pinching flow-stop assembly having a flow stop with an arcuate slot in accordance with an embodiment of the present disclosure;

FIGS. 175-178 show several views of the flow stop of the pinching flow-stop assembly of FIGS. 171-174 in accordance with an embodiment of the present disclosure;

FIGS. 179-181 show several views of the housing of the pinching flow-stop assembly of FIGS. 171-174 in accordance with an embodiment of the present disclosure;

FIGS. 182-184 show the pinching flow-stop assembly of FIGS. 171-174 being inserted into a carriage in accordance with an embodiment of the present disclosure;

FIG. 185 shows a perspective view of the internal mechanism of the carriage when the end effector is engaged with a flange of the pinching flow-stop assembly of FIGS. 171-174 in accordance with an embodiment of the present disclosure;

FIG. 186 shows a perspective view of the internal mechanism of the carriage when the end effector is engaged with a flange of the pinching flow-stop assembly of FIGS. 171-174 in accordance with an embodiment of the present disclosure;

FIG. 187 shows a block diagram of a modular pump system having a central unit and a plurality of medical device assemblies coupled together in accordance with an embodiment of the present disclosure;

FIG. 188 shows a block diagram of a modular pump system to illustrate the power circuitry of the system in accordance with an embodiment of the present disclosure;

FIG. 189 shows a state diagram of the central unit power circuitry in accordance with an embodiment of the present disclosure;

FIG. 190 shows a state diagram of the medical device assembly power circuitry in accordance with an embodiment of the present disclosure;

FIGS. 191A-191B show a timing diagram of the modular pump system as two medical device assemblies are coupled to the central unit to illustrate the powering-up sequence of the system in accordance with an embodiment of the present disclosure

FIGS. 192A-192C show a block diagram of a modular pump system in accordance with an embodiment of the present disclosure;

FIGS. 193A-193J show a circuit of the modular pump system in accordance with an embodiment of the present disclosure;

FIG. 194 shows a block diagram of the communication circuitry of the modular pump system in accordance with an embodiment of the present disclosure;

FIG. 195 shows a diagram of the circuitry for interfacing into the communications bus of the modular pump system in accordance with an embodiment of the present disclosure;

FIG. 196 shows an antenna design to couple a module to another module to extend the communications bus of the modular pump system in accordance with an embodiment of the present disclosure;

FIG. 197 shows another embodiment of a peristaltic pump having a relief mechanism in accordance with an embodiment of the present disclosure;

FIGS. 198A-198C show an illustration of the transition from a hold state to a triggered state of the relief mechanism shown in FIG. 197 in accordance with an embodiment of the present disclosure;

FIGS. 199A-199C show the first rigid member of the relief mechanism shown in FIG. 197 in accordance with an embodiment of the present disclosure;

FIGS. 200A-200C show the second rigid member of the relief mechanism shown in FIG. 197 in accordance with an embodiment of the present disclosure;

FIG. 201 shows a keyed end effector that is part of a plunger in accordance with an embodiment of the present disclosure;

FIGS. 202A-202D show another embodiment of an adjustable end effector of a plunger in accordance with an embodiment of the present disclosure;

FIGS. 202C-202D show first and second shaft adjusters that move a pivot shaft along two directions of a pivot shaft in accordance with an embodiment of the present disclosure;

FIGS. 203A-203G show an adjustable platen of a peristaltic pump in accordance with an embodiment of the present disclosure;

FIGS. 204A-204B show a multi-stage, spring-biased plunger of a peristaltic pump in accordance with an embodiment of the present disclosure;

FIG. 205 shows a flow chart diagram of a method for actuating the multi-stage, spring-biased plunger of FIGS. 204A-204B in accordance with an embodiment of the present disclosure;

FIG. 206 shows a back side of a peristaltic pump having a thermal dissipation assembly with a heatsink in accordance with an embodiment of the present disclosure;

FIGS. 207A-207G show several views of the thermal dissipation assembly of FIG. 206 in accordance with an embodiment of the present disclosure;

FIGS. 208A-208B show additional views of the planar-thermal connector of the thermal dissipation assembly of FIGS. 207A-207G in accordance with an embodiment of the present disclosure;

FIGS. 209A-209E show several view of the heat-strap bracket of the planar-thermal connector of the thermal dissipation assembly of FIGS. 207A-207G in accordance with an embodiment of the present disclosure;

FIG. 210A shows another embodiment of a planar thermal connector in accordance with another embodiment of the present disclosure;

FIG. 210B shows an embodiment of a heatsink having a braided-wire to transfer heat from the motor and the powerbar to dissipate heat in accordance with an embodiment of the present disclosure;

FIG. 211A shows a first portion of a thermal dissipation assembly in accordance with an embodiment of the present disclosure;

FIG. 211B shows a second portion of the thermal dissipation assembly of FIG. 211A;

FIG. 211C shows the first portion of the thermal dissipation assembly of FIG. 211A coupled to the second portion of the thermal dissipation assembly of FIG. 211B;

FIG. 211D is a perspective view of a heatstrap bracket of the thermal dissipation assembly of FIG. 211A;

FIG. 211E is a perspective view of a heatstrap lower portion of the thermal dissipation assembly of FIG. 211A;

FIG. 212 shows a flow chart diagram of a method for dislodging bubbles in an IV line in accordance with an embodiment of the present disclosure;

FIG. 213 shows a flow chart diagram of a method 1408 for detecting a bubble in accordance with an embodiment of the present disclosure;

FIGS. 214-216 show several ultrasonic-based bubble sensors in accordance with several embodiments of the present disclosure;

FIGS. 217-219 show a method for estimating air pumped downstream by a peristaltic pump in accordance with an embodiment of the present disclosure;

FIGS. 220A-220C show several views of an in-line pressure sensor in accordance with an embodiment of the present disclosure;

FIGS. 220D-220F show the in-line pressure sensor of FIGS. 202A-202C with a clip in accordance with an embodiment of the present disclosure;

FIG. 221 shows an in-line pressure sensor in accordance with yet another embodiment of the present disclosure;

FIG. 222 shows a downstream bladder in accordance with an embodiment of the present disclosure;

FIG. 223 show a tube with impeders to slightly increase pressure of the fluid flowing through the tube in some embodiments of the present disclosure;

FIG. 224 shows another embodiment of a downstream bladder in accordance with another embodiment of the present disclosure;

FIG. 225 shows a section of tubing that includes nucleation sites and an air trap in accordance with an embodiment of the present disclosure;

FIG. 226 shows a captive screw in accordance with an embodiment of the present disclosure;

FIGS. 227A-227D show a pole clamp in accordance with an embodiment of the present disclosure;

FIG. 228 shows a block diagram that illustrates a system for pumping fluid from a primary IV bag and a secondary IV bag in accordance with an embodiment of the present disclosure;

FIG. 229 shows a block diagram that illustrates a system for pumping fluid in conjunction with a check valve to prevent outgassing in accordance with an embodiment of the present disclosure

FIG. 230A shows a system for pumping fluid having a retainer configure to secure the tube to the pump body in accordance with an embodiment of the present disclosure;

FIG. 230B shows a close up view of the retainer of FIG. 230A in accordance with an embodiment of the present disclosure; and

FIG. 231 shows a flowchart diagram of a method for pumping fluid and adjusting fluid delivery estimates to account for air or bubbles in accordance with an embodiment of the present disclosure.

### DETAILED DESCRIPTION

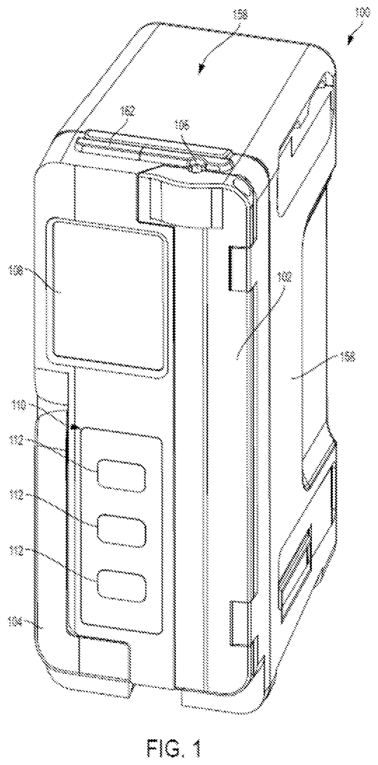

FIG. 1 shows the front of a pump 100. The pump 100 may be a standalone device that couples to an IV pole (not shown) directly, e.g., by using a clamp (not shown). Additionally or alternatively, the pump 100 may be modular such that one or more pumps 100 can be coupled together with a central unit and/or with other medical devices. Although a peristaltic pump 100 is described throughout this specification, additional embodiments may include syringe pumps or other pump types where applicable or where it would be apparent to one of ordinary skill in the relevant art.

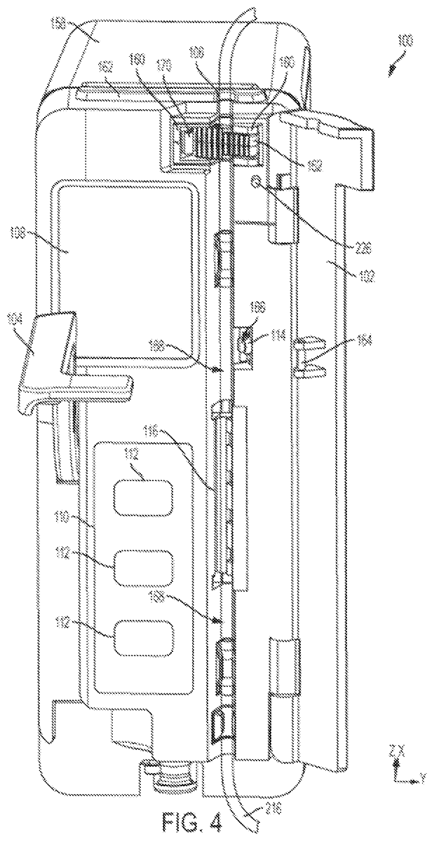

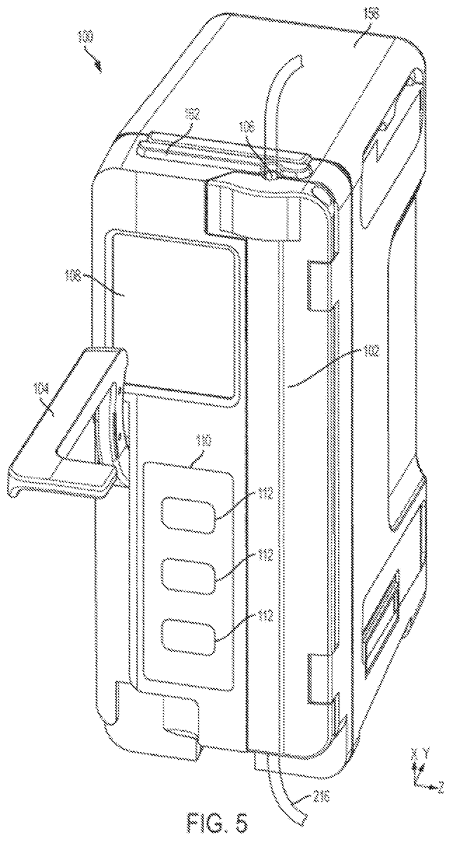

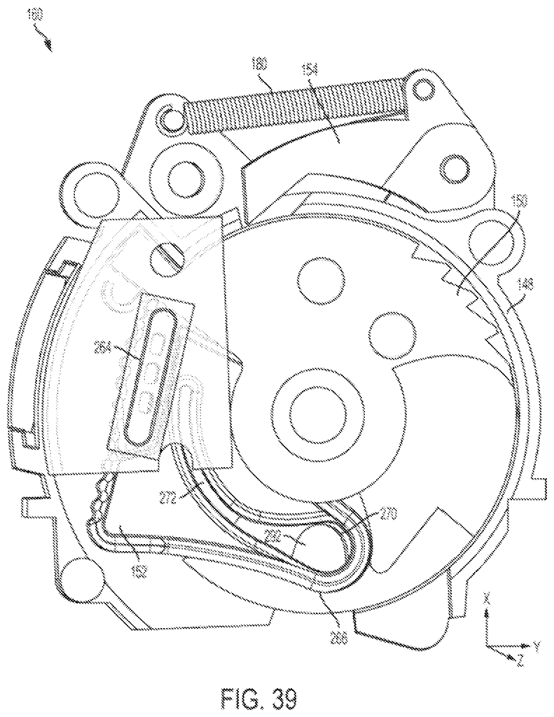

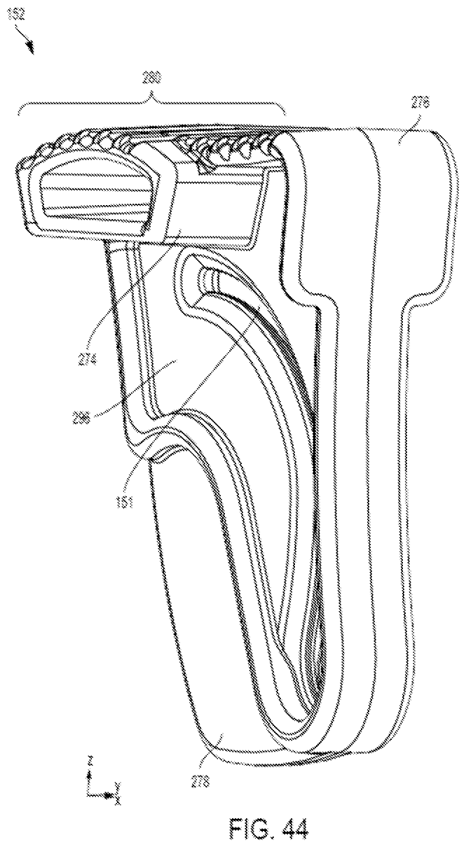

The pump 100 includes a pump housing 158 and a door 102 coupled to the pump housing 158. The door 102 is pivotably coupled to the pump 100 such that an infusion set having a flow stop 152 (see FIGS. 39-44) and a tube 216 (see FIGS. 4-5) may be loaded and secured within the pump 100 by the door 102 (described in more detail below). A hole 106 is shown so that the door 102 may be shut without pinching the tube 216. Kinks or pinches within the tube 216 may occlude fluid flow within the tube 216.

The pump 100 includes a button panel 110 with buttons 112 for user input and a screen 108. The screen 108 provides visual information, such as menus and status information that can be used by a caregiver to program and interact with the control software of the pump 100 using the buttons 112. In some embodiments, the screen 108 may be a touch screen configured to receive user input via user touch. The pump 100 also includes a lever 104 that can be used to open the door 102 and lock the door 102 as described in more detail below.

The pump 100 also includes a light bar 162. The light bar 162 may illuminate based upon the status of the pump 100. For example, the light bar 162 may blink green when the pump 100 is infusing fluid into a patient and blink red when the pump 100 is not operating or is experiencing an error condition or fault. The light bar 162 may blink yellow when an occlusion is detected and intervention is needed to clear the occlusion, etc.

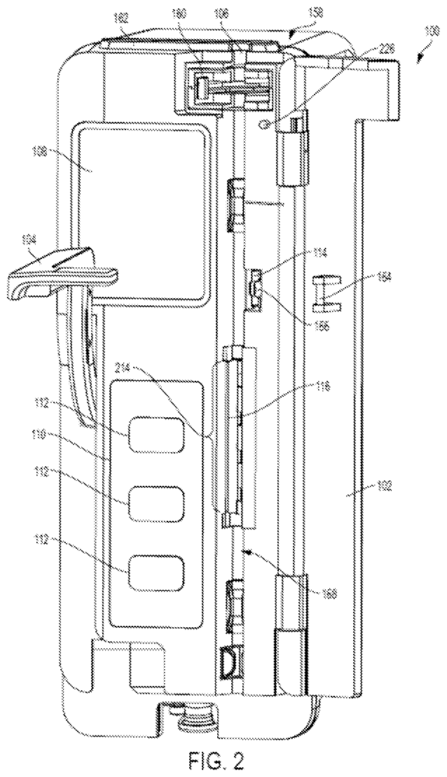

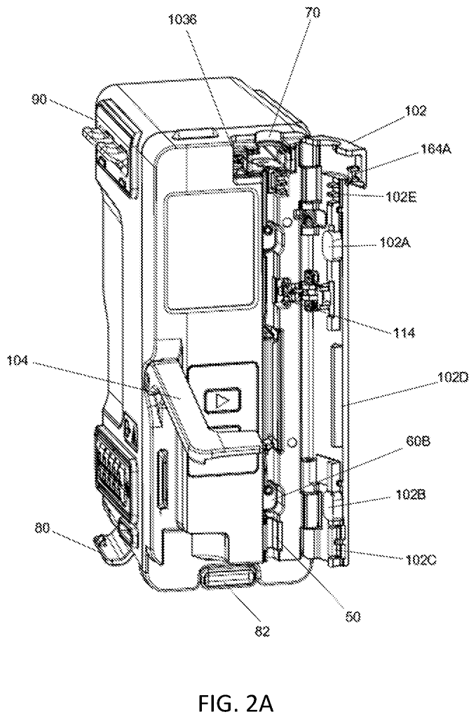

FIG. 2 shows the peristaltic pump 100 of FIG. 1 with the door 102 open and the lever 104 in the open position. When the lever 104 is shut and the door 102 is properly closed, a door catch 114 secures the door 102 shut by holding on to a hold 164. The hold 164 may be a pin that interfaces with a pin catch 166. When the lever 104 is actuated to the open position as shown in FIG. 2, the door catch 114 releases the hold 164. The door 102 may be spring biased such that the door 102 swings open when the door catch 114 releases the hold 164.

Actuation of the lever 104 into the open position also retracts the spring-biased plunger 116. Actuation of the spring-biased plunger 116 allows a tube 216 to be loaded into a platen 168. Having the spring-biased plunger 116 actuated into the platen 168 would make insertion of a tube 216 into the platen 168 more difficult or impossible because it would block the platen 168.

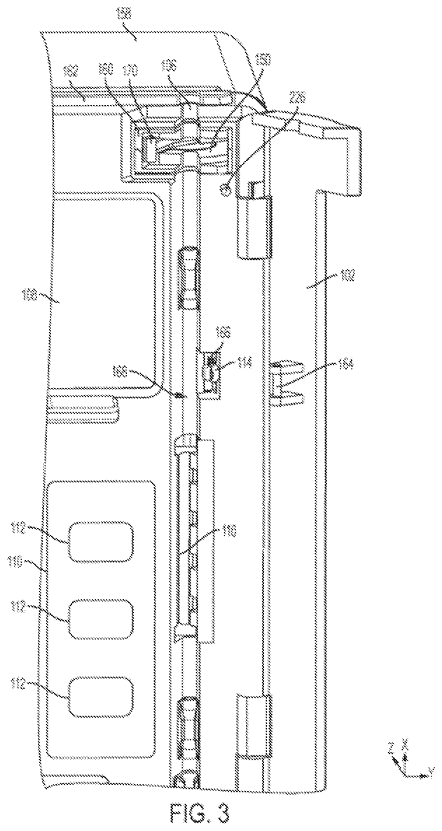

FIG. 3 shows a close up view of the door 102 of the peristaltic pump 100 (see FIG. 1) in the open position. The carriage assembly 160 is also easily seen in FIG. 3. A flow stop 152 (see FIGS. 39-44) may be inserted into the carriage assembly 160 so that a carriage 150 retains the flow stop 152. A flow-stop retainer 170 can retain the flow stop 152 in the carriage 150. FIG. 4 shows the flow stop 152 loaded into the carriage 150 of the peristaltic pump 100. Thereafter, the door 102 may be shut with the flow stop 152 inserted therein as shown in FIG. 5. Because the lever 104 is still in the open position, the door 102 may be reopened because the door catch 114 has not locked the door 102. When the lever 104 is actuated down into the closed position, then the door 102 will be locked by the door catch 114.

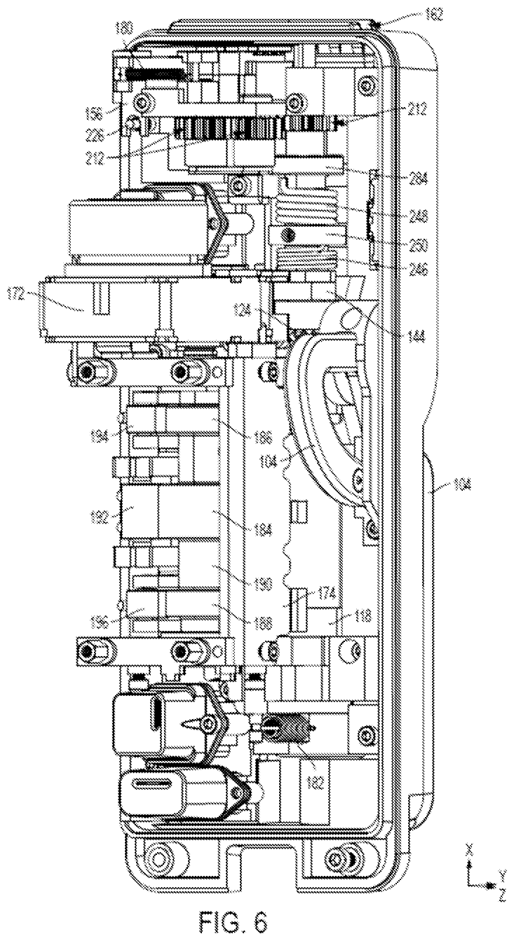

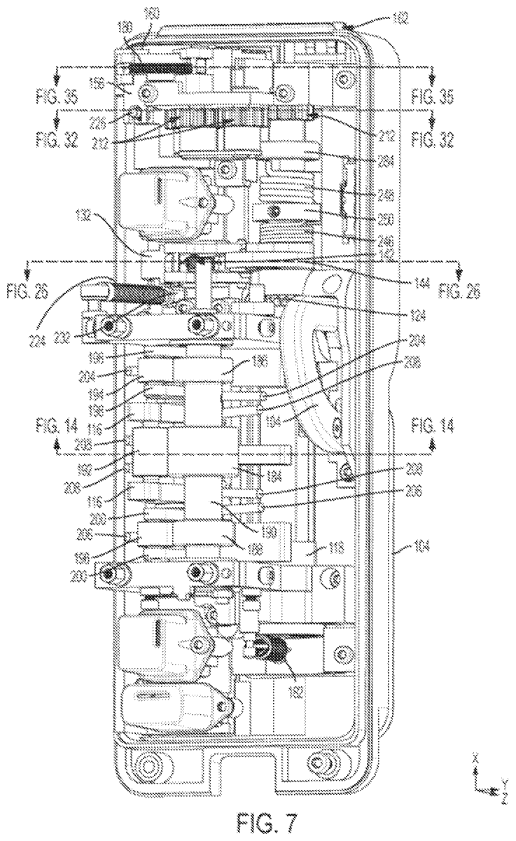

FIG. 6 shows the back of the pump 100 of FIG. 1 with the back housing, cabling and electronic circuit boards, removed. However, in FIG. 6, a motor 172 and a brace 174 are visible. FIG. 7 shows the pump 100 as shown in FIG. 6, but with the motor 172 and the brace 174 removed for additional clarity.

In FIG. 7, a cam shaft 190 is shown with a plunger cam 184, an inlet-valve cam 186, and an outlet-valve cam 188 disposed on the cam shaft 190. A plunger-cam follower 192 pivots along a pivot shaft 202 (see FIG. 14) as the plunger-cam follower 192 follows the plunger cam 184. The inlet valve 198 pivots along the pivot shaft 202 (see FIG. 14) as the inlet-valve cam follower 194 follows the inlet-valve cam follower 194. And, the outlet valve 200 pivot along the pivot shaft 202 (see FIG. 14) as the outlet-valve cam follower 196 follows the outlet-valve cam 188.

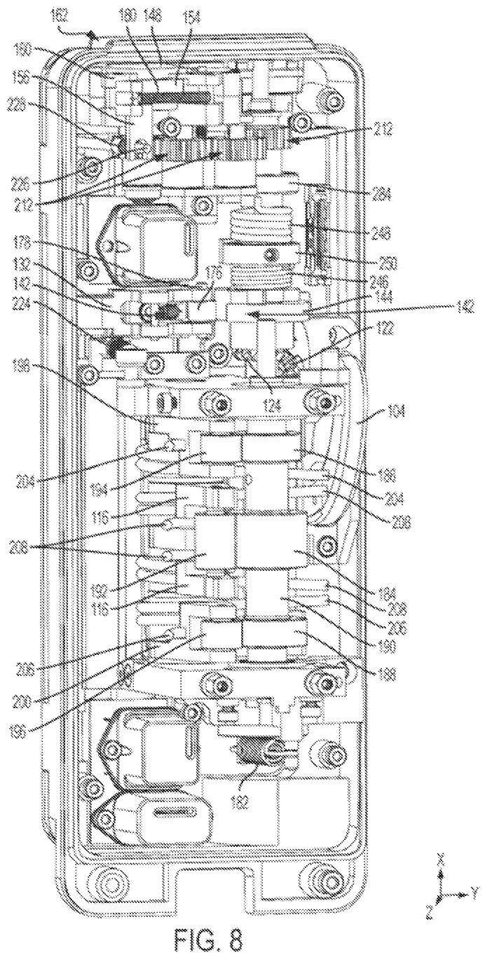

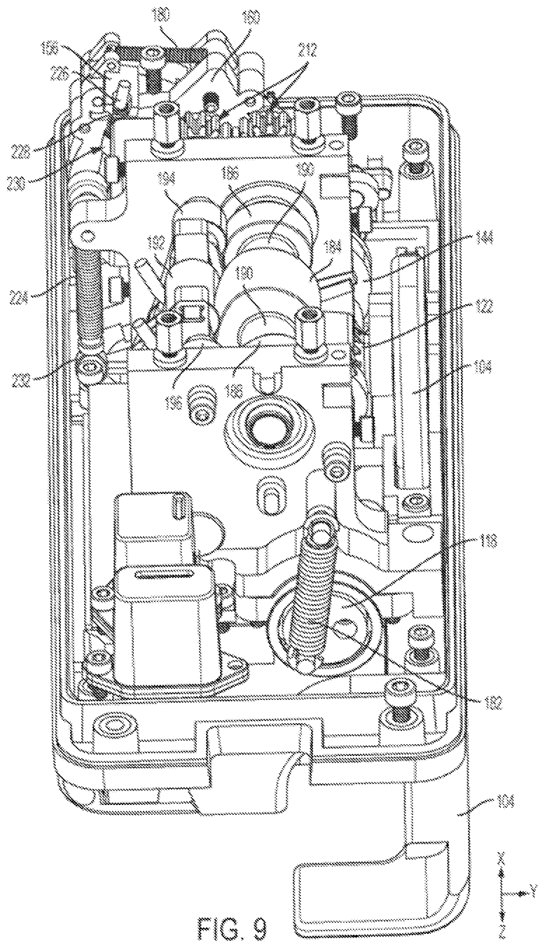

An inlet-valve torsion spring 204 biases the inlet-valve cam follower 194 against the inlet-valve cam 186 and toward the tube 216. An outlet-valve torsion spring 206 biases the outlet-valve cam follower 196 against the outlet-valve cam 188. Also, a pair of plunger torsion springs 208 biases the plunger-cam follower 192 against the plunger cam 184 and therefore also biases the spring-biased plunger 116 toward the tube 216. FIG. 8 shows the pump 100 as shown in FIG. 7 but at another angle, and FIG. 9 shows the pump 100 as shown in FIG. 7, but at a bottom-up angle from the back of the pump 100.

Actuation of the lever 104 actuates the main shaft 118. A shaft spring 182 is shown that pulls the main shaft 118 into one of two positions making the lever 104 actuate toward one of the open or closed position depending upon the angle of the main shaft 118. That is, the shaft spring 182 makes the lever 104 operate with an over-center action with regard to the force the shaft spring 182 exerts on the main shaft 118. The force from the shaft spring 182 exerts on the main shaft 118 is also exerted on the lever 104 because of the mechanical coupling between the main shaft 118 and the lever 104. This over-center action biases the main shaft 118 such that the lever 104 is biased toward either the closed position or the open position, depending upon if the lever 104 is between an intermediate position and the closed position or is between the intermediate position and the open position.

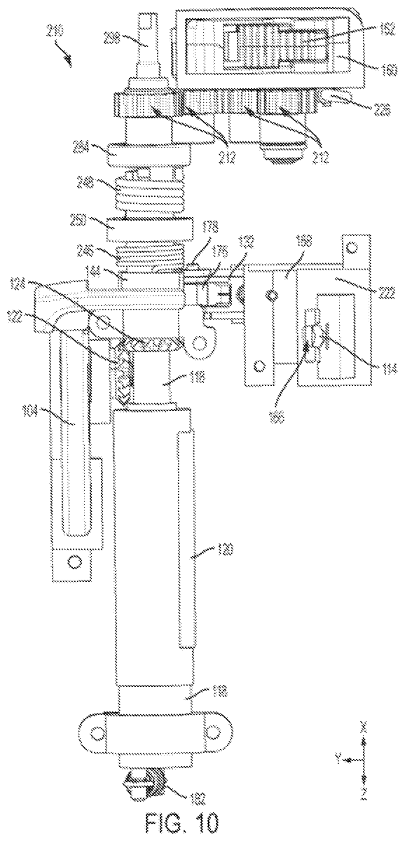

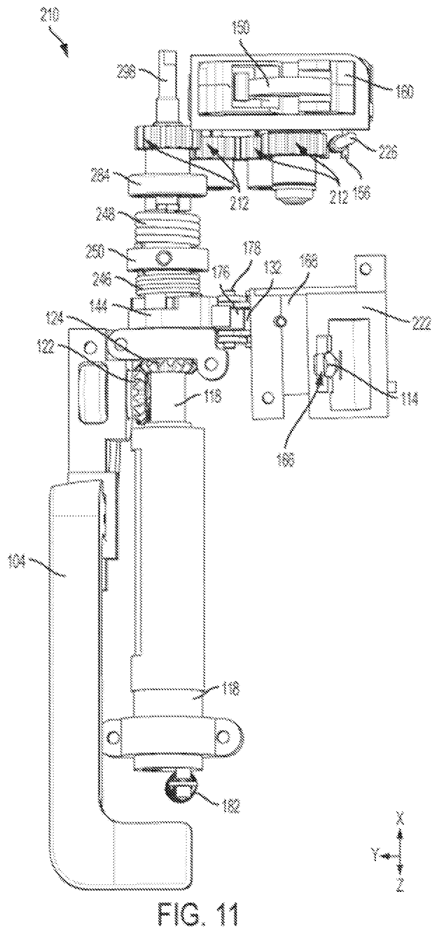

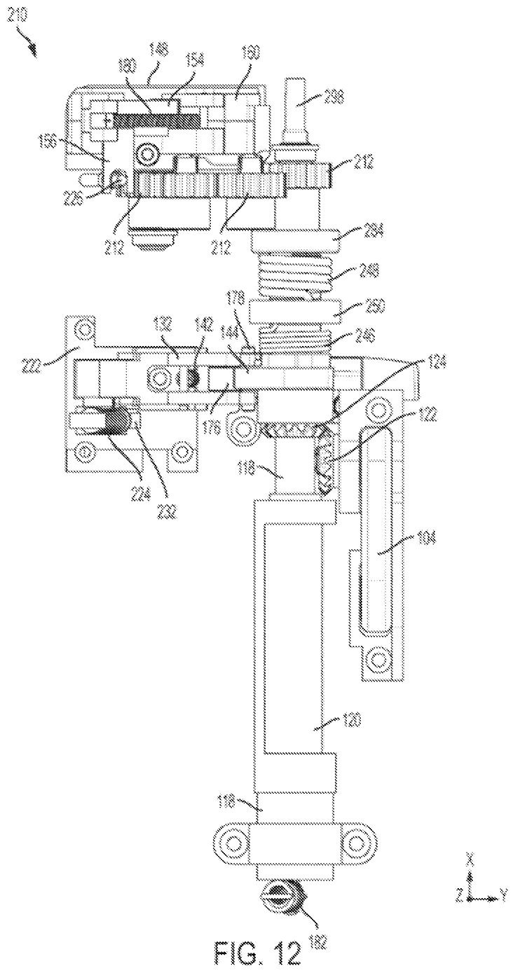

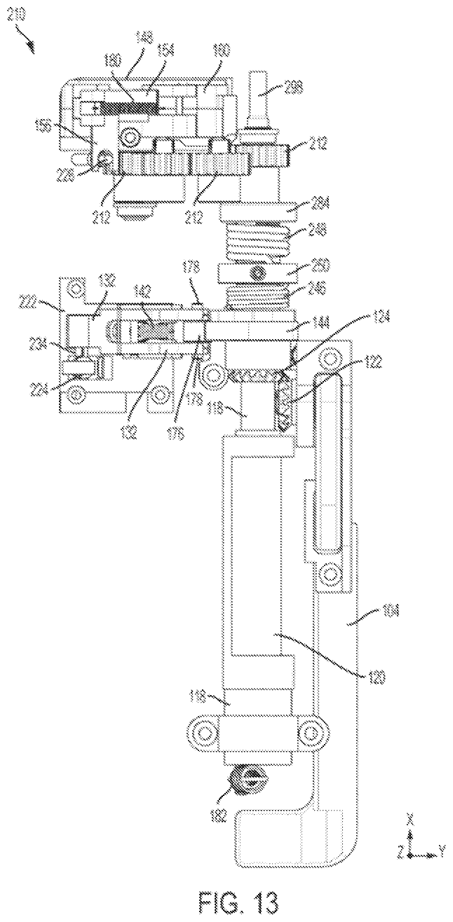

Referring to FIGS. 10-13, FIG. 10 shows a front view of a mechanical assembly 210 including the main shaft 118 coupled to the lever 104 with the lever 104 in the open position, and FIG. 11 shows the mechanical assembly 210 of FIG. 10 with the lever 104 in the closed position. FIG. 12 shows the back view of the mechanical assembly 210 of FIG. 10 with the lever 104 in the open position and FIG. 13 shows the back view of the mechanical assembly 210 of FIG. 10 with the lever 104 in the closed position. The mechanical assembly 210 may be found within the pump 100 of FIG. 1.

The lever 104 is coupled to the first bevel gear 122 and rotates with movement of the lever 104. That is, the lever 104 is coupled to the first bevel gear 122 to actuate the first bevel gear 122. The first bevel gear 122 is coupled to the second bevel gear 124, and the second bevel gear 124 is coupled to the main shaft 118. In combination, actuation of the lever 104 causes the main shaft 118 to rotate around its central axis.

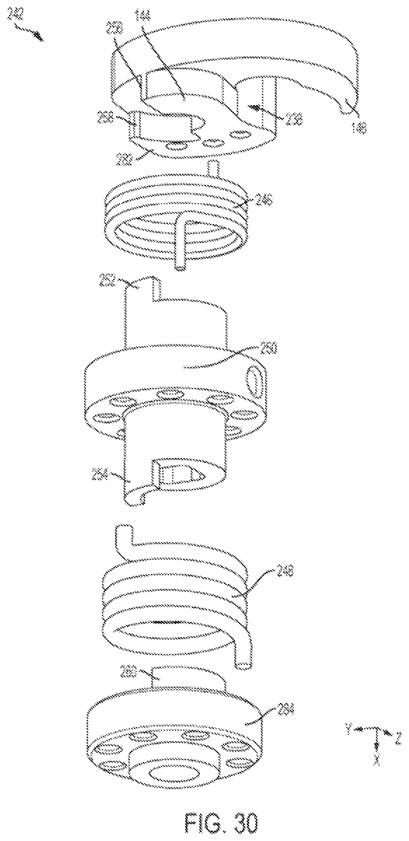

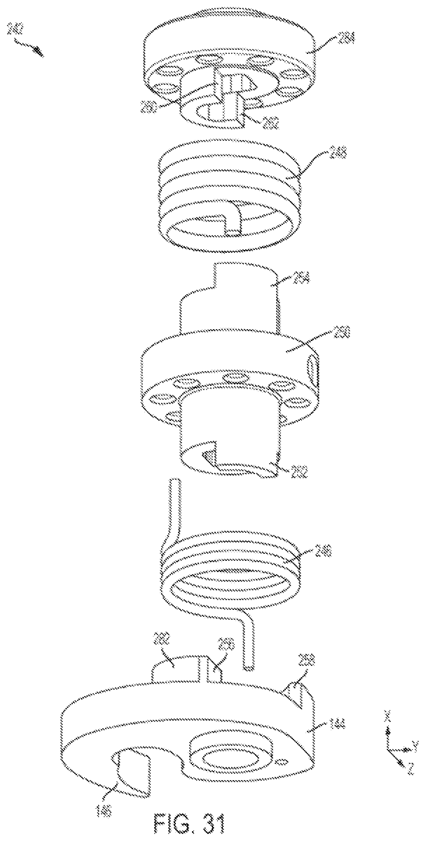

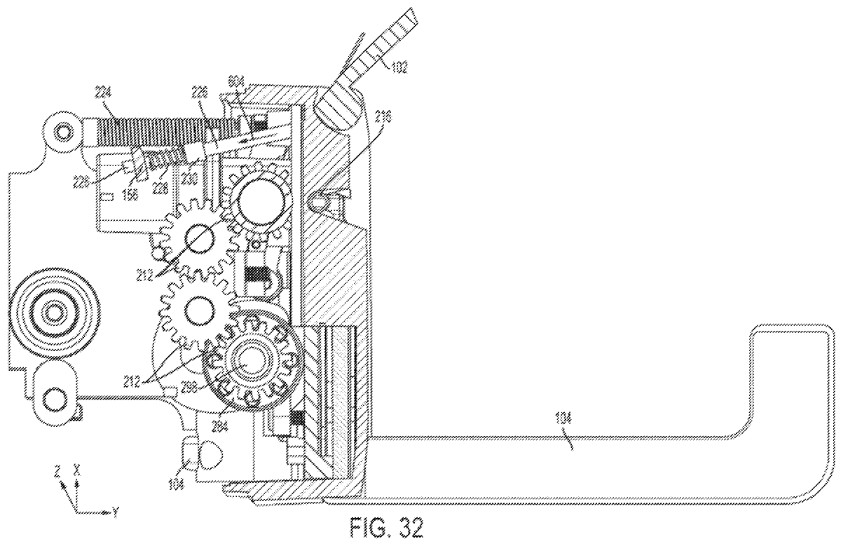

Generally, an upper shaft 298 rotates with the main shaft 118. However, the upper shaft 298 is not directly coupled to main shaft 118 and may, in certain circumstances, rotate separately from the main shaft 118. A more detailed description of the circumstances in which the upper shaft 298 rotates apart from the main shaft 118 is described below with reference to FIGS. 31-32.

Rotation of the main shaft 118 causes a lift cam 120 to rotate. The rotation of the lift cam 120 can actuate the spring-biased plunger 116, the inlet valve 198, and the outlet valve 200 away from a tube 216 and out of the platen 168. That is, the spring-biased plunger 116, the inlet valve 198, and the outlet valve 200 are retracted away from the tube 216 and into the end-effector port 214 (See FIGS. 2-4). Additional details of the lift cam 120 are described below.

Referring again to FIGS. 10-13, when the lever 104 is in the open position, as shown in FIGS. 10 and 12, the latching sled 132 is configured so that the door catch 114 will allow the door 102 (see FIG. 1) to open and shut freely without locking the door 102. However, the door catch 114 is biased toward holding the door 102 or releasing the door 102. When the lever 104 is in the closed position (see FIGS. 11 and 13), the latching sled 132 allows the door 102 (see FIG. 1) to shut by allowing the door catch 114 to receive the hold 164 (see FIG. 4). However, when the lever 104 is in the closed position and the door 102 is shut, the latching sled 132 will lock the door 102 by preventing the door catch 114 from releasing the hold 164 (see FIG. 4) after it is locked by the latching sled 132. Details of the latching sled 132 are described below.

Also shown in FIGS. 10-13, the carriage assembly 160 can been seen. A carriage housing 148 receives a flow stop 152 within the carriage 150 for rotation therein. Gears 212 rotate the carriage 150 as the lever 104 is actuated such that the flow stop 152 can be inserted into the carriage 150 when the lever 104 is in the open position as shown in FIG. 10. After insertion of the flow stop 152, actuation of the lever 104 to the closed position (shown in FIGS. 11 and 13) rotates the carriage 150 and rotates the flow stop 152 to unkink the tube 216 so that fluid may flow through the tube 216. Details of the carriage assembly 160 are described below.

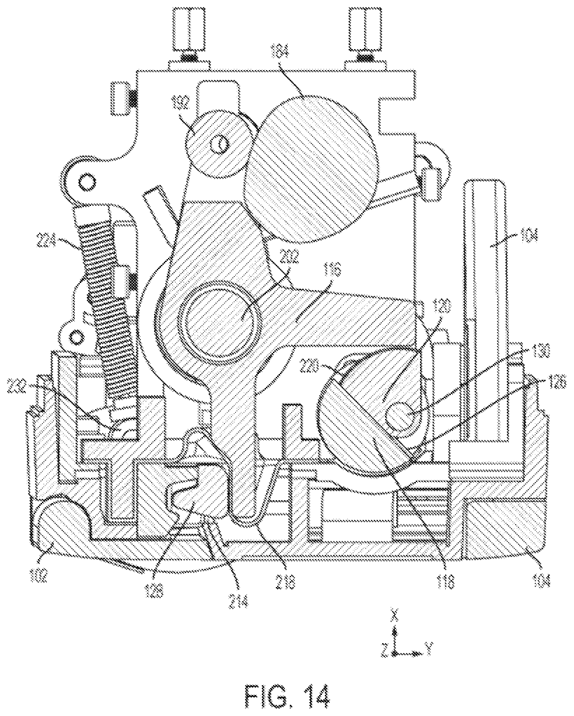

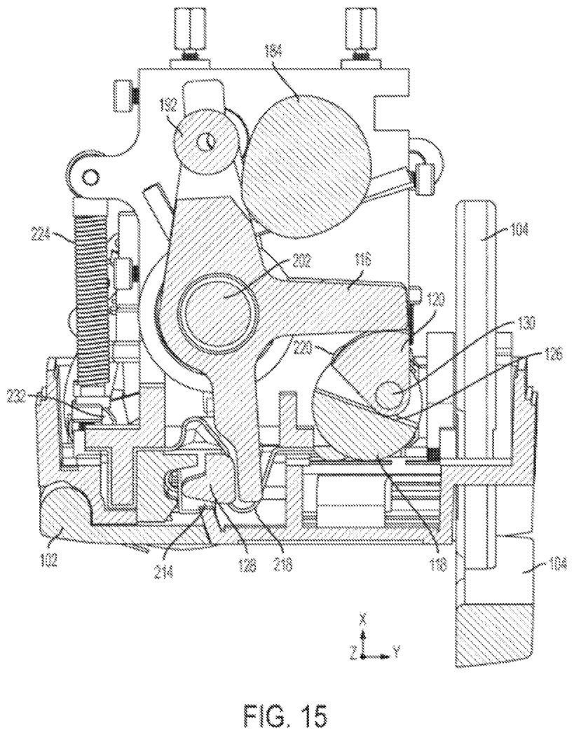

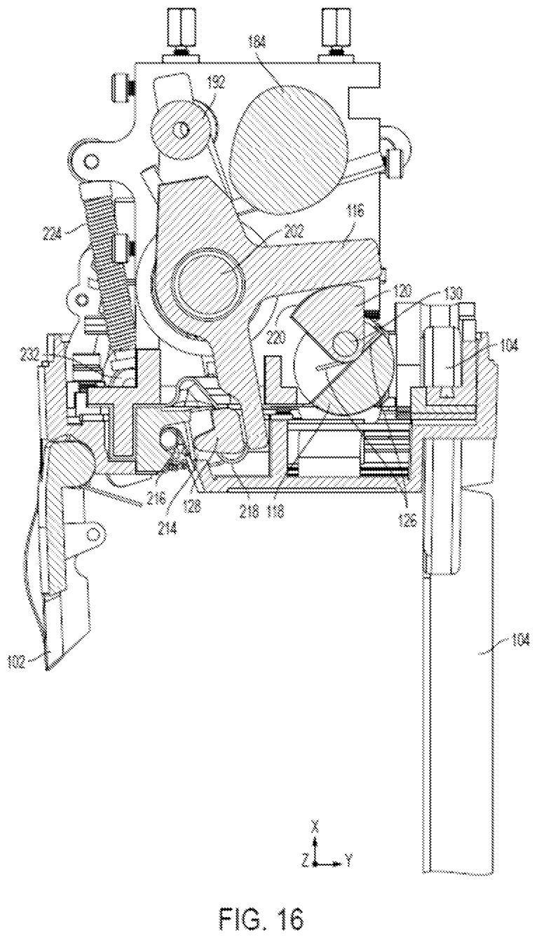

Please refer now to FIGS. 14-16 for reference with the following description of the operation of the lift cam 120. FIGS. 14-16 all show cross-sectional views along the same plane. FIG. 14 is a cross-sectional view of the peristaltic pump 100 showing the lift cam 120 when the lever 104 is in the closed position. FIG. 15 is a cross-sectional view of the peristaltic pump 100 showing the lift cam 120 when the lever 104 is in between the closed position and the open position; And FIG. 16 is a cross-sectional view of the peristaltic pump 100 showing the lift cam 120 when the lever 104 is in the open position.

As shown in FIG. 14, the lift cam 120 is disposed on the main shaft 118 for rotation along a lift-cam pin 130. The axis of the lift-cam pin 130 is offset from a central axis of the main shaft 118. The lift cam 120 is biased by a cam-lifter torsion spring 126 in a counter-clockwise direction as shown in FIG. 14, however, one of ordinary skill in the art would know how to configure the pump 100 for clockwise bias.

In FIG. 14, the lift cam 120 is not engaged with the spring-biased plunger 116 and the position of the spring-biased plunger 116 is based upon the rotational position of the plunger cam 184 and/or the fill volume of a tube 216. The spring-biased plunger 116 includes an end effector 128 that engages with the tube 216 disposed in the platen 168.

The end effector 128 of the spring-biased plunger 116 is shown in FIG. 14 as being in an extended position and thereby protrudes out of the end-effector port 214 (thus engaging with the tube 216). A seal 218 prevents fluid ingress or egress through the end-effector port 214 even though the end effector 128 is secured to the spring-biased plunger 116.

As is easily seen in FIG. 15, as the lever 104 is actuated toward the open position, the main shaft 118 rotates and the lift cam 120 engages with the spring-biased plunger 116. Because an outer surface 220 of the lift cam 120 frictionally engages the spring-biased plunger 116, the lift cam 120 rotates as the lever 104 is actuated into the open position as shown in FIG. 15.

FIG. 16 shows the lever 104 in the fully open position in which the lift cam 120 has fully lifted the spring-biased plunger 116 such that the end effector 128 is fully retracted within the end-effector port 214. The tube 216 is visibly present in FIG. 16 because of the retraction of the spring-biased plunger 116. Also, note that the plunger-cam follower 192 has been actuated away from the plunger cam 184 such that it no longer touches the plunger cam 184. The lift cam 120 actuates the inlet valve 198 and the outlet valve 200 in a similar manner. That is, the lift cam 120 also engages with the inlet valve 198 and the outlet valve 200, which are also spring biased.

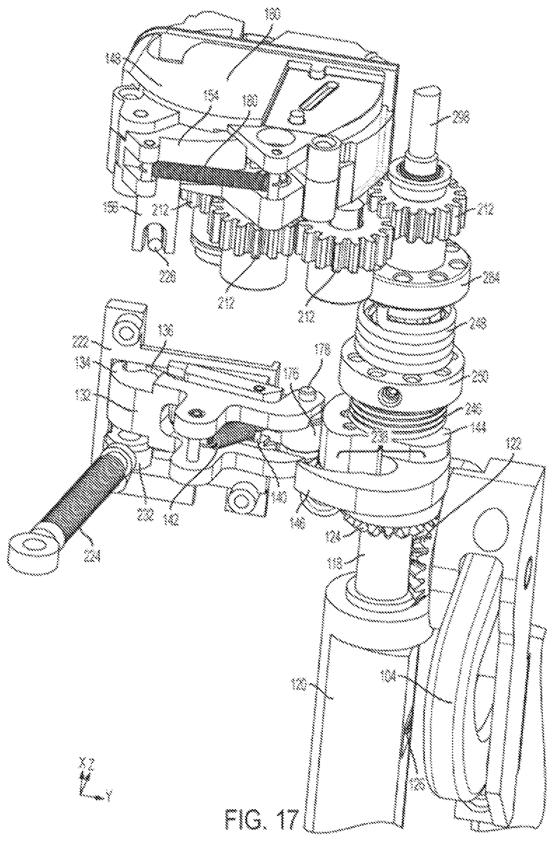

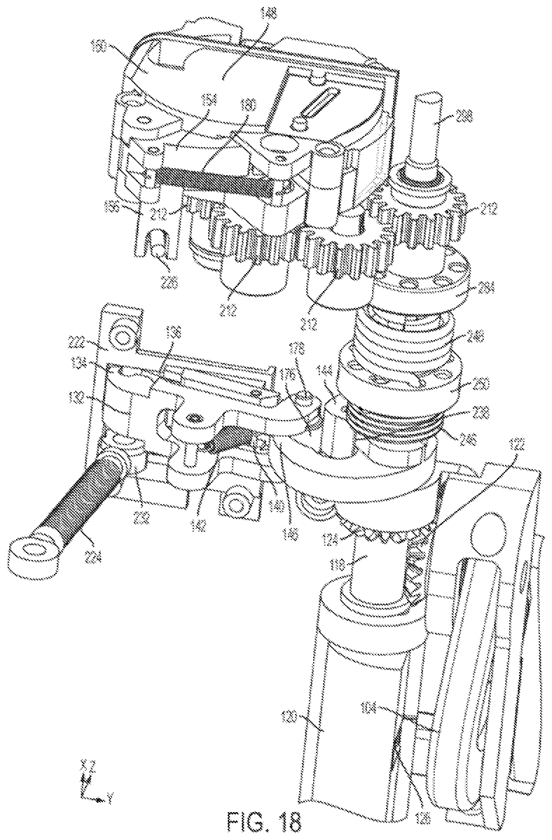

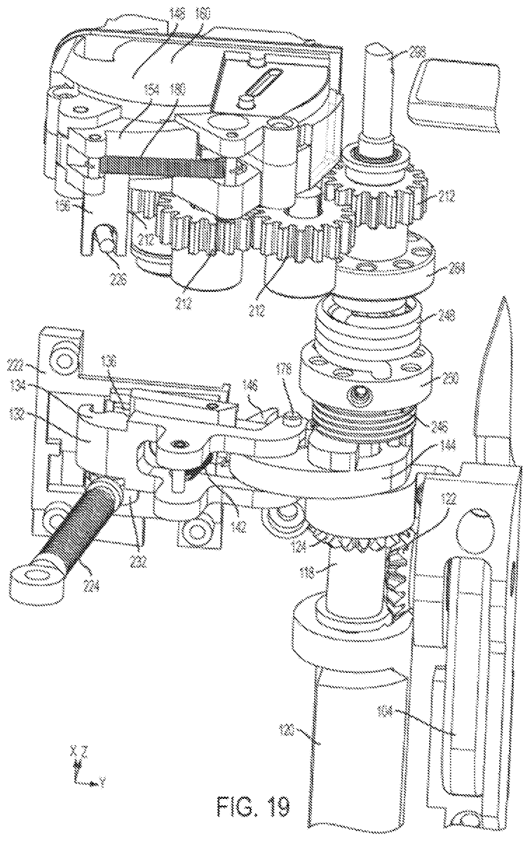

Referring to FIGS. 17-19, FIG. 17 shows a close-up view of the latching sled 132 of the mechanical assembly 210 of the peristaltic pump 100 of FIG. 1 when the lever 104 is in the closed position. FIG. 18 shows a close-up view of the latching sled 132 when the lever 104 is between the closed position and the open position, and FIG. 19 shows a close-up view of the latching sled 132 when the lever 104 is in the open position.

FIG. 17 shows the lever 104 in the closed position and hence the latching sled 132 is in the extended position. When the latching sled 132 is in the extended position, the claw 134 is actuated away from the main shaft 118 because of the abutment of the sled cam follower 176 with the hook cam 144. That is, the hook cam 144 engages with the sled cam follower 176 such that the hook cam 144 extends the sled cam follower 176 maximally away from the main shaft 118. Therefore, FIG. 17 shows the condition where the hook cam 144 has actuated the latching sled 132 to its fully extended position.

When the latching sled 132 is in the extended position, the door 102 and the door catch 114 may initially be unlocked, but as soon as the door catch 114 is actuated to the closed position (e.g., when the door 102 is shut), a door-catch hold 234 of the door catch 114 is locked between the claw 134 and the sled base 136. That is, once the door catch 114 has rotated into the locked position, the latching sled 132 prevents it from being opened because the latching sled 132 is in the extended (or locking) position.

FIG. 18 shows the lever 104 in a partially actuated position where the hook 146 of the hook cam 144 hooks onto the sled cam follower 176. The hook cam 144 includes a retraction space 238 so that the sled cam follower 176 can be pulled toward the main shaft 118. FIG. 19 shows the lever 104 in the fully open position such that the hook 146 of the hook cam 144 has fully retracted the latching sled 132. As the claw 134 was pushed toward the hook cam 144, the claw 134 pulled the door catch 114 into the open (or unlatched position), which in turn, opened the door 102.

Referring to FIGS. 2, 19 and 25, when the lever 104 was actuated from the closed position to the open position, the claw 134 pulled on the door-catch hold 234 such that the door catch 114 was rotated along its channel 236 which rotated the pin catch 166 to a position where it no longer locks the hold 164 of the door 102. Because the door 102 may be spring-biased open, the door 102 may swing open when the door catch 114 no longer locks onto the hold 164 of the door 102.

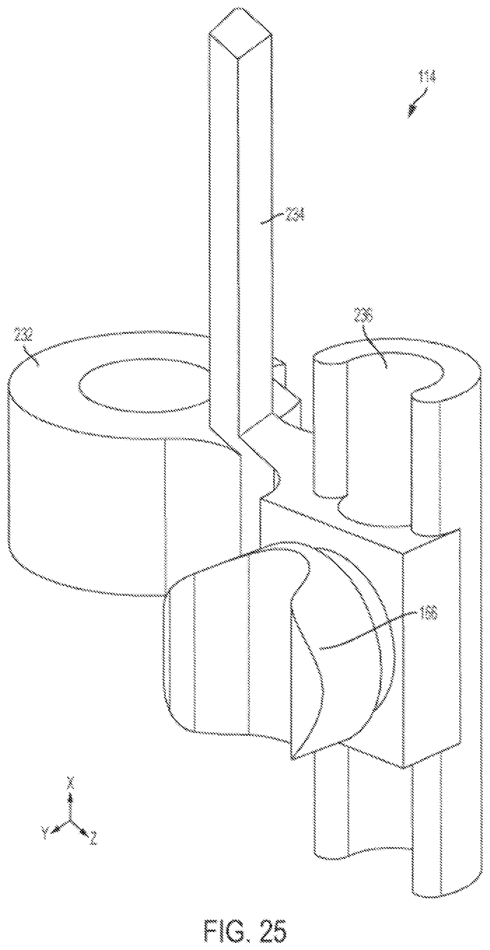

Referring again to FIG. 19, the latching sled 132 is coupled to a door-catch spring 224 that is coupled to the door-catch anchor 232. The door-catch spring 224 pushes against the door-catch anchor 232 which makes the door catch 114 actuate with an “over center” action. The over center action of the door-catch spring 224 makes the door catch 114 bi-stable in the locked position or in the open position. As shown in FIG. 19, when the claw 134 is in a retracted position, the door catch 114 is free to actuate freely between the open position and the locked (or closed) position because the claw 134 has been actuated free from the door-catch hold 234 (see FIG. 25)

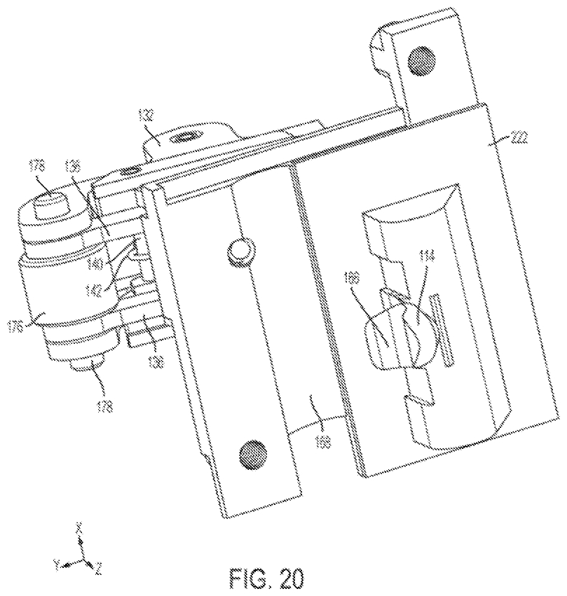

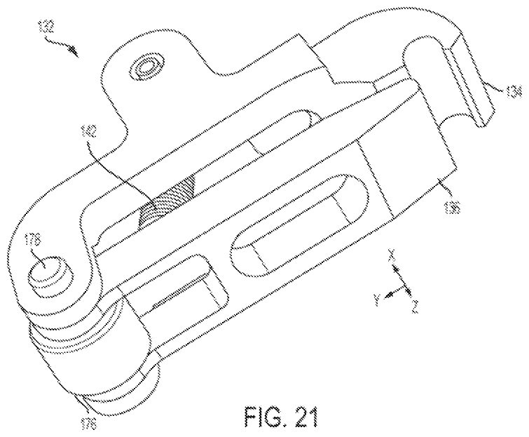

FIG. 20 shows the door catch 114 and latching sled 132 of the peristaltic pump 100 of FIG. 1 from the front side of the pump 100. A door-catch interface 222 separates the outside, in which the pin catch 166 protrudes outside the door-catch interface 222, from the internal parts of the door catch 114 in which the latching sled 132 operates on. FIG. 21 shows the latching sled 132 including a sled base 136 and a claw 134 pivotally coupled to the sled base 136 about an axis of the sled cam follower 176. The sled cam follower 176 is secured to both the sled base 136 and the claw 134 via a sled pin 178. A sled spring 142 is coupled to the claw 134. The sled base 136 slides back and forth in a block 138 of the door-catch interface 222 as shown in FIG. 22.

FIG. 22 shows the door catch 114 and latching sled 132 of the peristaltic pump 100 of FIG. 1 from the back side of the pump 100. The claw 134 of the latching sled 132 is in a locking position. The sled spring 142 is coupled to the claw 134 and to an anchor pin 140 of the block 138. The sled spring 142 biases the claw 134 toward the sled base 136 and biases the latching sled 132 toward the door-catch hold 234. However, the position of the sled base 136 within the block 138 is controlled by the hook cam 144 (See FIG. 19).

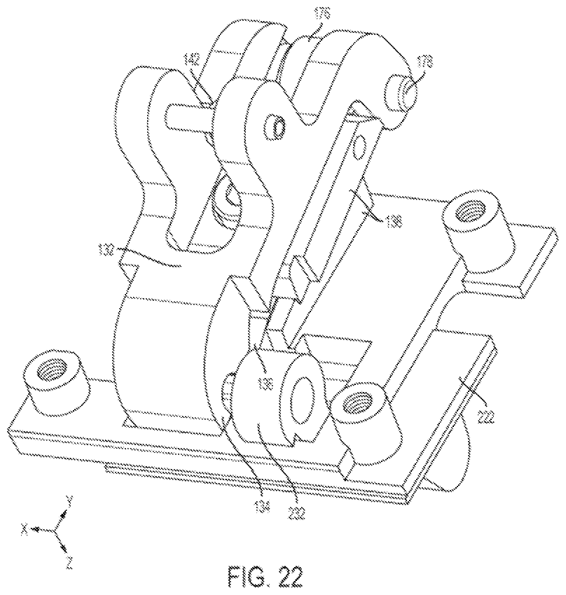

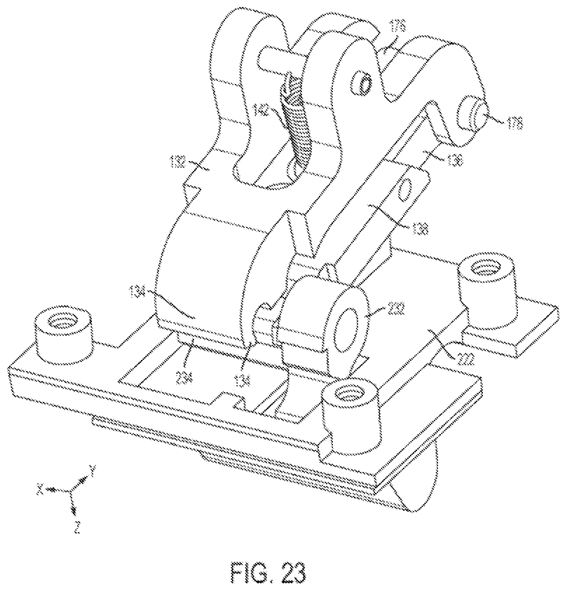

FIG. 23 shows the door catch 114 and latching sled 132 where the claw 134 of the latching sled 132 is in a retracted position. As is easily seen in FIG. 23, the door-catch hold 234 has been pulled back by the claw 134. In this position, wherein the latching sled 132 has been pulled back because the lever 104 has been actuated to the open position, the door-catch hold 234 is free to be actuated between the two positions shown in FIGS. 22 and 23 because the claw 134 has been lifted up away from the door-catch hold 234. The force of the door-catch spring 224 on the door-catch anchor 232 pushes the door-catch hold 234 into one of the positions of FIGS. 22 and 23.

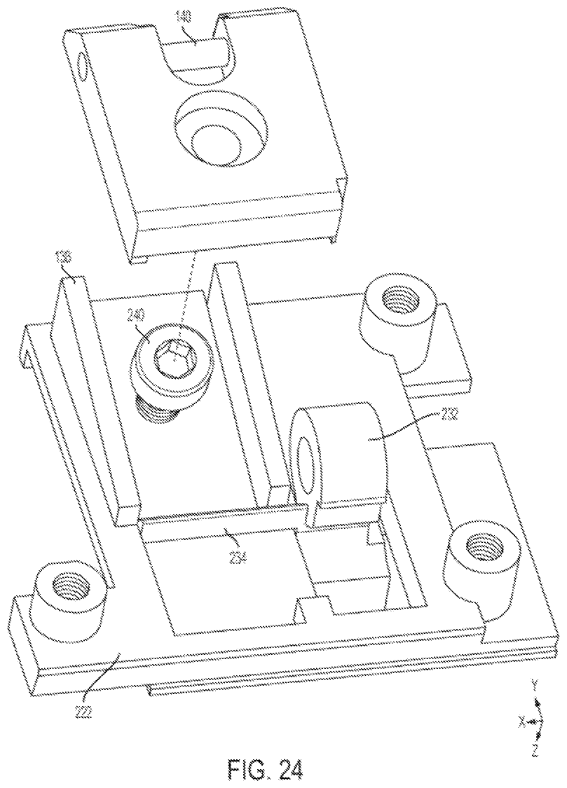

FIG. 24 shows the door catch 114 and a portion of the block 138 that seats the latching sled 132 for the peristaltic pump 100 of FIG. 1. Also show in exploded view is the anchor pin 140 on the top portion of the block 138 that is secured to the bottom portion of the block 138 by a screw 240. Easily seen in FIG. 24, the door-catch hold 234 is actuatable between the two position. FIG. 25 shows the door catch 114, which is rotatable along a pivot defined by the channel 236. The channel 236 may receive any device that makes the door catch 114 pivotable, such as a pin, flange, or protrusion on the door-catch interface 222.

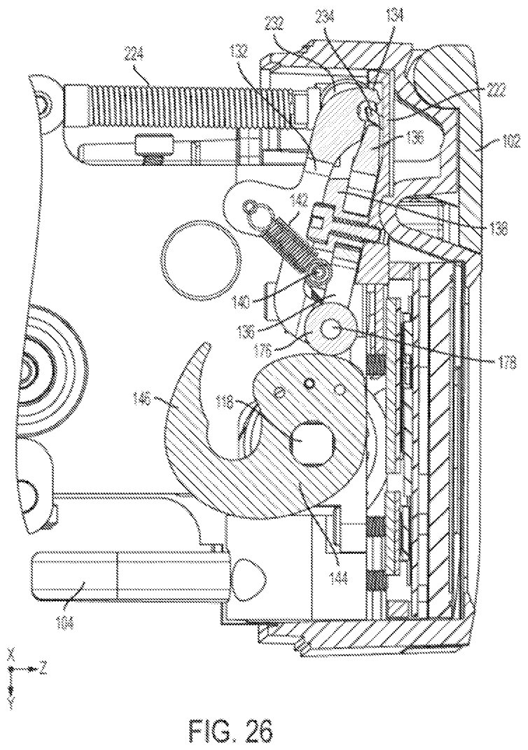

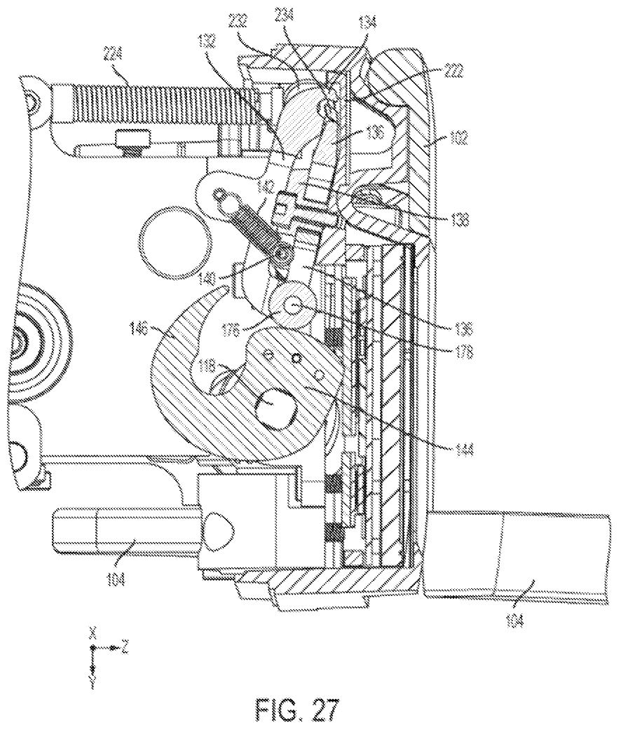

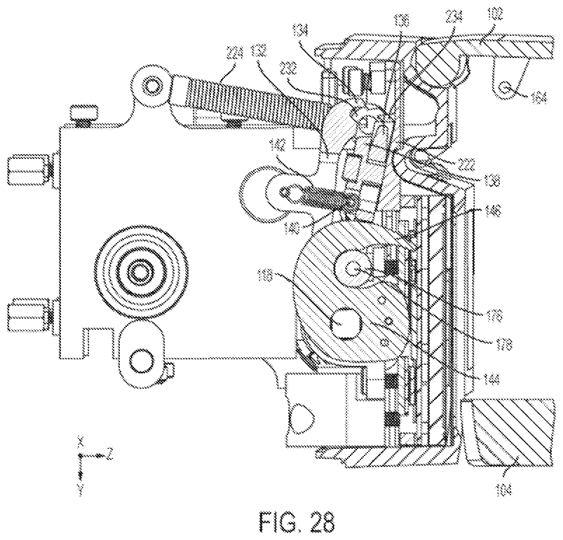

Refer now to FIGS. 26-28: FIG. 26 shows a cross-sectional view of the peristaltic pump 100 of FIG. 1 with a hook cam 144 in a non-hooking position; FIG. 27 shows the cross-sectional view of FIG. 26, but with the hook cam 144 partially actuated toward the cam follower of the latching sled 132; And FIG. 28 shows the cross-sectional view of FIG. 26, but with the hook cam 144 fully actuated such that the hook 146 has coupled to the cam follower of the latching sled 132 and has fully retracted the latching sled 132.

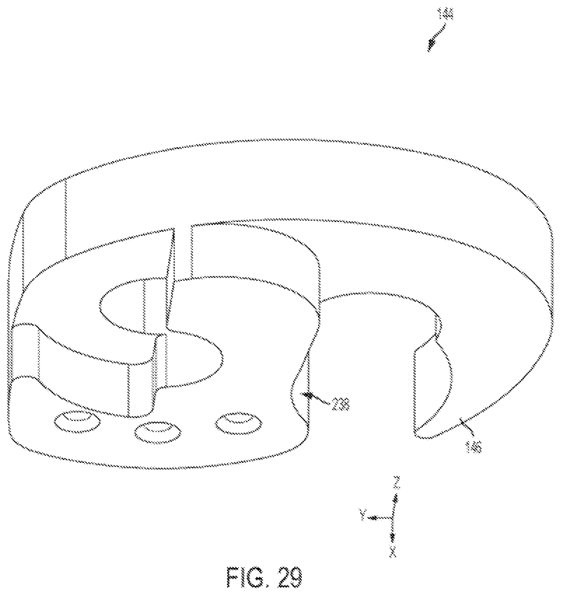

As can be seen through the sequence of FIGS. 26, 27, and 28, the hook 146 of the hook cam 144 grabs onto the sled cam follower 176 and retracts the latching sled 132. As the claw 134 is pulled back, the door-catch hold 234 is retraced within it. The door catch 114 is then in the unlocked state as shown in FIG. 28. When the door 102 is fully opened as shown in FIG. 28, the door-catch hold 234 is able to freely actuate between the open and closed position. The door-catch spring 224 pushes against the door-catch anchor 232 such that the door catch 114 is bi-stable between the positions shown in FIGS. 26 and 28. Also, it is easily viewable in FIG. 28 that the block 138 lifts up the claw 134 as it is retracted by the hook cam 144 despite the sled spring 142. That is, the surface of the block 138 provides a cam action against the claw 134 to lift up the claw 134 when the latching sled 132 is retracted by the hook cam 144. The sled spring 142 biases the claw 134 toward the sled base 136. FIG. 29 shows the hook cam 144 with a close up to illustrate the retraction space 238 which allows a portion of the claw 134 to retract more closely to the main shaft 118.

FIG. 30 shows an exploded view of a coupling 242 for coupling together the main shaft 118 to the upper shaft 298 of the peristaltic pump 100 of FIG. 1, and FIG. 31 shows an exploded view of the coupling 242 of FIG. 30 but from another viewing angle.

Referring to both FIGS. 30 and 31, the coupling 242 includes a middle connector 250, a first connector 282, and a second connector 284. The embodiment shown herein shows the hook cam 144 and the first connector 282 integrated together. The middle connector 250 is rigidly coupled to the main shaft 118. The hook cam 144 rotates around the main shaft 118 (see FIG. 19). The second connector 284 is rigidly coupled to the upper shaft 298 (see FIG. 19).

The middle connector 250 includes a first flange 252 that can interface with one of a first stop 256 of the first connector 282 or a second stop 258 of the first connector 282. The middle connector 250 also includes a second flange 254 that can interface with a third stop 260 or a fourth stop 262 of the second connector 284. The first flange 252 engages with the first stop 256 of the first connector 282 such that when the lever 104 is actuated from the closed position to the open position, the rotation of the main shaft 118 rotates the middle connector 250 (via direct coupling) to press the first flange 252 against the first stop 256 to thereby actuate the hook cam 144 to retract the latching sled 132. Likewise, the second flange 254 engages with the third stop 260 such that when the lever 104 is actuated from the closed position to the open position, the rotation of the main shaft 118 rotates the middle connector 250 (via direct coupling) to press the second flange 254 against the third stop 260 to rotate the second connector 284 with the main shaft 118; because the upper shaft 298 is directly coupled to second connector 284, the interface of the second flange 254 with the third stop 260 causes the main shaft 118 and the upper shaft 298 to rotate with each other when the lever 104 is actuated from the closed position to the open position.

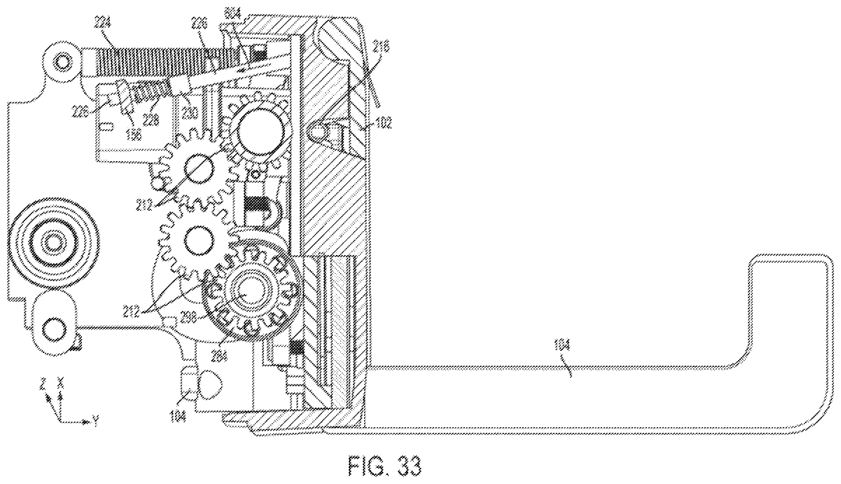

A first shaft spring 246 torsionally biases the middle connector 250 relative to the first connector 282, and the second shaft spring 248 torsionally biases the middle connector 250 relative to the second connector 284. The coupling 242 allows the main shaft 118 to continue to rotate a predetermined amount when the gears 212 are locked and thereby causing the upper shaft 298 to remain stationary. Although described in greater detail below, a pawl 154 of the carriage assembly 160 (see FIG. 33) can prevent the carriage 150 from rotating and can prevent the gears 212 (see FIGS. 32-33) from also rotating. Because the gears 212 are rigidly coupled to the upper shaft 298, when the gears 212 are prevented from rotating, the upper shaft 298 is also prevented from rotating.

That is, a user trying to actuate the lever 104 to the closed position while the door 102 is open will be prevented from closing the lever 104 to keep it closed because once a user lets go of the lever 104, the lever 104 will quickly spring back to the open position. Rather than rigidly stopping any actuation of the lever 104 as the user attempts to actuate the lever 104 to the closed position while the door 102 is open, the coupling 242 provides a spring resistance until the lever 104 is in the fully closed position. The main shaft 118 is not shown in FIGS. 30-31, however as previously mentioned, the main shaft 118 is rotationally disconnected from the upper shaft 298 thereby allowing them to rotate independently. In other embodiments, the main shaft 118 may be rigidly coupled to the upper shaft 298. When the door 102 is open, the coupling 242 allows a predetermined amount of actuation of the lever 104 toward the closed position until the lever 104 is fully closed, or in other embodiments, the coupling 242 prevents any additional actuation. When the door 102 is closed, the upper portion of the main shaft 118 is not locked and the lever 104 can be freely actuated to the closed position.

When the door 102 is open and the user tries to actuate the lever 104 from the open position to the closed position, the main shaft 118 continues to rotate. Because the main shaft 118 is coupled to the middle connector 250, the middle connector 250 will rotate with actuation of the lever 104; however, the second connector 284 will not rotate because the gears 212 are locked by virtue of the door 102 being open which thereby locks the upper shaft 298 and the first connector 282 will also not rotate because the hook cam 144 cannot overcome the bias of the door-catch spring 224 that holds the latching sled 132 in the retracted position. Referring to FIGS. 30-31, in this situation, the middle connector 250 will rotate because it is connected to the main shaft 118 and the first connector 282 and the second connector 284 will remain stationary as the user attempts to close the lever 104 with the door 102 open. The hook cam 144 does not rotate in this situation because it is rigidly connected to the first connector 282. The first flange 252 will leave the first stop 256 thereby charging the first shaft spring 246 and the second flange 254 will leave the third stop 260 thereby charging the second shaft spring 248. If the user lets go of the lever 104, it will quickly open because of the charging of the first shaft spring 246 and the second shaft spring 248. Alternatively, if the user, while holding the lever 104 in the fully closed position against the biasing of the first shaft spring 246 and the second shaft spring 248, attempts to close the door 102, the lifter pin 226 will actuate causing the lifter spring 228 to press against the lift 156. However, because the pawl 154 (see FIG. 33) is locked under force (via the first shaft spring 246 and the second shaft spring 248), the lifter spring 228 cannot overcome the force needed to lift the lift 156 and release the carriage 150 (described in more detail below). Nonetheless, the latching sled 132 may overcome the spring 224 (through assistance of the door 102 causing actuation of the door catch 114) thereby allowing the hook cam 144 to rotate such that the first stop 256 again engages the first flange 252; however, as soon as the user lets go of the lever 104, the lever 104 will quickly open causing the hook cam 144 to quickly retract the latching sled 132 again because of charge of the second shaft spring 248.

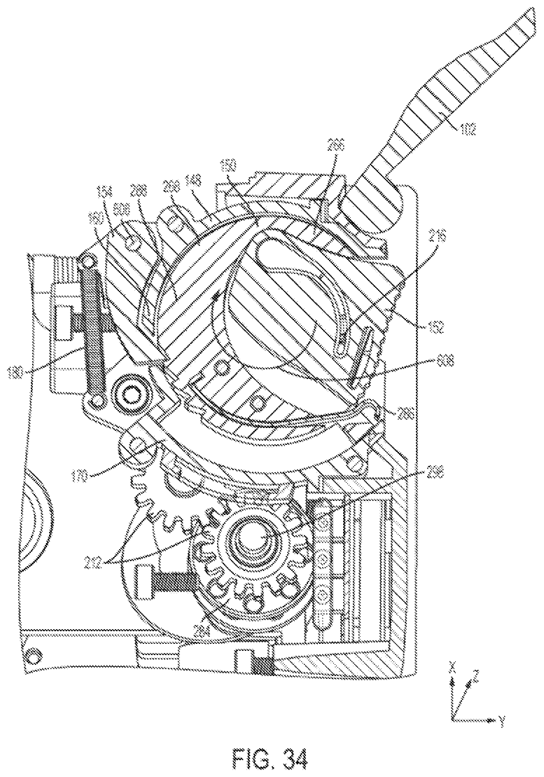

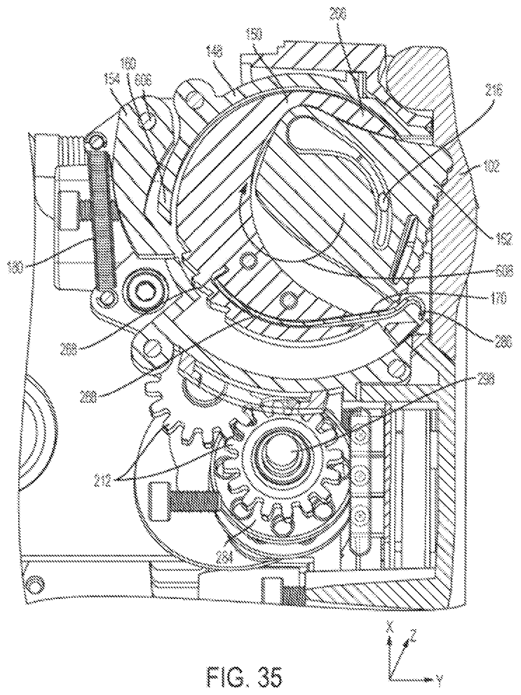

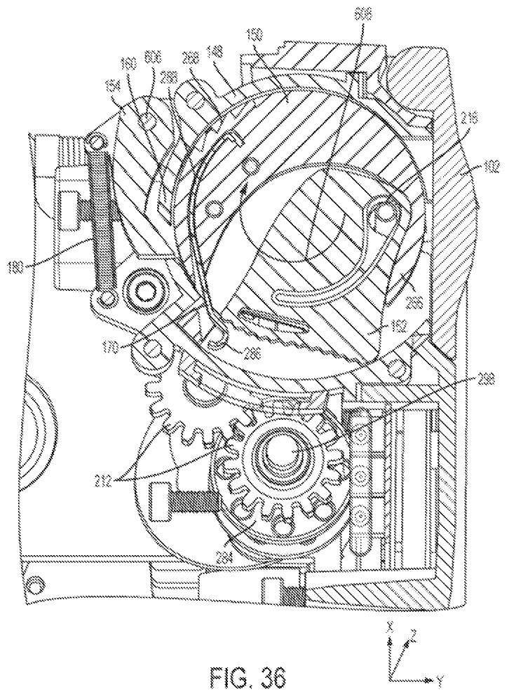

FIG. 32 shows a cross-sectional view of the peristaltic pump 100 of FIG. 1. The gears 212 can actuate the carriage 150 by actuation of the main shaft 118. That is, the gears 212 couple the main shaft 118 to the carriage 150 (see FIGS. 34-36) so that the carriage 150 (see FIGS. 34-36) can rotate. Rotation of the carriage 150 causes the tube 216 to either be in an occluding position or a non-occluding position within the flow stop 152. FIGS. 32, 34, 35 correspond to the carriage 150 being in a position that positions the tube 216 to be occluded within the flow stop 152, while FIG. 36 corresponds to the carriage 150 being in a position that positions the tube 216 to be non-occluded within the flow stop 152. FIG. 33 shows the lifter pin 226 in the position that can correspond to either FIG. 35 or FIG. 36.

FIG. 32 shows the lifter pin 226 in a position that prevents the carriage 150 from rotating when a user attempts to shut the lever 104 with the door 102 open. FIG. 33 shows the lifter pin 226 in a position that allows the carriage 150 to rotate in response to a user closing the lever 104 when the door 102 is closed.

When the door 102 is open as shown in FIG. 32, the lifter pin 226 sticks out of a hole (see FIGS. 2-4 for a clear view of the end of the lifter pin 226) to ensure that the carriage 150 is locked and is prevented from rotation in direction 608 as shown in FIG. 34. As shown in FIG. 34, the pawl 154 is located in a groove of the notches 268 which prevents the carriage 150 from rotating to the position shown in FIG. 36. That is, the pawl 154 has locked the carriage 150. When the door 102 is open as shown in FIG. 32, the pawl 154 is engaged with the notches 268 as shown in FIG. 34. Because the door 102 is open, the lifter pin 226 is not pushing on the lift 156 through the lifter spring 228. This prevents the lever 104 from being actuated toward the closed position because the carriage 150 is coupled to the gears 212, which in turn is mechanically coupled to the main shaft 118. This feature prevents the user from actuating the lever 104 closed while the door 102 is open. Closing the door 102 actuates the pawl 154 out of the notches 268 (via the lifter pin 226).

FIG. 33 shows the same cross-sectional view as in FIG. 32 but with the door 102 closed which thereby actuates the lifter pin 226 away from the door 102 to compress the lifter spring 228 which actuates the lift 156. That is, as shown in FIG. 33, when the door 102 is shut, the door 102 presses on an end of the lifter pin 226 (see FIGS. 2-4) which actuates the lifter pin 226 in a direction that is illustrated by an arrow 604 in FIG. 33. The lifter-pin collar 230 is rigidly coupled to the lifter pin 226 and thus both the lifter-pin collar 230 and the lifter pin 226 move in the direction of the arrow 604 when the door 102 is shut to the position shown in FIG. 33.

As previously mentioned, the door 102 impinges on the end (see FIGS. 2-4) of the lifter pin 226 when the door 102 is shut thereby actuating the lifter pin 226 in the direction of arrow 604 as shown in FIGS. 32-33. As the lifter pin 226 actuates away from the door 102, the lifter-pin collar 230 also moves away from the door 102 to thereby compress a lifter spring 228 against the lift 156. Compression of the lifter spring 228 applies a force against the lifter pin 226 which actuates the lift 156 away from the door 102 because the lift 156 is coupled to a pawl 154 as shown in FIGS. 34-36. The pawl 154 is pivotably coupled to the carriage assembly 160 via a pawl pivot 606.

When the door 102 is open as shown in FIG. 32, the lifter pin 226 is actuated away from the lift 156 such that the pawl 154 engages with the notches 268 as is shown in FIG. 34. FIG. 34 shows a cross-sectional view of the peristaltic pump 100 of FIG. 1 to show a cross-sectional view of the carriage assembly 160 with the door 102 open and the lever 104 open. As shown in FIG. 33, when the lift 156 is actuated away from the carriage 150 by closing the door 102, the pawl 154 is also actuated away from the carriage 150 as is shown in FIG. 35 by compression of the lifter spring 228 against the lift 156 coupled to the pawl 154. FIG. 35 shows the same cross-sectional view as in FIG. 34 but the door 102 is closed which actuates the pawl 154 out of the notches 268.

That is, actuation of the lift 156 away from the carriage 150 actuates the pawl 154 such that the carriage 150 can freely rotate. When the pawl 154 is lifted by the lift 156, the pawl 154 cannot engage with the notches 268 of the carriage 150 as shown in FIG. 35 and therefore the carriage 150 can freely rotate. When the pawl 154 is engaged with the notches 268 as shown in FIG. 34 of the carriage 150, the carriage 150 cannot rotate to the position shown in FIG. 36. The carriage 150 can rotate in the direction 608 shown as the clockwise arrow in FIGS. 34 and 35 into the position shown in FIG. 36 when the lever 104 is closed. FIG. 36 shows the same cross-sectional view as in FIG. 35 but with the carriage 150 in a rotated position which is caused by closure of the lever 104.

As shown in FIG. 34, the flow-stop retainer 170 includes a retainer hook 286 and a spring body 288. The flow-stop retainer 170 allows the flow stop 152 to be snap-fitted in the carriage 150 and also provides resistance when pulling the flow stop 152 out of the carriage 150.

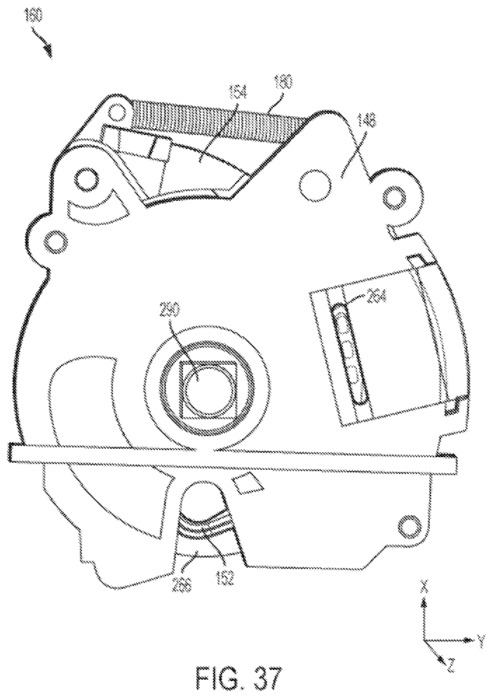

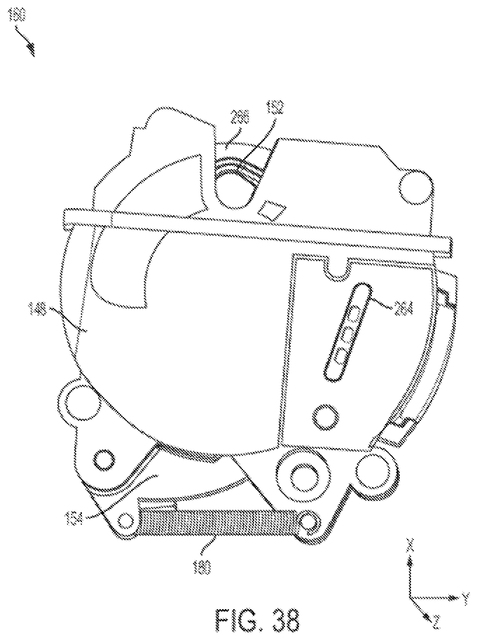

FIG. 37 shows the carriage assembly 160 of the peristaltic pump 100 of FIG. 1 from a bottom side of the carriage 150, and FIG. 38 shows the carriage assembly 160 of the peristaltic pump 100 of FIG. 1 from a top side of the carriage 150. FIG. 37 shows the gear connector 290 that mechanically couples the carriage 150 to the main shaft 118. The carriage assembly 160 includes a carriage housing 148, a pawl 154, a pawl spring 180, the gear connector 290, and a window 264. The window 264 allows light (e.g., generated by an LED) to shine through the window 264. A sensor on the other side of the window 264 can sense which portions of the window 264 are blocked and/or which positions of the window 264 has light shining therethrough. Flow-stop ID holes 294 on a flow stop 152 can indicate a binary number which can be used to identify the flow stop 152 and/or the set the flow stop 152 is attached to. As shown in FIG. 37, when the carriage 150 is in the closed position, a cover 266 blocks the entrance to the carriage assembly 160 (also see FIG. 37).

FIG. 39 shows the carriage assembly 160 of the peristaltic pump 100 of FIG. 1 from a bottom side of the carriage assembly 160 with the bottom portion of the carriage housing 148 removed for clarity. As shown, the cover 266 can be easily seen as blocking the entrance of the carriage assembly 160, which in turn prevents insertion of anything into the carriage 150 while the carriage 150 is rotated to the closed position. The flowing portion 270 of the flow stop 152 is over the carriage-assembly hole 292 which allows fluid to flow through the tube 216. When the carriage 150 is in the open position, the carriage-assembly hole 292 holds the tube 216 such that the tube 216 is positioned between the occluding portion 272 of the flow stop 152. This requires the flow stop 152 to be loaded and unloaded into the carriage 150 by the user only when the flow stop 152 is occluding the tube 216.

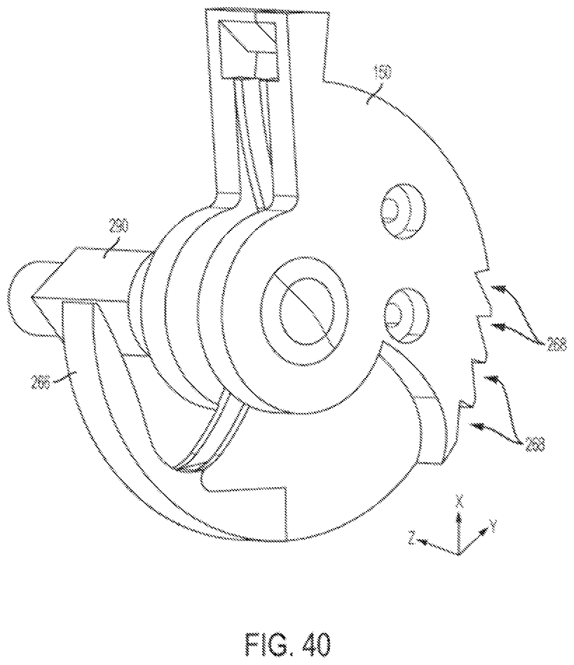

After the flow stop 152 is secured within the carriage 150 and the door 102 is shut, actuation of the lever 104 to the closed position rotates the carriage 150 such that the carriage-assembly hole 292 holds the tube 216 such that the tube 216 can reside within the flowing portion 270 of the flow stop 152. When the tube 216 is positioned within the flowing portion 270, fluid may easily flow through the tube 216. FIGS. 40 and 41 show views of the carriage 150 of the peristaltic pump 100 of FIG. 1. The notches 268 are easily seen as is the cover 266.

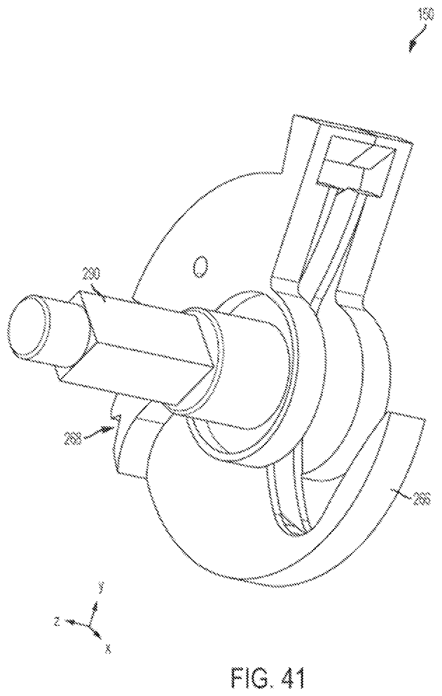

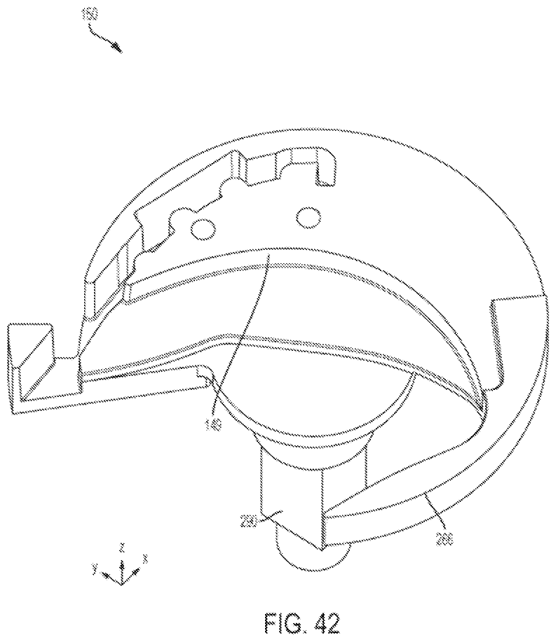

FIG. 42 shows the carriage 150 with the top portion removed to illustrate a guide surface 149 of the carriage 150. The guide surface 149 is configured to allow the stabilizer 278 of the flow stop 152 to translate insertion force applied to the thumb rest 280 into sliding of the tube 216 within an arcuate slot 151 of the flow stop 152 which is described in greater detail below.

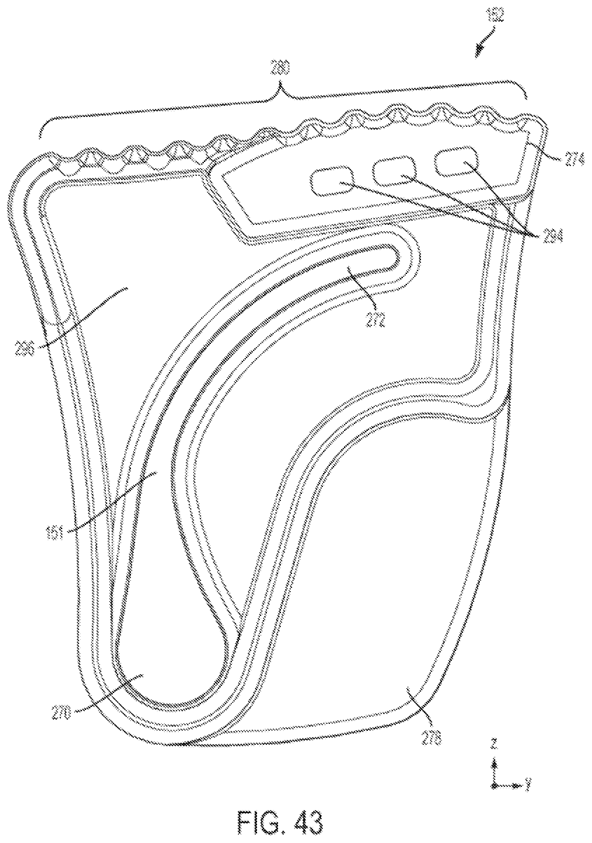

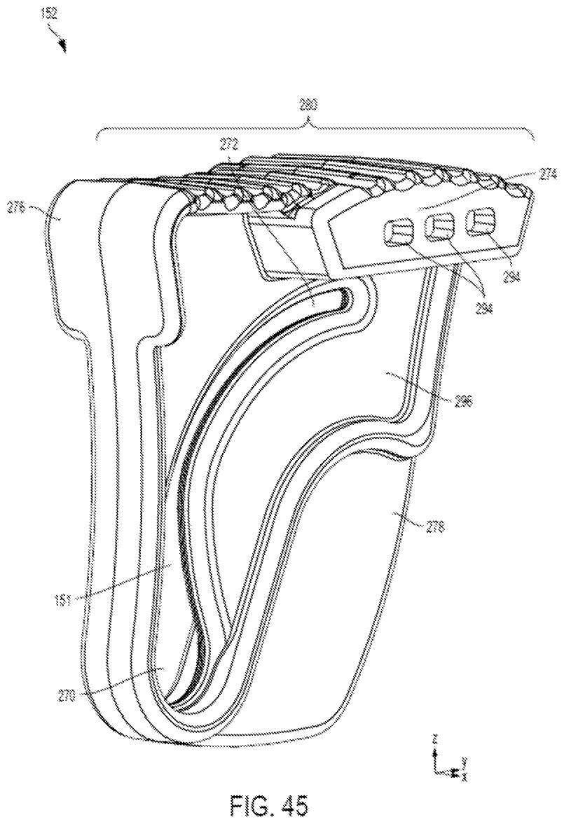

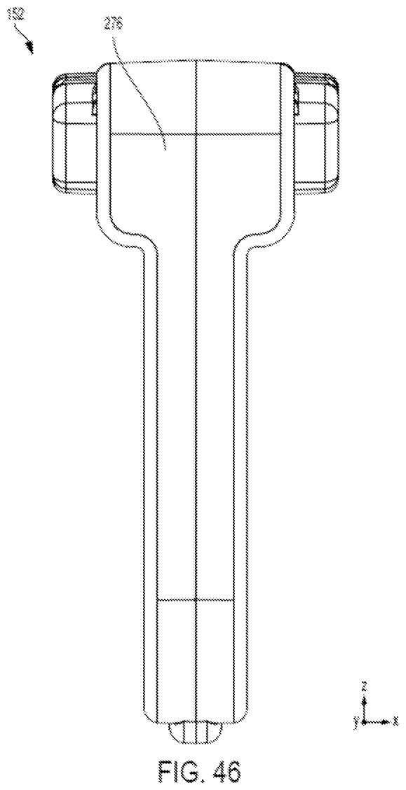

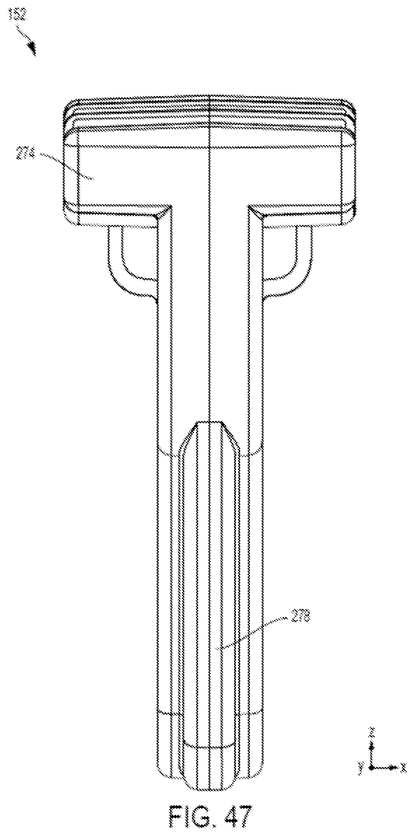

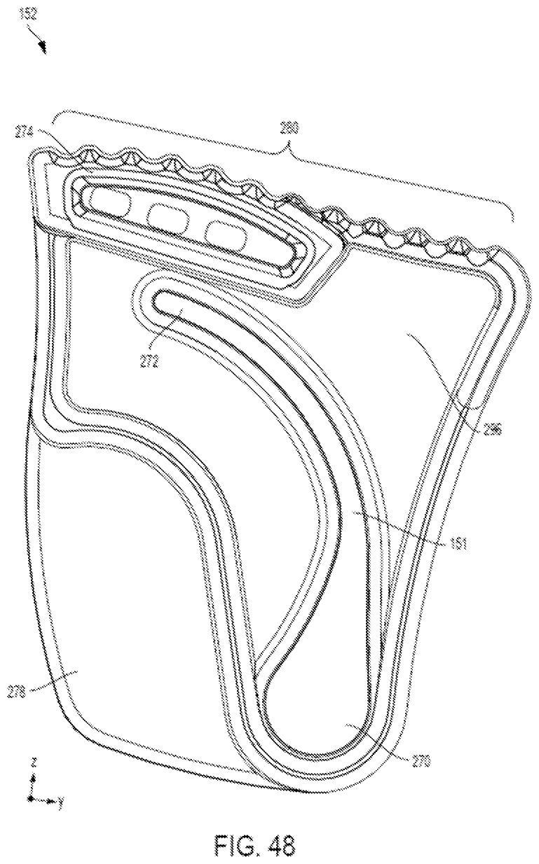

FIGS. 43-48 show several views of the flow stop 152 that can be inserted into the carriage 150 of the peristaltic pump 100 of FIG. 1. The flow stop 152 includes a body 296 defining an arcuate slot 151 that receives a tube 216 therein. The arcuate slot 151 includes an occluding portion 272 and a flowing portion 270. The flow stop 152 also includes a stabilizer 278. The stabilizer 278 facilitates insertion of the flow stop 152 into the carriage 150. A thumb rest 280 is shown that provides a frictional area for a person to press the flow stop 152 into the carriage 150. As is easily seen in FIG. 43, the thumb rest 280 includes an extension 274. Within the extension 274 are the flow-stop ID holes 294 for the light to identify the flow stop 152. The flow-stop ID holes 294 are easily seen in FIG. 43. The back 276 is easily seen in FIG. 45.

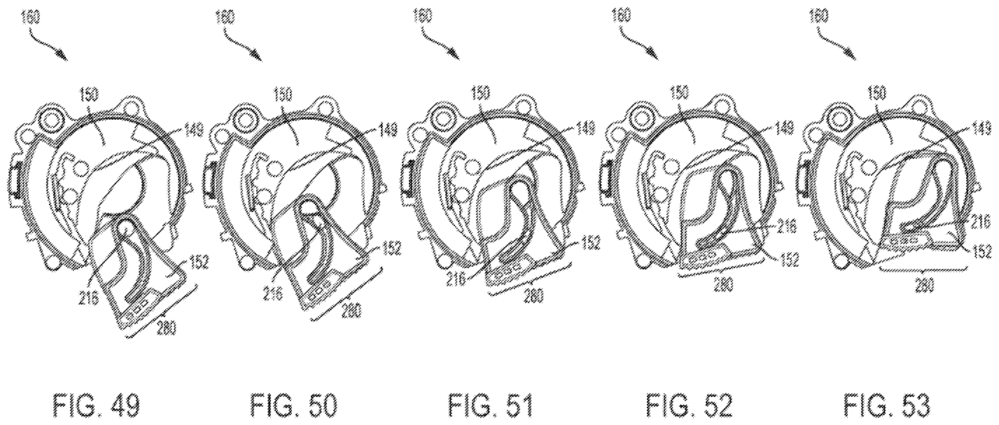

FIGS. 49-53 show a sequence of events to illustrate the flow stop 152 of FIGS. 43-48 being inserted in the carriage assembly 160 of the peristaltic pump 100 of FIG. 1. The carriage 150 as shown in FIGS. 49-53 is shown with the top removed for easy viewing of the interaction of the stabilizer 278 and the guide surface 149. The stabilizer 278 and the guide surface 149 interact with each other in order to prevent the flow stop 152 from being inserted into the carriage at an angle that would pinch the tube 216.

Initially, prior to insertion of a flow stop 152 of an administration set, a user may place the tube 216 anywhere within the arcuate slot 151. If the user places the tube 216 within the end of the occluding portion 272 of the arcuate slot 151, the carriage 150 can receive the flow stop 152 with the tube 216 being occluded without moving or repositioning the tube 216 within the arcuate slot 151.

However, if the user has the tube 216 positioned in the flowing portion 270 or partially between the flowing portion 270 and the end of the occluding portion 272, the carriage assembly 160 will reposition the tube 216 to the end of the occluding portion 272 as the flow stop 152 is inserted into the carriage 150.

FIG. 49 shows the initial insertion of the flow stop 152 where the tube 216 is in the flowing portion 270. As can be seen in the sequence of events from FIG. 49 to FIG. 53, as the flow stop 152 is inserted, the tube 216 slides into the end of the occluding portion 272 as shown in FIG. 34. During this process, the stabilizer 278 and the guide surface 149 interact with each other to prevent the tube 216 from getting pinched or damaged from forces orthogonal to the center line of the arcuate slot 151.

That is, as a user pushes on the thumb rest 280, the guide surface 149 causes the flow stop 152 to be guided to the fully-inserted position in the carriage 150 as shown in FIG. 53 while adjusting the angle of the flow stop 152 to translate forces on the thumb rest 280 to the tube 216 so that the tube 216 experiences a force substantiality parallel with the center line of the arcuate slot 151. The stabilizer 278 is guided by the guide surface 149 because the stabilizer 278 will abut the guide surface 149 if the user attempts to rotate the flow stop 152 counterclockwise (from the perspective shown in FIGS. 49-53) while attempting to insert the flow stop 152. Thus, the stabilizer 278 of the flow stop 152 prevents the tube 216 from becoming pinched or damaged by the interface between the carriage 150 and the flow stop 152. The stabilizer 278 and the guide surface 149 mitigate the force of the user pressing on the flow stop 152 from being translated on the tube 216 to push the tube orthogonal with the center line of the arcuate slot 151 which would cause the tube 216 to become pinched because the tube 216 would be trapped within the channel defined by the hole 106 (see FIG. 2) if the tube 216 was forced to move orthogonally to the center line of the arcuate slot 151.

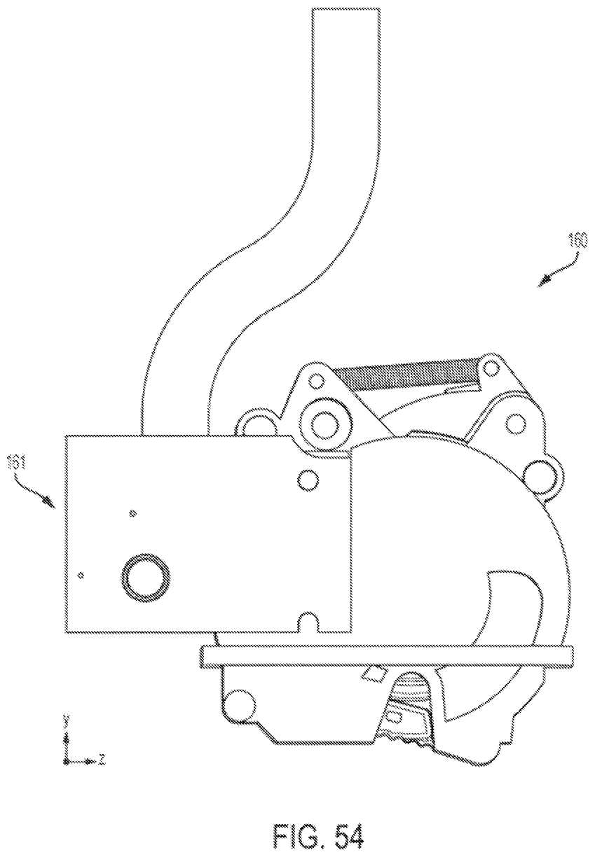

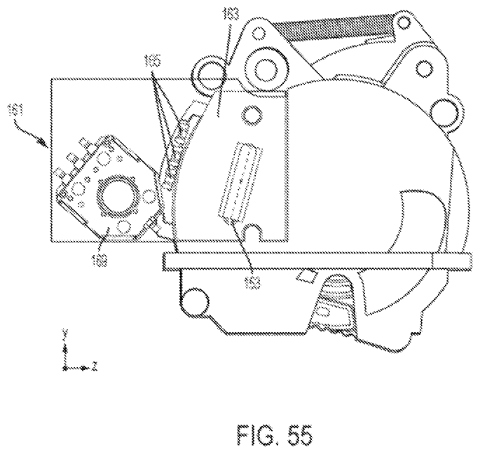

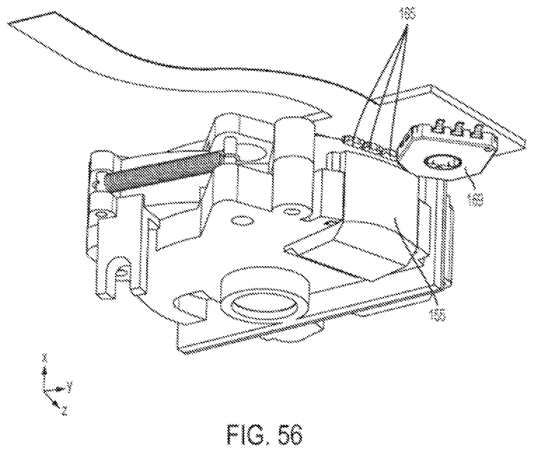

FIG. 54 shows the carriage assembly 160 from the top side with a sensor board 161 coupled thereto of the peristaltic pump 100 of FIG. 1. FIG. 55 shows the same view as FIG. 54 but with the sensor board 161 shown as being transparent to show a group of LEDs 165 which are part of a flow-stop ID sensor 163. The flow-stop ID sensor 163 includes the LEDs 165 that are used to generate light, which may be visible light, non-visible light, infrared light, near infrared light, ultraviolet light, narrow-band light, wide-band light, within an optical portion of the electromagnetic spectrum, or some suitable combination thereof. The flow-stop ID sensor 163 also includes an optical sensor 153, which may be a linear array of light sensitive elements, e.g., 128 grayscale detectors. Also, as is easily seen in FIG. 56, the flow-stop ID sensor 163 includes a light pipe 155.

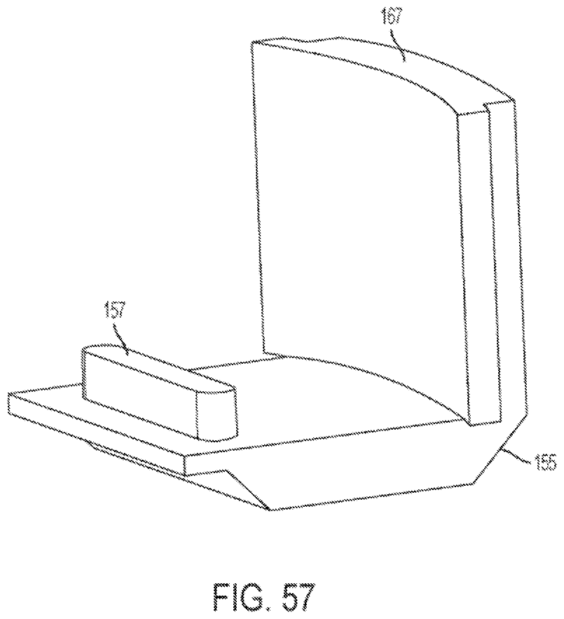

The LEDs 165 emit light that is transmitted within the light pipe 155 to route light to the side of the carriage assembly 160 opposite to the side that the sensor board 161 is coupled to. FIG. 57 shows the light pipe 155 including a receiver aperture 167 that receives light from the LEDs 165 (see FIG. 56) and a transmission aperture 157 that transmits light through a window 264 of the carriage assembly 160 on the bottom side of the carriage assembly 160 (see FIG. 37 for the window 264 on the bottom side). The light is transmitted through any of the flow-stop ID holes 294 of the extension 274 (see FIG. 43) when the flow stop 152 is in the carriage 150 and the carriage 150 is positioned in the lever-closed position (as shown in FIG. 39) when the lever 104 is closed.

Referring again to FIGS. 55 and 56, as is easily seen, using the light pipe 155 allows a single sensor board 161 to house the LEDs 165 and the optical sensor 153. The sensor board 161 also includes a rotation sensor 169 that may be a rotary encoder coupled to an end of the upper shaft 298 (see FIG. 11).

FIG. 58 shows a flow-chart diagram to illustrate a method 400 of using the peristaltic pump 100 of FIG. 1. The method 400 may include acts 401-415. Act 401 actuates the lever 104 to the open position by a user. That is, if the lever 104 was previously closed, the user can actuate the lever 104 open, which will open the door 102 as described above and is illustrated as Act 402 in the method. Act 402 opens the door 102 and rotates the carriage 150 to a position to receive a flow stop 152 in response to actuation of the lever 104 to the open position (see FIG. 34). In this position, if the carriage 150 already includes a flow stop 152 (e.g., from a previous therapy), the user can remove the flow stop 152 and replace it with a new flow stop 152 because in Act 402, the carriage 150 was rotated to a position where a user can remove or insert the flow stop 152. Act 403 actuates the spring-biased plunger 116, the inlet valve 198, and the outlet valve 200 into a retracted position in response to actuation of the lever 104 to the open position. This facilitates easy insertion of the tube 216 into the platen 168 without being impeded by one or more of the spring-biased plunger 116, the inlet valve 198, and/or the outlet valve 200.

Act 404 moves the flow stop 152 to an occluding position on the tube 216 by the user. Act 404 is optional because during Act 405 the user will insert the flow stop 152 into the carriage 150 and, as described above with reference to FIGS. 49-53, the tube 216 may be moved to the occluding position within the arcuate slot 151 automatically during flow stop 152 insertion into the carriage 150.

Act 406 prevents actuation of the lever 104 to the closed position if attempted by the user while the door 102 remains open. Act 407 closes the door 102 by the user. Act 408 unlocks the carriage 150 in response to closing the door 102. Act 409 actuates the lever 104 to the closed position by the user. Act 410 rotates the carriage 150 to position the tube 216 within the flow stop 152 to a non-occluding position in response to actuation of the lever 104 to the closed position.

Act 411 releases the spring-biased plunger 116, inlet valve 198, and the outlet valve 200 from the retracted position in response to actuation of the lever 104 to the closed position. That is, the lift cam 120 or (or 302) will no longer interact with the spring-biased plunger 116, inlet valve 198, and outlet valve 200. Act 412 illuminates a plurality of LEDs 165 onto a plurality of predetermined locations on the flow stop 152, such as on the flow-stop ID holes 294. Act 413 determine whether each of the plurality of predetermined locations on the flow stop 152 is optically blocked or unblocked by sensing the illuminations from the plurality of LEDs 165. Act 414 generates a binary number based upon of the predetermined locations on the flow stop 152. Act 415 authorizes or denies the pump 100 to permit infusion therapy based upon the binary number.

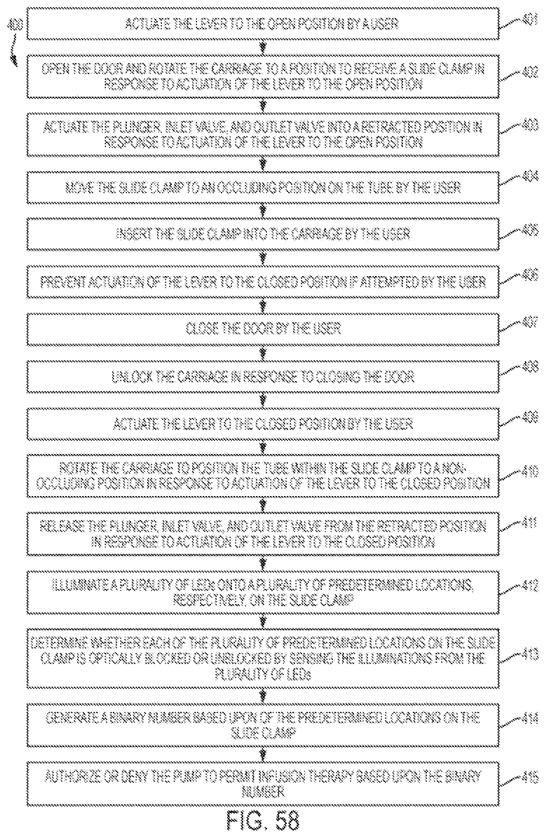

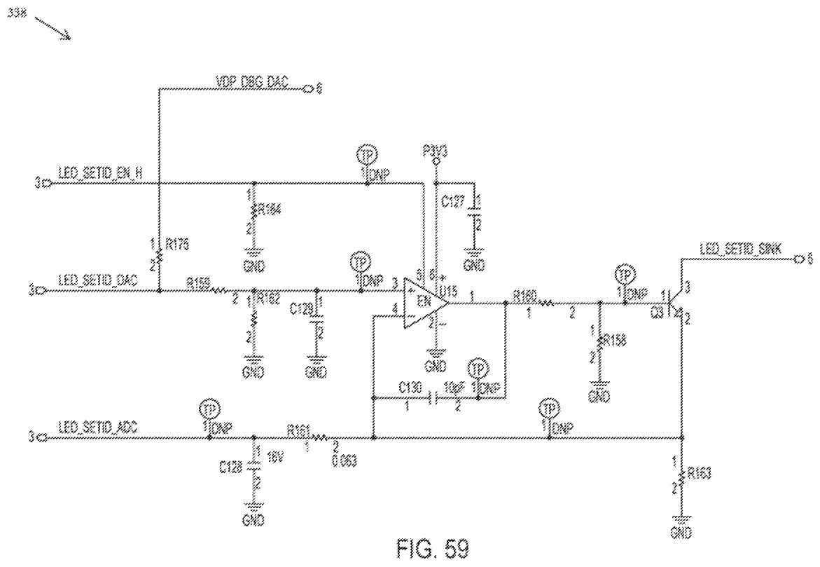

FIG. 59 shows a driver circuit 338 of the peristaltic pump 100 of FIG. 1 for driving the LEDs 165 of the flow-stop ID sensor 163. The driver circuit includes an op-amp U15 which is arranged in a negative feedback loop to a drive transistor Q3. The op-amp U15 drives its output such that a target voltage is achieved. This target voltage controls the base of the transistor Q3 which in turn causes the transistor Q3 to control for a constant current through resistor R163. This causes the current flowing from terminal 3 to terminal 2 of the transistor Q3 is be substantially constant. The schematic shows signal LED_SETID_ADC that is a voltage that directly correlates to the amount of current traveling through the LEDs. This voltage may be measured to verify that the LEDs' current consumption matches the commanded value. Using this measurement, a processor may detect some cases of shorted or open LEDs.

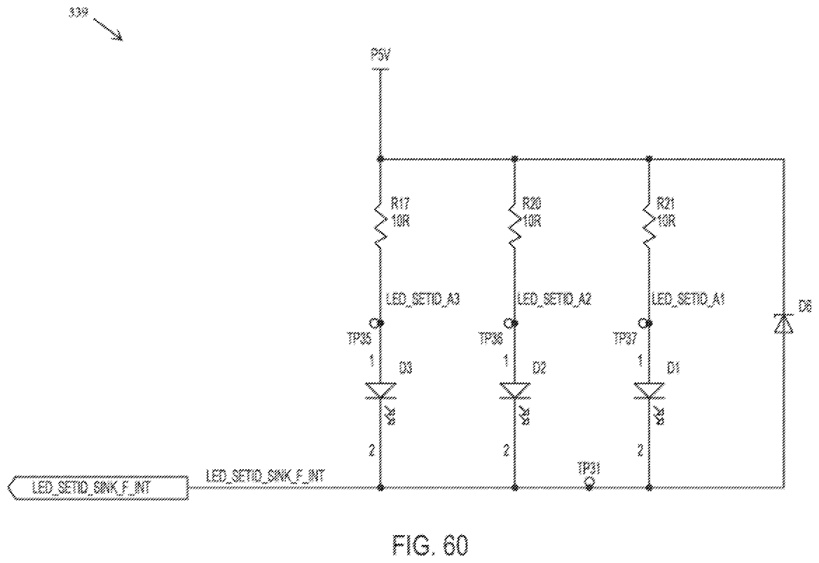

FIG. 60 shows an LED circuit 339 of the peristaltic pump 100 of FIG. 1 showing the arrangement of the LEDs 165 of the flow-stop ID sensor 163. The LED_SETID_SINK_F_INT pin is coupled to the output of the circuit of FIG. 59 which includes the same label. The constant current causes the LEDs D1, D2, D3 to generate visible light which is directed through the light pipe 155. The LEDs D1, D2, D3 may be the LEDs 165 shown in FIG. 55-56. In other embodiments, more LEDs (e.g., four) or fewer LEDs may be used as would be known to one of ordinary skill in the relevant art.

FIG. 61 shows an optical-sensor circuit 340 of the peristaltic pump 100 of FIG. 1 for sensing light received after light from the LEDs 165 has passed through the flow-stop ID holes 294 of the extension 274 of the flow stop 152. The optical sensor circuit of FIG. 61 uses a linear detector shown as IC U3. In some embodiments of the present disclosure, the IC U3 may be part number TSL1401CCS manufactured by ams AG of Tobelbader Strasse 308141, Premstaetten, Austria. In other embodiments, the sensor used may be part number LF1401 manufacturer by iC-Haus GmbH of Am Kuemmerling 18, 55294 Bodenheim, Germany. However, any suitable optical sensor 153 may be used including, but not limited to, other linear optical sensors. The IC U3 may be the optical sensor 153 shown in FIG. 55. Output of the IC U3 is sent to a processor via pin 6 of the IC U3 after being processed, e.g., by an analog-to-digital converter (not shown) that in some specific embodiments, is integrated into the processor. However, the analog-to-digital converter may be a separate integrated circuit from the processor.

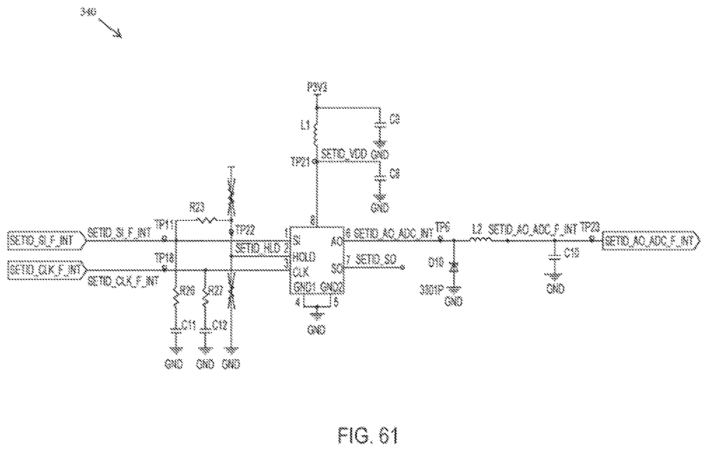

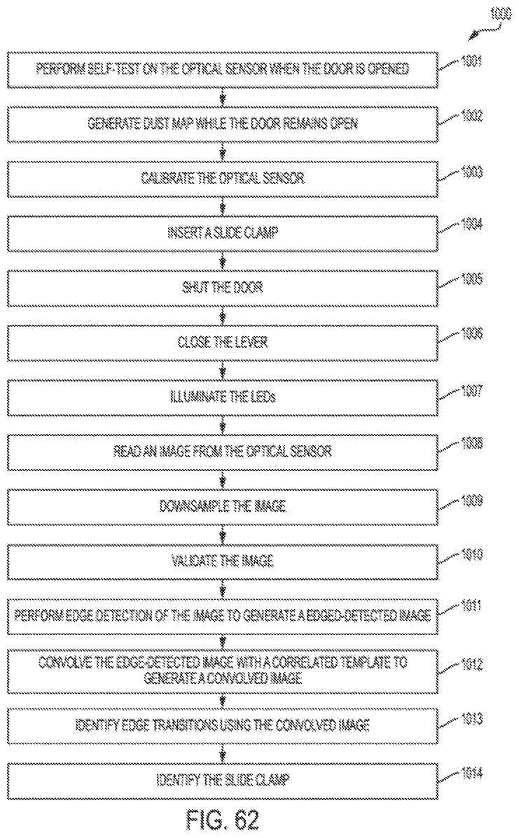

FIG. 62 shows a flow chart diagram 1000 illustrating a method of using data from the light sensor shown in FIG. 61 to identify a flow stop 152. The holes or absence of holes on the flow stop 152 may include 10 locations that correspond to 10 bits such that 10 different codes can be identified each of which corresponds to an infusion-set model number connected to the flow stop 152. The 10 codes can have a hamming distance of four relative to each other. And if any code is shifted to the left or right, the shifted code will have a hamming distance of three when the shift is less than 3 and a hamming distance of two when the shift is greater than or equal to 3. The codes may have an even number of ones and zeroes, e.g., 6/4 or 8/2. The codes may have at least six transitions from 1 to 0 or from 0 to 1.

The method includes Acts 1001-1014. Act 1001 performs a self-test on the optical sensor when the door 102 is opened. The optical sensor may be 128 pixels wide and each bit may be no less than 11 pixels wide. Act 1002 generates a dust map while the door 102 remains open. Act 1003 calibrates the optical sensor. Act 1004 inserts a flow stop. Act 1005 shuts the door 102. Act 1006 closes the lever. Act 1007 illuminates the LEDs. Act 1008 reads an image from the optical sensor. A PI controller may control the exposure so that a mean image intensity is at or close to a mid-range value or other predetermined value. Act 1009 downsamples the image. For example, each grayscale pixel in the image may be downsampled from 12 bits to 8 bits. Act 1010 validates the image. For example, variance and mean values must be within predetermined ranges to be validated. Act 1011 performs edge detection of the image to generate an edged-detected image. The edge detection may be performed using a modified Prewitt kernel with a kernel function of {−1, −2, −3, 0, 3, 2, 1}. Act 1012 convolves the edge-detected image with a correlated template to generate a convolved image. Act 1013 identifies edge transitions using the convolved image. An area of the highest intensity may be considered to be a center of a bit. Thereafter, a location is based upon fixed distances to the left and/or right where values are expected to be. That is, bit indices are used to sample the original image with a threshold value to determine whether a location is a ‘1’ or ‘0’. Each value is an average of five pixels centered around the sample point, in some specific embodiments. Act 1014 identifies the flow stop. A lookup table may be used to correspond values with infusion set part numbers.

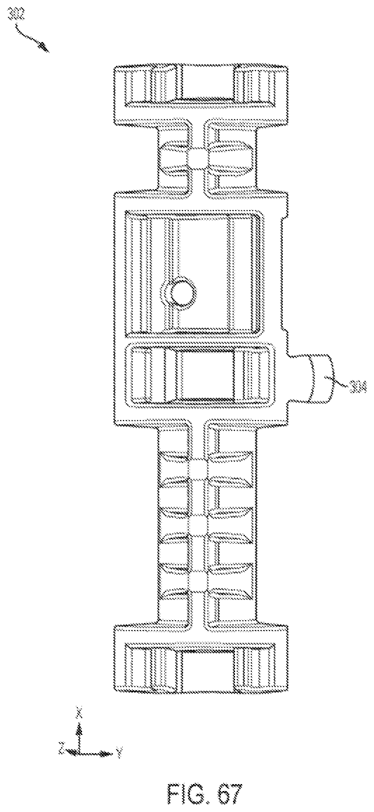

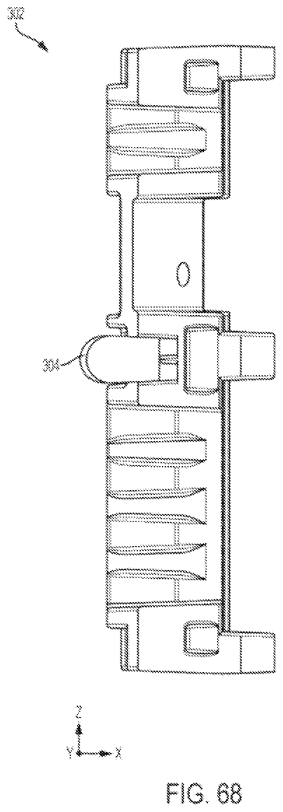

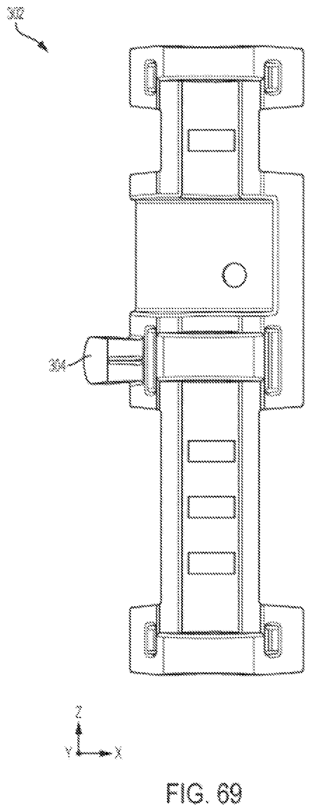

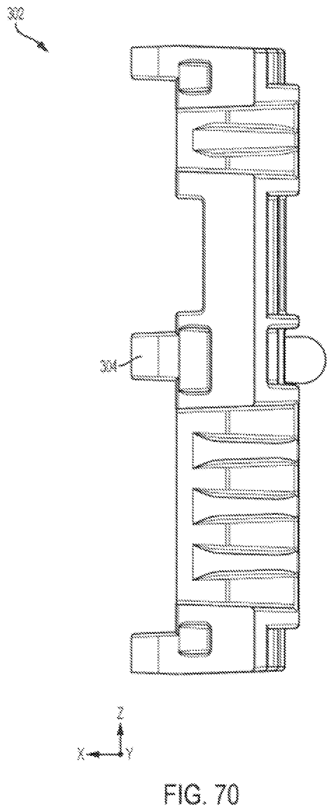

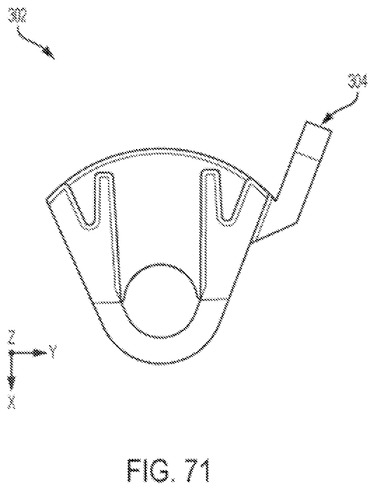

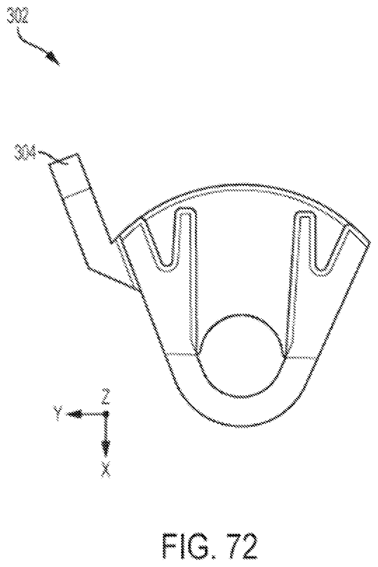

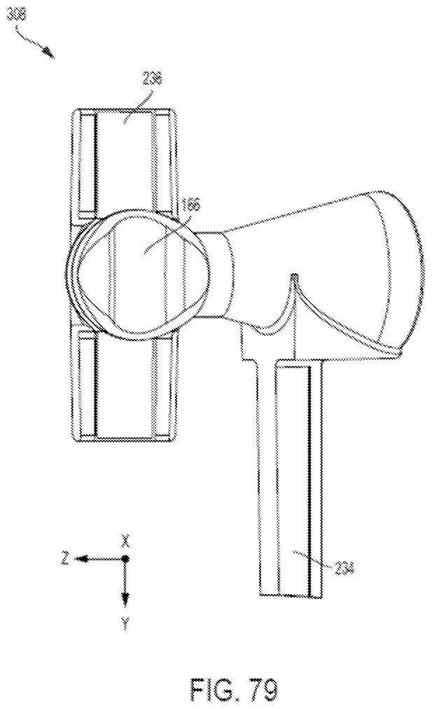

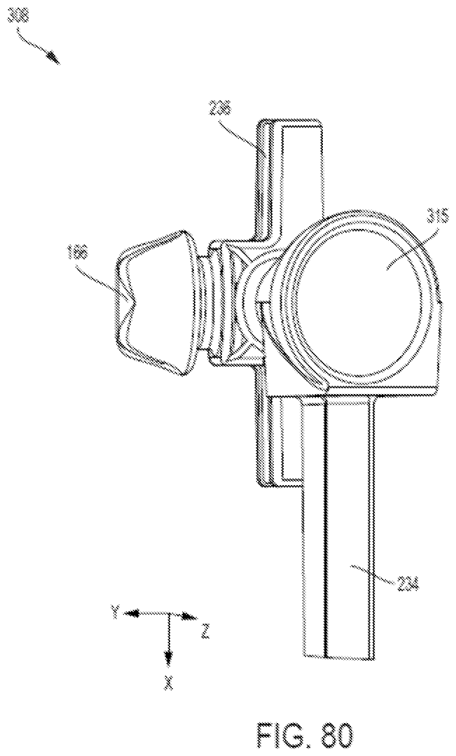

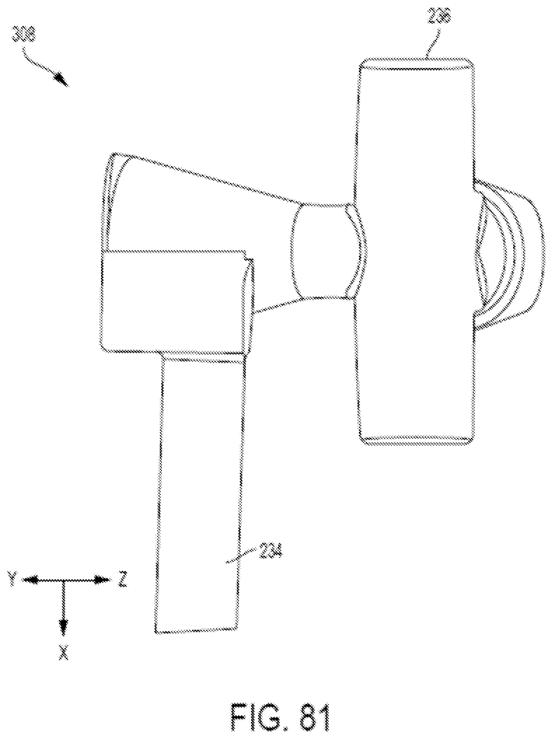

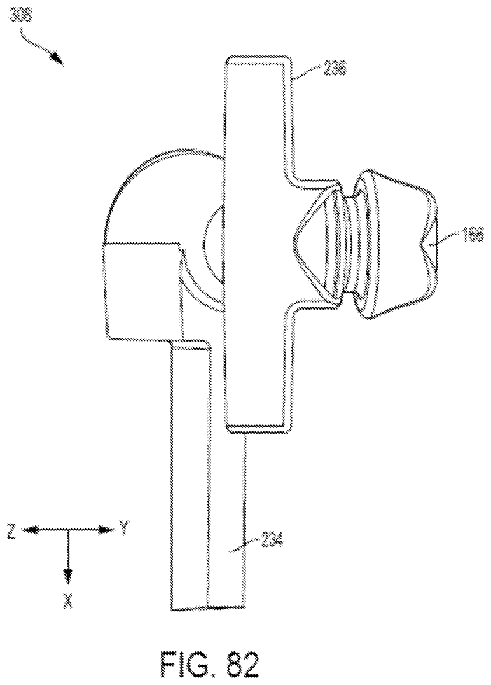

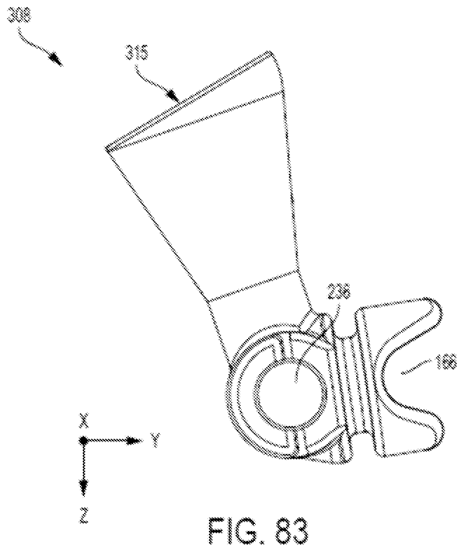

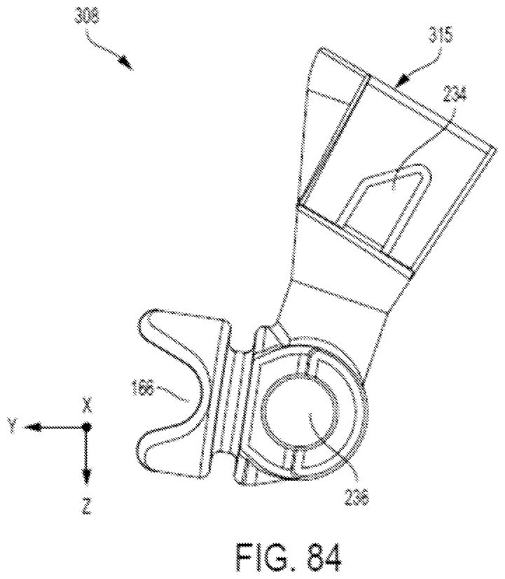

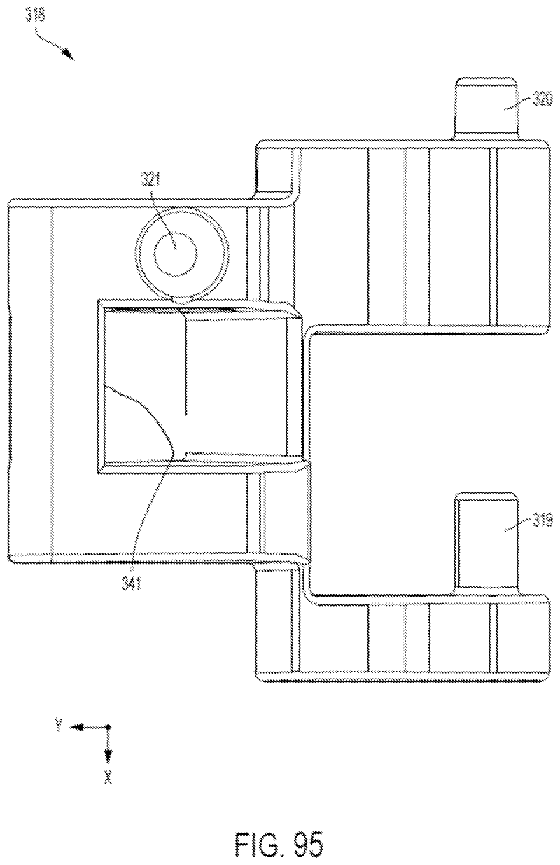

Referring generally to the drawings, FIGS. 63-96 show an alternative embodiment of the peristaltic pump 100 of FIG. 1 where an alternative lift cam 121, an alternative mechanical linkage between the shaft and carriage 150, and an alternative door catch 308 are used and is labeled generally as peristaltic pump 300.

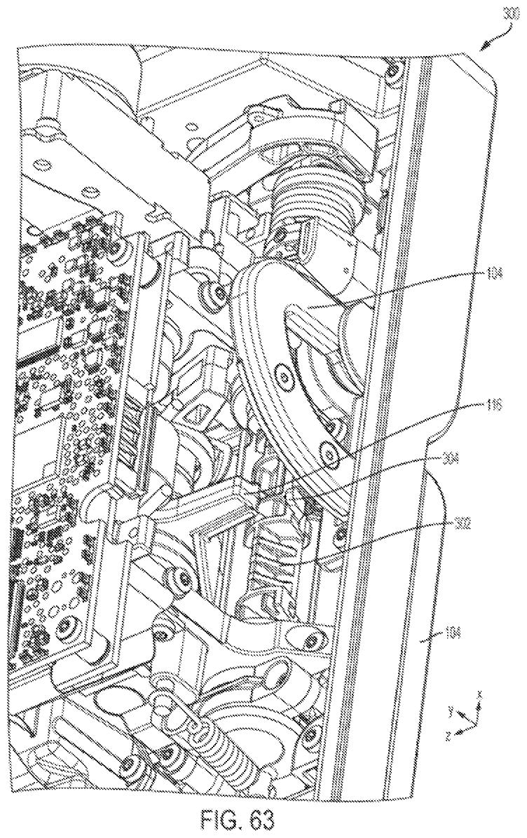

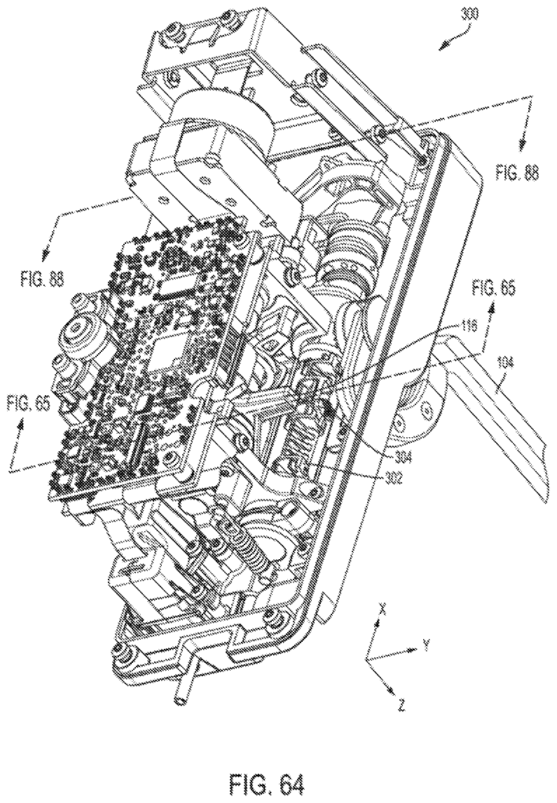

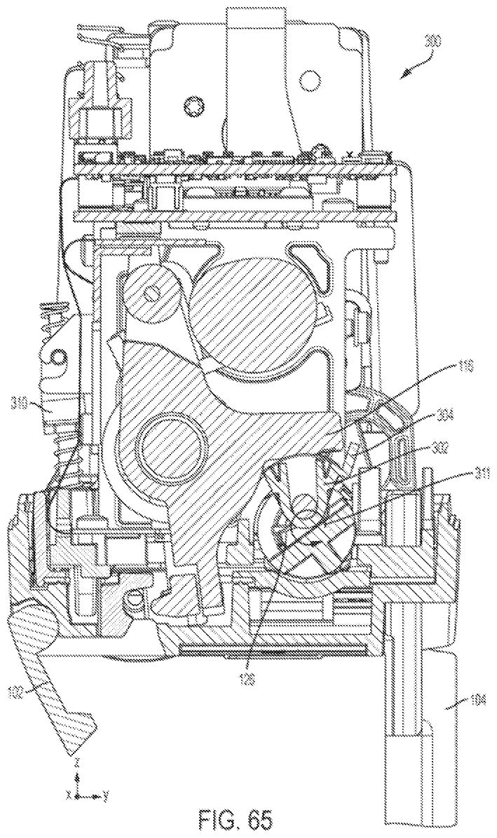

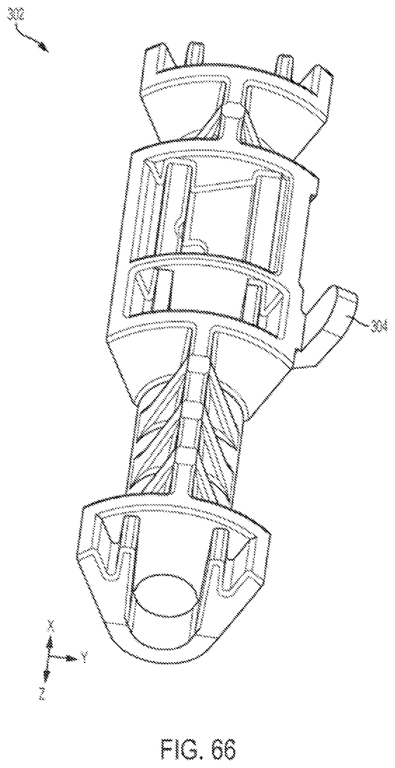

FIG. 63 shows a rear view of the peristaltic pump 300 with the rear cover removed. A lift cam 302 is shown and includes a flange 304. The flange 304 limits the movement of the lift cam 302 toward the spring-biased plunger 116. FIG. 64 shows another view of the peristaltic pump 300 of FIG. 63 to illustrate the operation of the lift cam 120 by showing the lever 104 in the open position. The lift cam 302 is rotated into a lifting position, but, as is shown in FIG. 64, the flange 304 prevents the lift cam 302 from slipping under the spring-biased plunger 116. FIG. 65 shows a cross-sectional view of the lift cam 120 of the peristaltic pump 300 of FIG. 63 when the lever 104 is in the open position. As shown in FIG. 65, the flange 304 prevents the lift cam 302 from slipping beyond a predetermined rotational angle. The lift cam 302 is biased by a cam-lifter torsion spring 126 in the direction of arrow 311. FIGS. 66-72 show the lift cam 120 of the peristaltic pump 300 of FIG. 63 from various viewing angles.

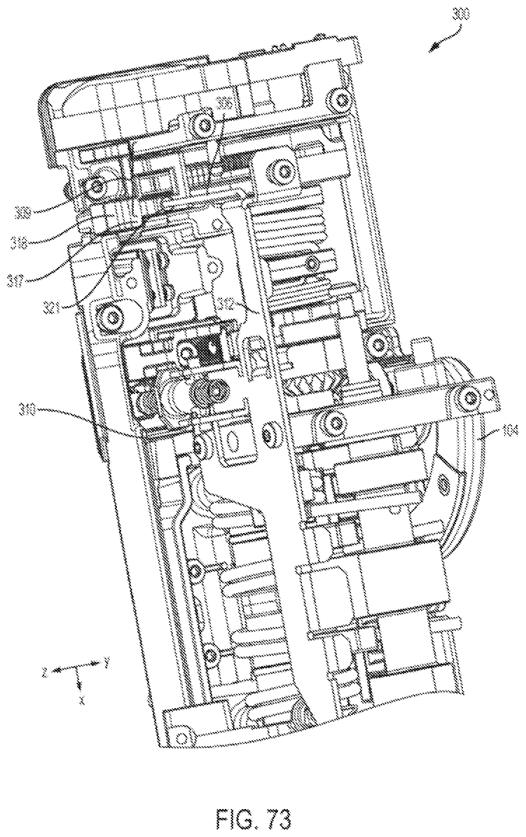

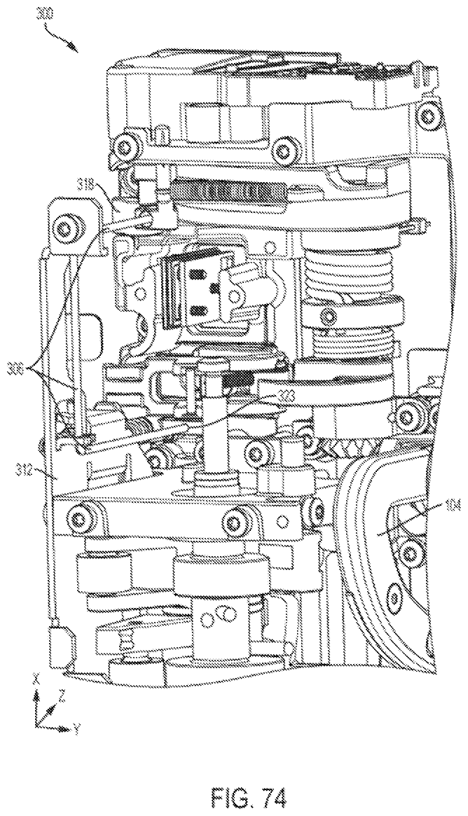

FIG. 73 shows the peristaltic pump 300 of FIG. 63 from a back view to show a door-catch linkage bar 306 between the door catch 308 and a linear ratchet 309. A door-catch spring 310 is coupled to the door-catch linkage bar 306 and the linear ratchet 309. The door-catch linkage bar 306 can rock back-and-forth because it is pivotally coupled to a frame 312. The door-catch spring 310 operates using an over-center action as described above which makes the door catch 308 bi-stable. FIG. 74 shows the peristaltic pump 300 of FIG. 63 to provide another view of the door-catch linkage bar 306 between the door catch 114 and a linear ratchet 309. As is shown in FIG. 74, a central span of the door-catch linkage bar 306 is rotatably coupled to the frame 312 so that actuation of the door catch 308 causes the linear ratchet 309 to change states. The linear ratchet 309 can be in a ratcheting state or in a non-ratcheting state. In the ratcheting state, the linear ratchet 309 can Act as a lock to prevent rotation of the carriage 150. That is, the linear ratchet 309 in the peristaltic pump 300 performs the locking action that is performed by the pawl 154 in the peristaltic pump 100 of FIG. 1. The linear ratchet 309 also includes a pawl 318 that locks the main shaft 118 via a carriage linkage bar 335 rather than directly acting on the carriage 150.

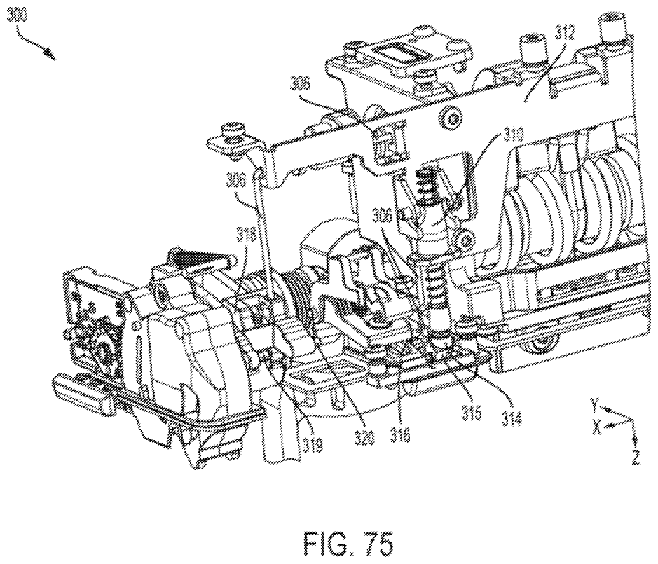

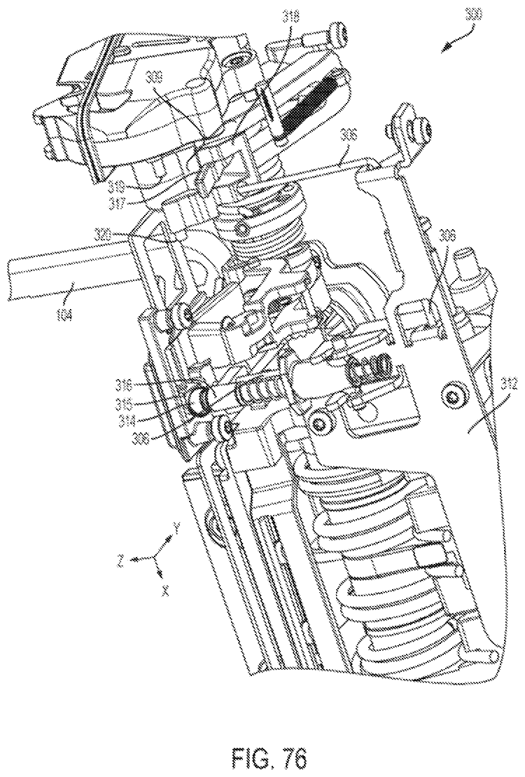

FIG. 75 shows a close-up view of the interface of the door-catch spring 310 and the door catch 308. FIG. 75 also shows the door-catch linkage bar 306 of the peristaltic pump 300 of FIG. 63 with a door catch 308 in the door 102 open position and the lever 104 in the open position. As can be seen, the door-catch spring 310 includes a ball 314 that interfaces with a socket 315 to form a ball-and-socket joint 316. When the door 102 is open, the door catch 308 can be in a position such that the door-catch linkage bar 306 has actuated the linear ratchet 309 to a ratcheting state. In the ratcheting state, the linear ratchet 309 prevents the main shaft 118 from rotating when a user attempts to close the lever 104 thereby preventing the user from closing the lever 104 while the door 102 remains open. FIG. 76 shows the same close-up view of FIG. 75 but with the door catch 308 in the door-shut position and the lever 104 in the open position. When a user shuts the door 102, it actuates the door catch 308, which actuates the door-catch spring 310, which actuates the door-catch linkage bar 306 which places the linear ratchet 309 in the non-ratcheting state. That is, the lever 104 can now be shut by the user because the door 102 is closed. FIG. 77 shows the same close-up view of FIG. 76 but after the lever 104 was actuated to the closed position. The lever 104 could be shut because the linear ratchet 309 was in the non-locking position when the lever 104 was actuated closed as described above

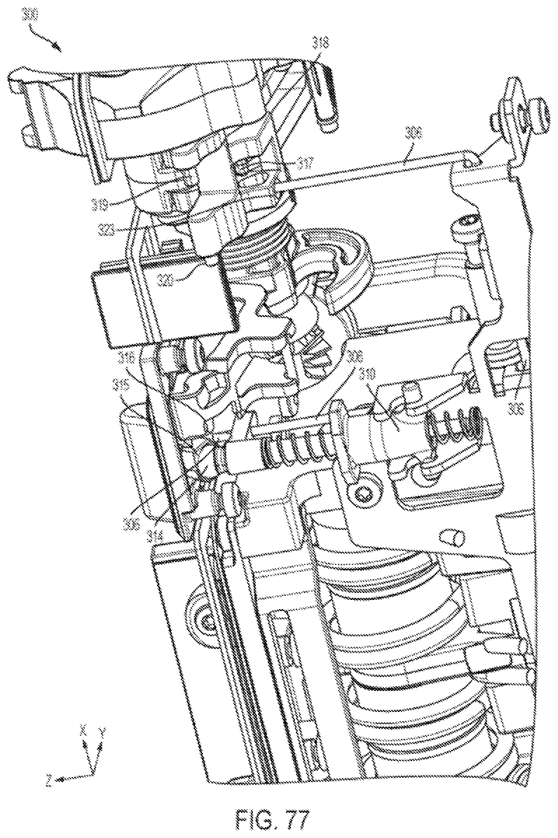

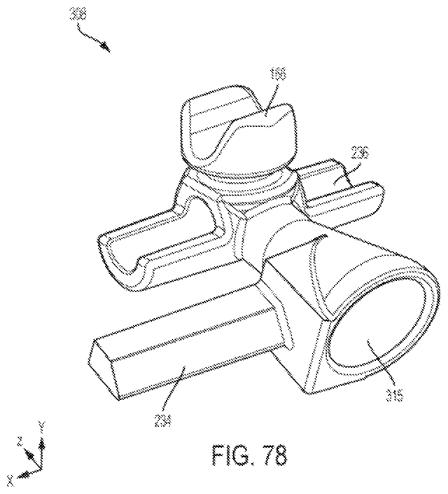

FIG. 78-84 show several views of the door catch 308 including the socket 315 that receives the ball 314 from the door-catch spring 310. The door catch 308 of FIGS. 78-84 operates in the same manner as the door catch 308 shown in FIG. 25; however, the door catch 308 has a socket 315 to connect to an the door-catch spring 310 rather than a door-catch anchor 232 as shown in FIG. 25. The door catch 308 includes a door catch 114, and the door catch 308 includes a pin catch 166, a door-catch hold 234, and a channel 236 to allow the door catch 308 to pivot.

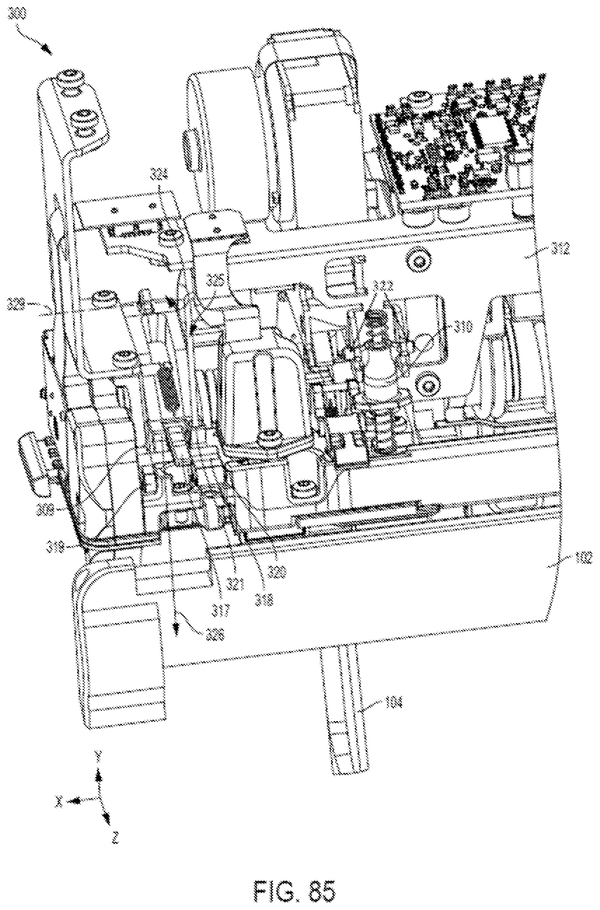

FIG. 85 shows a close-up view of the linear ratchet 309 when the door 102 is open and the lever 104 is open. The linear ratchet 309 includes a toothed linkage bar 317 and a pawl 318 that can be rotated along pivots 319, 320 so that the pawl 318 can engage or disengage with the toothed linkage bar 317. The pawl 318 is coupled to the linkage bar 325 through a pawl hole 321. The linkage bar 325 may slide through the pawl hole 321.

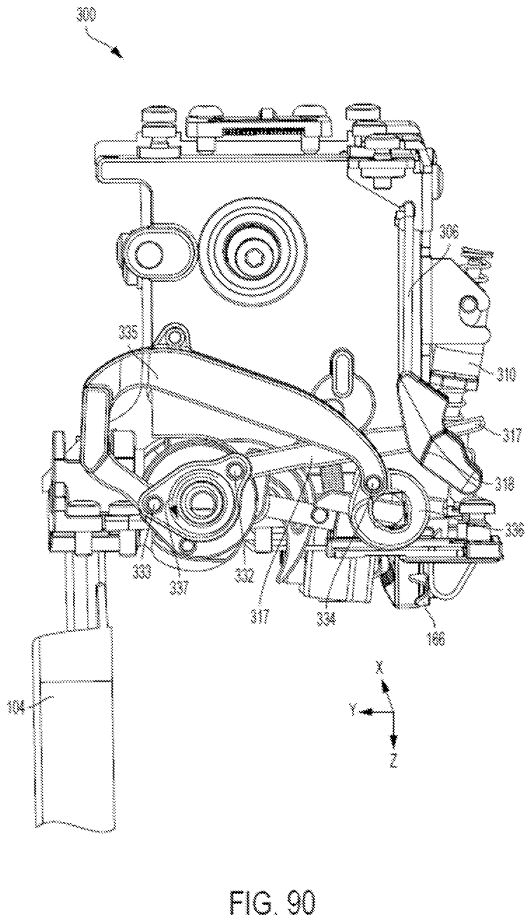

The pawl 318 includes a pivotable end that is coupled to the pivots 319, 320 and is configured to so that the an engagement end, such as a tooth 341 (see FIGS. 90-92) can pivot to engage the toothed linkage bar 317 or disengage the toothed linkage bar 317. The door-catch linkage bar 306 can rotate around an axis 329. Because the door-catch linkage bar 306 is in sliding engagement with the pawl hole 321 movement of the door-catch linkage bar 306 around an axis 329 can raise or lower the tooth 341 of the pawl 318 to engage or disengage the toothed linkage bar 317.

As shown in FIG. 85, the door catch 114 is in the open door 102 position which actuates the door-catch spring 310 to pivot along spring pivots 322. Because the door-catch spring 310 is coupled to the door-catch linkage bar 306 via a door-catch spring hole 323 (see FIG. 77), when the door-catch spring 310 is actuated to the door-open position, the linkage bar 325 is rotated along arrow 324 which in turn actuates the linkage bar 325 coupled to the pawl 318. The tooth 341 of the pawl 318 is actuated in direction of the arrow 326. This latching state of the pawl 318 means the pawl 318 is pivoted such that a tooth 341 of the pawl 318 engages with the toothed linkage bar 317 to prevent the user from closing the lever. That is, when the tooth 341 engages with the toothed linkage bar 317, the linear ratchet 309 is in the locking state

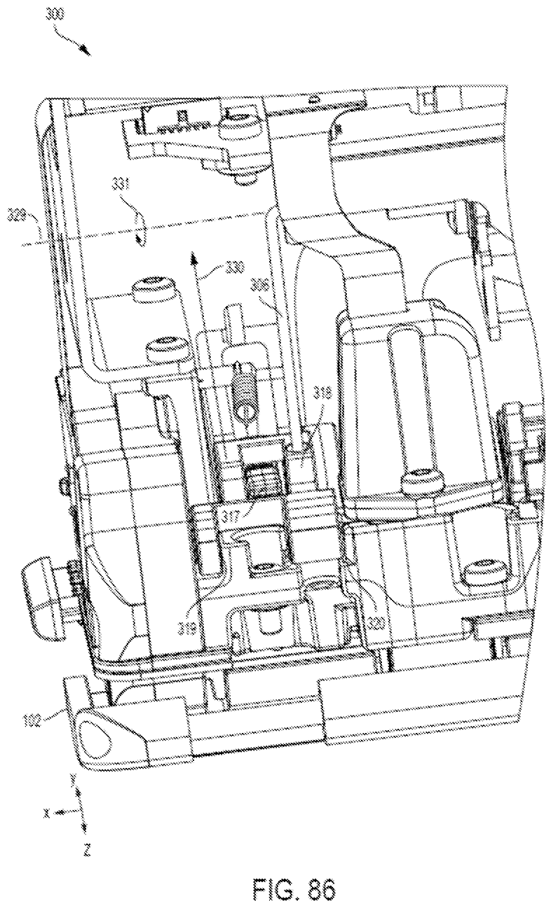

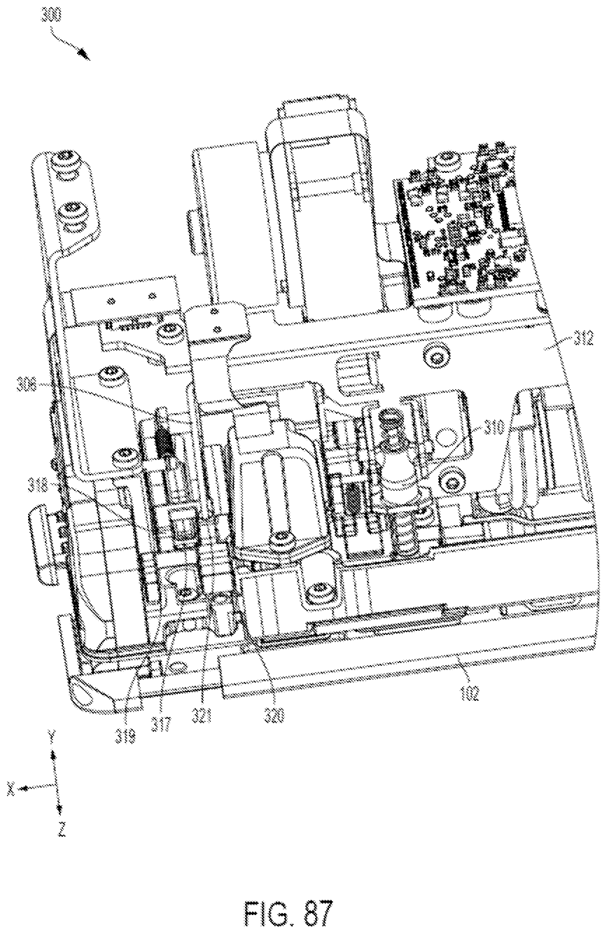

FIG. 86 shows a close-up view of the linear ratchet 309 when the door 102 is closed and the lever 104 is open. The tooth 341 of the pawl 318 has actuated in the direction of arrow 330 by rotation of the door-catch linkage bar 306 in the direction indicated by arrow 331. Actuation of the tooth 341 of the pawl 318 away from the toothed linkage bar 317 thereby disengages the pawl 318 from the toothed linkage bar 317 thereby making the linear ratchet 309 to be in the non-latching state. A user can close the lever 104 when the linear ratchet 309 is in the non-latching state as shown in FIG. 87. That is, FIG. 87 shows a close-up view of the linear ratchet 309 when the door 102 is closed and the lever 104 is also closed.

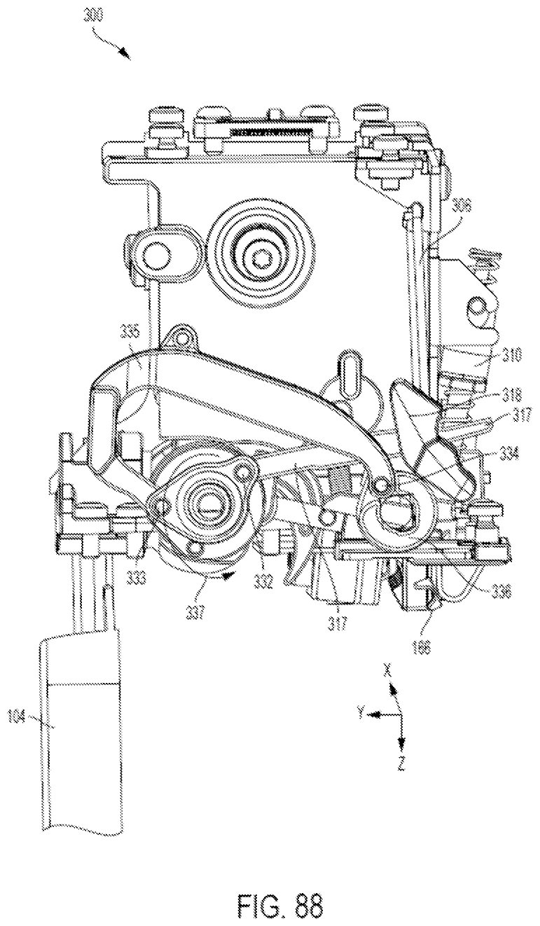

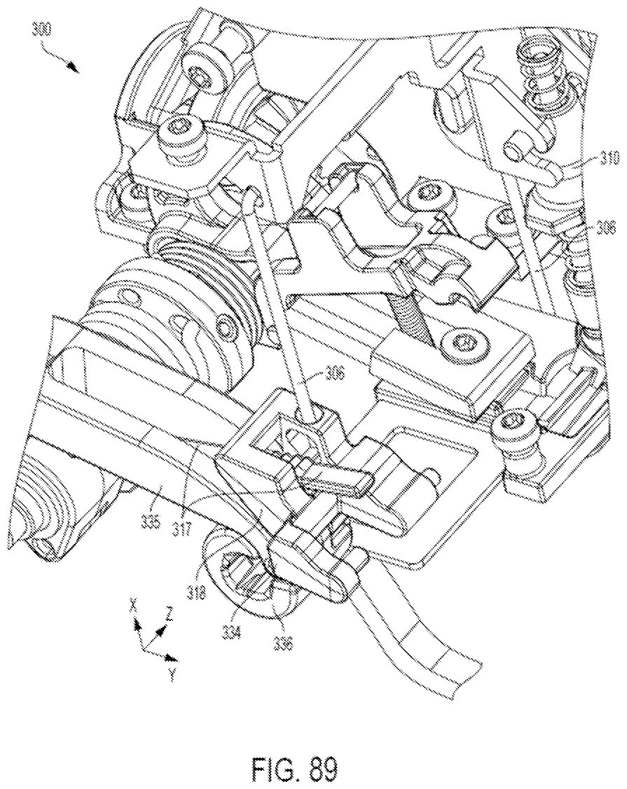

FIGS. 88-89 show the peristaltic pump 300 of FIG. 63 with some parts removed to illustrate the mechanical linkage between the main shaft and the carriage 150 where the door catch 308, the door 102, and the lever 104 are in the open position. The mechanical linkage includes the toothed linkage bar 317 that is coupled to the main shaft 118 via a first pin pivot 332. The toothed linkage bar 317 is only connected at one end (i.e., via the first pin pivot 332). The mechanical linkage also includes a carriage linkage bar 335 where one end is connected to the main shaft 118 via a second pin pivot 333 and to a carriage-shaft collar 336 via a third pin pivot 334.

As previously mentioned, when the door catch 114 is in the door-open position, the tooth 341 of the pawl 318 engages with the toothed linkage bar 317. As can be seen in FIG. 88, in this position, the toothed linkage bar 317 cannot be actuated toward the main shaft 118 when a user attempts to close the lever 104 because the main shaft 118 is prevented from being rotated in direction 337 because the toothed linkage bar 317 is locked by the pawl 318. This prevents the user from closing the lever 104 prior to the door 102 being closed.

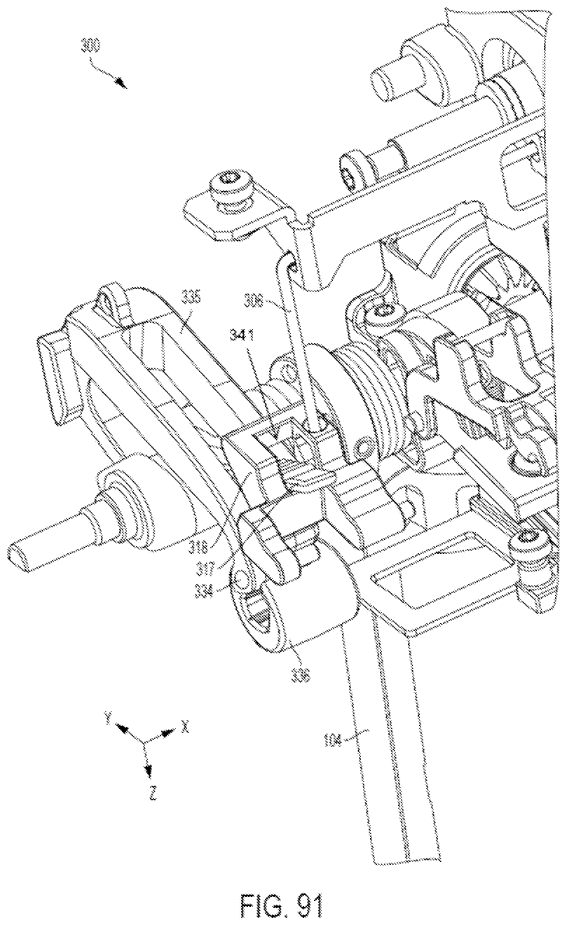

FIGS. 90-91 show the peristaltic pump 300 of FIG. 63 with some parts removed to illustrate the mechanical linkage between the shaft and the carriage 150 where the door 102 and the door catch 308 are in the closed position and the lever 104 is in open position. As can be seen, the tooth 341 of the pawl 318 has been actuated away from the toothed linkage bar 317 thereby allowing the toothed linkage bar 317 to retract toward the main shaft 118. Thus, a user can now actuate the lever 104 to the closed position.

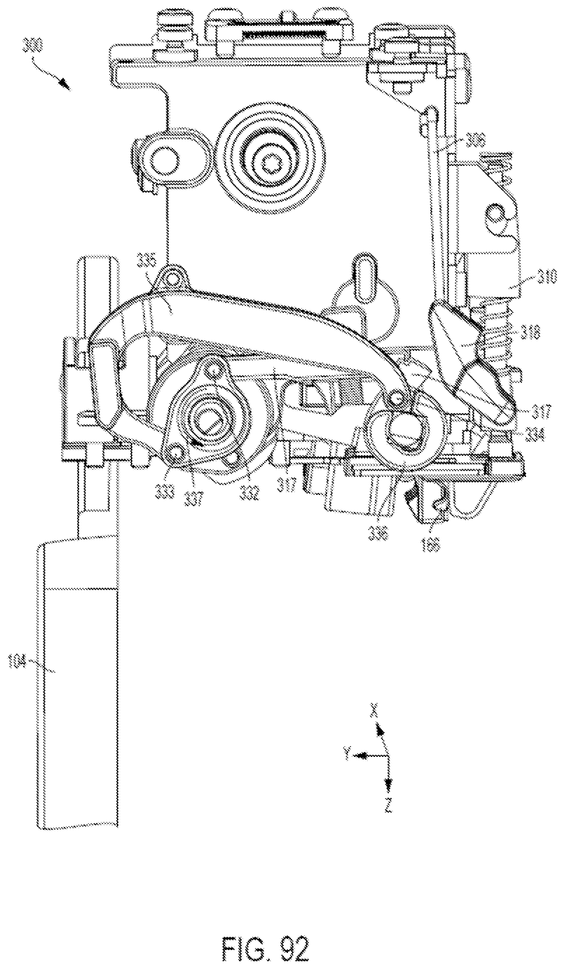

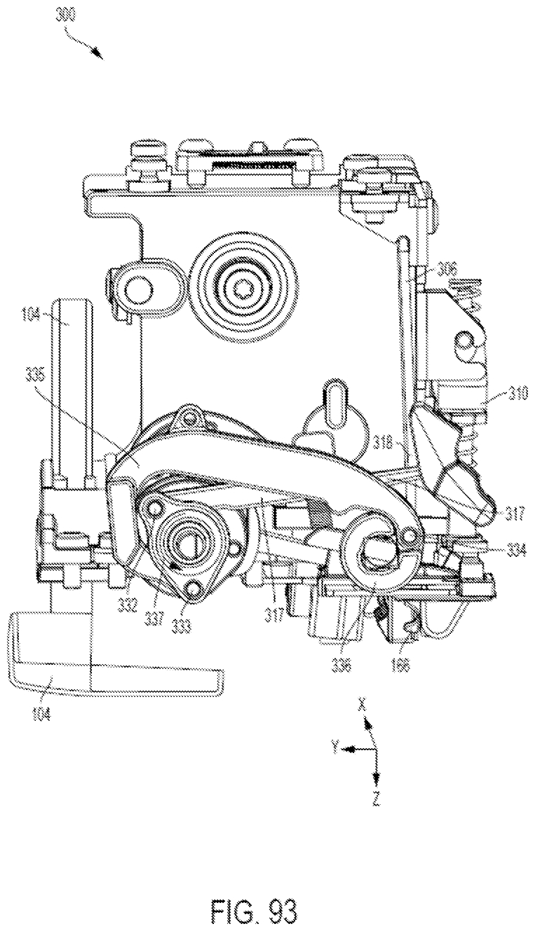

FIG. 92 shows the peristaltic pump 300 of FIG. 63 with some parts removed to illustrate the mechanical linkage between the shaft and the carriage 150 where the door 102 and the door catch 114 are in the closed position while the lever 104 is between the open and closed position. Because the main shaft 118 has partially rotated in direction 337 by actuation of the lever 104, the carriage linkage bar 335 has pulled on the carriage-shaft collar 336 such that it is rotated along with the carriage 150 attached thereto. FIG. 93 shows the peristaltic pump 300 of FIG. 63 when the lever 104 has been closed. As can be seen, the carriage-shaft collar 336 has been fully rotated such that the carriage 150 is now in the position as shown in FIG. 36.

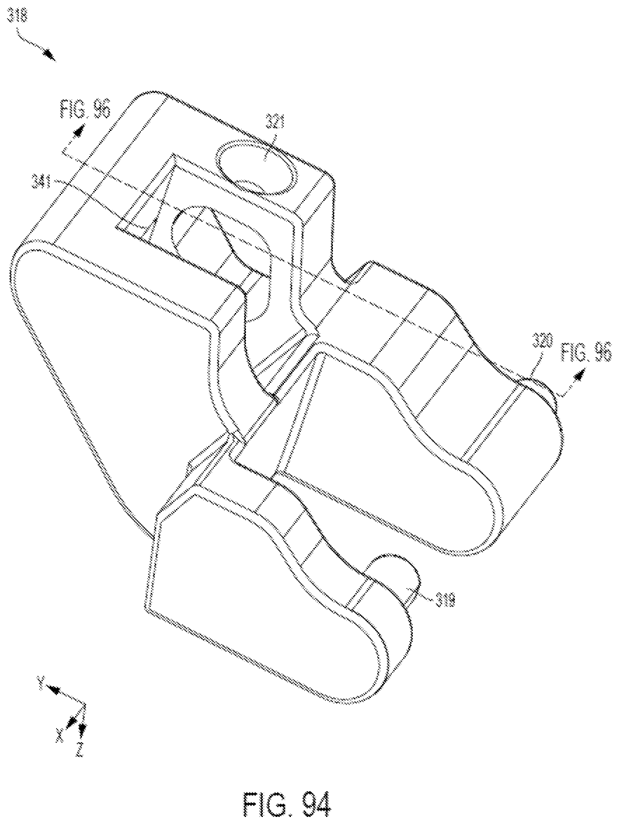

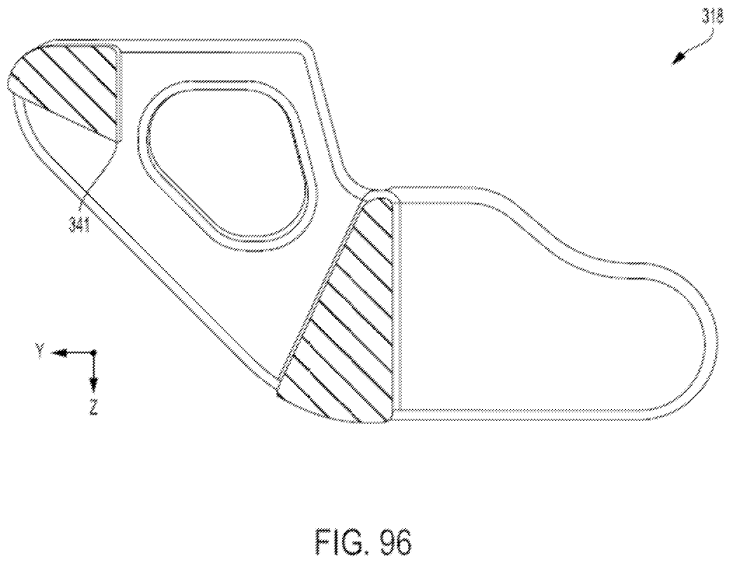

FIG. 94-96 show the pawl 318 of the peristaltic pump 300 of FIG. 63 from several views. FIG. 96 shows a cross-sectional view of the pawl 318 along the view indicated in FIG. 94. In FIG. 96, a tooth 341 is shown that engages with the teeth of the toothed linkage bar 317 shown above in FIGS. 73-93.

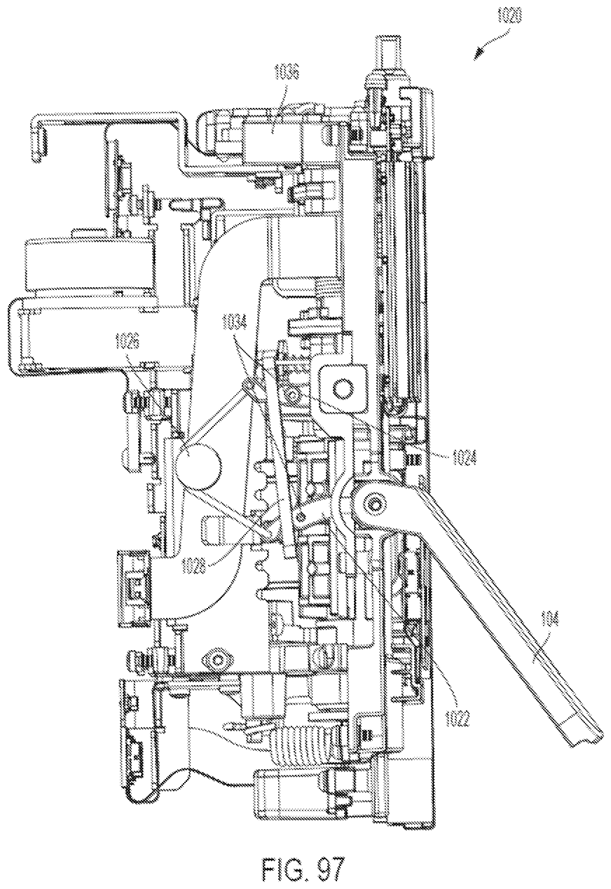

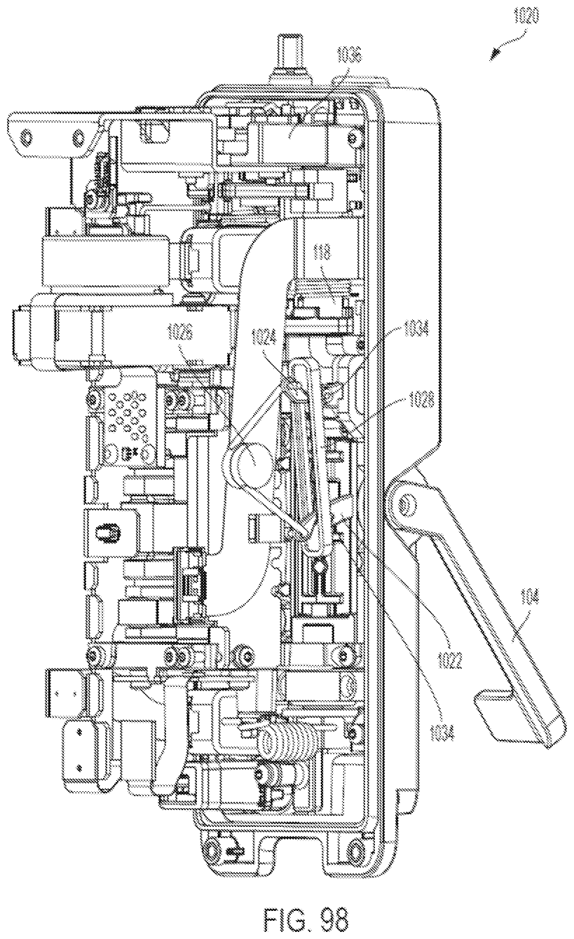

FIGS. 97-98 shows an alternative embodiment of the peristaltic pump 10200 where an alternative mechanical assembly 1021 between the lever 104 and the main shaft 118 is used. FIGS. 97-98 also shows an embodiment of the peristaltic pump 10200 where an alternative carriage 1036 is used. The peristaltic pump 10200 provides resilience between the lever 104 and a main shaft 118 via a spring 1026. The spring 1026 is a torsion spring, in some specific embodiments.

As shown in FIG. 97, when in operation the spring 1026 provides resilience such that the spring 1026, via its ends, urges the first linkage 1022 and the second linkage 1024 outward toward the ends of the track 1028. When the ends of the spring 1026 remain at the ends of the track 1028, the track 1028 moves when the lever 104 is actuated which moves the second linkage 1024. That is, the first linkage 1022 and the second linkage 1024 remain at a predetermined distance from each other at a respective end of the track 1028 when the spring 1026 maintains the first linkage 1022 and the second linkage 1024 at a maximal distance between each other in the track 1028. However, if the door 102 is open, the main shaft 118 (see FIG. 98) cannot be rotated because it is effectively locked. Therefore, the spring 1026 can become compressed as described below. The guides 1034 are configured to guide the linkages 1022, 1024 along the track 1028. Each of the linkages 1022, 1024 includes guides 1034 to keep the linkages 1022, 1024 disposed on a predetermined position on the track 1028.

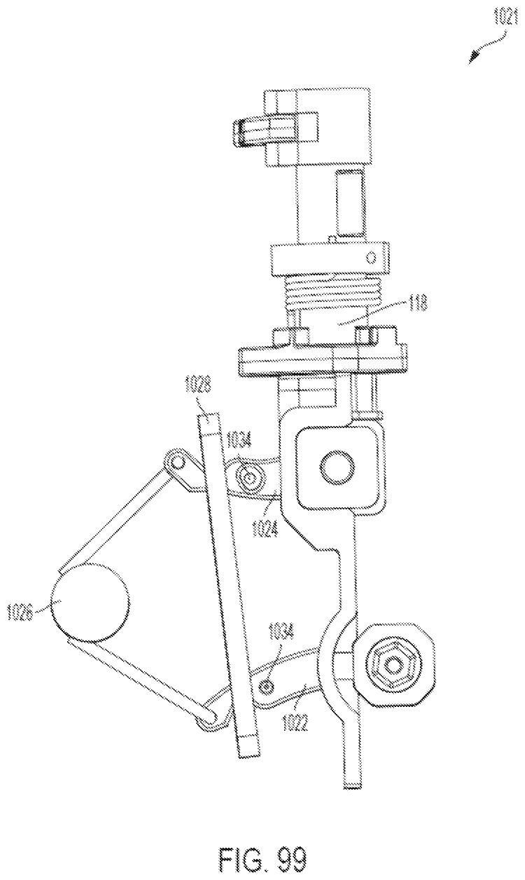

Referring now to FIG. 99, when the lever 104 is actuated, the first linkage 1022 applies a force to the spring 1026; but, when the second linkage 1024 is locked (because, for example, the carriage is locked because the door 102 is open), the first linkage 1022 approaches the second linkage 1024 as guided by the track 1028 as the spring 1026 compresses. Eventually, the first linkage 1022 will engage with the second linkage 1024, in which case, the lever 104 will be stopped by a hard stop.

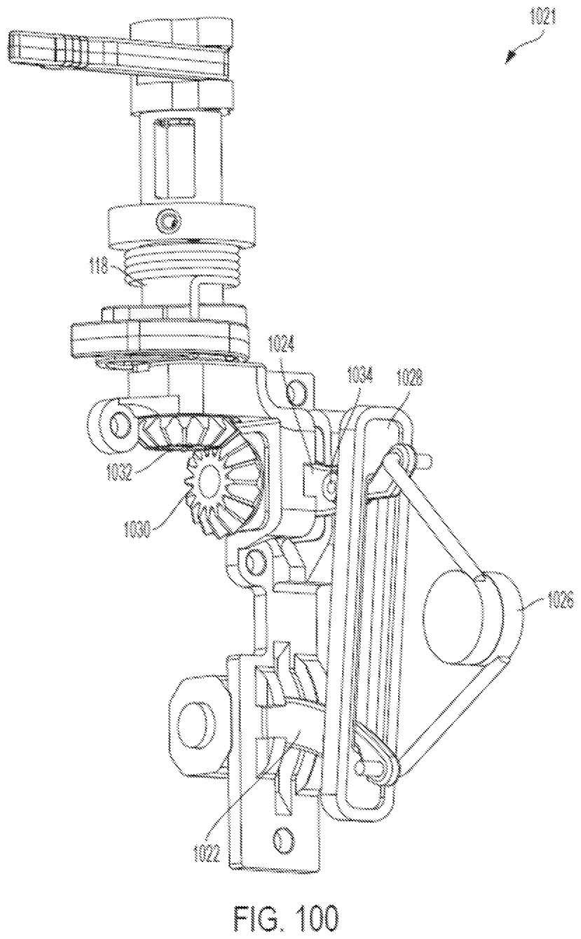

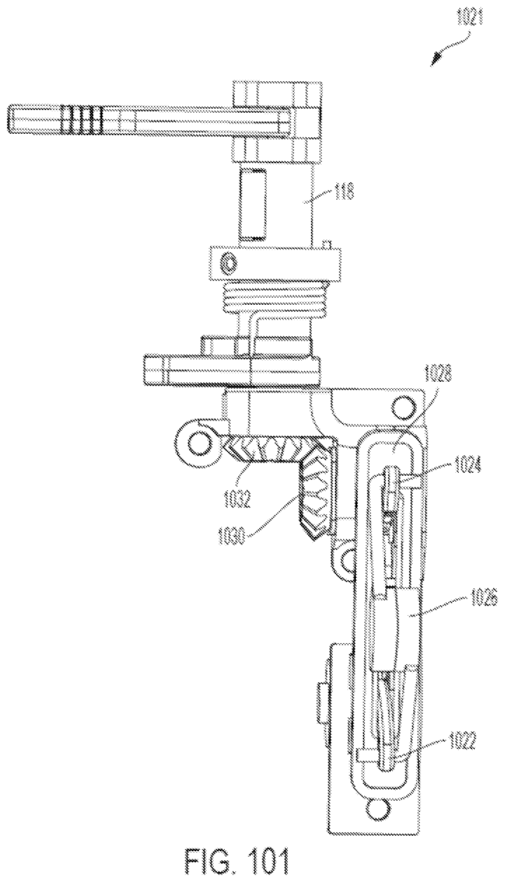

The lever 104 can pivot to actuate a first linkage 1022. When the main shaft 118 is not locked, this actuation also actuates the second linkage 1024. As shown in FIGS. 100-101, actuating the second linkage 1024 rotates the first bevel gear 1030, which in turn rotates a second bevel gear 1032. The second bevel gear 1032 is attached to the main shaft 118. The lower portion of the shaft may extend from the second bevel gear 1032 by being attached to the second bevel gear 1032 thereto (not shown in FIGS. 100-101). The first linkage 1022 slides along the track 1028 when the main shaft 118 is unable to rotate thereby compressing the spring 1026. Additionally or alternatively, the second linkage 1024 slides along the track when the main shaft 118 is unable to rotate.

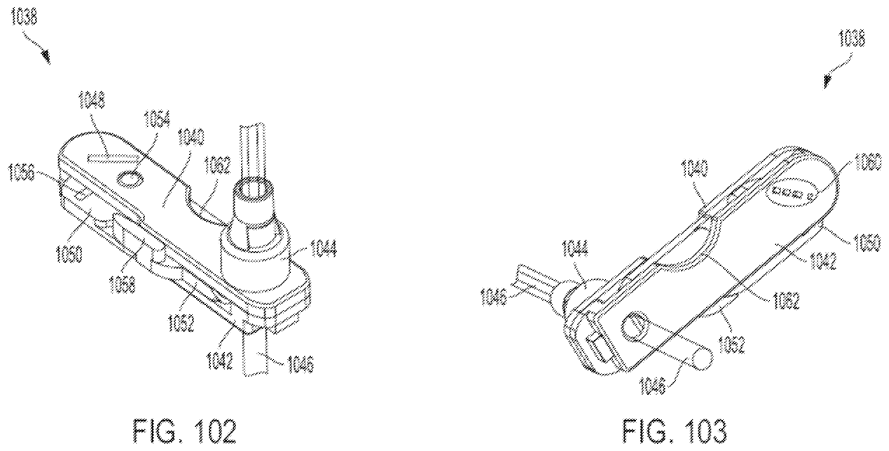

FIGS. 102-105 show several views a flow-stop assembly 1038 in accordance with an embodiment of the present disclosure. The flow-stop assembly 1038 includes a top housing 1040 and a bottom housing 1042. A tube 1046 is coupled to the flow-stop assembly 1038 via a tube coupling 1044. The flow-stop assembly 1038 can occlude fluid flow through the tube 1046 or can allow fluid to flow freely therethrough.

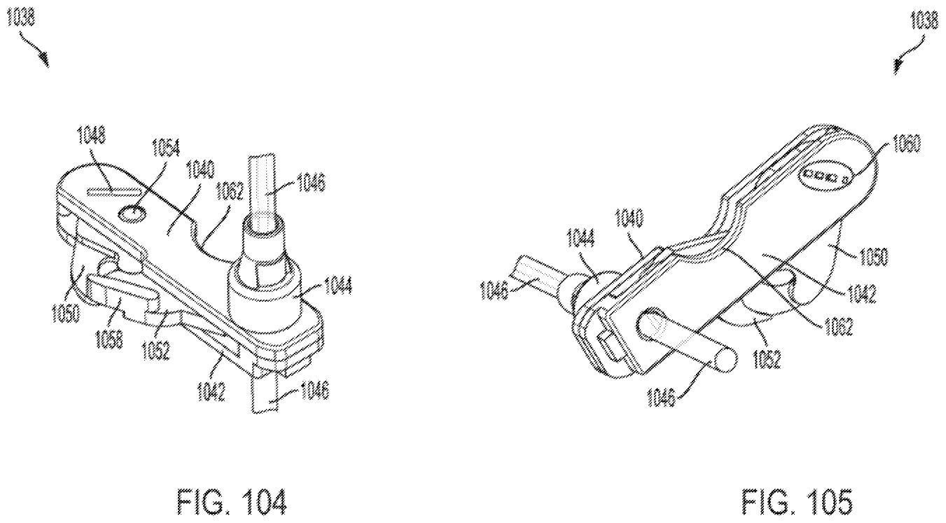

Non-occluded and occluded fluid flow may be effected through the tube 1046 via actuation of a first link 1052 and a second link 1050. FIGS. 102-103 show the flow-stop assembly 1038 in the occluding position and FIGS. 104-105 show the flow-stop assembly 1038 in the non-occluding position. When the flow-stop assembly 1038 is in the occluding position, as shown in FIGS. 102-103, a user can press on the first link 1052 via a finger groove 1062 to actuate the second link 1050 and first link 1052 to the non-occluding position as shown in FIGS. 104-105. Likewise, when the flow-stop assembly 1038 is in the non-occluding position as shown in FIGS. 104-105, a user can press on a flange 1058 to actuate the second link 1050 and first link 1052 to the occluding position as shown in FIGS. 102-103.

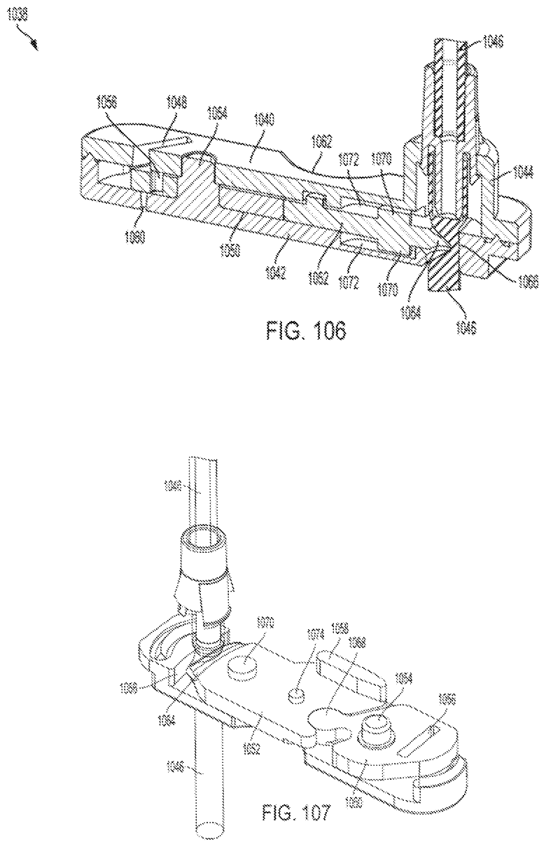

The flow-stop assembly 1038 also includes a housing aperture 1048 which can be used to sense the configuration of an identification aperture 1060, can be used to determine if the flow-stop assembly 1038 is loaded properly or improperly, and can be used to determine the configuration of the flow-stop assembly 1038 (e.g., the occluding vs. non-occluding position, etc.). The identification may take place as is described herein using optical recognition of a pattern of the identification aperture 1060. FIG. 106 shows a cross-sectional view of the flow-stop assembly 1038, which shows a pivot post 1054 about which the second link 1050 can pivot. When the first link 1052 and the second link 1050 are in the occluding position, as shown in FIG. 106, a plunger 1064 occludes a tube 1046 by wedging the tube 1046 between the plunger 1064 and a backstop 1066. A shutter aperture 1056 is shown which blocks or allow light to pass through depending whether or not the flow-stop assembly 1038 is in the occluding position or the non-occluding position.

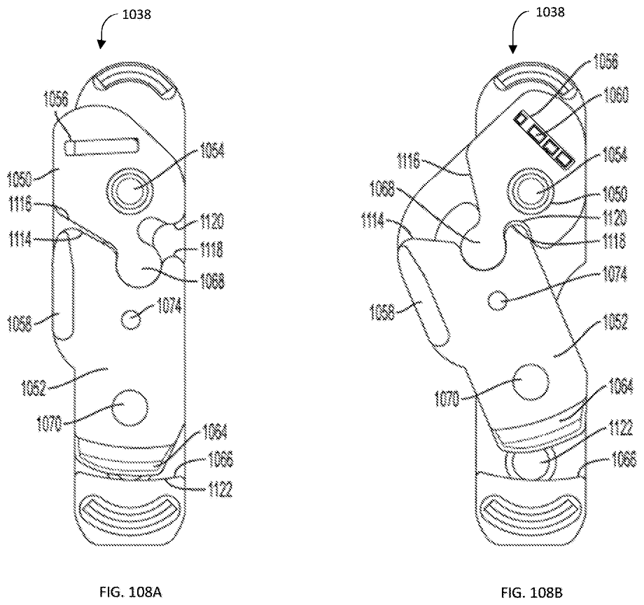

The second link 1050 pivots around the pivot post 1054. The first link 1052 is coupled to the second link 1050 via a ball-and-socket joint 1068 (see FIG. 107). As the first link 1052 is actuated, it is guided within a track 1072 by guides 1070. FIG. 108A shows the flow-stop assembly 1038 with the top housing 1040 removed while in the occluding position and FIG. 108B shows the flow-stop assembly 1038 with the top housing 1040 while in the non-occluding position. As is shown in FIG. 108A, when the first link 1052 is in the occluding position, the plunger 1064 is closer to the backstop 1066 and when the second link 1050 is in the non-occluding position, the plunger 1064 is a predetermined distance from the backstop 1066. The second link 1050 and the first link 1052 are coupled together via a ball-and-socket joint 1068. The guides 1070 positions the first link 1052 such that rotation of the second link 1050 along the pivot post 1054 translates to linear motion of the guides 1070 along the track 1072 as shown in FIGS. 110-114. FIGS. 108A-108B also illustrate how the position of the shutter aperture 1056 is positioned in different locations based upon the position of the second link 1050. FIGS. 110-114 show several views of the bottom housing 1042 of the flow-stop assembly 1038 including the track 1072. Please note the identification aperture 1060 where identification of the flow-stop assembly 1038 can be made, as described herein.

The first link 1052 includes a first contacting surface 1114 and a third contacting surface 1118. The second link 1050 includes a second contacting surface 1116 and a fourth contacting surface 1120. As shown in FIG. 108A, when the flow-stop assembly 1038 is in the occluding position, the first contacting surface 1114 contacts the second contacting surface 1116. As shown in FIG. 108B, when the flow-stop assembly 1038 is in the non-occluding position, the third contacting surface 1118 contacts the fourth contacting surface 1120. In some embodiments, a secondary guide 1074 can limit the movement of the first link 1052 via limiting the range of motion the secondary guide 1074 can travel within a secondary track 1076 (see FIG. 119).

Referring again to FIGS. 108A-108B, in some specific embodiments of the present disclosure, the compliance of the tube 1046 may make the flow-stop assembly 1038 bi-stable, with one stable configuration being the occluding position as shown in FIG. 108A and the other stable configuration being the non-occluding configuration as shown in FIG. 108B (a hole 1122 is shown in FIGS. 108A and 108B where the tube 1046 is positioned (see FIG. 107). In alternative embodiments, a spring or springs may be used to urge the second link 1050 and first link 1052 into the two bi-stable configurations.

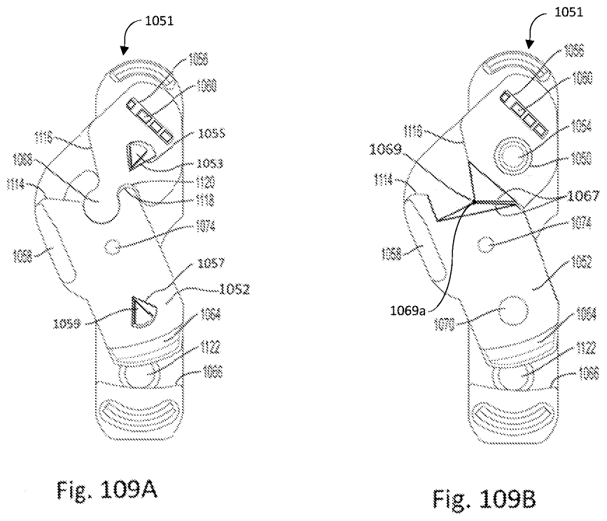

FIG. 109A shows an alternative embodiment of the flow-stop assembly 1051 of FIG. 108B with knife-edge pivots in accordance with an embodiment of the present disclosure. The knife-edge pivots may each includes a v-shaped member 1055, 1057 that interface with a v-shaped pivot surface 1053, 1059, respectively. The v-shaped pivot surfaces 1053, 1059 may be configured to limit movement of the v-shaped members 1055, 1057 to thereby limit their actuation to between two positions. The materials of the v-shaped pivot surfaces 1053, 1059 and/or the v-shaped members 1055, 1057 should have sufficient hardness and modulus characteristics.

FIG. 109B shows an alternative embodiment of the flow-stop assembly 1051 of FIG. 108B with a flexure 1069 including arms 1067 in place of a ball-and-socket joint 1068. The flexure 1069 would allow the first link 1050 and the second link 1052 to be molded by a single piece. The flexure 1069 in FIG. 109B is known as an X flexure, where two arms 1067 from each of link 1052 and 1050 connect at a joint 1069a. Other flexures may be used in place of the flexure 1069 such as a simple living hinge.

FIGS. 110-114 show the bottom housing 1042 of the flow-stop assembly 1038. As shown in in FIGS. 110-114, a flow-stop assembly 1038 may be substantially similar to the flow-stop assembly 1051 described herein except in the ways that are now described. The bottom housing 1042 may include: pivot post 1054 about which second link 1050 may pivot; include track 1072 for guiding movement of first link 1052 and backstop 1066.

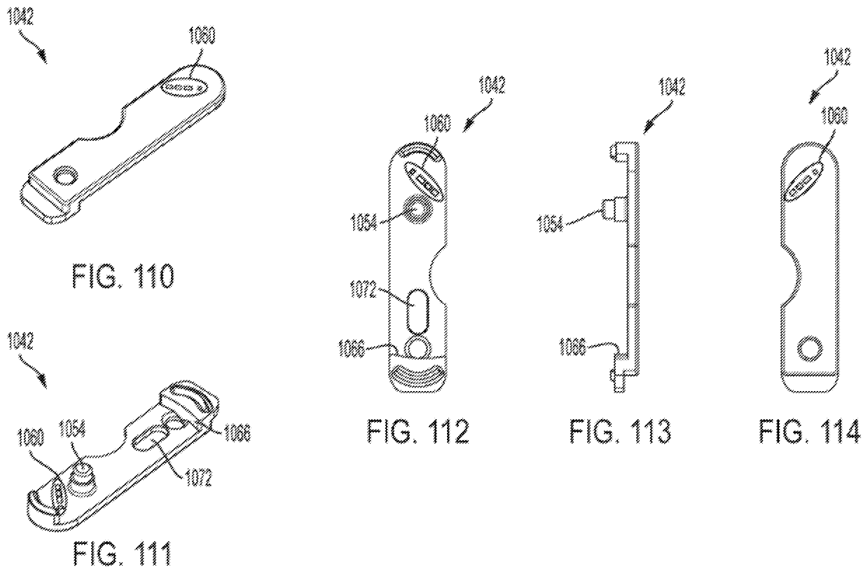

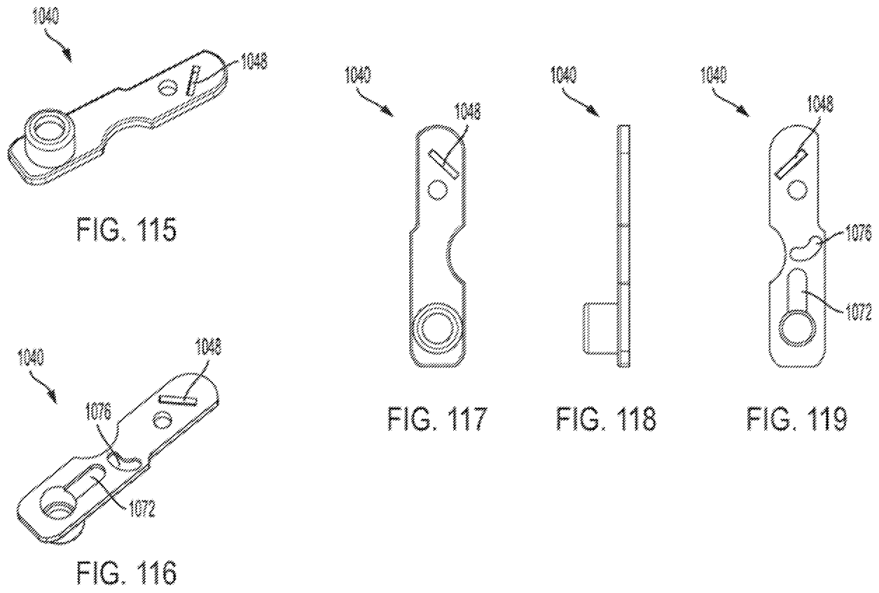

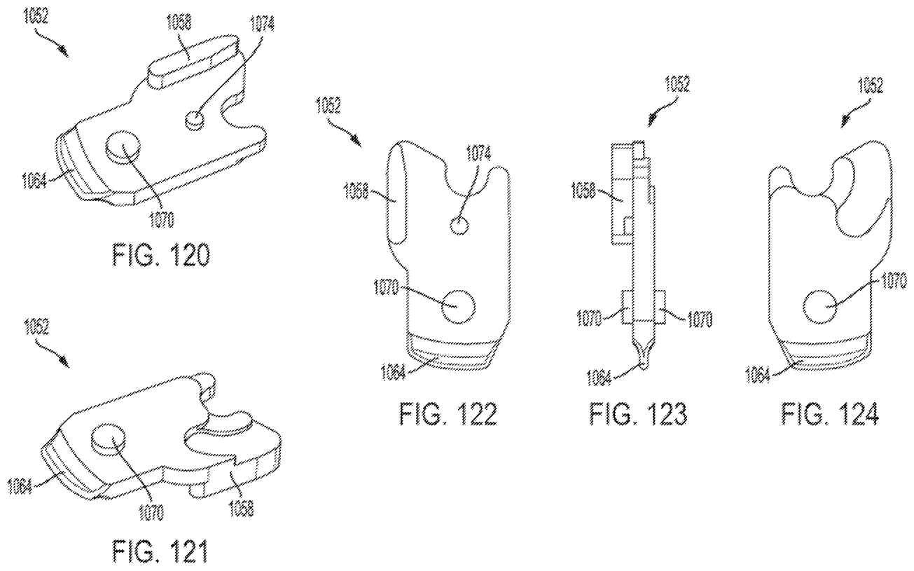

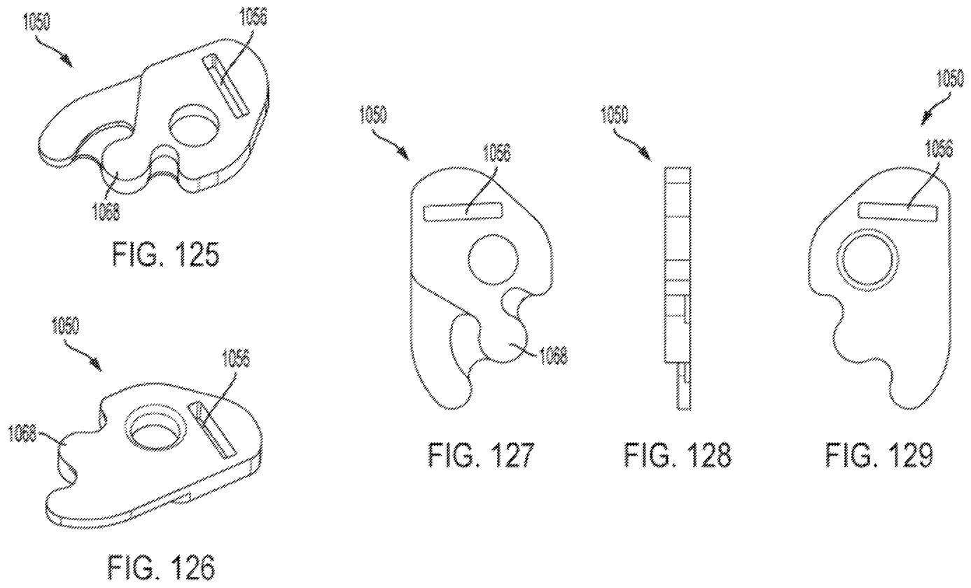

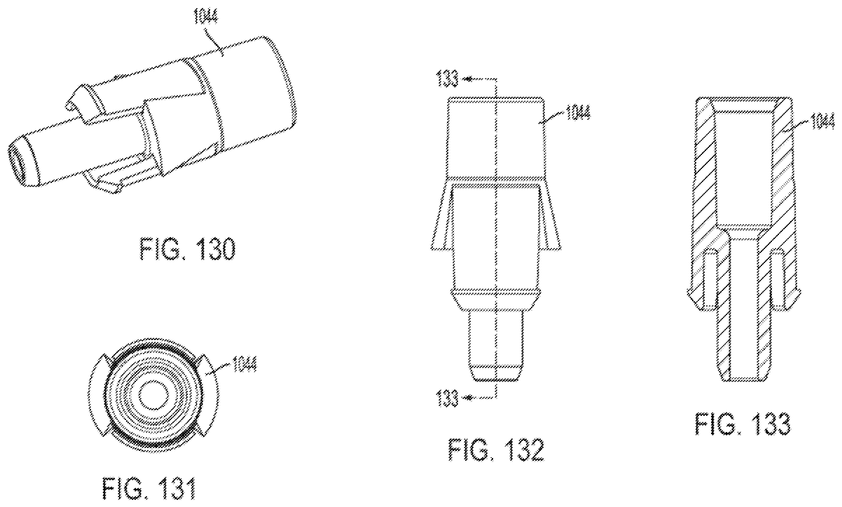

FIGS. 115-119 show several views of the top housing 1040 of the flow-stop assembly 1038. A track 1072 is shown which can guide the movement of first link 1052 via a secondary guide 1074 (see FIGS. 108A and 108B in conjunction with FIGS. 115-119). FIGS. 120-124 show several views of a first link 1052 of the flow-stop assembly 1038 having the plunger 1064 and FIGS. 125-129 show several views of a second link 1050 of the flow-stop assembly 1038. FIGS. 130-133 show several views of a tube coupling 1044 of the flow-stop assembly 1038.

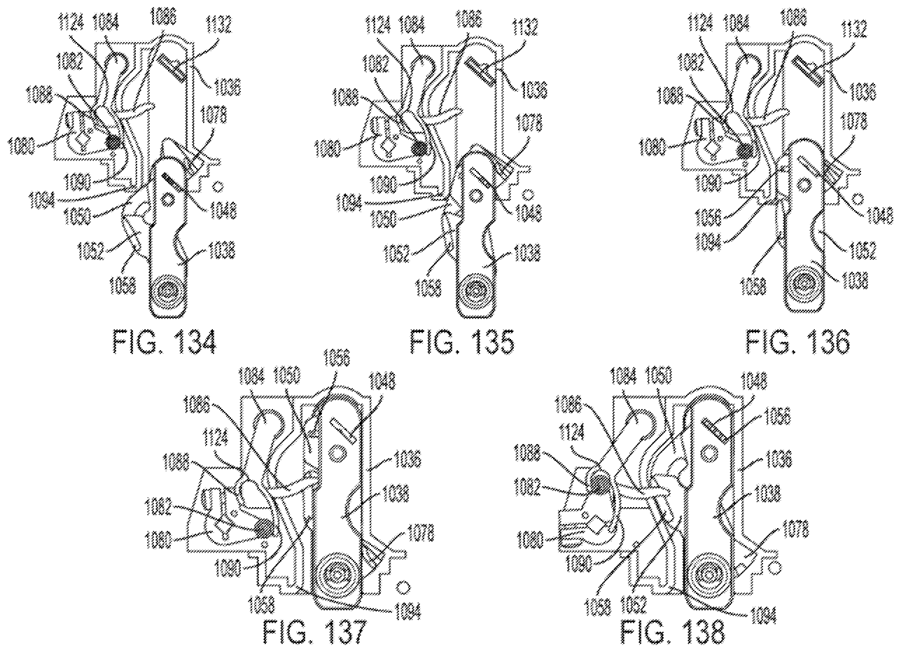

FIGS. 134-138 show the flow-stop assembly 1038 being inserted into a carriage 1036. In FIG. 134, the flow-stop assembly 1038 is in the non-occluding configuration. As the flow-stop assembly 1038 is inserted into the alternative carriage 1036, a cooperating surface 1094 interacts with the second link 1050 to actuate both the second link 1050 and the first link 1052 to place the flow-stop assembly 1038 in the occluding position illustrated in FIGS. 135-136. As shown in FIG. 136, the flow-stop assembly 1038 will be actuated into the occluding position prior to insertion into the alternative carriage 1036. Thus, in some embodiments of the present disclosure, the peristaltic pump 1020 is configured to only receive a flow-stop assembly 1038 in the occluding position; And, if the flow-stop assembly 1038 is not in the occluding position prior to insertion, the peristaltic pump 1020 will actuate the flow-stop assembly 1038 into the occluding position prior to being received (in some specific embodiments, before being partially received and in others, before or during being fully received).

FIG. 137 shows the flow-stop assembly 1038 fully inserted where the gripper finger 1086 engages with the flange 1058. Also shown, is a tube shutter 1078 that actuates when the flow-stop assembly 1038 engages with it. A shaft coupler 1080 is coupled to a shaft of the peristaltic pump 10200. The shaft coupler 1080 may be coupled directly to the main shaft 118, to the main shaft 118 via one or more gears or linkages, through another shaft, or through any other mechanical mechanism known to one of ordinary skill in the relevant art. In yet additional embodiments, the shaft coupler 1080 may be coupled to an upper end of the upper shaft 298.

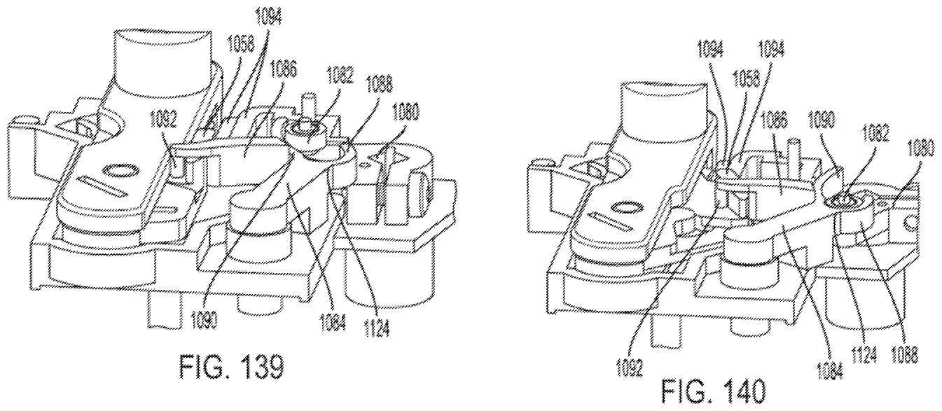

When the flow-stop assembly 1038 is fully inserted into the carriage 1036, a user can actuate the lever 104 thereby causing the shaft coupler 1080 to rotate along with a pin 1082. An interlock arm 1084 includes a second finger 1088 and a first finger 1090 such that actuation of the pin 1082 into a catch well 1124 causes actuation of the interlock arm 1084, which actuates a gripper finger 1086. Because the gripper finger 1086 engages with the flange 1058, actuation of the gripper finger 1086 actuates the first link 1052 and the second link 1050 into the non-occluding position by pulling the flange 1058 in a direction away from the flow-stop assembly 1038. FIG. 139 shows a perspective view of the internal mechanism of the carriage 1036 when the end effector 1092 is engaged with a flange 1058 of the flow-stop assembly 1038 and FIG. 140 shows a perspective view of the internal mechanism of the carriage 1036 when the end effector 1092 is engaged with a flange 1058 of the flow-stop assembly 1038 in the non-occluding position. The end effector 1092 may apply a force on the flange 1058 to actuate the flow-stop assembly 1038 to the non-occluding position as shown in FIG. 140. In some embodiment of the present disclosure, the first finger 1090 and the second finger 1088 may be integrated together as a single structure, e.g., and may form a loop around the pin 1082. The pin 1082 may be a protraction, a roller wheel, a roller bearing, a cam, a rolling cam, a wheel, a slidable protrusion, or any suitable device known to one of ordinary skill in the relevant art.

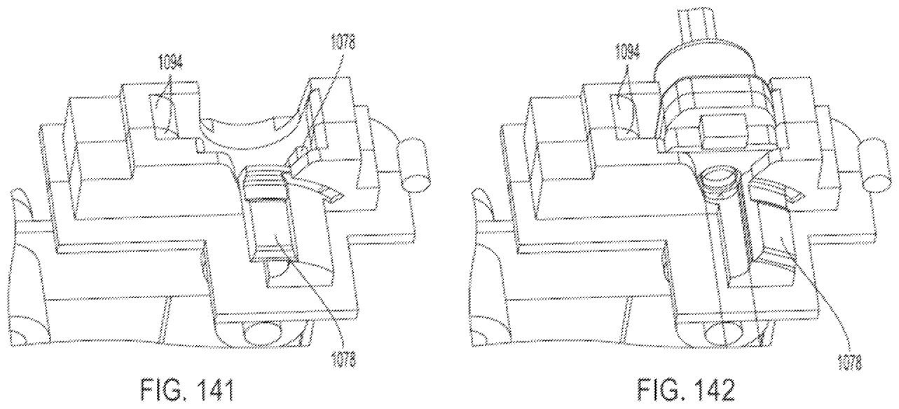

FIGS. 134-138 also show the actuation of the tube shutter 1078. FIG. 141 shows the front of the carriage orifice with a cooperating surface 1094 and a tube shutter 1078. FIG. 142 shows the front of the carriage orifice when the flow-stop assembly 1038 has been inserted and the tube shutter 1078 has been opened. In some embodiments of the present disclosure, a small magnet may be coupled to the interlock arm 1084 such that an adjacent hall-effect sensor is configured to measure the position (e.g., angular or rotational position) of the interlock arm 1084 for use by the processor.

In some embodiments of the present disclosure, the tube shutter 1078 can detect “dark loading” to detect certain user actions that occur when (1) the user mis-loads the administration set having the flow-stop assembly 1038 into the pump, and/or 2) the device is powered off (i.e., dark). For example, if a user loads an administration set having the flow-stop assembly 1038, but neglects to stick the flow-stop assembly 1038 into the device, the tube shutter 1078 can detect that the flow-stop assembly 1038 was not inserted and/or can be configured to pinch any tube within the platen 168 to prevent a free-flow condition.

In some embodiments of the present disclosure, the shaft coupler 1080 can rotate in FIG. 138 (clockwise as seen in FIG. 138) to actuate the flow-stop assembly 1038 to the occluding position when the end effector 1092 of the gripper finger is suitably shaped and configured. In yet an additional embodiment of the present disclosure, when a user pulls the flow-stop assembly 1038 out of the carriage in FIG. 138, the walls of the carriage actuate the first and second links 1050, 1052 to the occluding position.

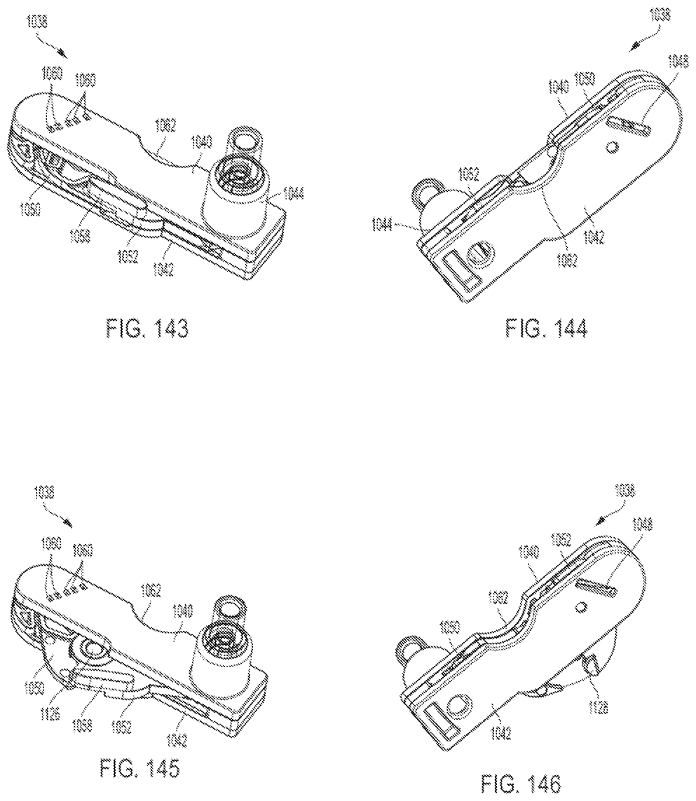

FIGS. 143-146 show several views of another embodiment of the flow-stop assembly 1038. The flow-stop assembly 1038 of FIGS. 143-146 is similar to the flow-stop assembly 1038 of FIGS. 102-105 described supra; however, alternative features are described herein or are readily apparent to one of ordinary skill in the relevant art.

As shown in FIG. 143, the identification aperture 1060 is on the top housing 1040. FIG. 144 shows the housing aperture 1048 on the bottom housing 1042. Non-occluded and occluded fluid flow may be effected through the tube 1046 via actuation of a first link 1052 and second link 1050. FIGS. 143-144 shows the flow-stop assembly 1038 in an occluding position, and FIGS. 145-146 show the flow-stop assembly 1038 in the non-occluding position. When the flow-stop assembly 1038 is in the occluding position, as shown in FIGS. 143-144, a user can press on the first link 1052 via a finger groove 1062 to actuate the second link 1050 and first link 1052 to the non-occluding position as shown in FIGS. 145 and 146. Likewise, when the flow-stop assembly 1038 is in the non-occluding position as shown in FIGS. 145-146, a user can press on a flange 1058 to actuate the second link 1050 and first link 1052 to the occluding position as shown in FIGS. 143-144.

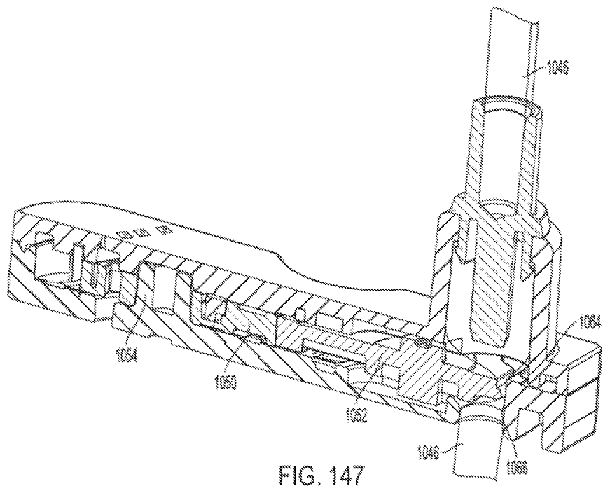

The flow-stop assembly 1038 also includes a housing aperture 1048 which can be used to sense the configuration of an identification aperture 1060, can be used to determine if the flow-stop assembly 1038 is loaded properly or improperly, and can be used to determine the configuration of the flow-stop assembly 1038 (e.g., the occluding vs. non-occluding position, etc.) using an optical sensor as described herein. FIG. 147 shows a cross-sectional view of the flow-stop assembly 1038, which shows a pivot post 1054 about which the second link 1050 can pivot. When the first link 1052 and the second link 1050 are in the occluding position, as shown in FIG. 166, a plunger 1064 occludes the tube 1046 by wedging the tube 1046 between the plunger 1064 and a backstop 1066.

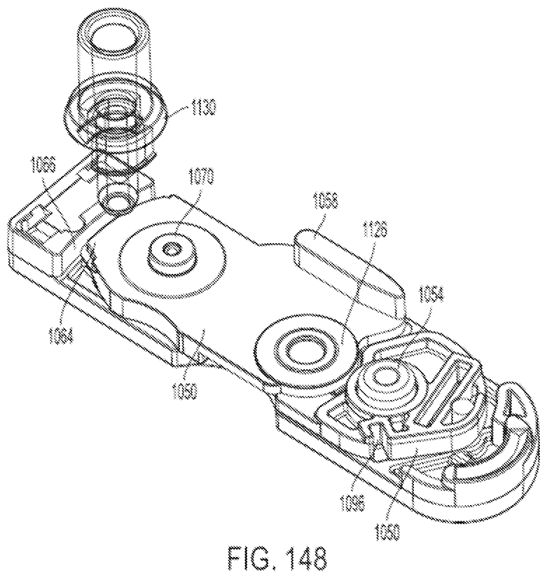

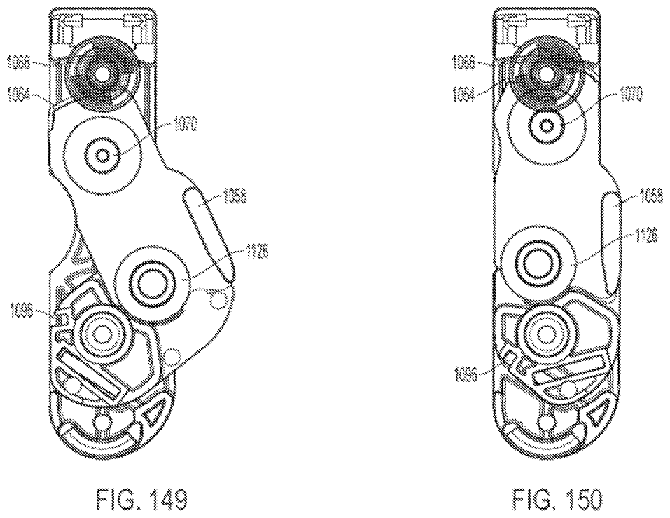

The second link 1050 pivots around the pivot post 1054. The first link 1052 is coupled to the second link 1050 via a hinge 1126. As the first link 1052 is actuated, it is guided within a track 1072 by guides 1070. FIG. 148 shows the flow-stop assembly 1038 with the top housing 1040 removed while in the occluding position and FIG. 149 shows the flow-stop assembly 1038 with the top housing 1040 while in the non-occluding position. As is shown in FIG. 150, when the first link 1052 is in the occluding position, the plunger 1064 is closer to the backstop 1066 and when the second link 1050 is in the non-occluding position as shown in FIG. 149, the plunger 1064 is a predetermined distance from the backstop 1066. The second link 1050 and the first link 1052 are coupled together via a ball-and-socket joint 1068. The guides 1070 positions the first link 1052 such that rotation of the second link 1050 along the pivot post 1054 translates to linear motion of the guides 1070 along the track 1072.

In some embodiments, the flow-stop assembly 1038 includes a notch 1096 configured to use optical recognition to determine when the flow-stop assembly 1038 is in the occluding or non-occluding position. As shown in FIG. 150, the notch 1096 aligns with the housing aperture 1048 such that the optical recognition determines that the flow-stop assembly 1038 is inserted into the carriage 1036 and is in the occluding position.

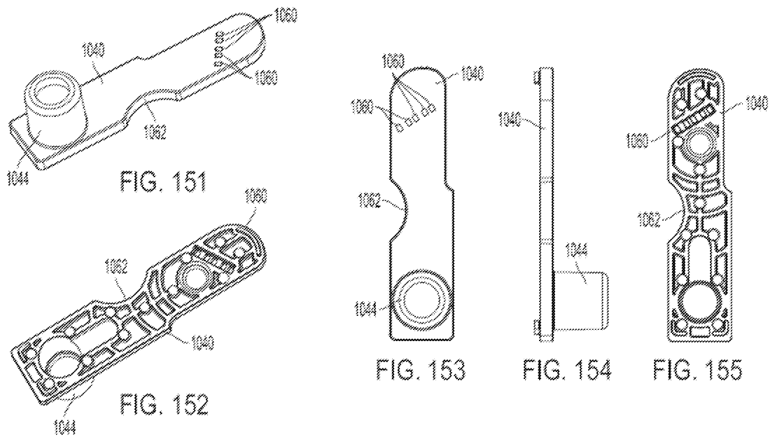

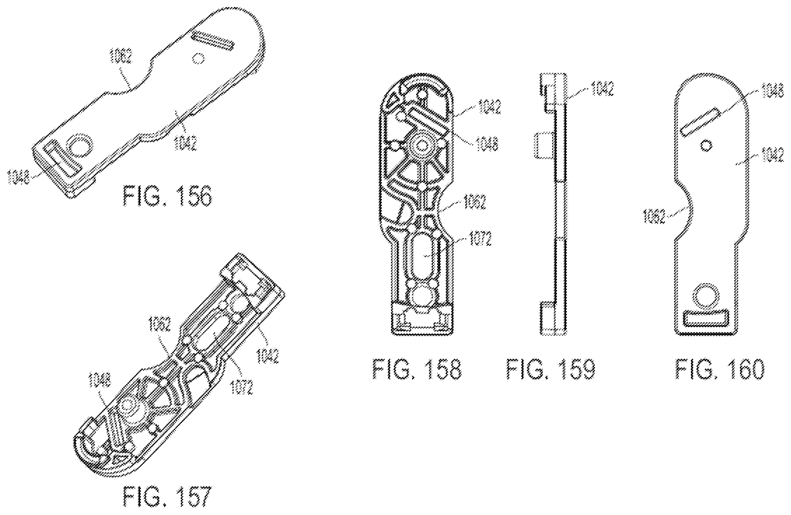

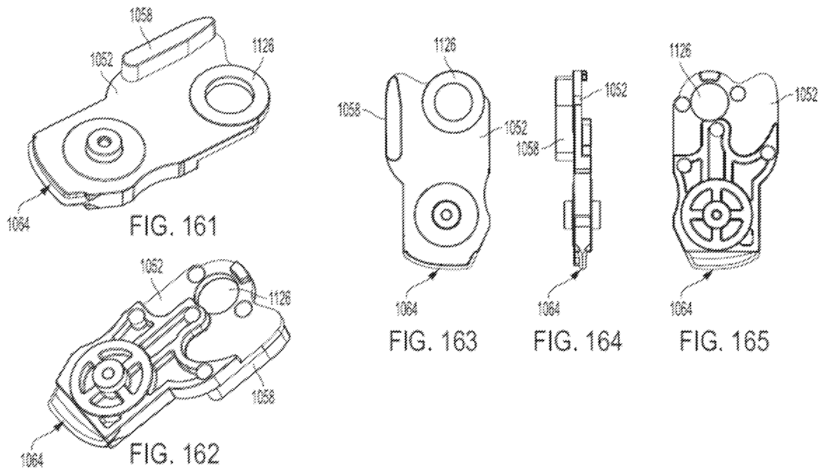

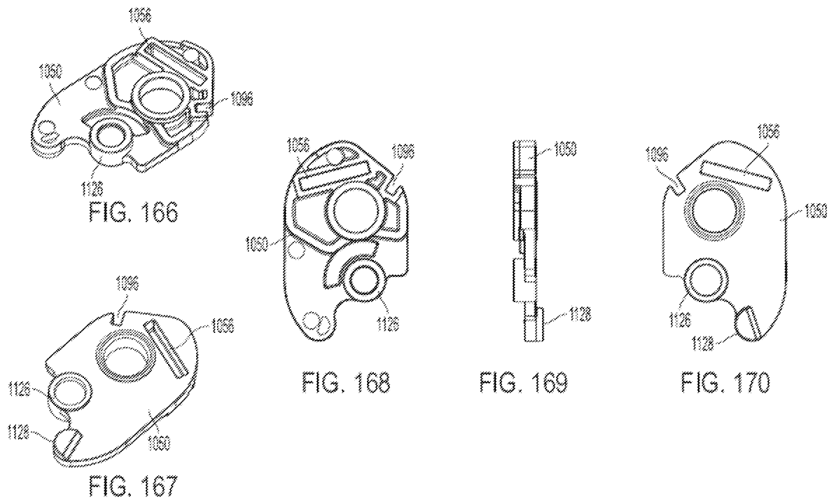

FIGS. 151-155 show several views of the top housing 1040 of the flow-stop assembly 1038. The tube coupling 1044 is integrated with top housing 1040 in the embodiment shown in FIGS. 151-155. In some embodiments, a tube 1046 may include a snap-fit adapter 1130 (see FIG. 148) configured to interface with the tube coupling 1044 of FIGS. 151-155. FIGS. 156-160 show several views of the bottom housing 1042 of the flow-stop assembly 1038 of FIGS. 143-146. As shown in FIGS. 157-158, the bottom housing 1042 includes a secondary track 1076. The secondary track 1076 is configured such that a flange 1128 of a second link 1050 serves a guide and stops movement of the second link 1050 in one (or both) directions of actuation as is readily apparent by one of ordinary skill in the art. FIGS. 161-165 show several views of a first link 1052 of the flow-stop assembly 1038 having a plunger 1064 and FIGS. 166-170 show several views of the second link 1050 of the flow-stop assembly 1038 of FIGS. 143-146.

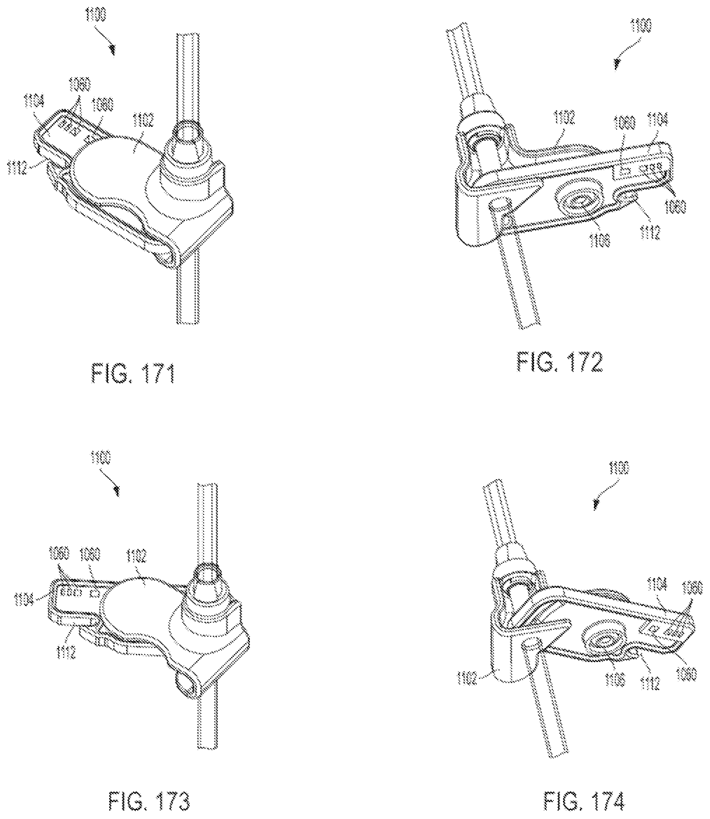

FIGS. 171-174 shows several views of a pinching flow-stop assembly 1100 having a flow stop 1104 with an arcuate slot 1110 FIGS. 171-172 show the pinching flow-stop assembly 1100 when the flow stop 1104 is in the occluding position. The flow stop 1104 can pivot around a pivot post 1054. FIGS. 173-174 show the pinching flow-stop assembly 1100 in the non-occluding position. A user can actuate the flow stop 1104 to transition the pinching flow-stop assembly 1100 to one of the occluding position or the non-occluding position.

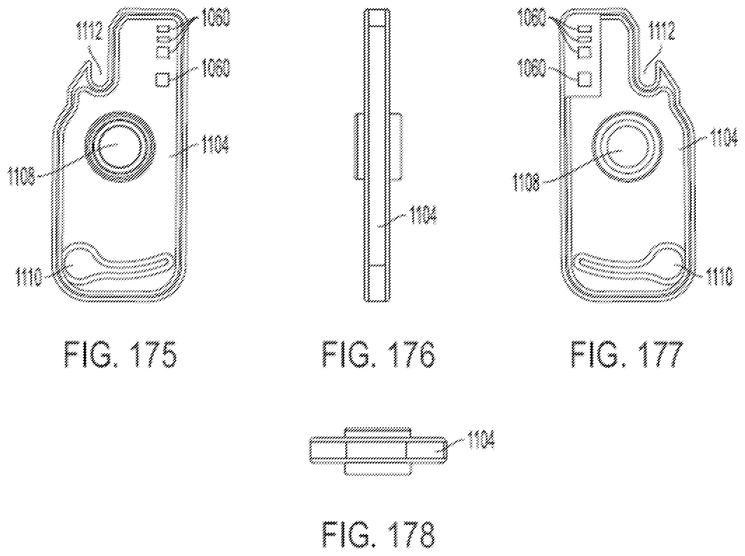

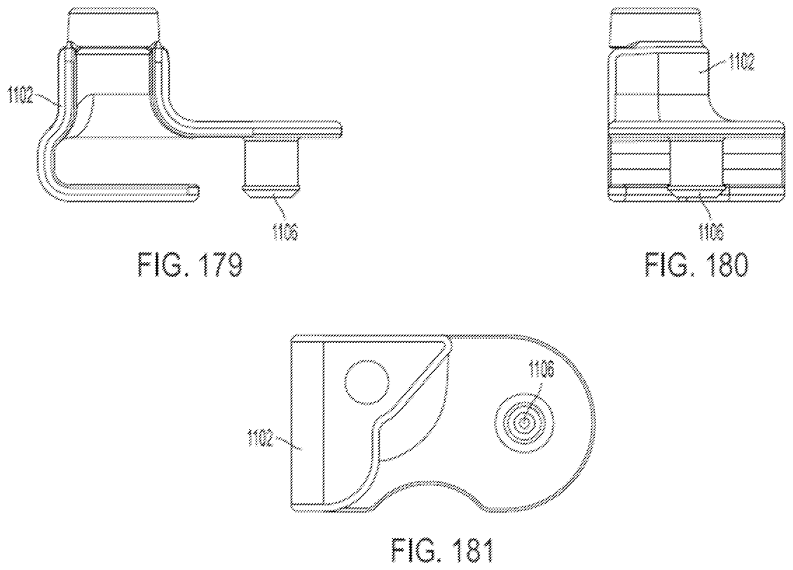

FIGS. 175-178 show several views of the flow stop 1104 of the pinching flow-stop assembly 1100. A tube 1046 can be placed within an arcuate slot 1110 between a narrow portion or a wider portion based upon a pivot of the flow stop 1104 relative to a housing 1102 via the pivot hole 1108. As shown in FIGS. 175-178, the flow stop 1104 includes a notch 1096 that can be engaged by an end effector 1092 of a gripper finger 1086. FIGS. 179-181 show several views of the housing 1102 of the pinching flow-stop assembly 1100. The housing 1102 may be on a top side of the flow stop 1104, a bottom side of flow stop 1104, surrounding both, and in some embodiments, integrated together in a single piece that partially surrounds the flow stop 1104. The pivot hole 1108 of the flow stop 1104 engages with the pivot post 1106 to pivot relative to each other.

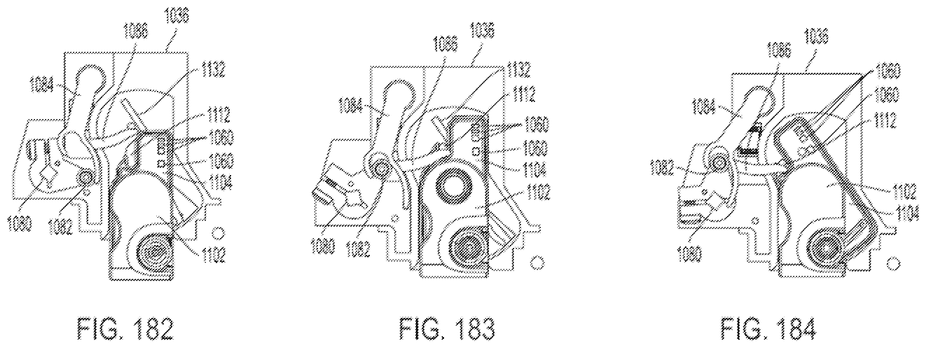

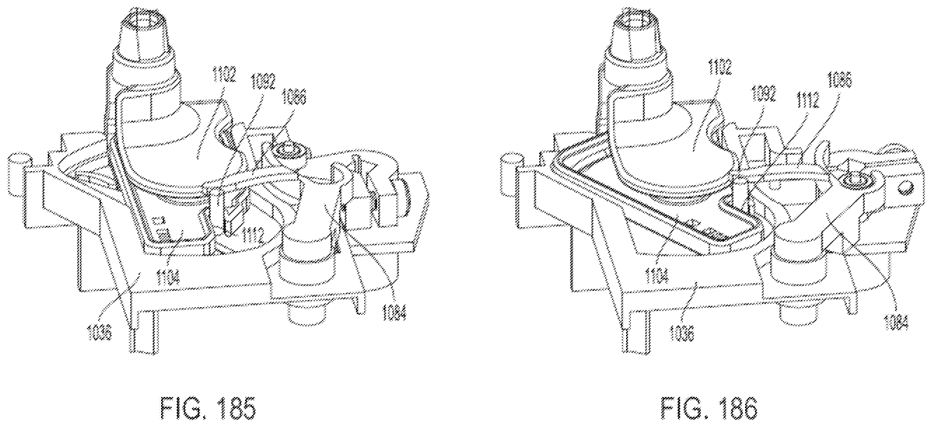

FIGS. 182-184 show the pinching flow-stop assembly 1100 being inserted into a carriage 1036. As shown in FIG. 182, when the pinching flow-stop assembly 1100 is inserted, a notch 1112 approaches and can engage with the end effector 1092 of the gripper finger 1086. As shown in FIG. 182, the alternative carriage 1036 also includes an optical sensor 1132. FIG. 183 shows the pinching flow-stop assembly 1100 fully inserted, but with the flow stop 1104 in the occluding position. FIG. 184 shows the gripper finger 1086 actuating the flow stop 1104 into the non-occluding position to treat a patient. The identification aperture 1060 is now aligned such that the pinching flow-stop assembly 1100 can be identified. A shutter can also be used as part of the alternative carriage 1036 with the pinching flow-stop assembly 1100.

In some embodiments of the present disclosure, the shaft coupler 1080 can rotate in FIG. 184 (clockwise as seen in FIG. 184) to actuate the pinching flow-stop assembly 1100 to the occluding position when the end effector 1092 of the gripper finger is suitably shaped and configured. In yet an additional embodiment of the present disclosure, when a user pulls the pinching flow-stop assembly 1100 out of the carriage in FIG. 184, the walls of the carriage actuate the flow stop 1104 to the occluding position.

### Modular Pump System

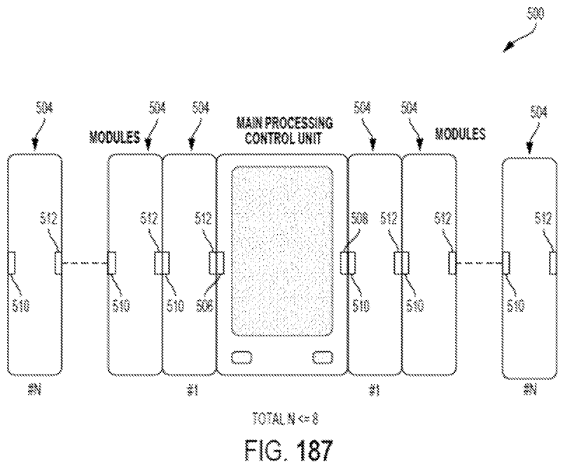

FIG. 187 shows a block diagram of a modular pump system 500 having a central unit 502 and a plurality of medical-device assemblies 504 coupled together. One or more of the medical-device assemblies 504 may be the peristaltic pump 100 or 300 shown and described herein. Additionally or alternatively, the medical-device assemblies 504 may include syringe pumps, battery packs, micropumps, or other medical devices.

The central unit 502 provides power to the medical-device assemblies 504. The central unit 502 includes a left central-unit electrical modular interconnector (MIC) 506 and a right central-unit electrical MIC 508. The left central-unit electrical MIC 506 and the right central-unit electrical MIC 508 each may include a power pin, a communications pin, and one or more ground pins. The central unit 502 provides power to the connected medical-device assemblies 504 through the left central-unit electrical MIC 506 when activated and/or the right central-unit electrical MIC 508 when activated.

The central unit 502 further includes a left mechanical modular interconnector (MIC) 510A and a right mechanical modular interconnector (MIC) 512A. The left mechanical MIC 510A on the center unit provides an attachment interface for a right mechanical MIC 512A on module #1. The right mechanical MIC 512A on the center unit provides an attachment interface for a left mechanical MIC 510A on module #2. The left and right mechanical MICs 510A, 512A provide a detachable mechanical interface between the central unit and medical device assemblies or modules 504 that support the weight of the modules.

All of the medical-device assemblies 504 includes a left mechanical medical interface connector 510 and a right medical-device connector 512 which allow the medical-device assemblies 504 to be connected to the modular pump system 500 from the left side or the right side to receive power and communicate using a common bus. Additionally, the connected medical-device assemblies 504 may be configured to connect the power from the central unit 502 to power a connected medical-device assembly 504 downstream. For example, a medical-device assembly 504 connected just to the right of the central unit 502 may be configured to subsequently power another medical-device assembly 504 connected to it on the right.

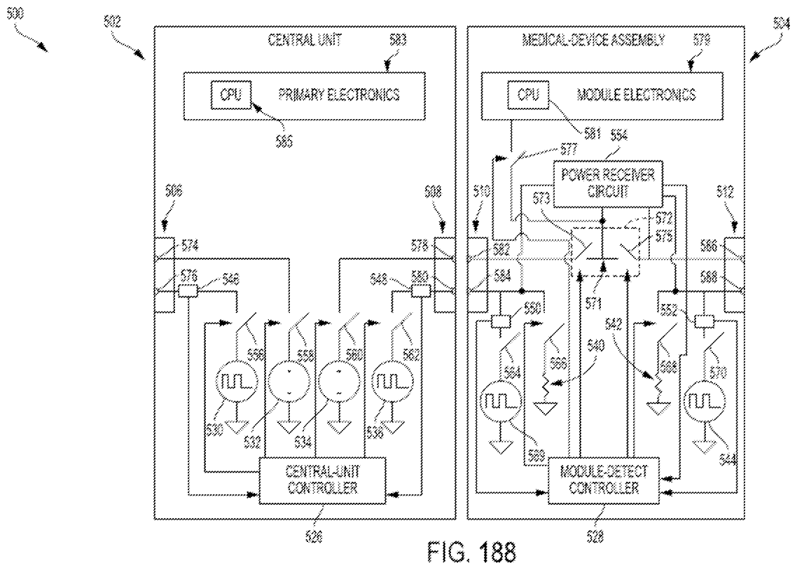

FIG. 188 shows a block diagram of a modular pump system 500 to illustrate the power circuitry of the modular pump system 500. The modular pump system 500 includes a central unit 502 and one or more medical-device assemblies 504. Although one medical-device assembly 504 is shown in FIG. 188, one or more medical-device assemblies 504 may be attached to the right of the medical-device assembly 504 shown in FIG. 188 and/or to the left of the central unit 502. Also, medical-device assemblies 504 may be serially coupled together such as is shown in FIG. 187 to the left or right side of the central unit 502.

The central unit 502 includes primary electronics 583 including a CPU 585. The primary electronics 583 includes addition functions beyond the power circuitry illustrated in FIG. 188. The medical-device assembly 504 includes module electronics 579 that includes a CPU 581. The module electronics 579 includes an electric motor for pumping fluid, power circuits, and other electronics.

The modular pump system 500 is configured such that each of the medical-device assemblies 504 can be coupled to either a right central-unit connector 508 of the central unit 502, a left central-unit connector 506 of the central unit 502, a left medical-device connector 510 or a right medical-device connector 512 of another medical-device assembly 504 (not shown in FIG. 188) to establish communication prior to receiving power through a power pin. For example, the right power pin 578 is not powered until after the medical-device assembly 504 is connected to the right central-unit connector 508 via left medical-device connector 510. Initially, the medical device assembly 504 can power itself sufficiently using the signal received via the communications pin 584. The medical device assembly 504 can request power by using the signal received via a communications pin 584 to power the medical device assembly suitably to passively request power from the device (e.g., the central unit or a medical-device assembly 504) through the communications pin 584. Power can thereafter be received via the left power pin 582 by the medical-device assembly 504 from the central unit 502 when using the system as shown in FIG. 188.

When the central unit 502 is powered up, the central-unit controller 526 may turn on a left signal switch 556 to apply a signal generated by the left signal generating circuit 530 to a left communications pin 576 of a left medical-device connector 510. Also after power up, the central-unit controller 526 may switch the right signal switch 562 into the on position to apply a signal from the right signal generating circuit 536 to the right communications pin 580 of the right central-unit connector 508. In additional embodiments of the present disclosure, the left signal generating circuit 530 and the right signal generating circuit 536 may be combined into a single circuit that generates a single signal for application to the left communications pin 576 and to the right communications pin 580. Additionally or alternatively, enable/disable circuits may be used in place of switches 556, 562, respectively, where the central-unit controller 526 can signal to enable or disable the signal generating circuits 530, 536.

The central-unit controller 526 is coupled to a left load-detect circuit 546 and a right load-detect circuit 548. The left load-detect circuit 546 is configured to detect a passive indication of a request for power of a left connected medical-device assembly 504 (none is shown in FIG. 188). The right load-detect circuit 548 is configured to detect a passive indication of a request for power of a right connected medical-device assembly 504 (one is shown in FIG. 188). The central-unit controller 526 keeps the left power switch 558 open until a request for power by a left connected medical-device assembly 504 has been received and likewise keeps the right power switch 560 open until a request for power by a right connected medical-device assembly 504 has been received. The left load-detect circuit 546 and the right load-detect circuit 548 may be current sense circuits in some embodiments. However, any circuit known to one of ordinary skill in the art may be used to detect a passive indication of a request for power. In some embodiments of the present disclosure, the passive indication of a request for power may be a change in impedance, e.g., a coupling of a resistor to the communications pin 584. Load detection may be done by monitoring current, voltage, frequency response, decay rate, an RC constant, the like, or some combination thereof.

As previously mentioned, the right load-detect circuit 548 may in some embodiments be a current sensor. Thus, if the signal from the right signal generating circuit 536 is a voltage waveform (e.g., a square waveform), the current of the right signal generating circuit 536 may be monitored by the right load-detect circuit 548 to determine if an impedance change (e.g., a decreased resistance) has occurred on the load impedance as detected by the right load-detect circuit 548.

As previously mentioned, after power up, the central-unit controller 526 switches the right signal switch 562 into the on position to apply a signal from the right signal generating circuit 536 to the right communications pin 580 of the right central-unit connector 508. When the medical-device assembly 504 is initially coupled to the central unit 502, a signal is received from the right signal generating circuit 536 through the right communications pin 580 of the right central-unit connector 508 via the left communications pin 584 of the left medical-device connector 510. The signal is used by the power receiver circuit 554 to initially power the power receiver circuit 554. That is, energy harvesting, such as a rectifier, a charge pump, etc., may be used by the power receiver circuit 554 to power itself.

The power receiver circuit 554 powers the module-detect controller 528. Upon determination by the module-detect controller 528 that a signal is present on the left communications pin 584, the module-detect controller 528 signals the left load switch 566 to close so that the left resistor 540 is now coupled to the left communications pin 584. That is, the left load switch 566 is closed thereby connecting the left resistor 540 to the left communications pin 584. This change in impedance is detected by the right load-detect circuit 548 of the central unit 502 which is communicated to the central-unit controller 526. The central-unit controller 526 takes this change in impedance to be a passive request for power. Therefore, the central-unit controller 526 switches the right power switch 560 ON so that the right power circuit 534 supplies power to the right power pin 578 through the right central-unit connector 508 via the left power pin 582 of the left medical-device connector 510. Then a switch 573 can be closed to provide power to the cross-bar bus 571 which is receivable by the power receiver circuit 554. The power is received by the power receiver circuit 554 which is then used to power the module electronics 579 by closing the switch 577. The power receiver circuit 554 can use its power to power the module-detect controller 528. In some embodiments, the switch 577 may be replaced by a diode or other circuitry to allow power to flow to the module electronics 579 anytime power is supplied to the crossbar bus 571.

After the module-detect controller 528 determines that power is being supplied from the left power pin 582, the module-detect controller 528 can configure the right side of the medical-device assembly 504 to accept another medical-device assembly 504 on its right as seen from FIG. 188 and in this example. The module-detect controller 528 may set the frequency of the right signal generating circuit 536 to half of the frequency it receives via the right signal generating circuit 536 of the central unit 502. Thereafter, the module-detect controller 528 closes the right signal switch 570 and monitors the right communications pin 588 load by monitoring the right load-detect circuit 552. Please note that load-detect circuit 550 performs the same function, but on the other side of the medical-device assembly 504. If or when the module-detect controller 528 detects a passive request for power, the module-detect controller 528 may close a right cross-bar switch 575 of a crossbar 572 so that power is supplied downstream, i.e., to the right from the view of FIG. 188. Also a right resistor 542 is coupled to a right load switch 568 that are used to passively request power, e.g., when the medical-device assembly 504 is connected to the other side of the central unit 502 from what is shown in FIG. 188.

Because the central-unit controller 526 generates a fixed frequency by the signal generating circuits 530, 536, and each medical-device assembly 504 reduces the frequency sent downstream by half, each of the medical-device assemblies 504 coupled to the modular pump system 500 can determine its position relative to the central unit 502 by monitoring the frequency of the signal coming in on respective communications pin 584, 588 because the frequency of the signals generated by 530 and 536 are predetermined and known by all of the medical-device assemblies 504. For example, the frequency values of the signals generated by 530 and 536 may be stored in non-volatile memory within the module electronics 579. Also, the side on which the medical-device assembly 504 initially receives the signal via a communication pin 584, 588 may be used by the module-detect controller 528 to know on which side of the central unit 502 it resides and by monitoring the frequency of the signal initially incoming, the medical-device assembly 504 will know how many other medical-device assemblies 504 (if any) reside between it and the central unit 502. Thus, a medical-device assembly's 504 position may be used as a bus-communications address to communicate with other medical-device assemblies and/or with the central unit 502, e.g., using on-off keying modulated signal carrying a Controller Area Network (“CAN”)-protocol signal.

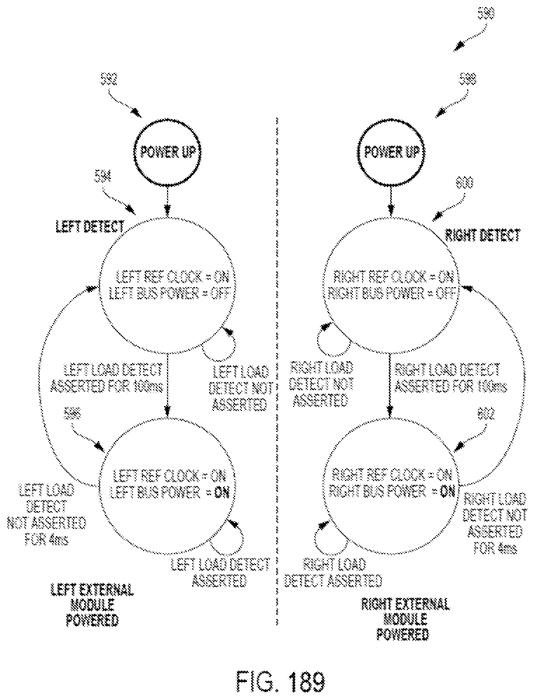

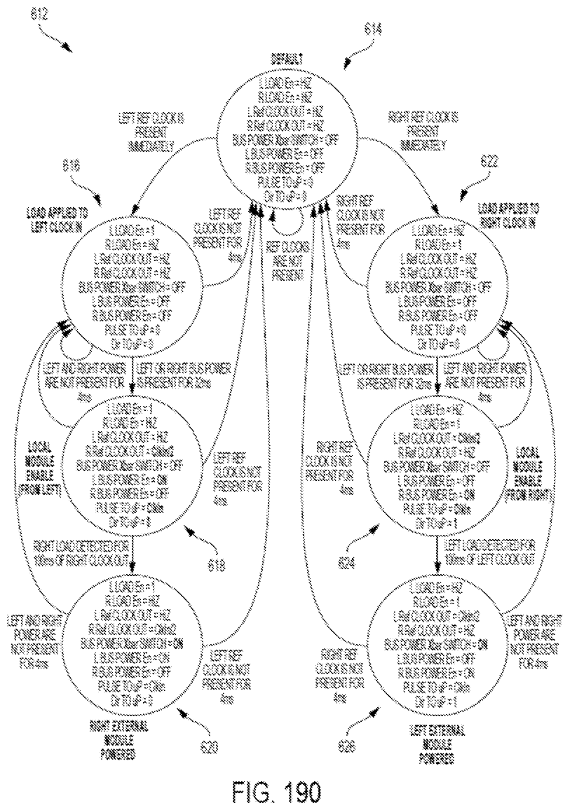

FIG. 189 shows a power-on state diagram 590 of the central unit 502 power circuitry shown in FIGS. 97-98. A state 592, a state 594, and a state 596 illustrate the left power circuitry of the central unit 502 which can provide power to an attached medical-device assembly 504 through the left central-unit connector 506. A state 598, a state 600, and a state 602 illustrate the right power circuitry of the central unit 502 which can power an attached medical-device assembly 504 through the right central-unit connector 508. Please note that the two sides of power-on state diagram 590 can occur in parallel, and, in some embodiments, out of sync with each other.

In state 592, designated as POWER UP, the circuitry of the central unit 502 is powered up, for example, when a user turns on a power switch and/or plugs the central unit 502 into an A/C outlet. Thereafter, state 594 is entered into, which is designated as LEFT DETECT. In state 594, a left reference clock (e.g., signal generating circuit 530 of FIG. 188) will be turned on (e.g., the switch 556 is closed) and a left bus power (e.g., the left power circuit 532) will remain off (e.g., switch 558 remains open). The left reference clock may be created and/or controlled by a signal generating circuit 530 that is coupled to a left communications pin 576 of the left central-unit connector 506. The left bus power is a left power circuit 532 that can send power to a left power pin 574 of the left central-unit connector 506. As described in greater detail below, the left reference clock signal is monitored via left load-detect circuit 546 to sense if an impedance change indicates a passive indication of a request for power of a left connected medical-device assembly 504. For example, a left connected medical-device assembly 504 can change a resistance, e.g., by grounding (e.g., sinking) a resistor, to the communications pin 588 of the right medical-device connector 512 that is coupled to the left communications pin 576 of the left central-unit connector 506 to indicate a request for power.

As shown in FIG. 189, state 594 will continue to transition to itself as long as the passive request for power is not detected as indicated by the LEFT LOAD DETECT NOT ASSERTED transition. In state 594, if the left signal detects a load for 100 milliseconds, it is interpreted as a passive request for power, after which, the state 594 transitions to the state 596. This transition is indicated by the “LEFT LOAD DETECT ASSERTED FOR 100 ms” transition in the state diagram 590. In state 596, the central unit 502 switches to a left power-on mode and applies power to the left power pin 574 of the left central-unit connector 506 (indicated as LEFT BUS POWER=ON). The central unit 502 will continue to apply power as long as the passive request for power is detected; this is illustrated as “LEFT LOAD DETECT ASSERTED” transition in the state diagram 590. The LEFT BUS POWER=ON may signify that the left power switch 558 is closed to connect the left power circuit 532 to the left power pin 574 of the left central-unit connector 506.

The right side of the power-on state diagram 590 operates in a similar manner as the left side of the power-on state diagram 590. The two sides of the power-on state diagram 590 may operate independently and/or in parallel. As shown in FIG. 189, state 598, state 600, and state 602 illustrate the right power circuitry of the central unit 502 which can provide power to an attached medical-device assembly 504 through the right central-unit connector 508.

In state 598, designated as POWER UP, the circuitry is powered up, for example, when a user turns on a power switch and/or plugs the central unit 502 into an A/C outlet. Thereafter, the state 600 is entered into, which is designated as RIGHT DETECT. In the state 600, a right reference clock (e.g., signal generating circuit 536 of FIG. 188) will be turned on and a right bus power (e.g., the right power circuit 534) will remain off or unconnected via the right power switch 560. The right reference clock may be created and/or controlled by a signal generating circuit 536 that is coupled to a communications pin 588 of the right central-unit connector 508. The right bus power is a right power circuit 534 that can send power to a right power pin 578 of the right connector 508. The right reference clock signal is monitored via right load-detect circuit 548 to sense if an impedance change indicates a passive indication of a request for power of a right connected medical-device assembly 504. For example, a right connected medical-device assembly 504 can apply a resistance, e.g., by grounding a resistor, to the communications pin 580 of the left medical-device connector 510 that is coupled to the right communications pin 580 of the right central-unit connector 508 to indicate a passive request for power.

As shown in FIG. 189, the state 600 will continue to transition to itself as long as the passive request for power is not detected and is indicated by the “RIGHT LOAD DETECT NOT ASSERTED” transition. In the state 600, if the right signal detects a load for 100 milliseconds, it is interpreted as a passive request for power, after which, the state 600 transitions to the state 602. This transition is indicated by the “RIGHT LOAD DETECT ASSERTED FOR 100 ms” transition in the state diagram 590. In the state 602, a central-unit switchable power circuit switches to a power-on mode and applies power to a power pin of the right central unit connector 508 (indicated as RIGHT BUS POWER=ON). The right power circuit 534 will continue to apply power as long as the passive request for power is detected and is designated as RIGHT LOAD DETECT ASSERTED in the state diagram 590. The RIGHT BUS POWER=ON may signify that the right power switch 560 is closed to connect the right power circuit 534 to the right power pin 578 of the right central-unit connector 508.

FIG. 190 shows a state diagram 612 of the medical device assembly 504 power circuitry. The state diagram 612 includes states 614, 616, 618, 620, 622, 624, and 626. Within each state of the state diagram 612, Table 1 defines the output values as follows:

TABLE 1

| | | | Correspondence |
|---|---|---|---|
| Label | Description | Possible Values | to FIG. 188 |
| L LOAD | Controls whether | HiZ (high | Signal from Module- |
| En | a resistive load is | impedance) | detect controller 528 |
| | coupled to a left | or 1 (resistor | to left load switch |
| | communications | connected) | 566. |
| | pin. | | |
| R LOAD | Controls whether | HiZ (high | Signal from Module- |
| En | a resistor | impedance) | detect controller 528 |
| | is coupled | or 1 (resistor | to right load switch |
| | to a right | connected) | 568. |
| | communications | | |
| | pin. | | |
| L Ref | Controls a left | HiZ (high | Signal from Module- |
| CLOCK | clock | impedance) | detect controller 528 |
| OUT | signal to a left | Clkin/2 (outputs | to the left signal |
| | communications | a signal one- | switch 564 and |
| | pin. | half of the | frequency selection of |
| | | frequency | the right power pin |
| | | received via a | 578 by the module- |
| | | communications | detect controller 528. |
| | | pin | |
| R Ref | Controls a right | HiZ (high | Signal from Module- |
| CLOCK | clock signal | impedance) | detect controller 528 |
| OUT | to a right | Clkin/2 (outputs | to right signal switch |
| | communications | a signal one- | 570 and frequency |
| | pin. | half of the | selection of the signal |
| | | frequency | generator 544 by the |
| | | received via a | module-detect |
| | | communications | controller 528. |
| | | pin | |
| BUS | Controls | Off (both power | Signal from Module- |
| POWER | whether | pins are not | detect controller 528 |
| Xbar | both power | coupled to the | to crossbar 572 closes |
| SWITCH | pins are | cross-bar bus). | switches 573, 575 |
| | coupled to the | On (both power | |
| | cross-bar bus. | pins are | |
| | | coupled to the | |
| | | cross-bar bus). | |
| L BUS | Controls | Off (power not | Signal from Module- |
| POWER | whether | applied from the | detect controller 528 |
| En | the left | left power pin | to the left cross-bar |
| | cross-bar | to the cross- | switch 573. |
| | switch | bar bus) | |
| | couples the | On (power is | |
| | left power | applied from | |
| | pin to the | the left power | |
| | cross-bar bus. | pin to the cross- | |
| | | bar bus) | |
| | | Turns on power | |
| | | to device | |
| | | electronics in | |
| | | some | |
| | | embodiments. | |
| R BUS | Controls | Off (power not | Signal from Module- |
| POWER | whether | applied from | detect controller 528 |
| En | the right | the right power | to the right cross-bar |
| | cross-bar | pin to the | switch 575. |
| | switch | cross-bar bus) | |
| | couples the | On (power is | |
| | right power | applied from | |
| | pin to the | the right power | |
| | cross-bar bus. | pin to the cross- | |
| | | bar bus) | |
| | | Turns on power | |
| | | to device | |
| | | electronics in | |
| | | some | |
| | | embodiments. | |
| PULSE | A signal to the | ClkIn (signals | Signal from Module- |
| TO uP | processor | to the processor | detect controller 528 |
| | to indicate a | that a clock has | to CPU. |
| | presence of a | been received). | |
| | received | 0 (signals to | |
| | communications | the processor | |
| | signal. | that a clock | |
| | | has not been | |
| | | received. | |
| Dir | A signal to the | 0 (Clock signal | Signal from Module- |
| TO uP | processor to | received from | detect controller 528 |
| | indicate the | module coupled | to CPU. |
| | direction, e.g., | to the left | |
| | left or right, the | connector) | |
| | communications | 1 (Clock signal | |
| | signal comes | received from | |
| | from. | module coupled | |
| | | to the right | |
| | | connector) | |
| | | May be | |
| | | ignored if no | |
| | | pulse signal | |
| | | is present. | |

Initially, state 614 is entered into. In state 614, the medical device assembly 504 is a state where it is detached from all power sources, such as when it is resting within a cabinet. States 616, 618, and 620 correspond to the left side of the medical device assembly 504 being connected to a central unit 502 or another medical-device assembly 504 on its left side. Likewise, states 622, 624, and 626 correspond to the medical device assembly 504 being connected to a central unit 502 or another medical-device assembly 504 on its right side.

The transition “LEF REF CLOCK IS PRESENT IMMEDIATELY” from state 614 to state 616 occurs when the left connector 510 detects a signal from the left communications pin 584. In state 616, the “L LOAD En” is set to “1”, which means that the left resistor 540 is coupled to the left communications pin 584 (e.g., by closing the switch 566). The state 616 will continue to transition back to itself if no power is detected from either the left side from the left power pin 582 or the right side from the right power pin 586 after 4 ms, as indicated by the “LEFT AND RIGHT POWER ARE NOT PRESENT FOR 4 ms” transition. However, if the left clock signal is not detected via the left communications pin 584 for at least 4 ms, the medical-device assembly 504 transitions from state 616 to 614 by the transition labeled as “LEFT REF CLOCK IS NOT PRESENT FOR 4 ms”.

When the power is received from the left power pin for at least 32 ms, state 616 transitions to state 618 as indicated by the “LEFT OR RIGHT BUS POWER IS PRESENT FOR 32 ms”. In state 618, the “L BUS POWER En” is set to ON, which would close the left cross-bar switch 573 thereby sending power to the common bus 571. In some embodiments, the switch 577 is closed at state 618 to send power to the module electronics 579. Also in state 618, the “R Ref Clock Out” turns on the right clock at half the frequency received via the left communications pin 584. That is, the switch 570 is closed while signal generator 544 generates a square wave that is one-half the frequency received via the left communications pin 584. Also, the “Pulse to uP” CkIn signal is sent to the CPU 581 (connection not explicitly shown in FIG. 188, but it may be a wired connection) so that the CPU 581 knows that a clock signal has been received via the left communications pin 584. The “Dir To uP” signal is set to 0, which is sent to the CPU 581 so that the CPU 581 can determine which direction the signal is received from. In this exemplary embodiment, the 0 value indicates that the signal is coming from the left communications pin 584; however, the particular logic values used may be changed.

If the left clock is not present for 4 ms, the medical-device assemblies 504 transitions from state 618 to state 614 via transition labeled “LEFT REF CLOCK IS NOT PRESENT FOR 4 ms”. If neither the left power pin nor the right power pin is powered up for 4 ms, the medical-device assemblies 504 transition from state 618 to state 616. If a passive request for power is detected via the right communications pin of the medical-device assemblies 504, the medical-device assemblies 504 transitions from state 618 to state 620 when the load is detected for 100 ms via the right clock output. The transition is labeled “RIGHT LOAD DETECTED FOR 100 ms OF RIGHT CLOCK OUT,” which corresponds to the case in which the BUS POWER Xbar switch is turned ON, which means that both of switches 573 and 575 are closed thereby allowing power to flow from the left power pin to the right power pin.

At state 620, if the left and right power pins are ever not receiving power for 4 ms, then the medical-device assembly 504 transition from state 620 to state 616 via the transition labeled “LEFT AND RIGHT POWER ARE NOT PRESENT FOR 4 ms”. If, at state 620, the left reference clock is not present for 4 ms, the medical-device assemblies 504 transitions from state 620 to state 614 via the transition labeled “LEFT REF CLOCK IS NOT PRESENT FOR 4 ms”.

Referring again to FIG. 190, the right branch from state 614 will now be described. The transition “RIGHT REF CLOCK IS PRESENT IMMEDIATELY” from state 614 to state 622 occurs when the right connector 512 detects a signal from the right communications pin 588. In state 622, the “R LOAD En” is set to “1”, which means that the right resistor 542 is coupled to the right communications pin 588 (e.g., by closing the switch 568). The state 622 will continue to transition back to itself if no power is detected from either the left side from the left power pin 582 or the right side from the right power pin 586 after 4 ms, as indicated by the “LEFT AND RIGHT POWER ARE NOT PRESENT FOR 4 ms” transition. However, if the right clock signal is not detected via the right communications pin 588 for at least 4 ms, the medical-device assembly 504 transitions from state 622 to 614 by the transition labeled as “RIGHT REF CLOCK IS NOT PRESENT FOR 4 ms”.

When the power is received from the right power pin for at least 32 ms, state 616 transitions to state 624 as indicated by the transition label “LEFT OR RIGHT BUS POWER IS PRESENT FOR 32 ms”. In state 624, the “R BUS POWER En” is set to ON, which would close the right cross-bar switch 575 thereby sending power to the common bus 571. In some embodiments, the switch 577 is closed at state 624 to send power to the module electronics 579. Also in state 624, the “L Ref Clock Out” turns on the left clock at half the frequency received via the right communications pin 588. That is, the switch 564 is closed while signal generator 569 generates a square wave that is one-half the frequency received via the right communications pin 588. Also, the “Pulse to uP” CkIn signal is sent to the CPU 581 (connection not explicitly shown in FIG. 188, but it may be a wired connection) so that the CPU 581 knows that the clock signal has been received via the left communications pin 584. The “Dir To uP” signal is set to 1, which is sent to the CPU 581 so that the CPU 581 can determine which direction the signal is received from. In this exemplary embodiment, the 1 value indicate that the signal is coming from the right communications pin 588; however, the particular logic values used may be changed.

If the left clock is not present for 4 ms, the medical-device assemblies 504 transitions from state 624 to state 614 via transition labeled “RIGHT REF CLOCK IS NOT PRESENT FOR 4 ms”. If neither the left power pin nor the right power pin is powered up for 4 ms, the medical-device assemblies 504 transition from state 624 to state 622. If a passive request for power is detected via the left communications pin of the medical-device assemblies 504, the medical-device assemblies 504 transitions from state 624 to state 626 when the load is detected for 100 ms via the left communication pin. The transition is labeled “LEFT LOAD DETECTED FOR 100 ms OF RIGHT CLOCK OUT”, which corresponds to the case in which the BUS POWER XbarSWITH is turned ON, which means the both of switches 573 and 575 are closed thereby allowing power to flow from the left power pin to the right power pin. At state 626, if the left and right power pins are ever not receiving power for 4 ms, then medical-device assemblies 504 transition from state 626 to state 622 via the transition labeled “LEFT AND RIGHT POWER ARE NOT PRESENT FOR 4 ms”. If, at state 626, the right reference clock is not present for 4 ms, the medical-device assemblies 504 transitions from state 626 to state 614 via the transition labeled “RIGHT REF CLOCK IS NOT PRESENT FOR 4 ms”.

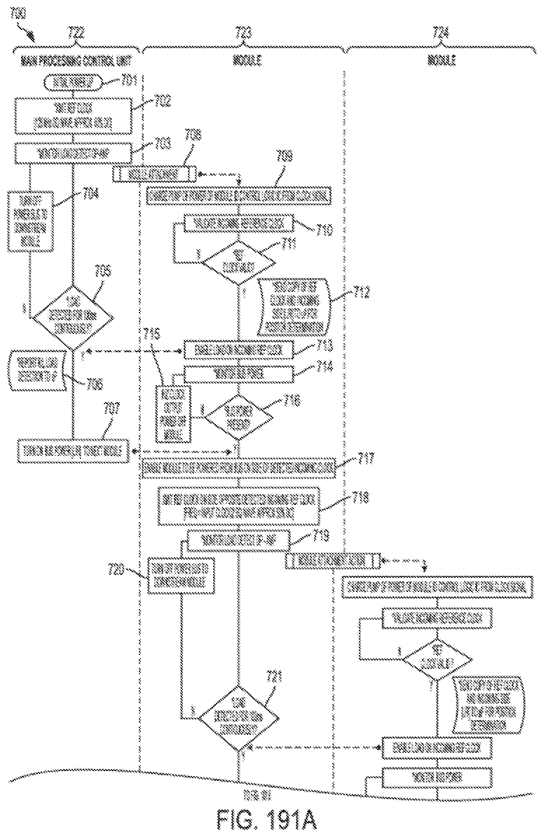

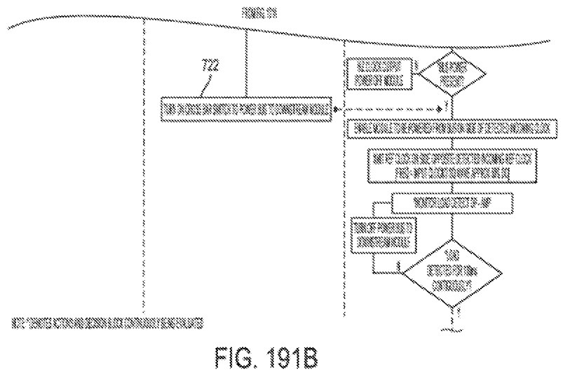

FIGS. 191A-191B show a timing diagram 700 of the modular pump system 500 as two medical device assemblies 504 are coupled to the central unit 502 to illustrate the powering-up sequence of the system. The timing diagram 700 shows a central unit 722, which may be the same as the central unit 502 described herein, and the timing diagram 700 shows two medical device assemblies or modules 723, 724 which may be the same as the medical device assembly 504 described herein.

At 701, the central unit 722 has an initial power up. At 702 the reference clock generates a square wave and couples it to a communication pin of the module 723 after the module is attached at 708. At 703, a passible indication of a request for power is determined by using an operational amplifier to detect impedance on the communications pin. If a load is not detected at 100 ms at 705, then at 704 the power applied to a right power pin is turned off (if already on). If it is detected, then at 706, a detected load is communication to the microprocessor and the right power bus is turned on as to supply power to the right power pin at 707.

The timing diagram 700 also shows the operation of the medical-device assembly 723 when it is coupled to the central unit 722. The attachment is shown as 708. At 709, the medical-device assembly 723 uses the signal received from the central unit 722 and harvests it using a charge pump. If the clock is validated 710 (e.g., a predetermined number of signals determines it is a clock having the proper characteristics), then 710 transitions through 711 to 713. Otherwise, 710 transitions to 711 and back to 710. For example, the first few samples of a square wave may be ignored so that transient signals generated by a users' touch does not cause a false positive for a passive request for power. Additionally or alternatively, a clock may start on the rising edge of a waveform and a predetermined amount of time may be required to pass where the clock is above a predetermined threshold until the square wave is considered valid. One of ordinary skill in the art will appreciate variations including the use of positive logic, negative logic, or inverted logic to implement this touch-detect feature. In some specific embodiments, a predetermined number of valid pulses must be detected until the signal is considered to be valid. At 712, a copy of the reference signal clock and incoming side of the signal is sent to the processor so that it can determine its position within the system 500.

At 713, a load is applied to the communications pin and then, the assembly 723 transitions to 714 where it waits for power via the power pin. That is, 714 transitions from 716 to 715 until power is received after which the assembly 723 transitions to 717. At 717, the module is powered from the power bus.

At 718, a signal is turned on the opposite side connected to the central unit 722 for application to the communications pin that is opposite to the central unit 722. At 719, an op-amp monitors the load on the communications pin and if a load is not detected for 100 ms continuously, then it will turn off the power bus at 720 and transitions back to 719. Otherwise, 721 transitions from 722 to turn on the crossbar to dower downstream to the assembly 724. The assembly 724 operates in the same way as the assembly 723 and as indicated by the timing diagram 700. Please note that the assembly 723, 724 operate the same way regardless as to whether a central unit 722 is applying the signal or another assembly 504 is applying the power (however the frequency changes of the clock to indicate relative position is used).

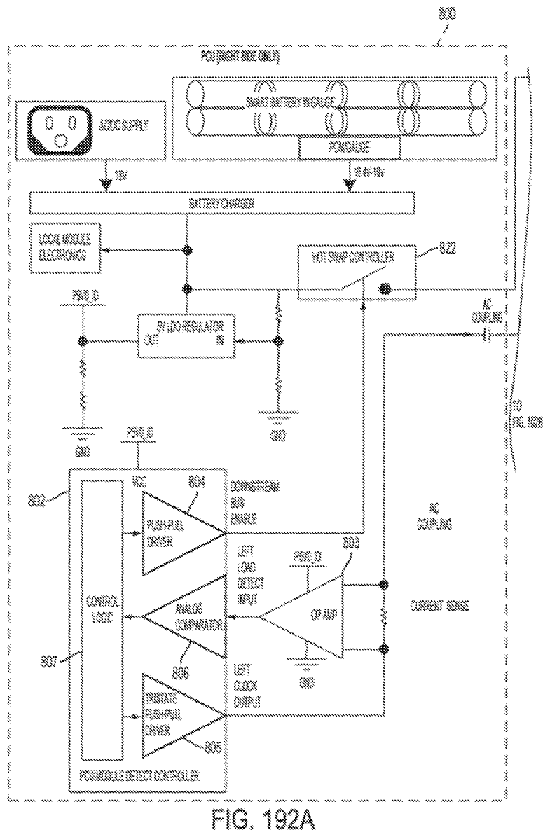

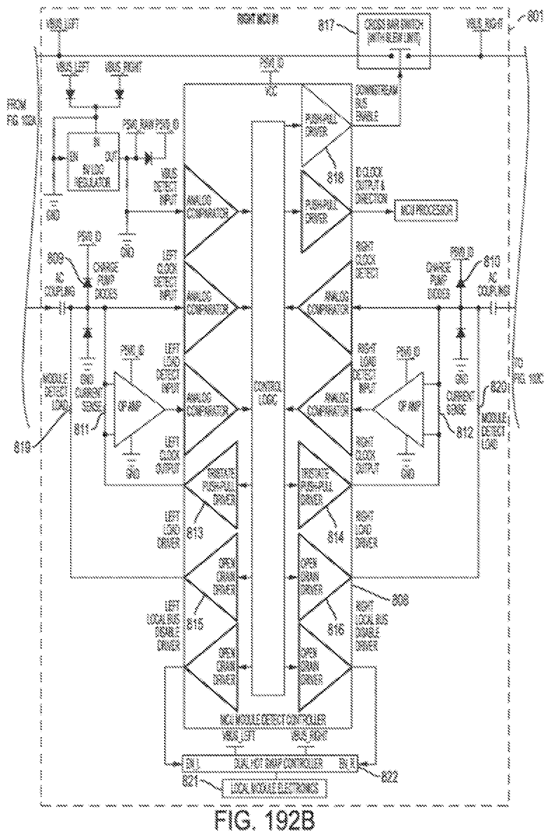

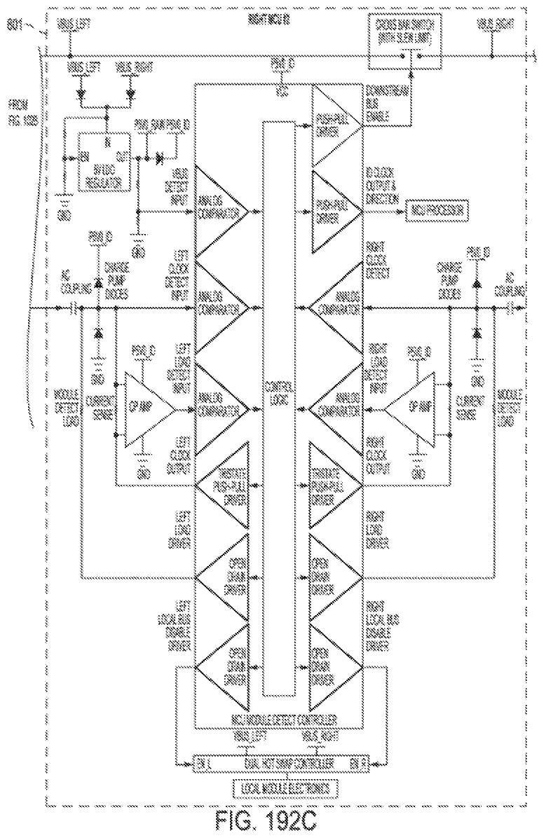

FIGS. 192A-192C show a block diagram of a modular pump system 500 including a central unit 800, and medical-device assemblies 801. The central unit 800 includes a dual hot swap controller 822 to apply power to a power pin, which is controlled by a controller 802 via a driver 804. The controller generates a clock signal via driver 805 which then uses the current sensor 803 to determine changes in impedance as described above. An analog comparator 806 communicates the output of the current sensor 803 (op-amp design) to the control logic 807. The controller 802 uses the state diagrams described above and/or the timing diagrams described above.

The assembly 801 (shown in FIG. 192B and another one is shown in FIG. 192C) includes a controller 808. The controller 808 controls the cross-bar switch 817 via a driver 818. The controller 808 can be powered via left charge pump diodes 809 or right charge pump diodes 810. A clock may be generated to apply to the left communications pin via driver 813 or a right communications pin via driver 814. A left current sense 811 detects changes in impedances of the left communications pin and the right current sense 812 detects changes in impedance as the clock is applied to the right communications pin.

A driver 815 controls whether or not a load 819 is coupled to the left communications pin while a driver 816 controls whether or not a load 820 is applied to the right communications pin. A dual hot swap controller 822 allows power to be applied to the module electronics 821 via a left power pin or right power pin.

FIGS. 103A-103J shows circuitry of the modular pump system 500 to illustrate the assembly ID circuitry, e.g., that may be used with the modular pump system described herein.

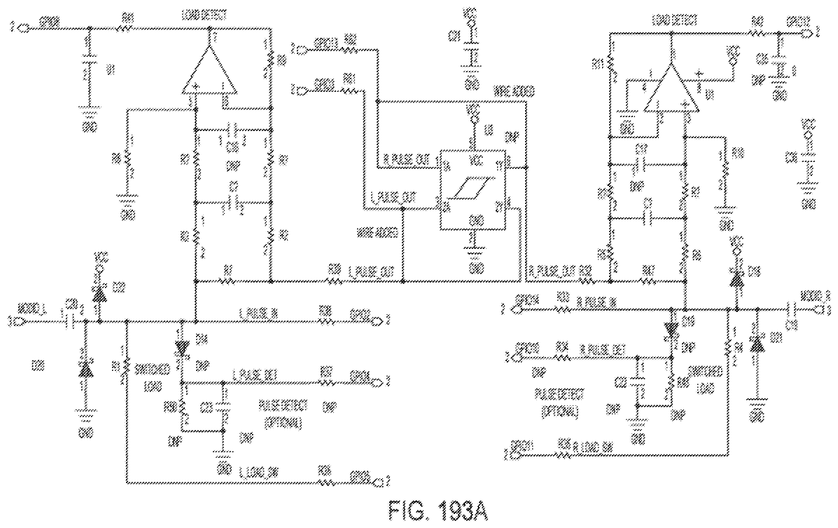

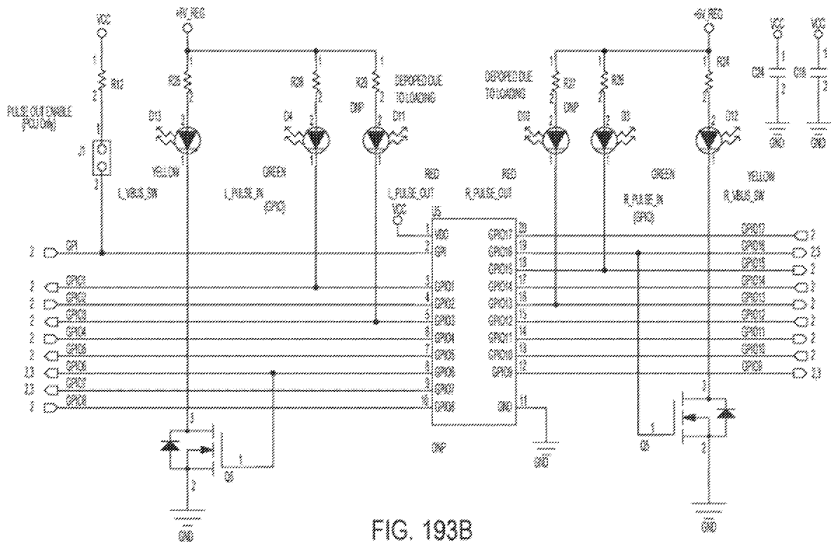

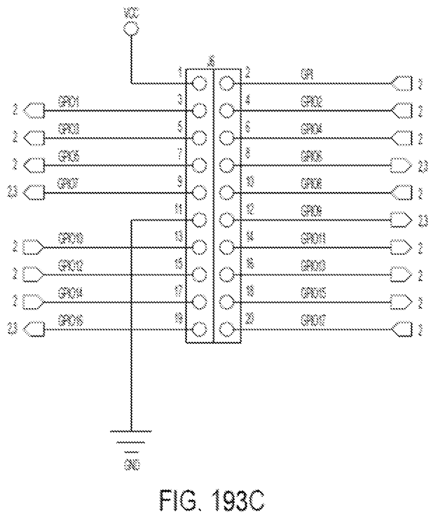

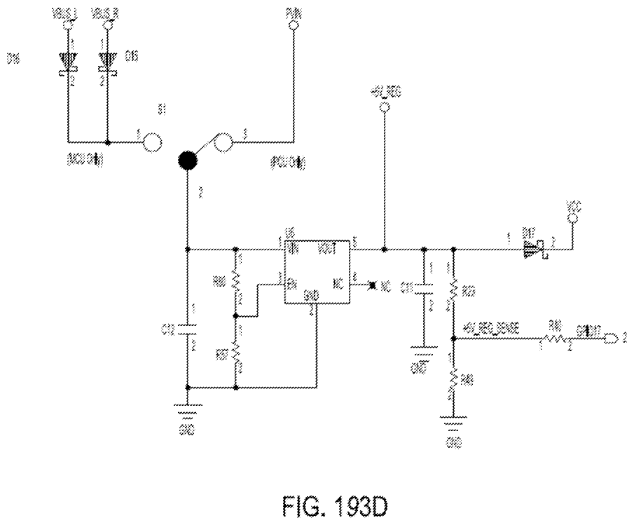

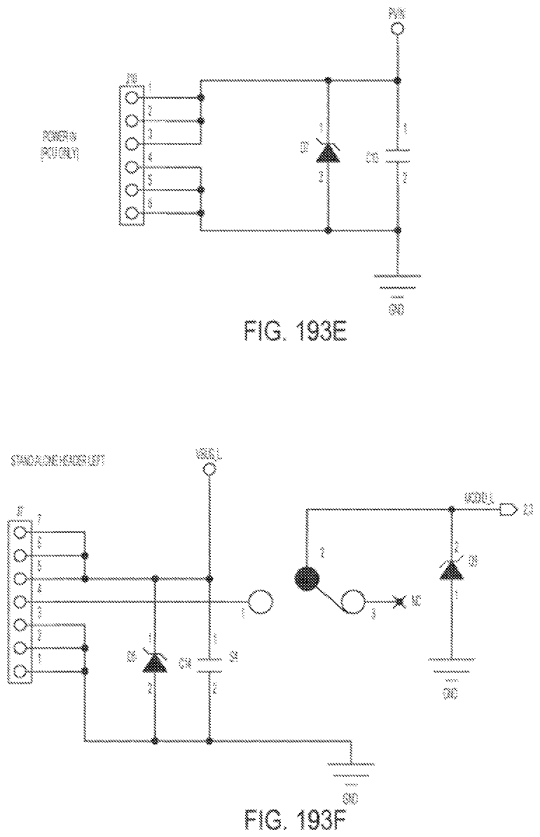

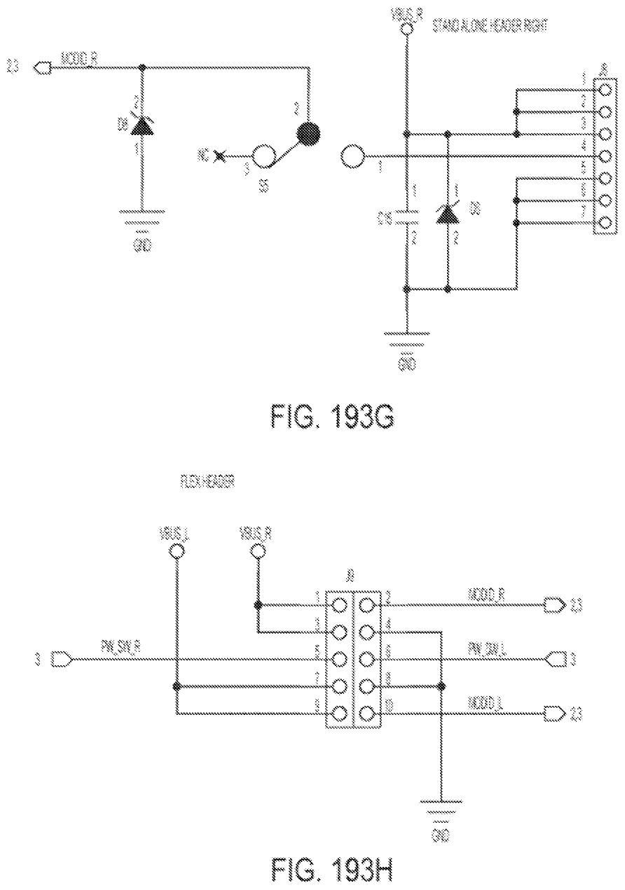

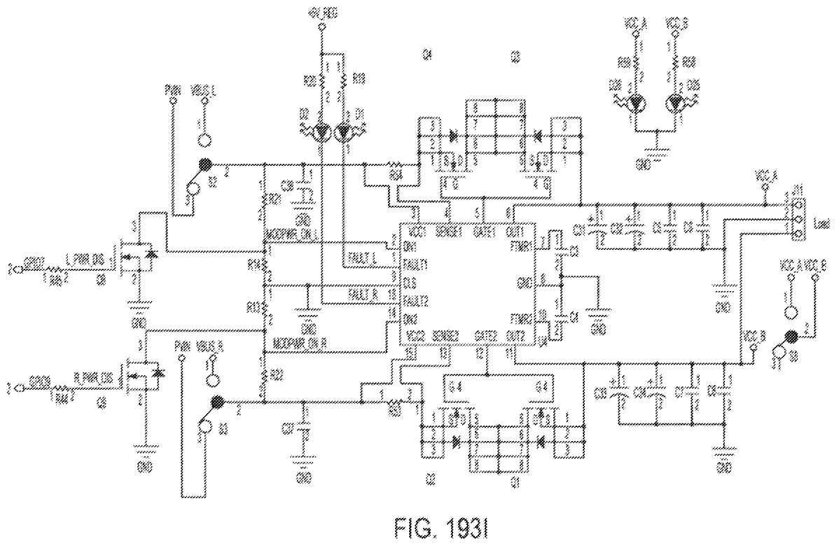

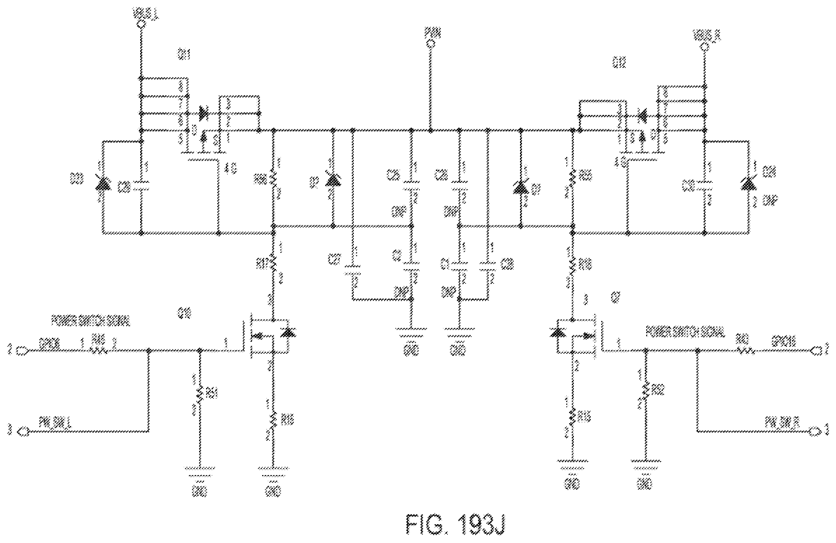

FIG. 193A shows a buffer circuit to buffer the output signal as applied to a communications pin. U3 may be a part number SN74LVC2G17DBVR manufactured by Texas Instruments of 12500 TI Blvd., Dallas, Tex. 75243.

FIG. 193B shows the controller U5. The controller U5 may be part number SLG46721V of Dialog Semiconductor of 100 Longwater Avenue, Green Park, Reading RG2 6GP, United Kingdom. FIG. 193C shows a debugging header. FIG. 193D shows the voltage regulator for the central unit or modular assembly. FIG. 193E shows a power conditioning circuit. FIGS. 193F and 103G shows power conditioning circuits. FIG. 193H shows another debugging header. FIG. 193I shows the dual hot-swap controller. The device U4, may be part number LTC4226IMS-2#PBF made by Analog Devices of One Technology Way, P. O. Box 9106, Norwood, Mass. 02062-9106, United States of America. FIG. 193J shows the cross-bar switch.

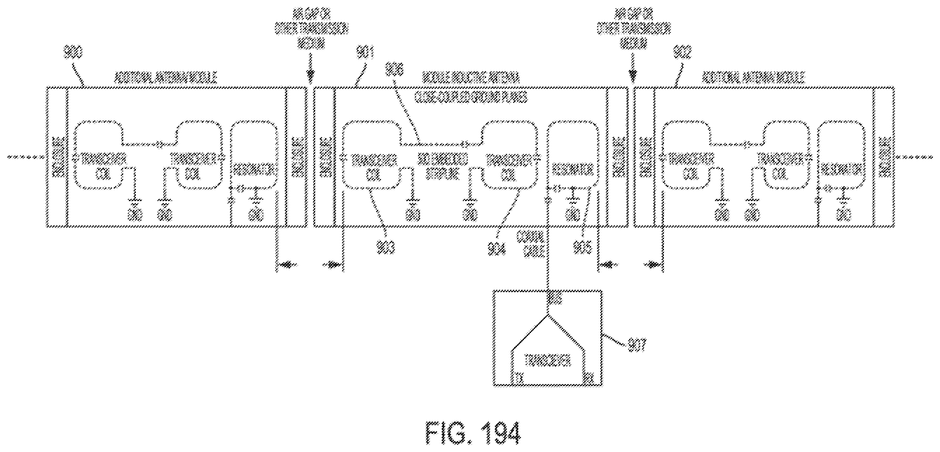

FIG. 194 shows a block diagram of the communication circuitry of the modular pump system. The communication module 900, 901 and 902 are shown. The modules 900, 901, and 902 may each be part of a central unit or an assembly. The module 901 includes a RF stripline 906 which forms the communications bus. The communications bus may be dual use with the start-up sequence described above. One end of the bus includes a transceiver coil 903. On the other end is another transceiver coil 904 coupled to a resonator 905. The resonator communication with another module via an air gap as shown in FIG. 194. A top is coupled to the resonator 905 to interface with the bus via transceiver 907.

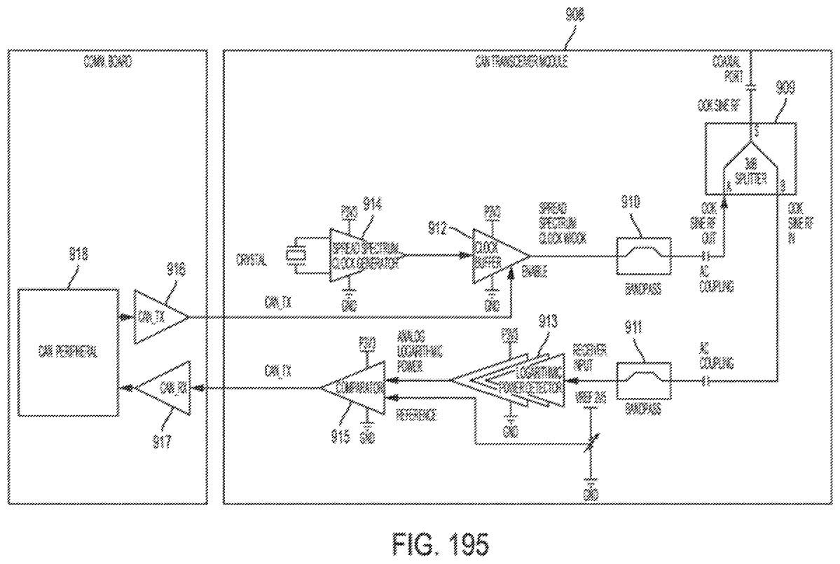

FIG. 195 shows a diagram of the circuitry for interfacing into the communications bus of the modular pump system. A CAN peripheral 918 is coupled to a buffer 916 for transmitting and another buffer 917 for receiving a signal.

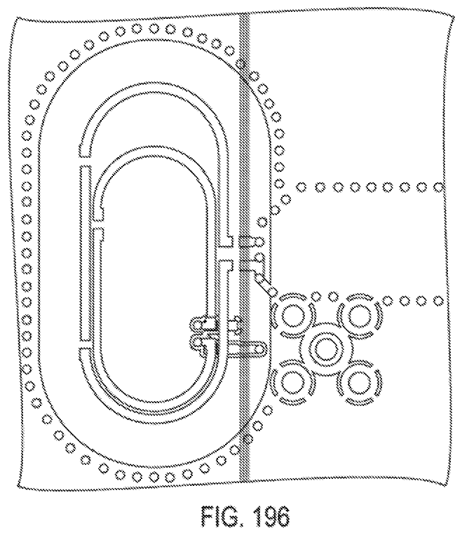

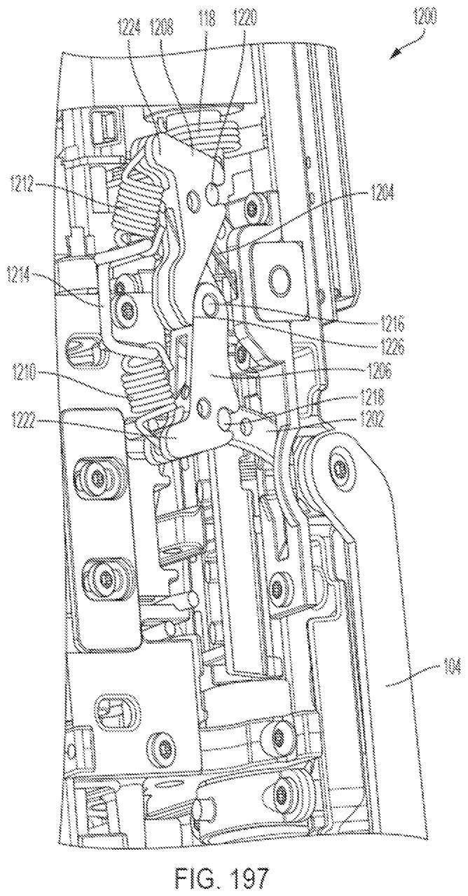

The transceiver module 908 modules the CAN values on an On-Off keying carrier signal. For transmission, the carrier frequency is generated using a spread-spectrum clock generator 914, which is on-off modulated with a clock buffer 912. A band pass filter 910 isolates the circuitry and the splitter 909 allows the signal to interface with the bus. The on-off carrier signal is also received by the splitter 909, which goes through a band pass filter 911 and is demodulated by a power detector 913. A comparator 915 translates the broadband signal to CAN on-off signals for being received by the buffer 917. FIG. 196 shows a PCB diagram of the resonator 905.

In alternative embodiments, the central unit generates the broad-spectrum signal and each of the assemblies grounds the signal to on-off keying modulation to communicate the on-off values needed for CAN communications.

FIG. 197 shows another embodiment of the peristaltic pump 10200 where a relief mechanism 1200 is used. The relief mechanism 1200 may also be referred to as, but is not limited to, a mechanical fuse. The relief mechanism 1200 between the lever 104 and a main shaft 118 includes a first linkage 1202, a second linkage 1204, a first rigid member 1206, and a second rigid member 1208. The relief mechanism 1200 also includes a first spring 1210, a second spring 1212, and a hold 1214. The relief mechanism 1200 operates to relieve high torsion forces. These high torsion forces may be applied to the main shaft 118 by excessive actuation force being applied to the lever 104. The relief mechanism 1200 includes the springs 1210, 1212 and the hold 1214 that form a general triangle shape. The springs 1210, 1212 have enough force to hold the relief mechanism 1200 in place up to a specific load, at which point the relief mechanism 1200 buckles. After buckling, raising the lever 104 causes the relief mechanism 1200 to reset automatically. For example, if the door 102 is open, the main shaft 118 (see FIG. 98) cannot be rotated because of an interlock that prevents a user from closing the lever 104 shut. The force applied to the lever 104, when the door 102 is open, is therefore translated to the relief mechanism 1200 when a user attempts to close the lever 104 with the door 102 open. The relief mechanism 1200 will buckle if a predetermined threshold amount of force is applied to the lever 104 with the door 102 open.

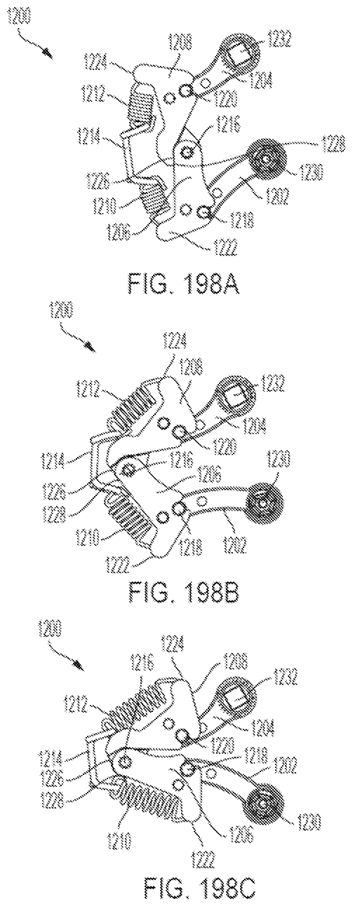

The springs 1210, 1212 bias the first rigid member 1206 and the second rigid member 1208 to rotate via a linkage pivot 1216 toward the hold 1214. The springs 1210, 1212 are coupled together via the hold 1214. Because the spring 1210 is attached to the first rigid member 1206 at a first protrusion 1222, the springs 1210, 1212 rotationally bias the first rigid member 1206 to rotate via the pivot 1218. Likewise, the springs 1210, 1212 rotationally bias the second rigid member 1208 to rotate via the pivot 1220 because the second spring 1212 is attached to the second rigid member 1208 at a second protrusion 1224. However, rotation of the first and second protrusions 1222, 1224 toward the hold 1214 is limited by interactions between a stop 1226 and a surface 1228 (see FIGS. 198A-198C) that occur between the first rigid member 1206 and the second rigid member 1208. The relief mechanism 1200 is in a hold state when the first protrusion 1222 and the second protrusion 1224 are rotated maximally toward each other such that the stop 1226 and surface 1228 are engaged together.

Refer now to FIGS. 198A-198C for an illustration of the transition from a hold state to a triggered state of the relief mechanism 1200. FIG. 198A shows the relief mechanism 1200 in the hold state, FIG. 198B shows the relief mechanism 1200 in an intermediate state, and FIG. 198C shows the relief mechanism 1200 in the triggered state. In FIG. 198A, the stop 1226 and the surface 1228 are interacting with each other because the springs 1210, 1212 apply a rotational force on the pivots 1218, 1220 thereby keeping a fixed distance between the two pivots 1218, 1220. This fixed distance between the pivot 1218 of the first linkage 1202 and the pivot 1220 of the second linkage 1204 correlates the rotations of the first linkage 1202 and the second linkage 1204 via their respective pivots 1230, 1232.

However, if the door 102 is open, the second linkage 1204 cannot fully rotate in the same direction as the first linkage 1202. FIG. 198B shows the effect of a non-moving second linkage 1204, where the first linkage 1202 continues to rotate thereby causing the surface 1228 and the stop 1226 to separate from each other. This separation is due to the rotation of the first rigid member 1206 and limited or no rotation of the second linkage 1204. As the first linkage 1202 rotates towards the second linkage 1204, the first rigid member 1206 and the second rigid member 1208 rotate along the linkage pivot 1216. Rotation along the linkage pivot 1216 in this manner causes the springs 1210, 1212 to stretch and increase their pulling force. FIG. 198C shows the relief mechanism 1200 when rotation along the linkage pivot 1216 has reached its maximum amount of rotation and the distance between pivots 1218, 1220 have reached their minimum distance. In some specific embodiments, if a user releases the lever 104 while the relief mechanism 1200 is in the triggered state, the relief mechanism 1200 will snap back into the hold 1214 state.

However, in some embodiments of the present disclosure, a predetermined amount of rotation of the linkage pivot 1216 may cause the relief mechanism 1200 to quickly actuate into the triggered state. For example, one or ordinary skill in the relevant art would know how to implement an over-center action of the relief mechanism. Additionally, alternatively, or optionally, the relief mechanism 1200 may be bi-stable where the stable states are the hold state and the triggered state.

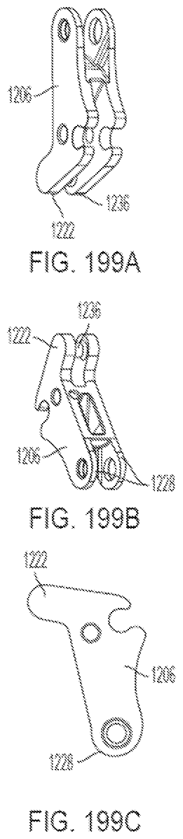

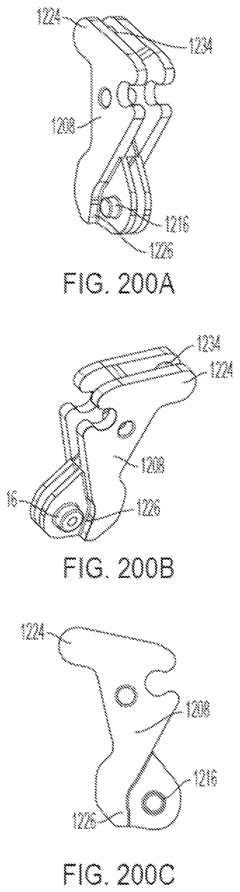

Referring now to FIGS. 199A-199C, which shows the first rigid member 1206. The first rigid member 1206 engages with the second rigid member 1208 (shown in FIGS. 200A-200C). The first rigid member 1206 also includes a fastening point 1236 configured to fasten to an end of a spring (e.g., spring 1210). As is easily seen in FIG. 199A, the first rigid member 1206 includes the surface 1228 that disengages with the stop 1226 of the second rigid member 1208. The surface 1228 is disengaged from the stop 1226 when the relief mechanism 1200 is in the triggered state.

FIGS. 200A-200C shows several views of the second rigid member 1208. The second rigid member 1208 includes the linkage pivot 1216 that engages with the first rigid member 1206 (not shown in FIGS. 200A-200C). The second rigid member 1208 also includes a fastening point 1234 configured to fasten to an end of a spring (e.g., spring 1212). As is easily seen in FIG. 199A, the second rigid member 1208 includes the stop 1226, which is some embodiments is a surface. The stop 1226 is engaged by the first rigid member 1206 when the relief mechanism 1200 is in the hold state.

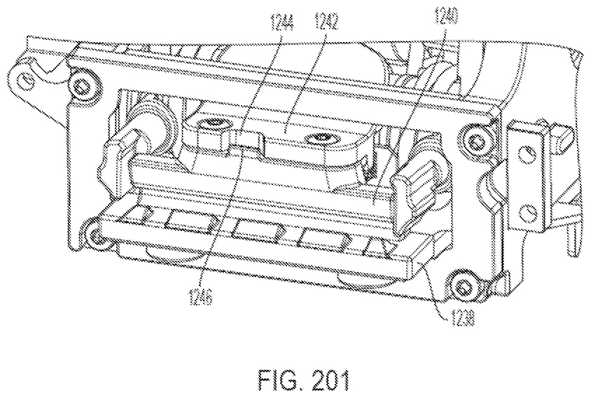

FIG. 201 shows a keyed end effector 1240 that is part of a plunger 1242 in accordance with an embodiment of the present disclosure. The end effector 1240 includes a key 1246 that cooperates with a notch 1244 of the plunger 1242. The end effector 1240 is mounted to the plunger 1242 at a right angle thus the end effector 1240 is coupled to the plunger 1242 at a surface opposite to the surface of the end effector 1240 that faces the platen 1238. That is, the end effector 1240 is attached to the side of the end effector 1240 that faces away from the platen 1238.

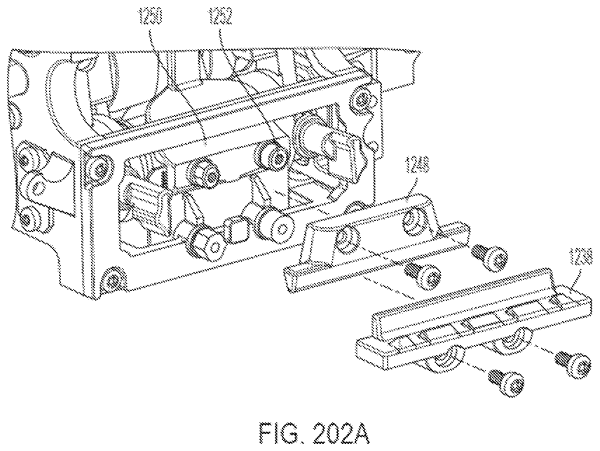

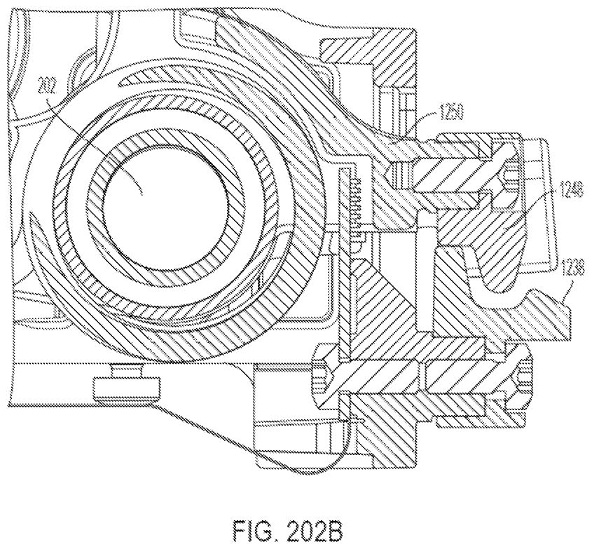

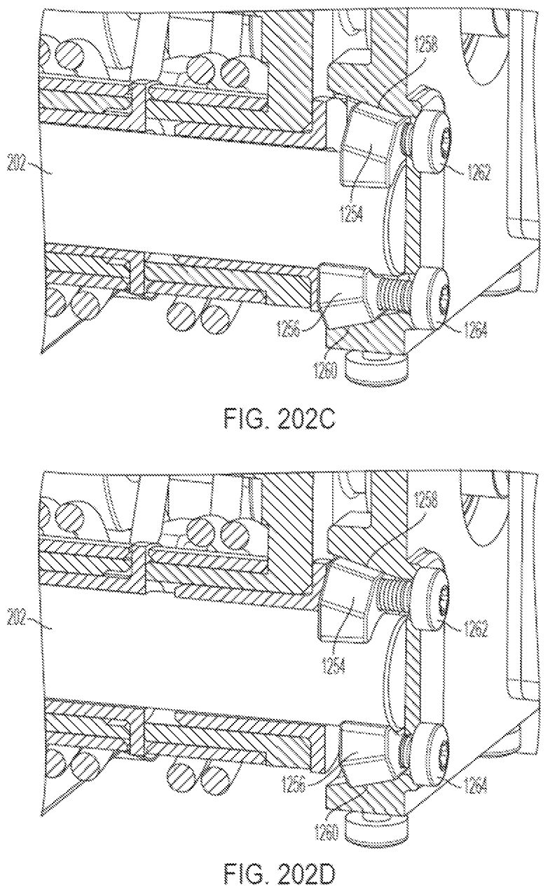

FIGS. 202A-202D show another embodiment of an adjustable end effector 1248 of the plunger 1250. The adjustable end effector 1248 is attached to the plunger 1250 at a side of the adjustable end effector 1248. In some embodiments of the present disclosure, the adjustable end effector 1248 may be made out of a thermal insulator, such as a plastic, polymer, rubber, etc. The thermal insulation may reduce or eliminate the transfer of heat to a fluid in an IV line thereby decreasing the occurrence of outgassing. The adjustable end effector 1248 attaches to the plunger 1250 at two attachment points 1252. Shown in FIG. 202B, the adjustable end effector 1248 is part of the plunger 1250 that pivots around the pivot shaft 202. And any change of angle, orientation, or position of the pivot shaft 202 in relation to the platen 1238 will change/affect the position of the end effector 1248 with respect to the platen 1238. That is, the pivot shaft 202 can be adjusted to thereby adjust the adjustable end effector 1248 during manufacturer and/or in field use.

FIGS. 202C-202D show a first shaft adjuster 1254 and a second shaft adjuster 1256 that move the end of pivot shaft 202 in a plane approximately aligned with axis of the pivot shaft 202. The first shaft adjuster 1254 is disposed adjacent to the pivot shaft 202 and a first ramp 1258. Likewise, the second shaft adjuster 1256 is disposed between the pivot shaft 202 and the second ramp 1260. The first shaft adjuster 1254 and the second shaft adjuster 1256 may be adjusted by a first adjustment screw 1262 and a second adjustment screw 1264, respectively.

As is shown in FIG. 202C, the first shaft adjuster 1254 is positioned closer to the first adjustment screw 1262 thereby moving the pivot shaft 202 away from the first shaft adjuster 1254 as it engages with the first ramp 1258. Also, the second adjustment screw 1264 has been set to position the second shaft adjuster 1256 away from the second adjustment screw 1264. Because the first shaft adjuster 1254 interfaces with the first ramp 1258 as it is actuated toward the first adjustment screw 1262, the pivot shaft 202 is moved away from the first shaft adjuster 1254 and toward the second shaft adjuster 1256.

Likewise, as is shown in FIG. 202D, the second shaft adjuster 1256 is positioned closer to the second adjustment screw 1264 thereby moving the pivot shaft 202 away from the second shaft adjuster 1256 as it engages with the second ramp 1260. Also, the first adjustment screw 1262 has been set to position the first shaft adjuster 1254 away from the first adjustment screw 1262. Because the second shaft adjuster 1256 interfaces with the second ramp 1260 as it is actuated toward the second adjustment screw 1264, the pivot shaft 202 is moved away from the second shaft adjuster 1256 and toward the first shaft adjuster 1254.

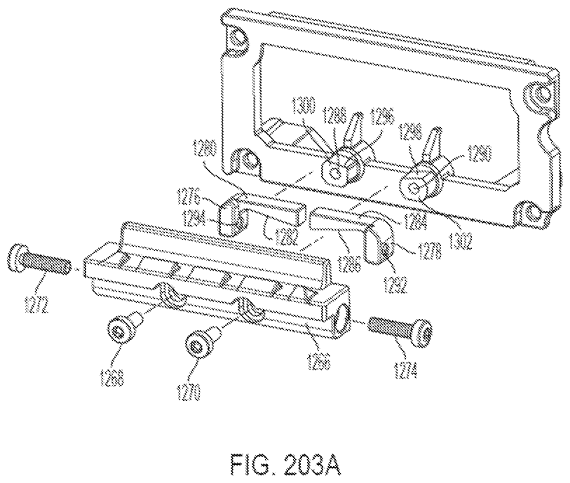

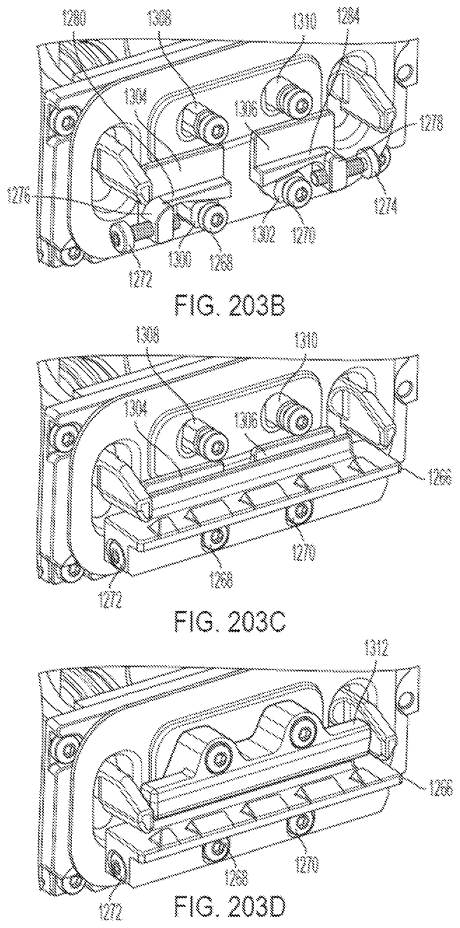

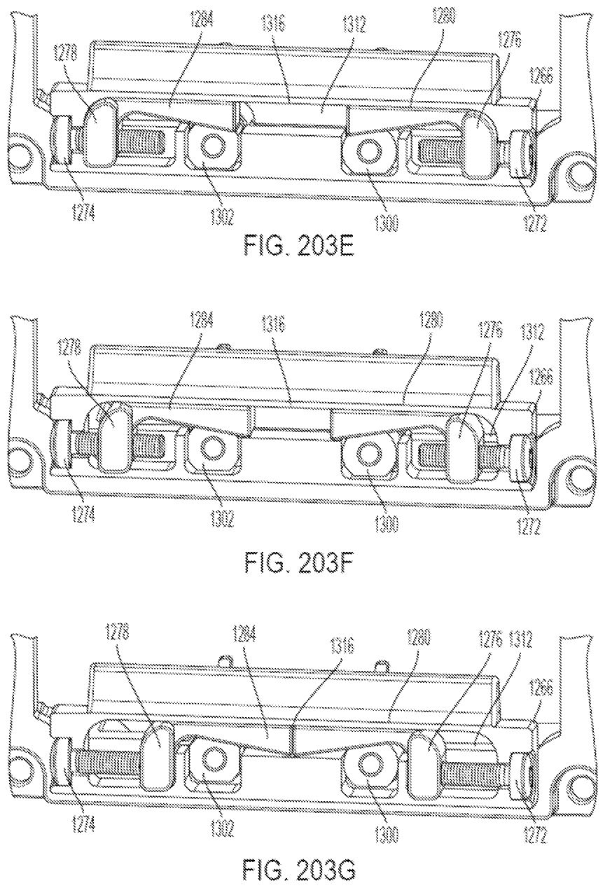

FIGS. 203A-203G illustrate an adjustable platen 1266 in accordance with an embodiment of the present disclosure. The adjustable platen 1266 may optionally, alternatively, or additionally be used in one or more embodiments described herein, or in no embodiments. Any platen described or disclosed herein, such as the adjustable platen 1266, may be made out of a thermal insulator, such as a plastic, polymer, or rubber. The thermal insulation (e.g., use of non-thermally conductive materials such as plastic wedges and/or washers between components and/or plastic plunger) may prevent heat from being transferred to a fluid in an IV line thereby decreasing the occurrence of outgassing.

FIG. 203A shows the adjustable platen 1266 that is mountable by a first mount 1300 and a second mount 1302. A first mount screw 1268 secures a first end of the adjustable platen 1266 to the first mount 1300. A second mount screw 1270 secures a second end of the adjustable platen 1266 to the second mount 1302. The first mount screw 1268 engages with a first threaded hole 1288 while the second mount screw 1270 engages with a second threaded hole 1290.

The first adjuster 1276 includes a threaded hole 1294, a first side 1280, and a second side 1282. The second side 1282 of the first adjuster 1276 engages with a first engagement surface 1296 of the first mount 1300. The first adjustment screw 1272 fastens to the first adjuster 1276 to control its distance relative to the first mount 1300. As the first adjuster 1276 is actuated away from the first mount 1300 by the first adjustment screw 1272, the distance between the first side 1284 and the second side 1282 increases along the thickness of the first adjuster 1276 that engages with the first engagement surface 1296. That is, the second side 1282 and the first engagement surface 1296 act as a ramp to actuate the adjustable platen 1266 away from the first mount 1300 as the adjuster is actuated away from the first mount 1300.

Similarly, the second adjuster 1278 includes a threaded hole 1292, a first side 1284, and a second side 1286. A second engagement surface 1298 of the second mount 1302 engages with the second side 1282 of the second adjuster 1278. The second adjustment screw 1274 fastens to the second adjuster 1278 to control its distance relative to the second mount 1302. As the second adjuster 1278 is actuated away from the second mount 1302 by the second adjustment screw 1274, the distance between the first side 1284 and the second side 1282 increases along the thickness of the second adjuster 1278 that engages with the second engagement surface 1298. That is, the second side 1286 and the second engagement surface 1298 Act as a ramp to actuate the adjustable platen 1266 away from the second mount 1302 as the second adjuster 1278 is actuated away from the second mount 1302.

In some embodiments, any fastener may be used for one or more of the first adjustment screw 1272, the second adjustment screw 1274, the first mount screw 1268, or the second mount screw 1270, such as bolts, latches, glues, epoxies, etc.

FIGS. 203B-203D show how an adjustment to the platen 1266 may be made. In FIG. 203B, the first adjuster 1276 is positioned adjacent to the first mount 1300 and the second adjuster 1278 is positioned adjacent to the second mount 1302. The first adjuster 1276 has an optional first planar portion 1304 that can be positioned between the adjustable platen 1266 and the pump, when assembled. The second adjuster 1278 also has an optional second planar portion 1306 that can be positioned between the adjustable platen 1266 and the pump.

As is easily seen in FIG. 203B, actuating the first adjustment screw 1272 into the first adjuster 1276 actuates the first adjuster 1276 toward the plunger 1312 (FIG. 203C shows the plunger 1312 mounted). In FIG. 203B, the plunger mount screws 1310, 1308 are shown. FIG. 203C shows the adjustable platen 1266 mounted and FIG. 203D shows the plunger 1312 mounted. Referring now to FIGS. 203B-203D, it can be easily seen that movement of the adjustable platen 1266 relative to the plunger 1312 adjusts how the plunger 1312 will interact with a tube placed within the adjustable platen 1266. The plunger 1312, in some embodiments, may have a repeatable motion that is stopped (e.g., it can be seen in FIG. 14 how an end effector 128 has actuation limited by contact with a fixed stop).

FIGS. 203E-203G show a cross-sectional view between the pump and the adjustable platen 1266. As can be seen, the adjustable platen 1238 includes a space 1314 that has a surface 1316 configured for engagement with the first side 1280 of the first adjuster 1276 and also for engagement with the first side 1284 of the second adjuster 1278. In accordance with one embodiment of the present disclosure, a head of the second adjustment screw 1274 and a head of the first adjustment screw 1272 may be in a fixed position relative to the adjustable platen 1266 such that actuation of the adjustment screws 1272, 1274 actuate the first and second adjusters 1276, 1278, respectively. As can be seen, FIG. 203E shows the first and second adjusters 1276, 1278 at a position to maximally raise the adjustable platen 1266, FIG. 203F shows an intermediate position, and FIG. 203G shows the first and second adjusters 1276, 1278 at a position to keep the adjustable platen 1266 maximally away from the plunger 1312.

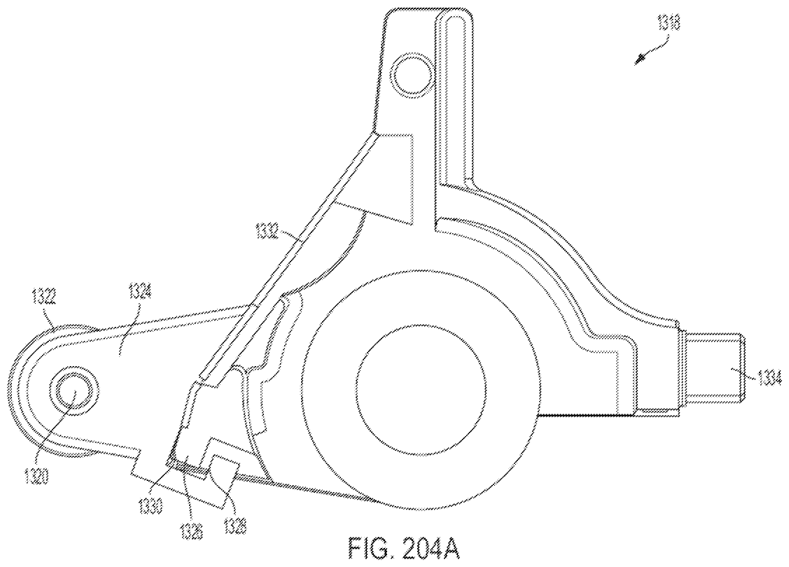

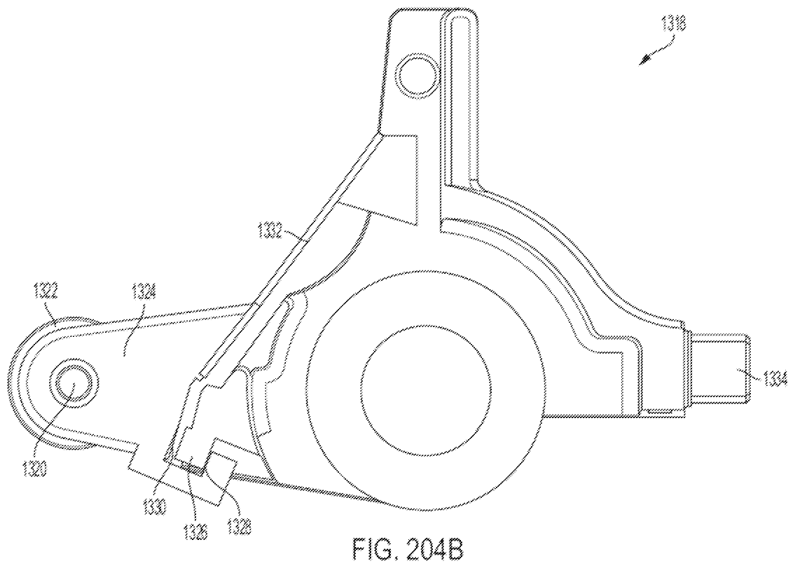

FIGS. 204A-204B show a multi-stage, spring-biased plunger 1318 of a peristaltic pump in accordance with an embodiment of the present disclosure. The plunger 1318 includes a follower 1324 that can follow a cam to actuate an end effector (not shown in FIGS. 204A-204B) coupled to an attachment point 1334. The plunger includes a roller 1322 that pivots around a pivot pin 1320. The roller 1322 is coupled to the rest of the plunger 1318 via a leaf spring 1332. The leaf spring 1332 allows the roller 1322 to actuate between two positions by interaction with a stop 1326. FIG. 204A shows a first position of the roller 1322 and FIG. 204B shows a second position of the roller 1322, both of which are based upon the position of the leaf spring 1332.

In operation, when the roller 1322 is in contact with a cam and the spring-biased plunger 1318 starts to interact with the tube, the stop 1326 will have contact with the first contact 1330. Initially, as the spring-biased plunger 1318 actuates the end effector toward the tube, the cam holds the follower 1324 against the stop 1326 at the first contact 1330 as shown in FIG. 204A. Also, as the spring-biased plunger 1318 actuates an end effector against the tube, the resilience of the tube will, after a threshold amount of force is applied to the end effector by the tube, cause the leaf spring 1332 to transition such that the follower 1324 contacts against the second contact 1328 as shown in FIG. 204B.

The amount of force needed to transition the leaf spring 1332 is predetermined and may be a function of the amount of force applied to the tube. For example, the leaf spring 1332 may transition based upon the amount of force applied to the tube when the inlet and outlet valve are closed.

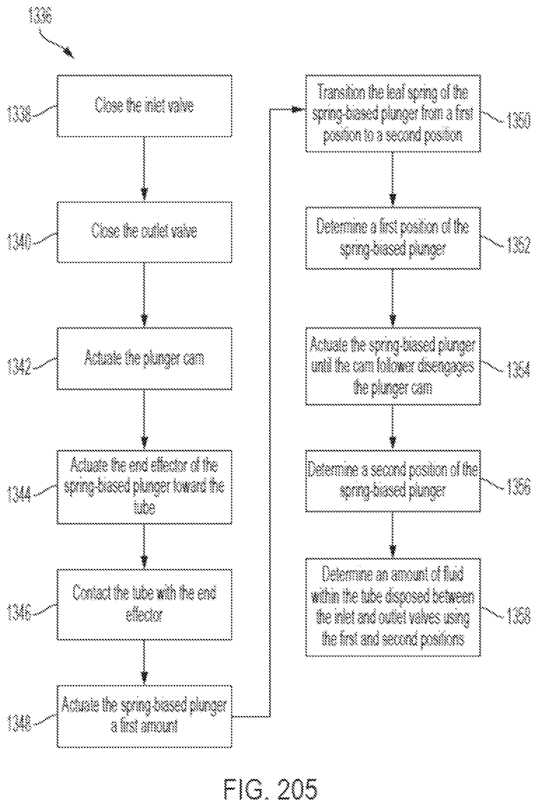

FIG. 205 shows a flow chart diagram of a method 1336 for actuating the spring-biased plunger 1319 that is biased by a multi-stage spring. Act 1338 closes the inlet valve. Act 1340 closes the outlet value. Act 1342 actuates the plunger cam, e.g., to lower the end effector onto the tube such as in 1344. Act 1346 contacts the tube with the end effector. Air may be in the tube. Thus, in Act 1348, the spring-biased plunger 1318 is actuated a first amount, during which time, the air, e.g., a bubble, will compress more readily than any liquid contained therein. In Act 1350, the leaf spring 1332 of the spring-biased plunger 1318 transitions from a first position to a second position, e.g., when a predetermined force is applied to the tube. This force may be determined such that, any air within the tube is substantially (e.g., 95% less volume) is compressed to a small volume. Thus, the position of the plunger and/or end effector during this transition can be used to determine how much liquid is within the tube. Thereafter, the spring-biased plunger 1318 may continue to be actuated until the cam follower disengages the plunger cam, such as in Act 1354. Act 1356 determines a second position of the spring-biased plunger 116. The first and second positions may be used to estimate the amount of air in the tube in Act 1358. The resulting amount of fluid calculated to remain in the tube thus can be used to estimate the amount of fluid delivered downstream when the outlet value is opened and/or the fluid is ejected past the outlet value. The two positions of the plunger 1318 may be linearly related to the amount of air in the tube, or any suitable model may be used to estimate the air in the tube, such as linear or polynomial regression from experimentally derived data. The first measurement position is a point where the leave spring is positioned such that 1326 is positioned between 1328 and 1330, not making contact with either of them. The measurement happens in that middle region where the force should be approximately constant.

### Heat Transfer Design

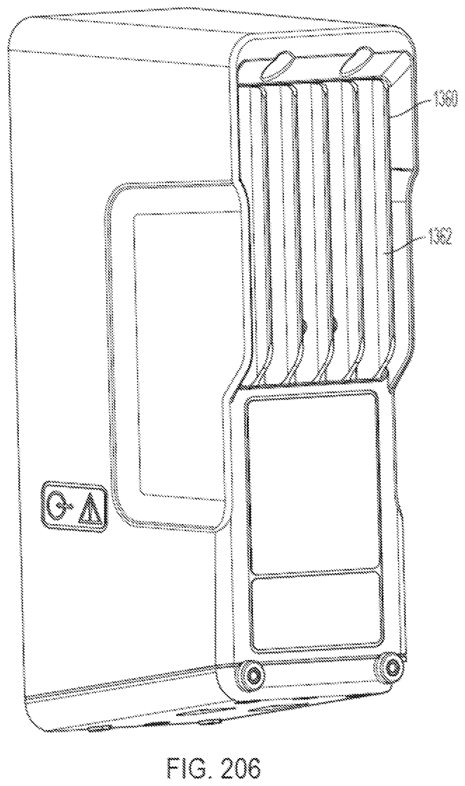

FIG. 206 shows a back side of the peristaltic pump 1020 having a thermal dissipation assembly 1360 including a heatsink 1362 in accordance with an embodiment of the present disclosure. FIG. 206 shows the overall positioning of the thermal dissipation assembly 1360 relative to the body of the peristaltic pump 1020.

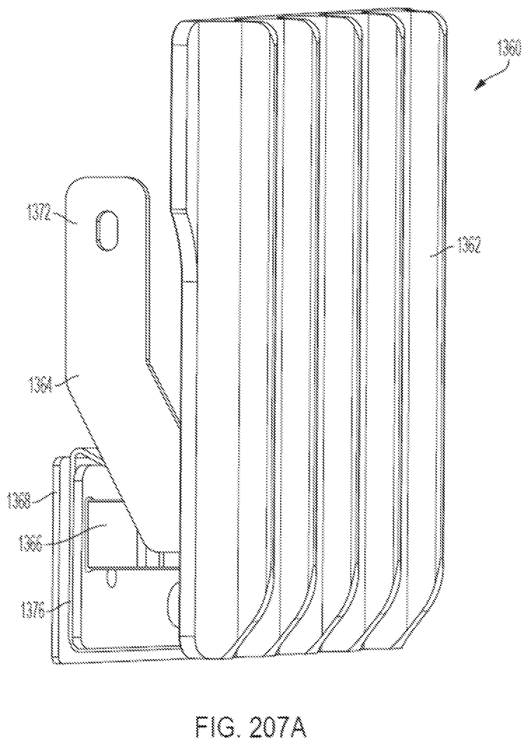

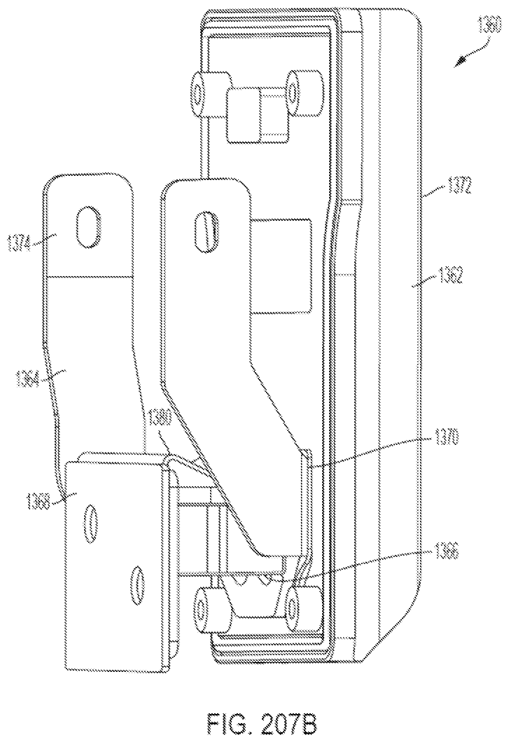

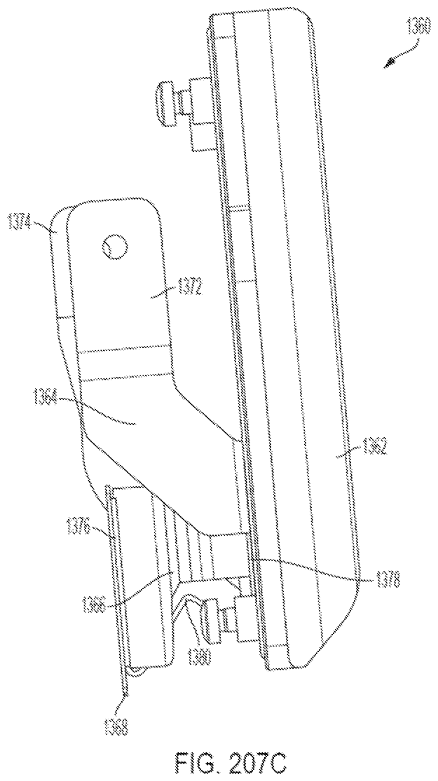

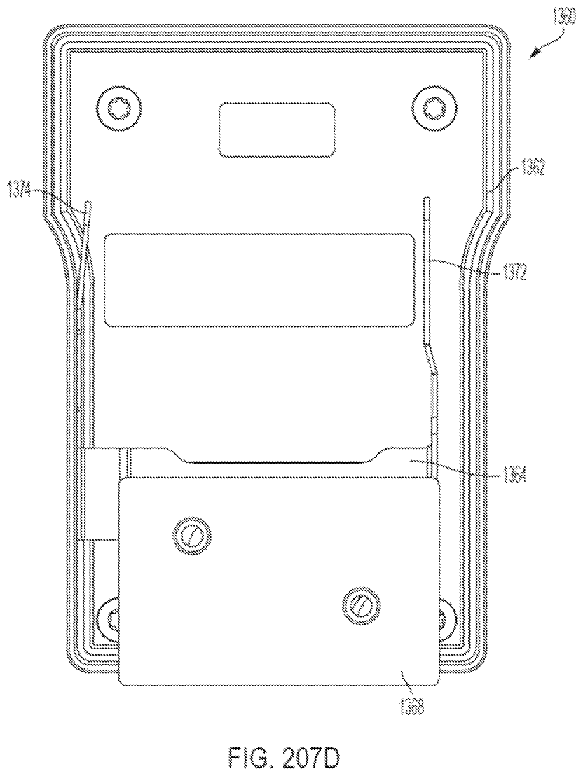

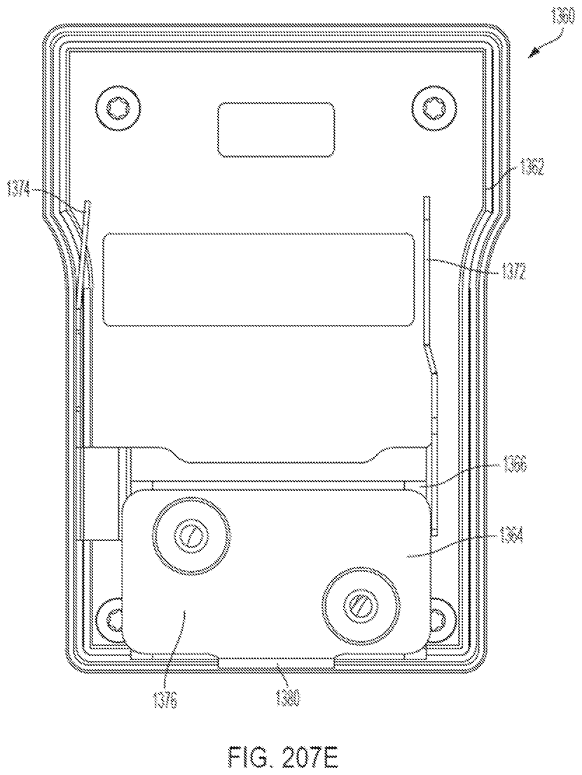

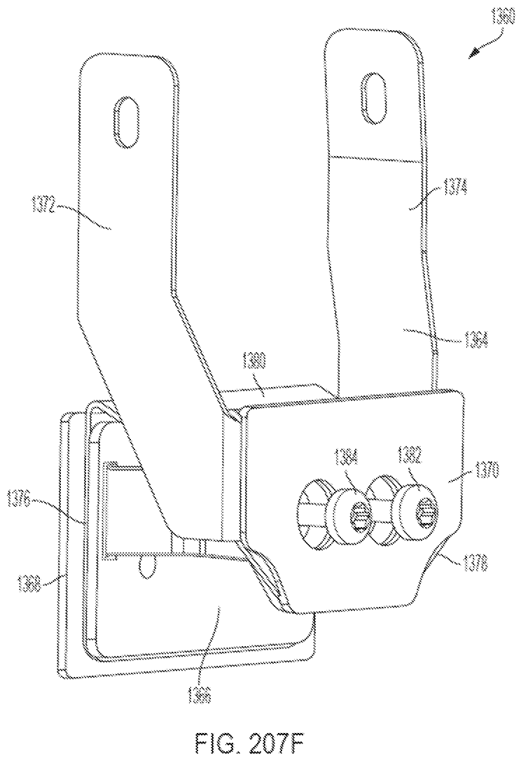

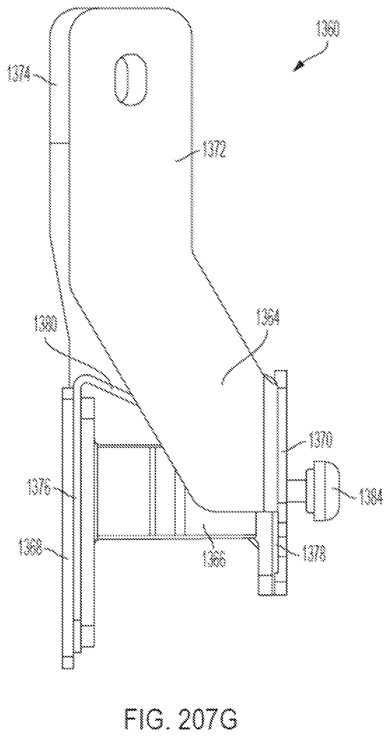

FIGS. 207A-207G show several views of the thermal dissipation assembly 1360. As is shown in FIG. 207A, the thermal dissipation assembly 1360 includes the heatsink 1362, a planar-thermal connector 1364, and a heat-strap bracket 1366. In FIG. 207B, it can be seen that the planar-thermal connector 1364 includes a first arm 1372 and a second arm 1374 both extending to connect to a motor (not shown) to absorb thermal energy therefrom and transfer the thermal energy to the heatsink 1362. The first arm 1372 and the second arm 1374 may be soldered onto the motor. The planar-thermal connector 1364 may be made of any metal, alloy, or thermally-conductive material, including, but not limited to, copper or aluminum. In some embodiments, a braided wire may be used in place of the planar metal portions of the planar-thermal connector 1364. In yet additional embodiments, the planar portion could be replaced by one or more heat pipes.

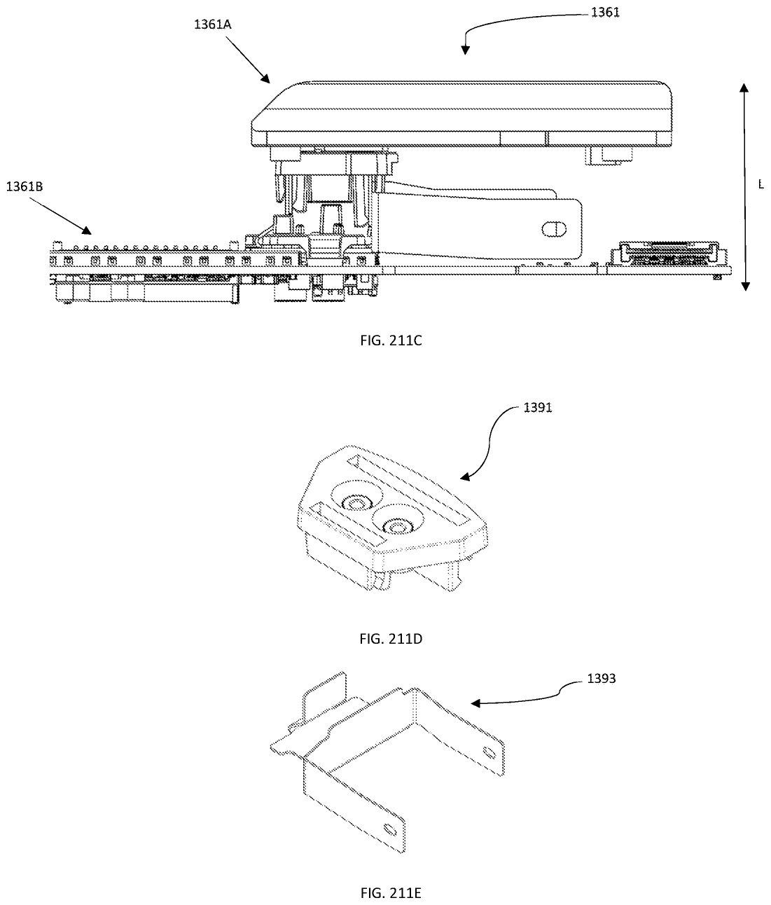

FIG. 207C shows a side-view of the thermal dissipation assembly 1360. The thermal dissipation assembly 1360 includes a first interface plate 1376 that can interface into a powerbar 1430 (see FIGS. 211A-211B) to absorb heat therefrom. The powerbar 1430 may serve to absorb heat from high-heat electronics, such as power MOSFET's or other power semiconductors. A powerbar 1430 may be, for example, an EMI shield surrounding power electronics that is also being used to dissipate thermal energy. In some embodiments, thermal paste may be used.

FIG. 207D shows the thermal pad 1368 of the thermal dissipation assembly 1360. The thermal dissipation assembly 1360 is attached to the first interface plate 1376, which can be seen in FIG. 207E in which the thermal pad 1368 is removed thereby exposing the first interface plate 1376. FIGS. 207F-207G show the thermal dissipation assembly 1360 with the heatsink 1362 removed. A second thermal pad 1370 is shown and it is disposed between a second interface plate 1378 and the heatsink 1362 to facilitate thermal flow between the heatsink 1362 and the rest of the planar-thermal connector 1364. In some embodiments, thermal paste may be used. Additionally, a heat-strap bracket 1366 may be disposed between the first interface plate 1376 and the second interface plate 1378 to ensure a spaced-relationship between the first and second interface plates 1376, 1378. The heat-strap bracket 1366 may be made of any insulating material, such as an insulating plastic or polymer. Screws 1382, 1384 can be used to secure the heatsink 1362 to the heat-strap bracket 1366.

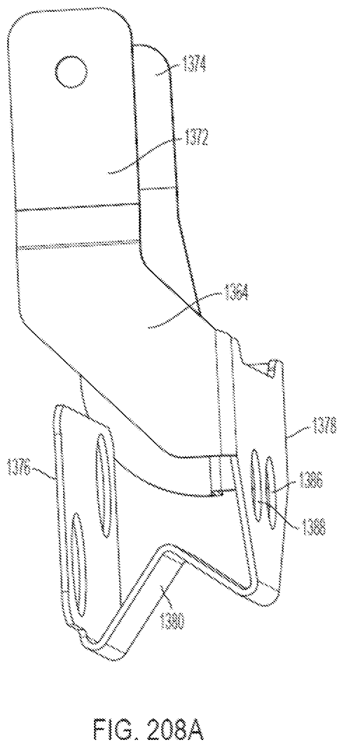

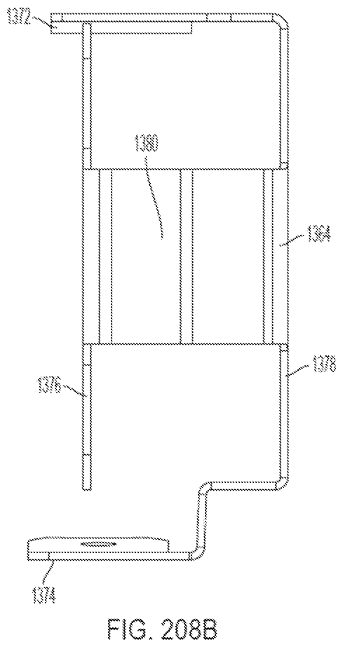

Refer now to both of FIGS. 208A-208B, which show additional views of the planar-thermal connector 1364. As is easily seen in FIG. 208A, a spring 1380 is used to resiliently secure the first and second interface plates 1376, 1378 thereby facilitating contact of the first and second thermal pads 1368, 1370. The spring 1380 of the planar-thermal connector 1364 facilitates the accommodation of varying manufacturing tolerances. That is, the spring 1380 can expand the distance between the first interface plate 1376 and the second interface plate 1378 to ensure a good fit within the peristaltic pump 1020. The holes 1386, 1388 allow for the screws 1382, 1384, respectively, to traverse through the first interface plate 1376 to the heat-strap bracket 1366 (see FIG. 207G).

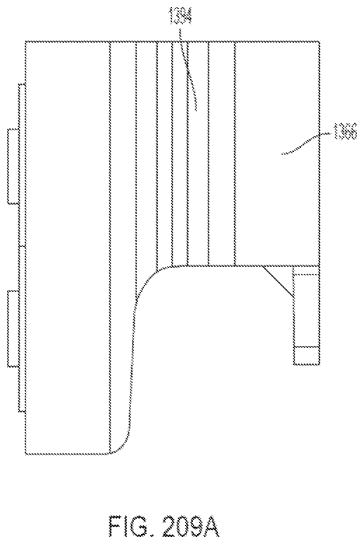

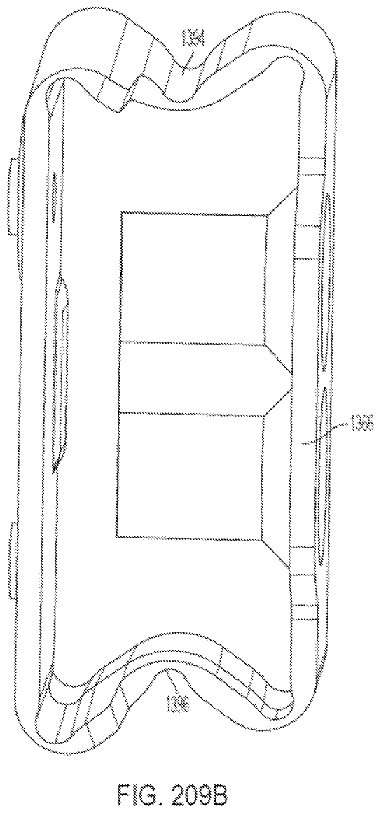

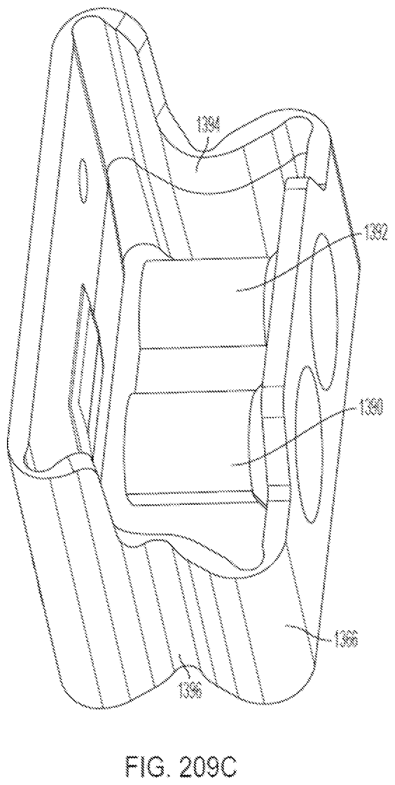

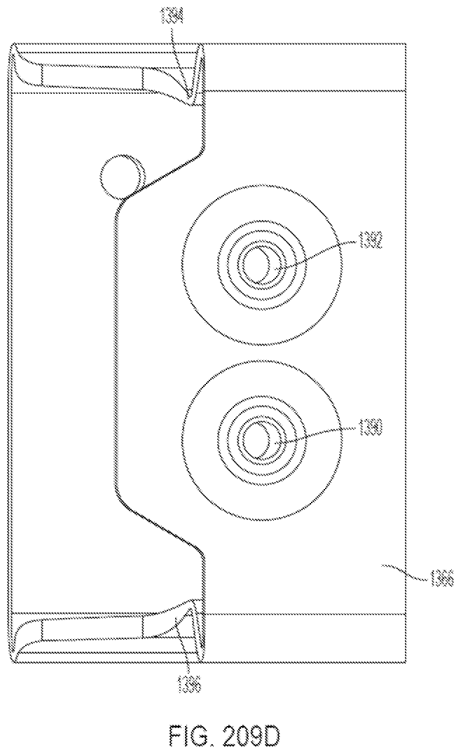

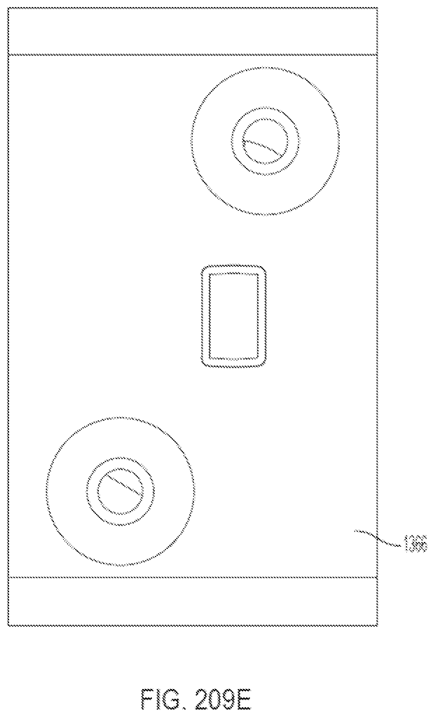

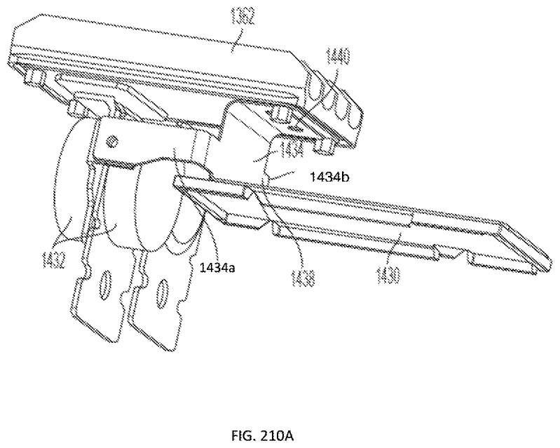

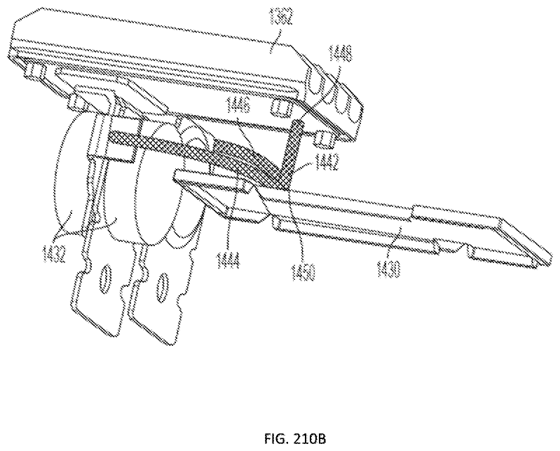

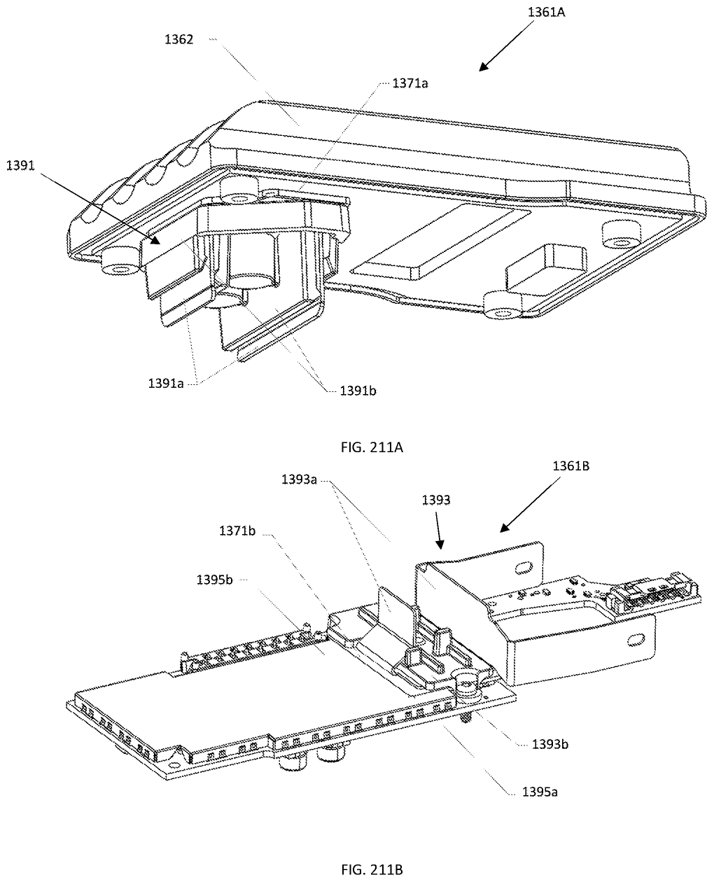

FIGS. 209A-209E show several views of the heat-strap bracket 1366. The heat-strap bracket 1366 includes threaded holes 1390, 1392, to receive the screws 1382, 1384, respectively. The heat-strap bracket 1366 also includes springs 1394, 1396, to provide resilience so that the heat-strap bracket 1366 can accommodate various manufacturing tolerances. Additionally, alternatively, or optionally, the springs 1394, 1396 can provide resilience to press the thermal pads 1368, 1370 against the powerbar 1430 and heatsink 1362, respectively.

FIG. 210A shows another embodiment of a planar thermal connector 1434 in accordance with an embodiment of the present disclosure. The planar thermal connector 1434 is thermally coupled to a motor 1432 and to the heatsink 1362. The planar thermal connector 1434 includes a first arm 1434a and a second arm 1434b, both thermally connected to the motor 1432 to dissipated heat toward the heatsink 1362. The planar thermal connector 1434 includes a first end 1438 thermally coupled to the powerbar 1430 and a second end 1440 thermally coupled to the heatsink 1362. The planar thermal connector 1434 can absorb heat from the motor 1432 and the powerbar 1430 to thereby dissipate heat energy into the surround ambient air through the heatsink 1362. The powerbar 1430 may be an EMI shield configured to shield power electronics that is also used to dissipate heat from the power electronics, for example.

FIG. 210B shows an embodiment of a heatsink 1362 having a braided-wire 1442 to transfer heat to the heatsink 1362 from the motor 1432 and the powerbar 1430. The braided-wire 1442 includes a first end 1450 coupled to the powerbar 1430 and a second end 1448 coupled to the heatsink 1362. The braided-wire 1443 is also coupled to a heatsink 1362 of the motor 1432 via third ends 1444, 1446. The third ends 1444, 1446 may also be referred to as arms. The heatsink 1362 may be formed from a thermally conductive material, e.g., a metal.

FIGS. 211A-211C show an embodiment of a thermal dissipation assembly in accordance with an embodiment of the present disclosure.

In some embodiments, the third ends 1444, 1446, are coupled directly to the heatsink 1362 to provide a direct connection between the heatsink 1362 and the motor 1432. Additionally or alternatively, the first end 1450 and the second end 1448 may be connected directly together. The braided-wire 1443 may be soldered onto the powerbar 1430, the heatsink 1362, the motor 1432, and/or each other. Additionally or alternatively, clips may be used to secure the braided-wire 1443 onto the powerbar 1430, the heatsink 1362, the motor 1432, and/or each other. It will be appreciated by one of ordinary skill in the relevant art, that in place of the braided-wire, any combination of braided-wire and/or heat pipes may be used to transfer heat to the heatsink 1362 or to any other part of the peristaltic pump 1020.

### Pumping Actions

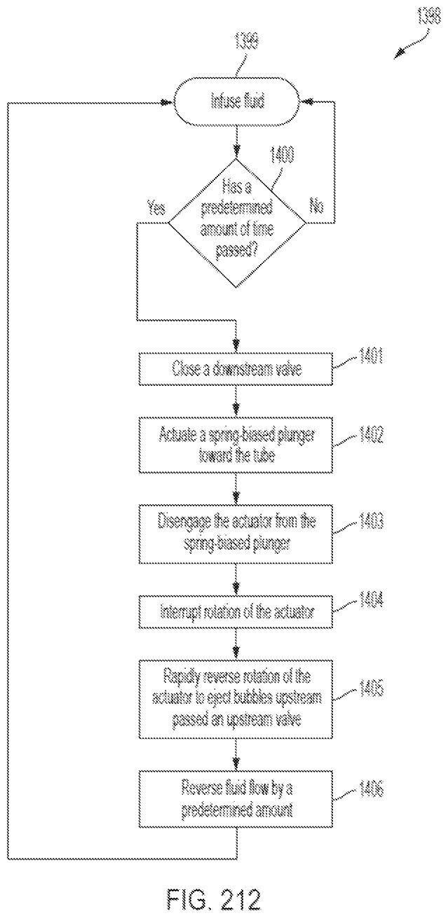

FIG. 212 shows a flow chart diagram of a method 1398 for dislodging bubbles in an IV line in accordance with an embodiment of the present disclosure. The method 1398 includes acts 1399-1406. Act 1399 infuses fluid into a patient, e.g., using a peristaltic pump 1020. Act 1400 determines if a predetermined amount of time has passed. In other embodiments, a predetermined amount of fluid volume, a number of peristaltic pumping cycle, and/or an amount of air pumped past a downstream air sensor may be used in place of a predetermined amount of time. If act 1400 determined a predetermined amount of time has passed, then Act 1401 closes a downstream valve. Act 1403 disengages the actuator from the spring-biased plunger 116. Act 1404 interrupts the actuation (e.g., rotation) of the actuator. Act 1405 rapidly reverses actuation (e.g., rotation) of the actuator (e.g., a cam shaft) to eject bubbles upstream and passed an upstream valve. Act 1406 may reverse the fluid flow by a predetermined amount. The predetermined amount may be a predetermined amount of time, fluid volume, and/or a number of peristaltic pumping cycles, including fractions thereof. In some embodiments, the peristaltic pump includes an inlet valve, an outlet valve, and a spring-biased plunger 116 where the spring-biased plunger 116 includes a spring configured to bias the plunger again the tube and the cam is configured to actuate the plunger away from the tube. The method 1398 may reverse fluid flow when the outlet valve is closed and the inlet valve in open, e.g., fluid flow may be reversed as the plunger is being actuated toward the tube. In yet additional embodiments, the test at Act 1400 may be done at other locations, e.g., somewhere between 1401 and 1404. In yet additional embodiments, Act 1403 may be removed.

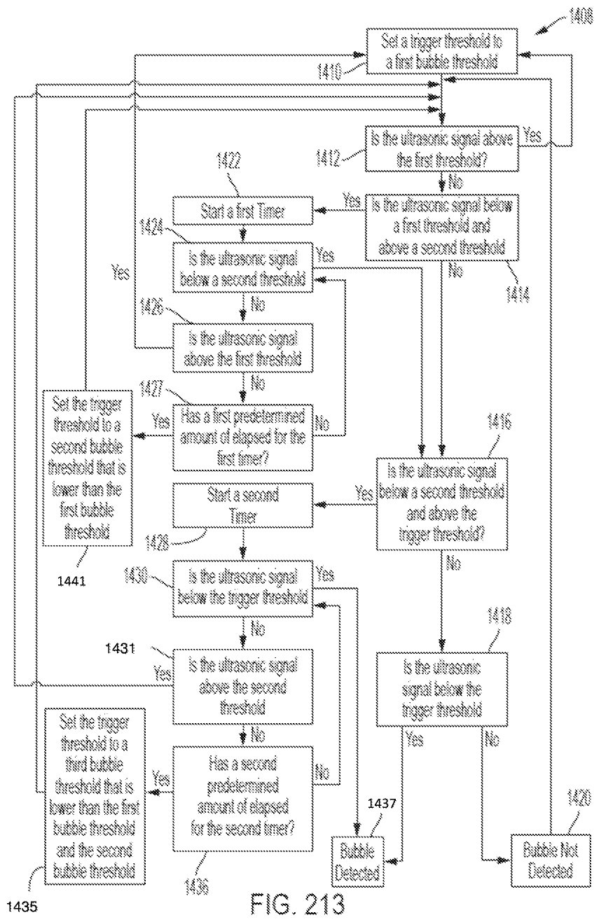

FIG. 213 shows a flow chart diagram of a method 1408 for detecting a bubble in accordance with an embodiment of the present disclosure. The method 1408 may be used on a pump, such as peristaltic pump 1020 or syringe pump. The pump may have an air sensor, such as an ultrasonic air sensor. The method 1408 uses two thresholds to determine whether or not a bubble has been detected and/or should be accounted for using an ultrasonic signal, e.g., signal strength, a drop in signal strength, a gain in signal strength, etc. The method includes acts 1410-1440. Act 1410 sets a trigger threshold to a first bubble threshold. Act 1412 determines if the ultrasonic signal is above the first threshold. If the ultrasonic signal is above the first threshold, then the method continues return to Act 1410. Otherwise, the method continues to Act 1414 to determine if the ultrasonic signal is below the first threshold and above a second threshold. If it is, the method transitions to Act 1422. Otherwise, the method 1408 transitions to Act 1416. Act 1416 determines if the ultrasonic signal is below a second threshold and above the trigger threshold. If it is true, the method 1408 transitions to Act 1428. If Act 1416 has a false determination, then the method 1408 transitions to Act 1418. Act 1418 determines if the ultrasonic signal is below the trigger threshold. If not, then the method 1408 continues to Act 1420 in which case it is determined that no bubble is detected, and the method 1408 proceeds back to Act 1412.

At Act 1422, a first timer is started. At Act 1424, if the ultrasonic signal is below a second threshold, then the method transitions to Act 1416. Otherwise, the method continues to Act 1426. If the ultrasonic signal is above the first threshold at Act 1426, then the method continues at Act 1410. Otherwise, the method continues to Act 1427 to determine if a predetermined amount of time has elapsed for the first timer (see Act 1422). If a predetermined amount of time has elapsed for the first timer, the method continues to Act 1441. Otherwise, the method continues to Act 1426.

After a first predetermined amount of time has elapsed for the first timer at Act 1427, Act 1441 sets the trigger threshold to a second bubble threshold that is lower than the first bubble threshold and the method continues to prior to Act 1412.

As previously mentioned, a second timer is started at Act 1428. At Act 1431, it is determined if the ultrasonic signal is below the trigger threshold. If it is, then a bubble is detected at Act 1437. Or, if the ultrasonic signal is not below the trigger threshold at Act 1431, then Act 1435 determines if the ultrasonic signal is above the second threshold. If it is, then the method 1408 proceeds to before Act 1412. Otherwise, if the ultrasonic sign is below the second threshold, Act 1436 determines if a second predetermined amount of time elapsed for the second timer. If the second predetermined amount of time has elapsed for the second timer, Act 1435 sets the trigger threshold to a third bubble threshold that is lower than the first bubble threshold and the second bubble threshold, after which, the method continues to before Act 1412. If a second predetermined amount of time has not elapsed for the second timer in Act 1436, then the method returns to Act 1431 as is shown in FIG. 213.

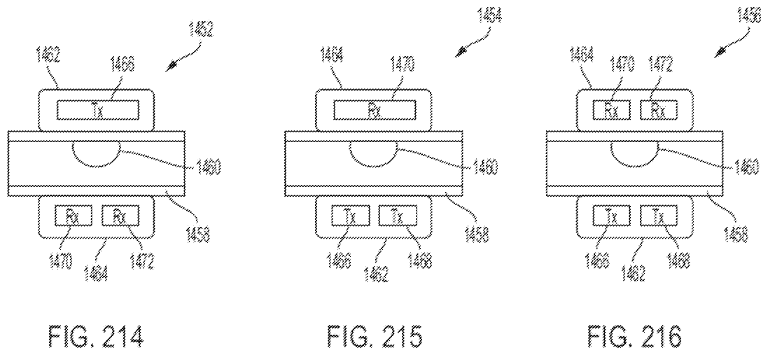

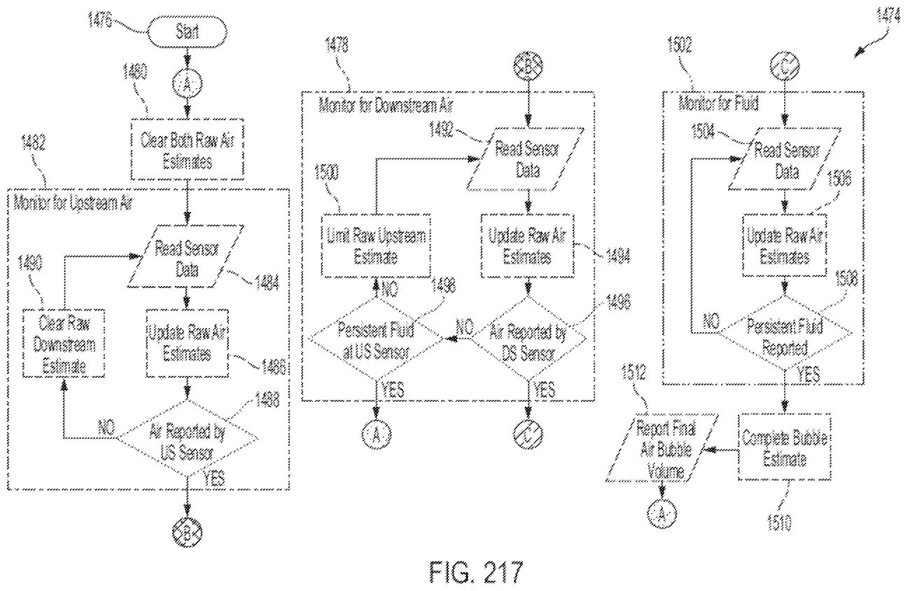

FIGS. 214-217 show various ultrasonic-based bubble sensors in accordance with several embodiments of the present disclosure. Each of these embodiments shown in FIGS. 214-217 illustrate a sensor with multiple acoustic paths, which provide additional means for detecting air in the tubing. FIG. 214 shows an ultrasonic-based bubble sensor 1452 that includes a single piezoelectric transmitter 1466 and two piezoelectric receiver 1470, 1472. FIG. 215 shows an ultrasonic-based bubble sensor 1454 that includes two piezoelectric transmitters 1466, 1468 and a piezoelectric receiver 1470. FIG. 216 shows an ultrasonic-based bubble sensor 1456 having two piezoelectric transmitters 1466, 1468 and two piezoelectric receivers 1470, 1472. FIG. 216 is shows as being configured in an “aligned”-configuration. FIG. 217 shows an ultrasonic-based bubble sensor 1453 that is an “anti-aligned” configuration.

FIG. 214 shows an ultrasonic-based bubble sensor 1452 having a transmit module 1462 and a receive module 1464. The transmit module 1462 includes a first piezoelectric transmitter 1466. The receive module 1464 includes a first piezoelectric receiver 1470 and a second piezoelectric receiver 1472. The first piezoelectric receiver 1470 is separated from second piezoelectric receiver 1472 by a gap 1471. The gap 1471 may be arbitrarily small or large, and filled with any medium such as, but not limited to, air, foam, plastic, epoxy, etc. The single piezoelectric transmitter 1466 may transmit an ultrasonic signal across the tube 1458 to be received by the receive module 1464. The two piezoelectric receivers 1470, 1472 each can receive the ultrasonic signal, which is then converted to an electronic signal to be analyzed by a processor. The two piezoelectric receivers 1470, 1472 can be used together to determine the presence, position, size, velocity, or travel direction (e.g., upstream or downstream) of the bubble 1460. Additionally or alternatively, the two piezoelectric receivers 1470, 1472 can be used in a redundant fashion to ensure the bubble 1460 is moving or to determine the size or velocity of the bubble.

FIG. 215 shows an ultrasonic-based bubble sensor 1454 having a transmit module 1462 and a receive module 1464. The transmit module 1462 includes a first piezoelectric transmitter 1466 and a second piezoelectric transmitter 1468 which can both transmit ultrasonic energy across the tube 1458. The ultrasonic energy may be received by the module 1464 that includes a piezoelectric receiver 1470. The first piezoelectric transmitter 1466 and the second piezoelectric transmitter 1468 may be utilized such that only one is transmitting ultrasonic energy across the tube 1458 at any given time interval, in some specific embodiments. By periodically alternating between the first piezoelectric transmitter 1466 and the second piezoelectric transmitter 1468, a bubble 1460 may be tracked to determine its direction of movement and/or size. For example, if fluid is being pumped through the tube 1458 at a known flow rate, the amount of time the bubble 1460 is sensed by the piezoelectric receiver 1470 can be used in conjunction with the flow rate to estimate the size of the bubble 1460.

FIG. 216 shows an ultrasonic-based bubble sensor 1456 also having a transmit module 1462 and a receive module 1464. The transmit module 1462 includes a first piezoelectric transmitter 1466 and a second piezoelectric transmitter 1468. The receive module 1464 includes a first piezoelectric receiver 1470 and a second piezoelectric receiver 1472. The two piezoelectric transmitters 1466, 1468 can be modulated and/or multiplexed in various configurations.

In some embodiments, both piezoelectric transmitters 1466, 1468 are active at the same time and the two piezoelectric receivers 1470, 1472 each receive a respective ultrasonic signal simultaneously. Each of the piezoelectric transmitters 1466, 1468 may be coded and/or modulated so that each of the two piezoelectric receivers 1470, 1472 can distinguish which piezoelectric transmitter of the transmitters 1466, 1468 sent a respective signal (e.g., through demodulation or decoding). Additionally, alternatively, or optionally, there may be sufficient distance between the piezoelectric transmitters 1466, 1468 and/or sufficient distance between the piezoelectric receivers 1470, 1472 to prevent or mitigate cross-talk. In some embodiments, there may be a physical shield or barrier to shape and/or block ultrasonic energy from crossing paths.

In yet additional embodiments, the first piezoelectric transmitter 1466 and second piezoelectric transmitter 1468 are modulated with different frequencies relative to each other. For example, the first piezoelectric transmitter 1466 may transmit ultrasonic energy at a first frequency and the second piezoelectric transmitters 1468 may transmit ultrasonic energy at a second frequency. The first piezoelectric receiver 1470 may include filtering to filter the signal to receive the ultrasonic energy generated by the first piezoelectric transmitter 1466 while filtering out ultrasonic energy generated by the second piezoelectric transmitter 1468. Likewise, the second piezoelectric receiver 1472 may include filtering to filter the signal to receive the ultrasonic energy generated by the second piezoelectric transmitter 1468 while filtering out the ultrasonic energy generated by the first piezoelectric transmitter 1466. In some embodiments, the first piezoelectric transmitter 1466 and second piezoelectric transmitter 1468 may be active simultaneously, asynchronously, or may employ time-division multiplexing to avoid transmitting at the same time. Additionally, alternatively, or optionally, coded-division multiplexing may be used.

The two piezoelectric receivers 1470, 1472 may be used to estimate the position of the bubble 1460. The first piezoelectric transmitter 1466 and second piezoelectric transmitter 1468 may alternate the generation of the ultrasonic energy such that the first piezoelectric receiver 1470 and the second piezoelectric receiver 1472 are used to determine the direction and/or position of the bubble 1460 using triangulation known to one of ordinary skill in the relevant art.

FIG. 217 shows an ultrasonic-based bubble sensor 1453 that is in an “anti-aligned” configuration. That is, the ultrasonic-based bubble sensor includes a first module 1455 and a second module 1457. The first module 1455 includes a piezoelectric receiver 1466 and a piezoelectric transmitter 1472. The second module 1457 includes a piezoelectric receiver 1470 and a piezoelectric transmitter 1468. The piezoelectric transmitter 1468 transmits to the piezoelectric receiver 1472. The piezoelectric transmitter 1466 transmits to the piezoelectric receiver 1470. The ultrasonic-based bubble sensor 1453 may utilizing may of the same techniques described above with regard to one or more of FIGS. 214-216. The ultrasonic-based bubble sensor 1453 is anti-aligned because each side has a piezoelectric receiver 1466 and a piezoelectric transmitter 1468 configured to cooperate with a piezoelectric receiver 1470 and piezoelectric transmitter 1468 on an opposite side thereof.

Referring to the drawings, FIGS. 217-219 show a method 1474 for estimating air pumped downstream toward a patient by a peristaltic pump 1020 in accordance with an embodiment of the present disclosure. The method 1474 includes acts 1476-1512. The method 1474 may be implemented by utilizing a processor associated with a peristaltic pump, e.g., a peristaltic pump 1020 as described herein. The method 1474 may be coupled to one or more bubble sensors as previously mentioned. The method 1474 may utilize the ultrasonic bubble sensor of FIG. 214 or 216 where the receiving module 1464 includes two piezoelectric receivers or by FIG. 215 where the first and second transmitters 1466, 1468 are modulated to have two different air sensor readings using a single receiver 1470

For the method 1474 described in FIGS. 217-219, please refer to the terminology described in Table 2 as follows:

TABLE 2

| Term | Description |
|---|---|
| Bubble Event | A single Bubble Event includes any series of bubbles |
| | observed by both sensors that are not separated by |
| | sufficient volume of fluid (see Fluid Clear Volume). |
| Fluid Clear | Volume of consecutive fluid seen by one or more |
| Volume | sensors to declare the end of a Bubble Event |
| Hold-up | Volume within the tube between the upstream and |
| Volume (V) | downstream sensing paths. Calculated by using |
| | the cross-sectional area of the tube and the |
| | distance between the centerline of both sensing paths. |
| Overall | Final air bubble volume estimate, comprised of the |
| Bubble | individual raw air volume estimates from both sensing |
| Estimate | elements. |
| Raw | The raw air volume estimate from the downstream |
| Downstream | sensor during one Bubble Event. Calculated by |
| Estimate | summing the volume delivered while air is reported |
| | by the downstream sensing element. |
| Raw | The raw air volume estimate from the upstream sensor |
| Upstream | during one Bubble Event. Calculated by summing the |
| Estimate | volume delivered while air is reported by the |
| | upstream sensing element. |
| Single Sensor | Volume of consecutive fluid seen by a single sensor |
| Fluid Clear | required to declare the end of a Bubble Event. |
| Volume | This is used in the case where a bubble becomes |
| | stuck in front of one of the sensing elements. This |
| | volume is larger than the Fluid Clear Volume |

Act 1476 is the start of the method 1474. Act 1476 may be a function call made by other software being executed by a processor, for example. A function call may also be called a subroutine call. A subroutine (also known as a procedure, function, method or routine) includes the operation where one piece of code causes another piece of code to execute, inter alia, and also may be a system call or a function call. After Act 1476, Act 1480 clears both raw air estimates, which may include setting an upstream raw estimate and a downstream raw air estimate to zero. These estimates may be stored in a memory location and/or may be a calculated variable. At Act 1482, the method 1474 monitors for upstream air. For example, Act 1482 may monitor the signal received by the first piezoelectric receiver 1470 that is generated by the first piezoelectric transmitter 1466 of FIG. 216 of the ultrasonic-based bubble sensor 1456.

Act 1482 may be a subroutine of processor executable code that implements method 1474. Act 1482 monitors for upstream air and may include acts 1484-1490. Act 1484 reads the sensor data (e.g., ultrasonic air sensor data). Act 1486 updates the raw air estimates. Act 1488 determines if the upstream sensor reports air (e.g., if the received ultrasonic signal drops by a predetermined amount). If no air is reported by the upstream sensor, Act 1490 clears (sets to zero) the raw downstream estimate and Act 1484 is repeated. If air is reported by the upstream sensor at Act 1488, the method executes Act 1478, e.g., by a method call, execution of software, etc.

Act 1478 monitors for downstream air. Act 1478 may include acts 1492-1500. Act 1492 reads the sensor data. Act 1494 updates the raw air estimates. Act 1496 determines if air is reported by the downstream sensor (e.g., second piezoelectric transmitter 1468 and second piezoelectric receiver 1472). If Act 1496 determines that air is reported by the downstream sensor, the method continues to Act 1502, otherwise, the method continues to Act 1498. Act 1498 determines if persistent fluid is determined at the upstream sensor. If not, Act 1500 limits the raw upstream estimate and returns to Act 1492. Otherwise, Act 1498 returns to Act 1480. Act 1502 is entered into if air is reported by the downstream sensor in Act 1496.

Act 1502 may include acts 1504-1508. Act 1504 reads the sensor data, e.g., the upstream ultrasonic sensor data. Act 1506 updates the raw air estimates. Act 1508 determines if there is persistent fluid reported. If no persistent fluid is reported in Act 1508, the method continues to Act 1504. Otherwise, the method continues to Act 1510 to complete a bubble estimate and at Act 1512, the final air bubble volume is reported, e.g., to the control system or operating system.

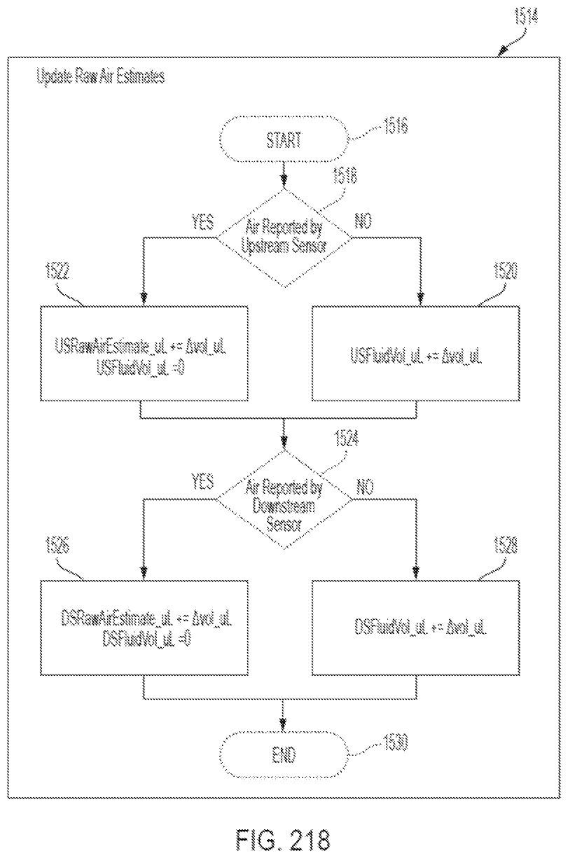

Referring now to FIGS. 217-218, Acts 1486, 1494, and/or 1506 may be Act 1514 as shown in FIG. 218. Act 1514 updates the raw air estimates and includes Acts 1516-1530. Act 1516 is the start, which may be a function call, for example. Act 1518 determines if the air is reported by the upstream ultrasonic air sensor. This may be a parameter passed as part of the function call (e.g., a subroutine). If no air is reported by the upstream sensor, Act 1518 continues to Act 1520. If air is reported by the upstream sensor, the method continues to Act 1522.

Act 1520 adds the delta volume of fluid to the upstream fluid volume total (e.g., USFluidVol_uL+=Δvol_uL). That is, Act 1520 adds the amount of fluid pumped downstream to a total value. If air was reported by the upstream sensor in Act 1518, Act 1522 adds the amount of air pump downstream by the upstream ultrasonic sensor to a raw air estimate (e.g., USRawAirEstimate_ul+=Δvol_uL) and sets the volume of fluid of the upstream sensor to 0 (e.g., USFluidVol_uL=0).

After either Act 1520 or Act 1522, Act 1524 determines if air has been reported by the downstream ultrasonic sensor. The downstream ultrasonic sensor may be the downstream one of 1470, 1472, the downstream one of 1466, 1468, or the downstream one of 1462 and 1472 or 1466 and 1470 of FIGS. 214, 215, and 216, respectively.

If air has been reported, Act 1514 continues to Act 1526 where the reported delta volume is added to the downstream raw air estimate (e.g., DSRawAirEstimate_uL+=Δvol_uL) and the downstream fluid volume is set to zero (e.g., DSFluidVol_uL=0). Otherwise, if no air is reported by the downstream sensor, then Act 1524 transitions to Act 1528 where the delta volume pumped downstream is added to the downstream fluid volume (e.g., DSFluidVol_uL+=Δvol_uL).

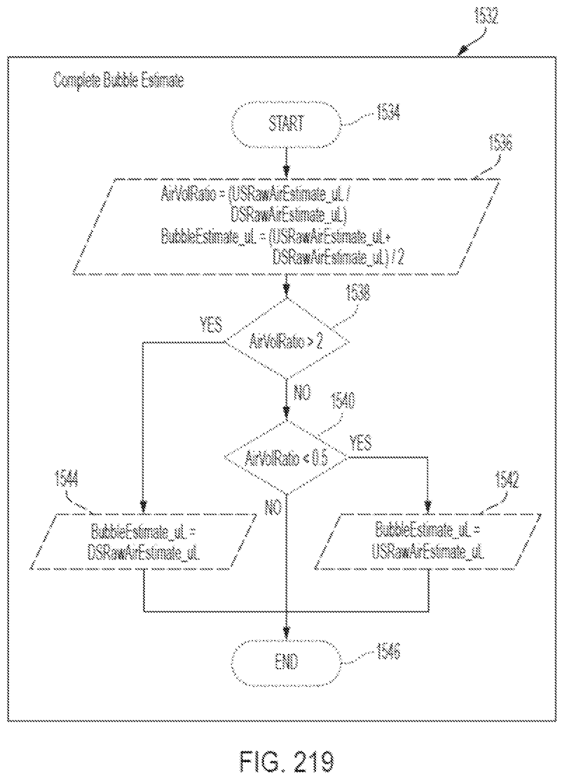

Referring now to FIG. 219, a method 1532 for completing a bubble estimate is shown. The method 1532 may be Act 1510 of FIG. 217. The method 1532 includes Act 1534 which may be the initial start of the method 1532. Act 1534 may be a function call, an API call, a subroutine call, a system call, etc. Act 1536 determines an air volume ratio and calculates a bubble estimate. The air volume ratio may be the upstream raw air estimate divided by the downstream raw air estimate (e.g., AirVolRatio=USRawAirEstimate_uL/DSRawAirEstimate_uL). Act 1536 also calculates an initial bubble estimate by taking an average of the upstream and downstream air estimates (e.g., (USRawAirEstimate_uL+DSRawAirEstimate_uL)/2). Act 1538 and Act 1540 work together to determine if the air volume ratio is outside of a predetermined range. If it is not, then the bubble estimate is reported by the method 1532 as is calculated by Act 1536.

Act 1538 determines if the air volume ratio is greater than 2, in which case Act 1544 sets the bubble estimate to the downstream raw air estimate. However, if the air volume ratio is less than 0.5, then Act 1542 sets the bubble estimate to the upstream raw air estimate. As previously mentioned, if the air volume ratio is less than 2 but greater than 0.5, the value set in 1536 is return by act 1546, which is the end. Act 1546 may be a return act, such as providing return values from a function call made by another routine, code, subroutine, etc.

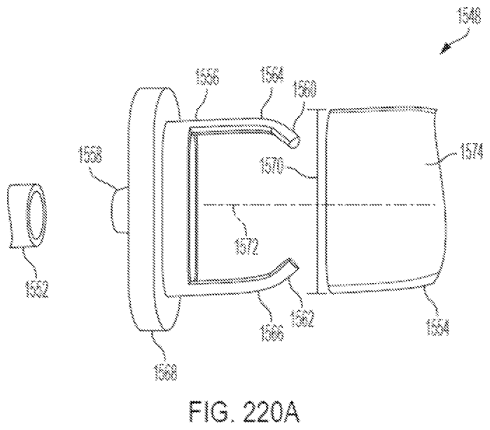

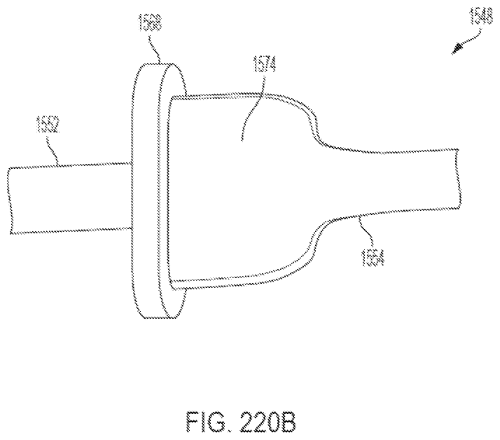

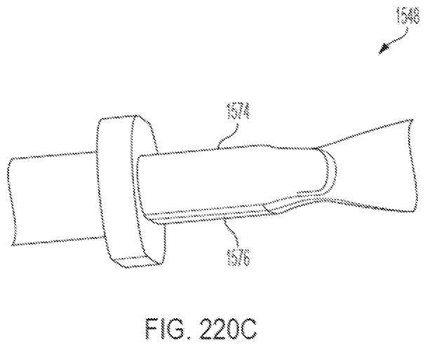

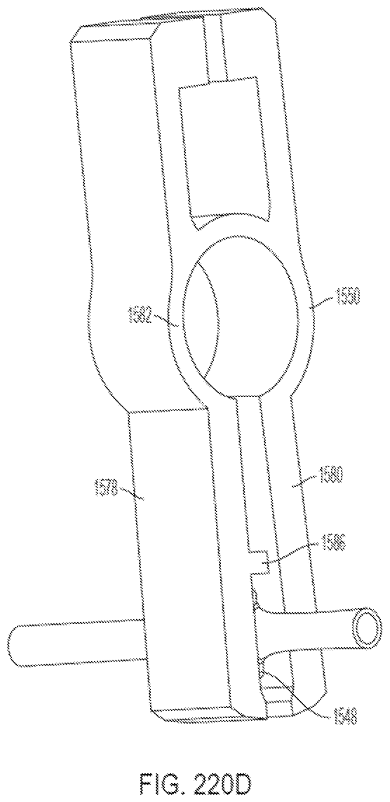

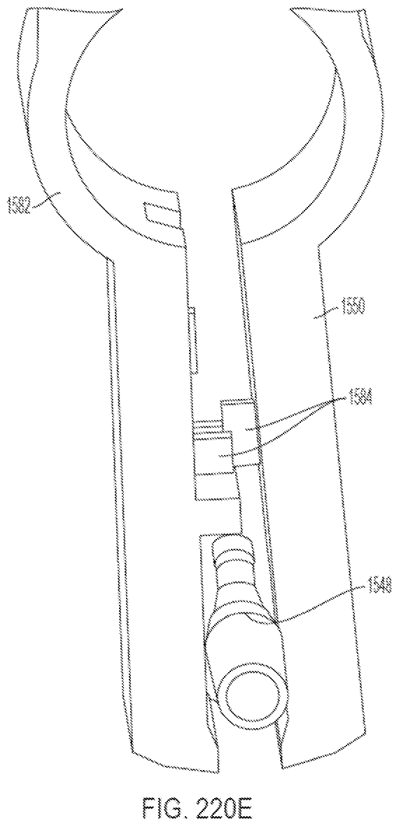

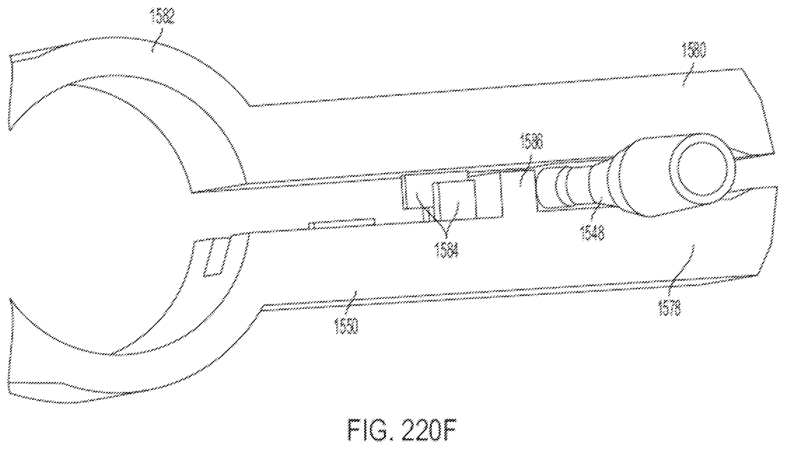

FIGS. 220A-220C show several views of an in-line pressure sensor 1548 and FIGS. 202D-202F show the in-line pressure sensor of FIGS. 220A-220C with a clip 1550 in accordance with an embodiment of the present disclosure.

Referring now to FIG. 220A, the in-line pressure sensor 1548 is shown and may be configured to measure a pressure inside an intravenous fluid line, or other fluid line. The in-line pressure sensor 1548 may be disposed between a first portion 1552 of an IV line and a second portion 1554 of the IV line.

The in-line pressure sensor 1548 includes a first port 1558 and a second port 1556. The second port 1556 includes a first arm 1564 and a second arm 1566. The distal end 1560 of the first arm 1564 arcs toward a central axis 1572 of the second portion 1554 of the IV line. Similarly, the distal end 1562 of the second arm 1566 arcs toward the central axis 1572. The second port 1556 is inserted into the second portion 1554 of the IV line to stretch the second portion 1554 out along length 1570. This stretching creates a first flat side 1574. The second port 1556 is inserted into the second portion 1554 until the second portion 1554 engages with a raised flange 1568. The second portion 1554 may be attached, glued, welded, ultrasonically welded, etc. to the raised flange 1568. The first portion 1552 is attached to a first port 1558 which may be attached, glued, welded, ultrasonically welded, etc. to the raised flange 1568.

Referring now to FIG. 220B, the in-line pressure sensor 1548 is shown assembled. Please note the first flat side 1574. Because fluid contained within the IV can cause deformation of the second portion 1554 of the IV tube, those deformations would be enlarged by the first flat side 1574 and the second flat side 1576, which are both easily seen in FIG. 220C.

FIG. 220D shows the in-line pressure sensor 1548 being coupled by a clip 1550 in accordance with an embodiment of the present disclosure. The clip 1550 includes a first elongated member 1578 and a second elongated member 1580. The first and second elongated members 1578, 1580 pivot along a living hinge 1582. As is easily seen in FIG. 220E, as the first and second elongated members 1578, 1580 expand due to increased pressure within the IV tube, the living hinge 1582. A sensor 1584 can be utilized to measure the pivot amount of the clip 1550. The sensor 1584 may be any sensor to measure a pivot, movement, displacement, etc., such as a beam-breaking sensor, a hall sensor, a potentiometer, any sensor known to one of ordinary skill in the relevant art, etc. FIG. 220F shows another view of the clip 1550 to illustrate a stop member 1586, which is optional. The stop member 1586 may prevent the first and second elongated members 1578, 1580 from contacting each other.

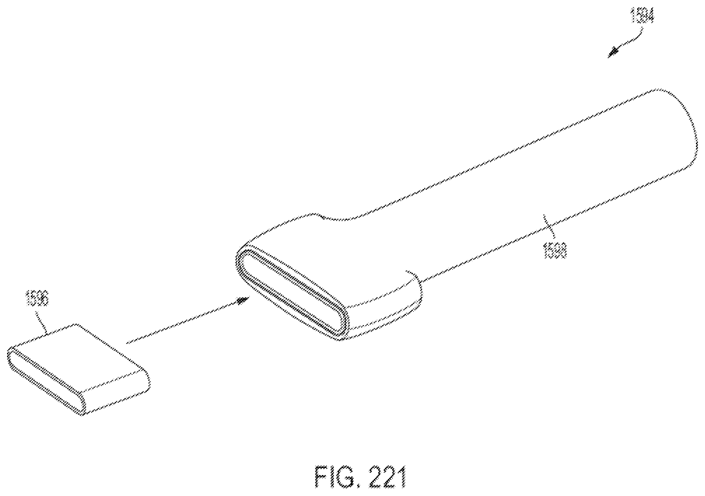

FIG. 221 shows another in-line pressure sensor 1594 in accordance with yet another embodiment of the present disclosure. The in-line pressure sensor 1594 includes an insert 1596. Fluid between the insert 1596 and the IV tube 1598 will cause the deflection of the material needed for pressure measurement by the clip 1550.

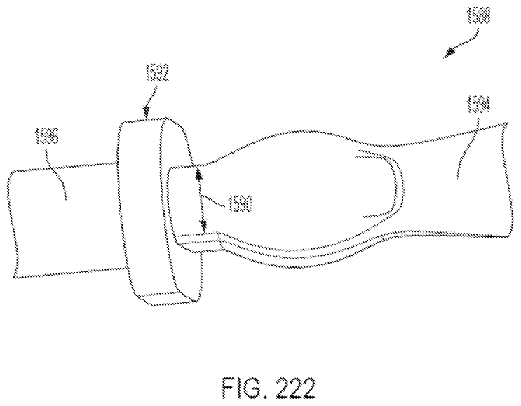

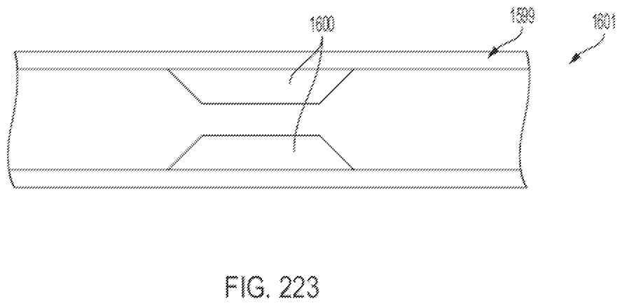

FIG. 222 shows a downstream bladder or an in-line pressure sensor 1588 in accordance with yet another embodiment of the present disclosure. The in-line pressure senor 1588 of FIG. 222 includes a narrow region 1590 adjacent to the flange 1592. In some embodiments, the item shown in FIG. 222 can be utilized as a downstream bladder 1588 in accordance with an embodiment of the present disclosure. The downstream bladder 1588 includes a narrow region 1590 adjacent to a flange 1592. The flange 1592 may be utilized as an attachment point by the tube 1594. The tube 1594 of the passageway in the flange 1592 connecting the first portion of the tube 1594 to a second portion of the tube 1596 may be of a reduced diameter relative to the tube 1594, 1596 to dislodge bubbles. For example, the tube portion 1599 of FIG. 223 may be part of the IV tube 1598 or may be embedded within the flange 1592. The tube portion 1599 may use the impeders 1600 to dislodge bubbles. The first portion of the tube 1594 may be coupled to an output of a peristaltic pump 1020, e.g., a peristaltic pump disclosed herein. The downstream bladder 1588 may smooth out irregular fluid flow caused by the pulsating process of a peristaltic pump. In some embodiments of the present disclosure, a very compliant tube may be used in place of a downstream bladder 1588. The impeders 1600 of the tube 1599 of FIG. 223 may be used to slightly increase pressure downstream of a highly compliant tube. In some embodiments of the present disclosure, the impeders 1600 may be configured to be a nucleation site for air bubbles. The nucleation site may be formed by roughing, scoring, or etching the impeders 1600. A heater 1601 may be utilized to increase the fluid temperature prior to having the fluid come into contact with a nucleation site.

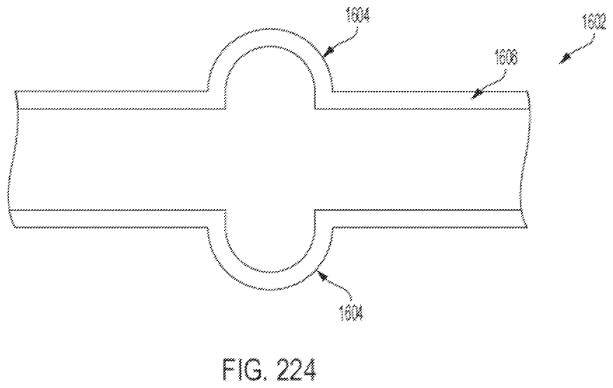

FIG. 224 shows another embodiment of a downstream bladder 1602 in accordance with another embodiment of the present disclosure. The downstream bladder 1602 includes a tube 1608. The tube 1610 includes a compliant section 1604 that allows for expansion to absorb irregular fluid flow. The rest of the tube 1608 may have standard IV compliance, while the compliant section 1604 may be substantially more compliant than the bulk of the tube 1608.

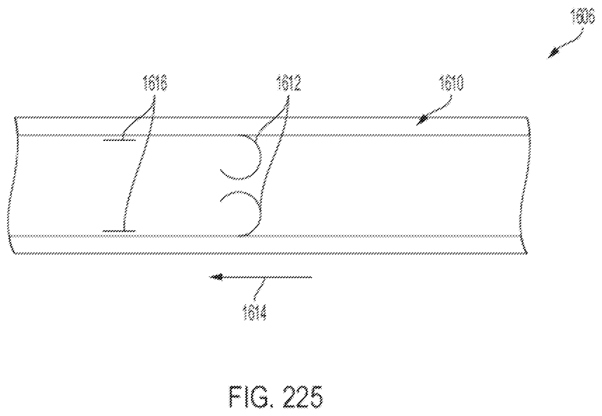

FIG. 225 shows a section of tubing 1606 that includes nucleation sites 1616 with an air trap 1612 in accordance with an embodiment of the present disclosure. Liquid fluid can flow downstream in a direction 1614. As the liquid fluid flows downstream, the liquid fluid passes an air trap 1612 and passes by nucleation sites 1616. The nucleation sites 1616 may be configured to encourage gasses within the liquid fluid to form bubbles at microscopic locations along the nucleation sites 1616. The bubbles may start to flow upstream, which is opposite to the direction 1614. The air trap 1612 may trap the bubbles as they flow upstream along a wall of the tube 1610. The air trap 1612 may be formed by interfacing together two tube sections 1610 such that one tube section folds back on itself thereby taking a general toroid shape for the air trap 1612. In yet an additional embodiment of the present disclosure, the liquid fluid may be heated upstream of the nucleation sites 1616 to help facilitate outgassing via bubble formation on the nucleation sites 1616.

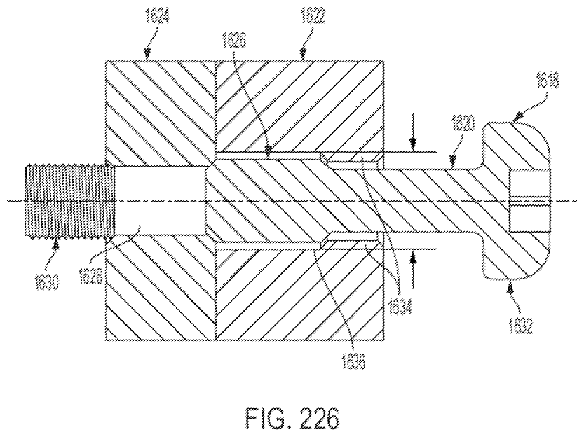

FIG. 226 shows a captive screw 1618 in accordance with an embodiment of the present disclosure. The captive screw 1618 includes a head 1632, a narrow neck 1620, an enlarged mid-body 1626, an unthreaded end 1628, and a threaded end 1630. The enlarged mid-body 1626 may be captured within an enlarged cavity 1636 of a first section of material 1622 (e.g., a metal panel). The narrow neck 1620 of the captive screw 1618 may be disposed within a narrow cavity 1634. The optional second section of material 1624 may surround the unthreaded end 1628. In some embodiments the threaded end 1630 is coupled directly to the enlarged mid-body 1626. The captive screw 1618 may be pressed into the narrow cavity 1634 and the enlarged cavity 1636 as part of an access panel to a peristaltic pump, e.g., a back or side panel of a peristaltic pump described here.

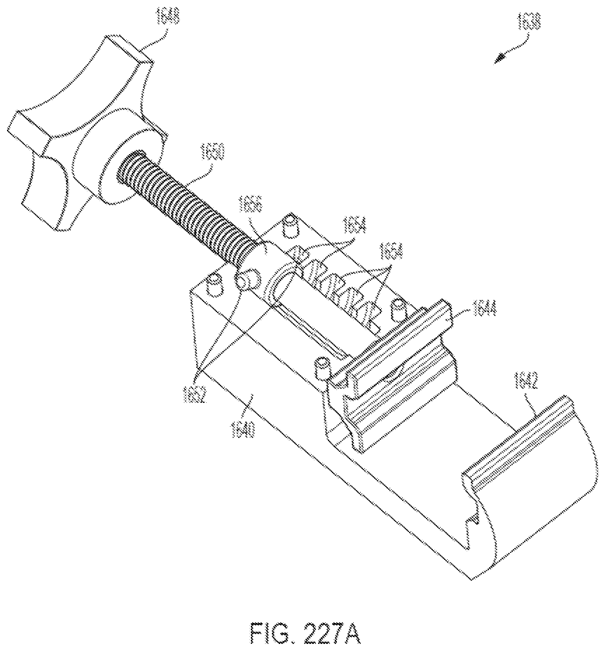

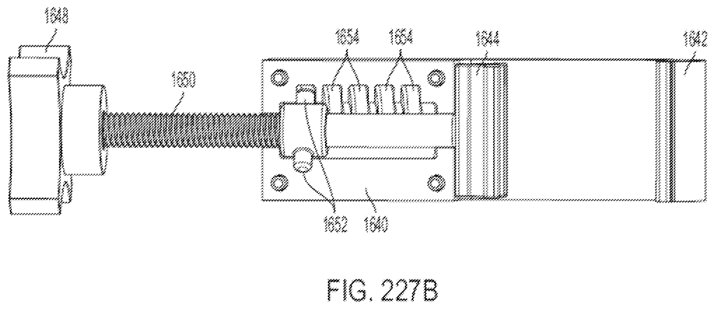

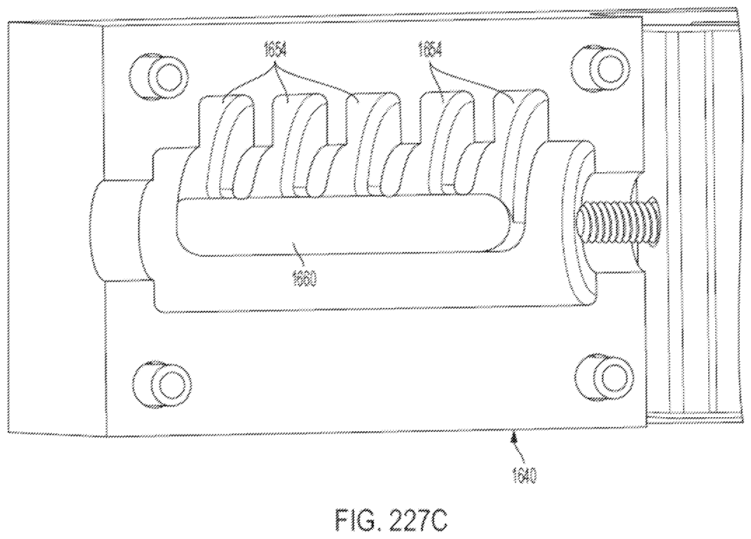

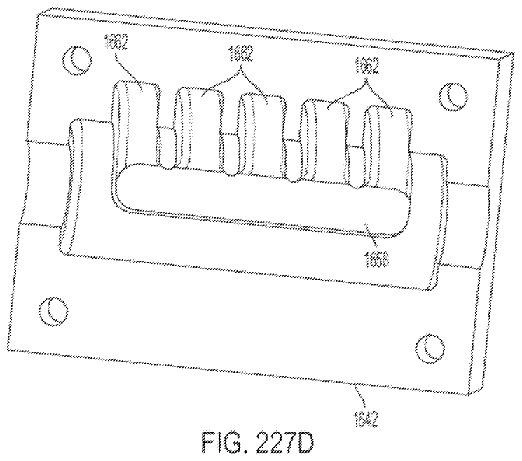

FIGS. 227A-227D illustrate a pole clamp 1638 in accordance with an embodiment of the present disclosure. The pole clamp 1638 is configured to allow for coarse and fine adjustments to allow a user to quickly attach, for example, a medical device to a pole.

The pole clamp 1638 includes a fixed jaw member 1642 and a moveable jaw member 1644. The pole clamp 1638 includes a body having a lower-half of the body 1640 and a upper-half of the body 1640 (see FIGS. 227C-227D). The pole clamp 1638 also includes a knob 1648 coupled to a threaded screw 1650. The threaded screw 1650 engages with a collar 1656. The collar 1656 includes cams 1652. The cams 1652 engage with the cam trenches 1654. The threaded screw 1650 can engage with internal threads of the collar 1656 to actuate the moveable jaw member 1644 as the knob 1648 is turned.

Prior to securing the pole clamp 1638 to a medical pole, a slight counter-clockwise turn of the knob 1648, the cams 1652 of the collar 1656 will rotate such that the cams 1652 are in the trench 1658 of FIG. 227D or the trench 1660 of FIG. 227C. In this position, the knob 1648 can easily be moved toward or away from the fixed jaw member 1642 thereby actuating the moveable jaw member 1644. When a coarse position has been made by the user, a clockwise turn of the knob 1648 will rotate the collar 1656 such that the cams 1652 actuate into the cam trenches 1654. Because the cam trenches 1654 are only long enough so that the collar 1656 is actuated about a quarter of a turn, the cams 1652 will stop rotating and the threaded screw 1650 will instead rotate. The rotation of threaded screw 1650 engages with internal threads of the collar 1656 such that the moveable jaw member 1644 is pushed toward the fixed jaw member 1642 thereby providing a fine adjustment mechanism. FIG. 227B shows the top view of the pole clamp 1638 with the upper-half of the body 1640 (see FIG. 227D) removed. FIG. 227C shows a close-up view of the lower-half of the body 1640 with the threaded screw 1650 removed to illustrate the trench 1660 while FIG. 227D shows a close-up view of the upper-half of the body 1643 to also illustrate the trench 1658 and the cam trenches 1662.

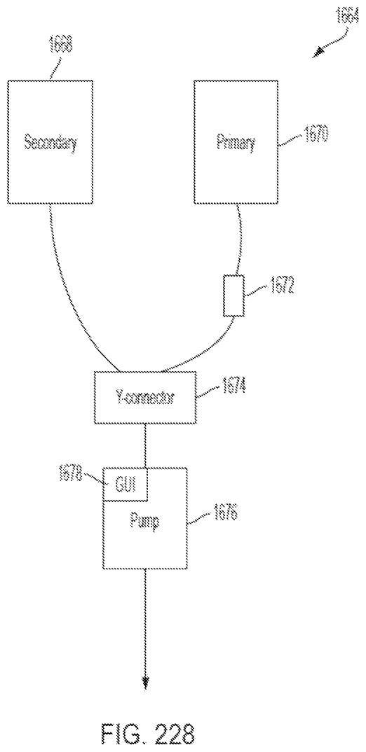

FIG. 228 shows a block diagram that illustrates a system 1664 for pumping fluid from a primary IV bag 1670 and a secondary IV bag 1668 in accordance with an embodiment of the present disclosure. The system 1664 includes the primary IV bag 1670 coupled to a check valve 1672. The check value 1672 may be a one-way valve, such as a duck-bill valve, configured to allow fluid to flow from the primary IV bag 1670 and a Y-connector 1674. The secondary IV bag 1668 is also connected to the Y-connector 1674 such that fluid may be pumped downstream by action for a pump 1676. The pump 1676 may be a spring-biased peristaltic pump 1020 as described herein.

The pump 1676 also includes a GUI 1678, which may be a touch screen, for example. A user can interact with the GUI 1678 to set the pump 1676 into a secondary-infusion mode. Alternatively, the user can interact with the GUI on the Main Processing Control Unit (PCU) (see FIG. 187) to achieve the same effect. The secondary-infusion mode may be a state of a control system implemented by a processor executing a plurality of executable instructions (e.g., a discrete PID control loop implemented in software).

After fluid has been discharged downstream, the outlet valve of the pump 1676 may be closed and the upstream inlet valve opened. The processor may limit the rise of the plunger to limit the amount of fluid that flows from the Y-connector 1674 to thereby control the pressure of the fluid within the upstream fluid lines. The processor may be configured to limit sympathetic flow by limiting the actuation of the plunger by keeping the upstream fluid pressure above the predetermined threshold. The predetermined threshold may be a pressure above a crack pressure of the check valve 1672, such as pressure between 1.5 to 5 lbs-per-square inch (or about 6,900 Pascals to about 34,500 Pascals).

The actuation limit may be a plunger speed limit or a rotation speed limit of a cam shaft. The processor may not limit the actuation when in the pump 1676 is in the secondary-infusion mode unless the fluid flow rate is above a predetermined fluid flow rate, e.g., 250 milliliters per hour, 500 milliliters per hour, or any rate in between. The flow rate may be limited when in the secondary-infusion mode, e.g., to 500 milliliters/hr or less. Is some embodiments, the pump 1676 may limit the actuation to ensure sympathetic flow is less than 5% of a volume to be infused (e.g., then volume to be infused may be between 50 milliliters/hr to 1000 milliliters/hr.

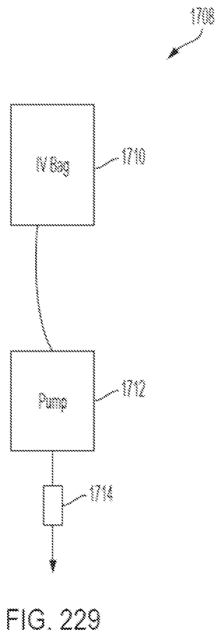

FIG. 229 shows a block diagram that illustrates a system 1708 for pumping fluid in conjunction with a check valve 1714 to prevent outgas sing in accordance with an embodiment of the present disclosure. The check valve 1714 may be a duck-billed valve. The system 1708 includes an IV bag 1710 with a fluid line coupled to a pump 1712 (e.g., the peristaltic pump 1020). A check valve 1714 may be configured to increase the fluid pressure inside the IV tube in the pump 1712. That is, the check valve 1714 may be configured to provide a nadir pressure within the tube. The crack pressure of the check valve 1714 may be configured to raise the nadir pressure above a predetermined threshold. The predetermined threshold may be above an outgassing pressure of the fluid within the IV line (e.g., between the valve 1714 and the pump 1712 and/or within the pump 1712 adjacent to a spring-biased plunger). In some embodiments the crack pressure is adjustable in the check valve 1714 and may be adjusted to account for temperature changes. The pump 1712 may electronically adjust the crack pressure of the check valve in some embodiments. Additionally, alternatively, or optionally, the check valve 1714 may be adjusted in accordance with measured ambient pressure, measured pressure of the fluid or the expected or measured amount of dissolved gas. In some embodiments, a pump 1712 may control the spring-biased plunger to keep the nadir pressure above the outgas pressure.

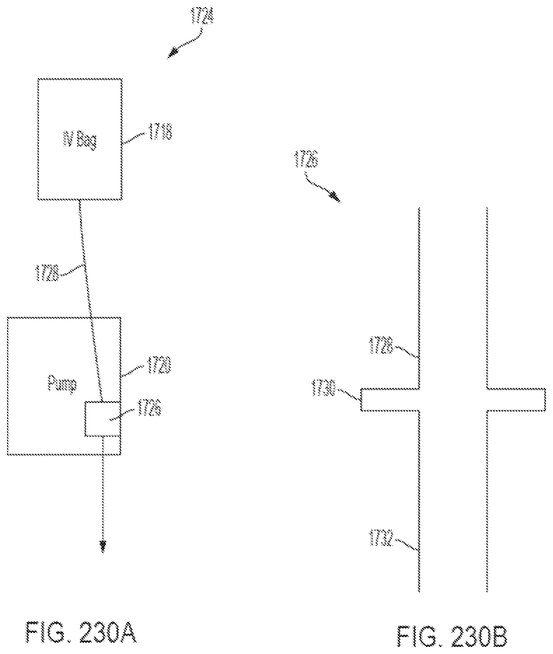

FIG. 230A shows a system 1724 for pumping fluid having a retainer 1726 configured to secure the retainer 1726 to the pump body. FIG. 230B shows a close up view of the retainer 1726. The retainer 1726 includes a first tube 1728 and a second tube 1732. The first and second tubes 1728, 1732 are coupled to a plastic retainer that may be concentrically surround the tube or may be rectangular shape for being secured within the peristaltic pump 1020.

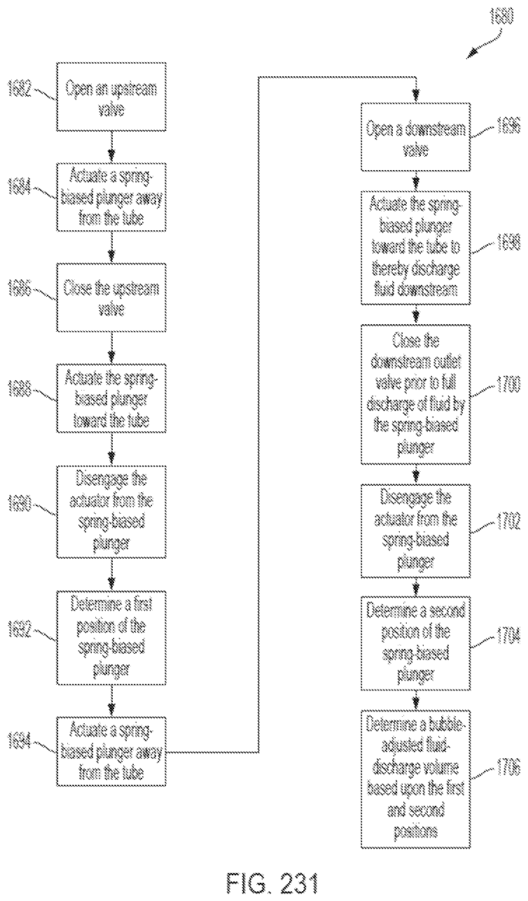

FIG. 231 shows a flowchart diagram of a method 1680 for pumping fluid and adjusting fluid delivery estimates to account for air or bubbles in accordance with an embodiment of the present disclosure. The method 1680 includes Acts 1682-1706. The method 1680 may be performed by a peristaltic pump 1020 having a spring-biased plunger where an actuator lift the plunger thereby charging the spring and the actuator and the plunger can disengage from each other.

Act 1682 opens an upstream valve. For example, after fluid has been discharged downstream by the plunger, the peristaltic pump needs to fill the tube adjacent of the plunger with fluid. Thus, in Act 1684, the method 1680 actuates (e.g., using a cam shaft) the spring-biased plunger away from the tube. Once fluid is filled within the tube adjacent to the spring-biased plunger, Act 1686 closes the upstream valve thereby fluidly sealing the filled tube adjacent to the spring-biased plunger. Act 1688 actuates the spring-biased plunger toward the tube and Act 1690 disengages the actuator from the spring-biased plunger. The spring-biased plunger is held against the tubing adjacent to the spring-biased plunger by a force of the spring after act 1690. Thus, Act 1692 determines a first position of the spring-biased plunger, which may deviate from the relative position of the actuator. Any air adjacent to the spring-biased plunger may be substantially compressed when the first position of the plunger is determined. Act 1694 actuates the spring-biased plunger away from the tube.

Act 1696 opens a downstream valve and act 1698 actuates the spring-biased plunger toward the tube to thereby discharge fluid downstream. However, Act 1700 closes the downstream outlet valve prior to full discharge of the fluid by the spring-biased plunger. Act 1702 disengages the actuator from the spring-biased plunger. As before, fluid is sealed between the inlet and outlet valves and the position of the spring-biased plunger now corresponds to the amount of fluid in the tubing adjacent to the spring-biased plunger. Also, any air or bubbles are substantially compressed during Act 1702. Act 1704 determines a second position of the spring-biased plunger.

Act 1706 uses the two positions to determine the amount of fluid that has been discharged downstream. Because both of the two positions were made with the spring applying pressure to the tube (via the spring-biased plunger) and thereby substantially compressing any air within the tube, the positions should “filter out” any air from the fluid downstream discharge calculations. Act 1700 may close the downstream outlet valve to ensure that a predetermined amount of fluid remains adjacent to the spring-biased plunger such that the spring-biased plunger does not engage a mechanical stop (that is, the spring-biased plunger could move further toward the tube if less fluid was in the tube).

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in the drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g., “a,” “an,” or “the,” this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B. This expression signifies that, with respect to the present disclosure, the only relevant components of the device are A and B.

Furthermore, the terms “first,” “second,” “third,” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

FIG. 1 shows the front of a pump 100. The pump 100 may be a standalone device that couples to an IV pole (not shown) directly, e.g., by using a clamp (not shown). Additionally or alternatively, the pump 100 may be modular such that one or more pumps 100 can be coupled together with a central unit and/or with other medical devices. Although a peristaltic pump 100 is described throughout this specification, additional embodiments may include syringe pumps or other pump types where applicable or where it would be apparent to one of ordinary skill in the relevant art.

The pump 100 includes a pump housing 158 and a door 102 coupled to the pump housing 158. The door 102 is pivotably coupled to the pump 100 such that an infusion set having a flow stop 152 (see FIGS. 39-44) and a tube 216 (see FIGS. 4-5) may be loaded and secured within the pump 100 by the door 102 (described in more detail below). A hole 106 is shown so that the door 102 may be shut without pinching the tube 216. Kinks or pinches within the tube 216 may occlude fluid flow within the tube 216.

The pump 100 includes a button panel 110 with buttons 112 for user input and a screen 108. The screen 108 provides visual information, such as menus and status information, which can be used by a caregiver to program and interact with the control software of the pump 100 using the buttons 112. In some embodiments, the screen 108 may be a touch screen configured to receive user input via user touch. The pump 100 also includes a lever 104 that can be used to open the door 102 and lock the door 102 as described in more detail below.

The pump 100 also includes a light bar 162. The light bar 162 may illuminate based upon the status of the pump 100. For example, the light bar 162 may blink green when the pump 100 is infusing fluid into a patient and blink red when the pump 100 is not operating or is experiencing an error condition or fault. The light bar 162 may blink yellow when an occlusion is detected and intervention is needed to clear the occlusion, etc.

FIG. 2 shows the peristaltic pump 100 of FIG. 1 with the door 102 open and the lever 104 in the open position. When the lever 104 is shut and the door 102 is properly closed, a door catch 114 secures the door 102 shut by holding on to a hold 164. The hold 164 may be a pin that interfaces with a pin catch 166. When the lever 104 is actuated to the open position as shown in FIG. 2, the door catch 114 releases the hold 164. The door 102 may be spring biased such that the door 102 swings open when the door catch 114 releases the hold 164.

Actuation of the lever 104 into the open position also retracts the spring-biased plunger 116. Actuation of the spring-biased plunger 116 allows a tube 216 to be loaded into a platen 168. Having the spring-biased plunger 116 actuated into the platen 168 would make insertion of a tube 216 into the platen 168 more difficult or impossible because it would block the platen 168.

FIG. 3 shows a close up view of the door 102 of the peristaltic pump 100 (see FIG. 1) in the open position. The carriage assembly 160 is also easily seen in FIG. 3. A flow stop 152 (see FIGS. 39-44) may be inserted into the carriage assembly 160 so that a carriage 150 retains the flow stop 152. A flow-stop retainer 170 can retain the flow stop 152 in the carriage 150. FIG. 4 shows the flow stop 152 loaded into the carriage 150 of the peristaltic pump 100. Thereafter, the door 102 may be shut with the flow stop 152 inserted therein as shown in FIG. 5. Because the lever 104 is still in the open position, the door 102 may be reopened because the door catch 114 has not locked the door 102. When the lever 104 is actuated down into the closed position, then the door 102 will be locked by the door catch 114.

FIG. 6 shows the back of the pump 100 of FIG. 1 with the back housing, cabling and electronic circuit boards, removed. However, in FIG. 6, a motor 172 and a brace 174 are visible. FIG. 7 shows the pump 100 as shown in FIG. 6, but with the motor 172 and the brace 174 removed for additional clarity.

In FIG. 7, a cam shaft 190 is shown with a plunger cam 184, an inlet-valve cam 186, and an outlet-valve cam 188 disposed on the cam shaft 190. A plunger-cam follower 192 pivots along a pivot shaft 202 (see FIG. 14) as the plunger-cam follower 192 follows the plunger cam 184. The inlet valve 198 pivots along the pivot shaft 202 (see FIG. 14) as the inlet-valve cam follower 194 follows the inlet-valve cam follower 194. And, the outlet valve 200 pivot along the pivot shaft 202 (see FIG. 14) as the outlet-valve cam follower 196 follows the outlet-valve cam 188.

An inlet-valve torsion spring 204 biases the inlet-valve cam follower 194 against the inlet-valve cam 186 and toward the tube 216. An outlet-valve torsion spring 206 biases the outlet-valve cam follower 196 against the outlet-valve cam 188. Also, a pair of plunger torsion springs 208 biases the plunger-cam follower 192 against the plunger cam 184 and therefore also biases the spring-biased plunger 116 toward the tube 216. FIG. 8 shows the pump 100 as shown in FIG. 7 but at another angle, and FIG. 9 shows the pump 100 as shown in FIG. 7, but at a bottom-up angle from the back of the pump 100.

Actuation of the lever 104 actuates the main shaft 118. A shaft spring 182 is shown that pulls the main shaft 118 into one of two positions making the lever 104 actuate toward one of the open or closed position depending upon the angle of the main shaft 118. That is, the shaft spring 182 makes the lever 104 operate with an over-center action with regard to the force the shaft spring 182 exerts on the main shaft 118. The force from the shaft spring 182 exerts on the main shaft 118 is also exerted on the lever 104 because of the mechanical coupling between the main shaft 118 and the lever 104. This over-center action biases the main shaft 118 such that the lever 104 is biased toward either the closed position or the open position, depending upon if the lever 104 is between an intermediate position and the closed position or is between the intermediate position and the open position.

Referring to FIGS. 10-13, FIG. 10 shows a front view of a mechanical assembly 210 including the main shaft 118 coupled to the lever 104 with the lever 104 in the open position, and FIG. 11 shows the mechanical assembly 210 of FIG. 10 with the lever 104 in the closed position. FIG. 12 shows the back view of the mechanical assembly 210 of FIG. 10 with the lever 104 in the open position and FIG. 13 shows the back view of the mechanical assembly 210 of FIG. 10 with the lever 104 in the closed position. The mechanical assembly 210 may be found within the pump 100 of FIG. 1.

The lever 104 is coupled to the first bevel gear 122 and rotates with movement of the lever 104. That is, the lever 104 is coupled to the first bevel gear 122 to actuate the first bevel gear 122. The first bevel gear 122 is coupled to the second bevel gear 124, and the second bevel gear 124 is coupled to the main shaft 118. In combination, actuation of the lever 104 causes the main shaft 118 to rotate around its central axis.

Generally, an upper shaft 298 rotates with the main shaft 118. However, the upper shaft 298 is not directly coupled to main shaft 118 and may, in certain circumstances, rotate separately from the main shaft 118. A more detailed description of the circumstances in which the upper shaft 298 rotates apart from the main shaft 118 is described below with reference to FIGS. 31-32.

Rotation of the main shaft 118 causes a lift cam 120 to rotate. The rotation of the lift cam 120 can actuate the spring-biased plunger 116, the inlet valve 198, and the outlet valve 200 away from a tube 216 and out of the platen 168. That is, the spring-biased plunger 116, the inlet valve 198, and the outlet valve 200 are retracted away from the tube 216 and into the end-effector port 214 (See FIGS. 2-4). Additional details of the lift cam 120 are described below.

Referring again to FIGS. 10-13, when the lever 104 is in the open position, as shown in FIGS. 10 and 12, the latching sled 132 is configured so that the door catch 114 will allow the door 102 (see FIG. 1) to open and shut freely without locking the door 102. However, the door catch 114 is biased toward holding the door 102 or releasing the door 102. When the lever 104 is in the closed position (see FIGS. 11 and 13), the latching sled 132 allows the door 102 (see FIG. 1) to shut by allowing the door catch 114 to receive the hold 164 (see FIG. 4). However, when the lever 104 is in the closed position and the door 102 is shut, the latching sled 132 will lock the door 102 by preventing the door catch 114 from releasing the hold 164 (see FIG. 4) after it is locked by the latching sled 132. Details of the latching sled 132 are described below.

Also shown in FIGS. 10-13, the carriage assembly 160 can been seen. A carriage housing 148 receives a flow stop 152 within the carriage 150 for rotation therein. Gears 212 rotate the carriage 150 as the lever 104 is actuated such that the flow stop 152 can be inserted into the carriage 150 when the lever 104 is in the open position as shown in FIG. 10. After insertion of the flow stop 152, actuation of the lever 104 to the closed position (shown in FIGS. 11 and 13) rotates the carriage 150 and rotates the flow stop 152 to unkink the tube 216 so that fluid may flow through the tube 216. Details of the carriage assembly 160 are described below.

Please refer now to FIGS. 14-16 for reference with the following description of the operation of the lift cam 120. FIGS. 14-16 all show cross-sectional views along the same plane. FIG. 14 is a cross-sectional view of the peristaltic pump 100 showing the lift cam 120 when the lever 104 is in the closed position. FIG. 15 is a cross-sectional view of the peristaltic pump 100 showing the lift cam 120 when the lever 104 is in between the closed position and the open position; And FIG. 16 is a cross-sectional view of the peristaltic pump 100 showing the lift cam 120 when the lever 104 is in the open position.

As shown in FIG. 14, the lift cam 120 is disposed on the main shaft 118 for rotation along a lift-cam pin 130. The axis of the lift-cam pin 130 is offset from a central axis of the main shaft 118. The lift cam 120 is biased by a cam-lifter torsion spring 126 in a counter-clockwise direction as shown in FIG. 14, however, one of ordinary skill in the art would know how to configure the pump 100 for clockwise bias.

In FIG. 14, the lift cam 120 is not engaged with the spring-biased plunger 116 and the position of the spring-biased plunger 116 is based upon the rotational position of the plunger cam 184 and/or the fill volume of a tube 216. The spring-biased plunger 116 includes an end effector 128 that engages with the tube 216 disposed in the platen 168.

The end effector 128 of the spring-biased plunger 116 is shown in FIG. 14 as being in an extended position and thereby protrudes out of the end-effector port 214 (thus engaging with the tube 216). A seal 218 prevents fluid ingress or egress through the end-effector port 214 even though the end effector 128 is secured to the spring-biased plunger 116.

As is easily seen in FIG. 15, as the lever 104 is actuated toward the open position, the main shaft 118 rotates and the lift cam 120 engages with the spring-biased plunger 116. Because an outer surface 220 of the lift cam 120 frictionally engages the spring-biased plunger 116, the lift cam 120 rotates as the lever 104 is actuated into the open position as shown in FIG. 15.

FIG. 16 shows the lever 104 in the fully open position in which the lift cam 120 has fully lifted the spring-biased plunger 116 such that the end effector 128 is fully retracted within the end-effector port 214. The tube 216 is visibly present in FIG. 16 because of the retraction of the spring-biased plunger 116. Also, note that the plunger-cam follower 192 has been actuated away from the plunger cam 184 such that it no longer touches the plunger cam 184. The lift cam 120 actuates the inlet valve 198 and the outlet valve 200 in a similar manner. That is, the lift cam 120 also engages with the inlet valve 198 and the outlet valve 200, which are also spring biased.

Referring to FIGS. 17-19, FIG. 17 shows a close-up view of the latching sled 132 of the mechanical assembly 210 of the peristaltic pump 100 of FIG. 1 when the lever 104 is in the closed position. FIG. 18 shows a close-up view of the latching sled 132 when the lever 104 is between the closed position and the open position; And FIG. 19 shows a close-up view of the latching sled 132 when the lever 104 is in the open position.

FIG. 17 shows the lever 104 in the closed position and hence the latching sled 132 is in the extended position. When the latching sled 132 is in the extended position, the claw 134 is actuated away from the main shaft 118 because of the abutment of the sled cam follower 176 with the hook cam 144. That is, the hook cam 144 engages with the sled cam follower 176 such that the hook cam 144 extends the sled cam follower 176 maximally away from the main shaft 118. Therefore, FIG. 17 shows the condition where the hook cam 144 has actuated the latching sled 132 to its fully extended position.

When the latching sled 132 is in the extended position, the door 102 and the door catch 114 may initially be unlocked, but as soon as the door catch 114 is actuated to the closed position (e.g., when the door 102 is shut), a door-catch hold 234 of the door catch 114 is locked between the claw 134 and the sled base 136. That is, once the door catch 114 has rotated into the locked position, the latching sled 132 prevents it from being opened because the latching sled 132 is in the extended (or locking) position.

FIG. 18 shows the lever 104 in a partially actuated position where the hook 146 of the hook cam 144 hooks onto the sled cam follower 176. The hook cam 144 includes a retraction space 238 so that the sled cam follower 176 can be pulled toward the main shaft 118. FIG. 19 shows the lever 104 in the fully open position such that the hook 146 of the hook cam 144 has fully retracted the latching sled 132. As the claw 134 was pushed toward the hook cam 144, the claw 134 pulled the door catch 114 into the open (or unlatched position), which in turn, opened the door 102.

Referring to FIGS. 2, 19 and 25, when the lever 104 was actuated from the closed position to the open position, the claw 134 pulled on the door-catch hold 234 such that the door catch 114 was rotated along its channel 236 which rotated the pin catch 166 to a position where it no longer locks the hold 164 of the door 102. Because the door 102 may be spring-biased open, the door 102 may swing open when the door catch 114 no longer locks onto the hold 164 of the door 102.

Referring again to FIG. 19, the latching sled 132 is coupled to a door-catch spring 224 that is coupled to the door-catch anchor 232. The door-catch spring 224 pushes against the door-catch anchor 232 which makes the door catch 114 actuate with an “over center” action. The over center action of the door-catch spring 224 makes the door catch 114 bi-stable in the locked position or in the open position. As shown in FIG. 19, when the claw 134 is in a retracted position, the door catch 114 is free to actuate freely between the open position and the locked (or closed) position because the claw 134 has been actuated free from the door-catch hold 234 (see FIG. 25)

FIG. 20 shows the door catch 114 and latching sled 132 of the peristaltic pump 100 of FIG. 1 from the front side of the pump 100. A door-catch interface 222 separates the outside, in which the pin catch 166 protrudes outside the door-catch interface 222, from the internal parts of the door catch 114 in which the latching sled 132 operates on. FIG. 21 shows the latching sled 132 including a sled base 136 and a claw 134 pivotally coupled to the sled base 136 about an axis of the sled cam follower 176. The sled cam follower 176 is secured to both the sled base 136 and the claw 134 via a sled pin 178. A sled spring 142 is coupled to the claw 134. The sled base 136 slides back and forth in a block 138 of the door-catch interface 222 as shown in FIG. 22.

FIG. 22 shows the door catch 114 and latching sled 132 of the peristaltic pump 100 of FIG. 1 from the back side of the pump 100. The claw 134 of the latching sled 132 is in a locking position. The sled spring 142 is coupled to the claw 134 and to an anchor pin 140 of the block 138. The sled spring 142 biases the claw 134 toward the sled base 136 and biases the latching sled 132 toward the door-catch hold 234. However, the position of the sled base 136 within the block 138 is controlled by the hook cam 144 (See FIG. 19).

FIG. 23 shows the door catch 114 and latching sled 132 where the claw 134 of the latching sled 132 is in a retracted position. As is easily seen in FIG. 23, the door-catch hold 234 has been pulled back by the claw 134. In this position, wherein the latching sled 132 has been pulled back because the lever 104 has been actuated to the open position, the door-catch hold 234 is free to be actuated between the two positions shown in FIGS. 22 and 23 because the claw 134 has been lifted up away from the door-catch hold 234. The force of the door-catch spring 224 on the door-catch anchor 232 pushes the door-catch hold 234 into one of the positions of FIGS. 22 and 23.

FIG. 24 shows the door catch 114 and a portion of the block 138 that seats the latching sled 132 for the peristaltic pump 100 of FIG. 1. Also show in exploded view is the anchor pin 140 on the top portion of the block 138 that is secured to the bottom portion of the block 138 by a screw 240. Easily seen in FIG. 24, the door-catch hold 234 is actuatable between the two positions. FIG. 25 shows the door catch 114, which is rotatable along a pivot defined by the channel 236. The channel 236 may receive any device that makes the door catch 114 pivotable, such as a pin, flange, or protrusion on the door-catch interface 222.

Refer now to FIGS. 26-28: FIG. 26 shows a cross-sectional view of the peristaltic pump 100 of FIG. 1 with a hook cam 144 in a non-hooking position; FIG. 27 shows the cross-sectional view of FIG. 26, but with the hook cam 144 partially actuated toward the cam follower of the latching sled 132; And FIG. 28 shows the cross-sectional view of FIG. 26, but with the hook cam 144 fully actuated such that the hook 146 has coupled to the cam follower of the latching sled 132 and has fully retracted the latching sled 132.

As can be seen through the sequence of FIGS. 26, 27, and 28, the hook 146 of the hook cam 144 grabs onto the sled cam follower 176 and retracts the latching sled 132. As the claw 134 is pulled back, the door-catch hold 234 is retraced within it. The door catch 114 is then in the unlocked state as shown in FIG. 28. When the door 102 is fully opened as shown in FIG. 28, the door-catch hold 234 is able to freely actuate between the open and closed position. The door-catch spring 224 pushes against the door-catch anchor 232 such that the door catch 114 is bi-stable between the positions shown in FIGS. 26 and 28. Also, it is easily viewable in FIG. 28 that the block 138 lifts up the claw 134 as it is retracted by the hook cam 144 despite the sled spring 142. That is, the surface of the block 138 provides a cam action against the claw 134 to lift up the claw 134 when the latching sled 132 is retracted by the hook cam 144. The sled spring 142 biases the claw 134 toward the sled base 136. FIG. 29 shows the hook cam 144 with a close up to illustrate the retraction space 238 which allows a portion of the claw 134 to retract more closely to the main shaft 118.

FIG. 30 shows an exploded view of a coupling 242 for coupling together the main shaft 118 to the upper shaft 298 of the peristaltic pump 100 of FIG. 1, and FIG. 31 shows an exploded view of the coupling 242 of FIG. 30 but from another viewing angle.

Referring to both FIGS. 30 and 31, the coupling 242 includes a middle connector 250, a first connector 282, and a second connector 284. The embodiment shown herein shows the hook cam 144 and the first connector 282 integrated together. The middle connector 250 is rigidly coupled to the main shaft 118. The hook cam 144 rotates around the main shaft 118 (see FIG. 19). The second connector 284 is rigidly coupled to the upper shaft 298 (see FIG. 19).

The middle connector 250 includes a first flange 252 that can interface with one of a first stop 256 of the first connector 282 or a second stop 258 of the first connector 282. The middle connector 250 also includes a second flange 254 that can interface with a third stop 260 or a fourth stop 262 of the second connector 284. The first flange 252 engages with the first stop 256 of the first connector 282 such that when the lever 104 is actuated from the closed position to the open position, the rotation of the main shaft 118 rotates the middle connector 250 (via direct coupling) to press the first flange 252 against the first stop 256 to thereby actuate the hook cam 144 to retract the latching sled 132. Likewise, the second flange 254 engages with the third stop 260 such that when the lever 104 is actuated from the closed position to the open position, the rotation of the main shaft 118 rotates the middle connector 250 (via direct coupling) to press the second flange 254 against the third stop 260 to rotate the second connector 284 with the main shaft 118; because the upper shaft 298 is directly coupled to second connector 284, the interface of the second flange 254 with the third stop 260 causes the main shaft 118 and the upper shaft 298 to rotate with each other when the lever 104 is actuated from the closed position to the open position.

A first shaft spring 246 torsionally biases the middle connector 250 relative to the first connector 282, and the second shaft spring 248 torsionally biases the middle connector 250 relative to the second connector 284. The coupling 242 allows the main shaft 118 to continue to rotate a predetermined amount when the gears 212 are locked and thereby causing the upper shaft 298 to remain stationary. Although described in greater detail below, a pawl 154 of the carriage assembly 160 (see FIG. 33) can prevent the carriage 150 from rotating and can prevent the gears 212 (see FIGS. 32-33) from also rotating. Because the gears 212 are rigidly coupled to the upper shaft 298, when the gears 212 are prevented from rotating, the upper shaft 298 is also prevented from rotating.

That is, a user trying to actuate the lever 104 to the closed position while the door 102 is open will be prevented from closing the lever 104 to keep it closed because once a user lets go of the lever 104, the lever 104 will quickly spring back to the open position. Rather than rigidly stopping any actuation of the lever 104 as the user attempts to actuate the lever 104 to the closed position while the door 102 is open, the coupling 242 provides a spring resistance until the lever 104 is in the fully closed position. The main shaft 118 is not shown in FIGS. 30-31, however as previously mentioned, the main shaft 118 is rotationally disconnected from the upper shaft 298 thereby allowing them to rotate independently. In other embodiments, the main shaft 118 may be rigidly coupled to the upper shaft 298. When the door 102 is open, the coupling 242 allows a predetermined amount of actuation of the lever 104 toward the closed position until the lever 104 is fully closed, or in other embodiments, the coupling 242 prevents any additional actuation. When the door 102 is closed, the upper portion of the main shaft 118 is not locked and the lever 104 can be freely actuated to the closed position.

When the door 102 is open and the user tries to actuate the lever 104 from the open position to the closed position, the main shaft 118 continues to rotate. Because the main shaft 118 is coupled to the middle connector 250, the middle connector 250 will rotate with actuation of the lever 104; however, the second connector 284 will not rotate because the gears 212 are locked by virtue of the door 102 being open which thereby locks the upper shaft 298 and the first connector 282 will also not rotate because the hook cam 144 cannot overcome the bias of the door-catch spring 224 that holds the latching sled 132 in the retracted position. Referring to FIGS. 30-31, in this situation, the middle connector 250 will rotate because it is connected to the main shaft 118 and the first connector 282 and the second connector 284 will remain stationary as the user attempts to close the lever 104 with the door 102 open. The hook cam 144 does not rotate in this situation because it is rigidly connected to the first connector 282. The first flange 252 will leave the first stop 256 thereby charging the first shaft spring 246 and the second flange 254 will leave the third stop 260 thereby charging the second shaft spring 248. If the user lets go of the lever 104, it will quickly open because of the charging of the first shaft spring 246 and the second shaft spring 248. Alternatively, if the user, while holding the lever 104 in the fully closed position against the biasing of the first shaft spring 246 and the second shaft spring 248, attempts to close the door 102, the lifter pin 226 will actuate causing the lifter spring 228 to press against the lift 156. However, because the pawl 154 (see FIG. 33) is locked under force (via the first shaft spring 246 and the second shaft spring 248), the lifter spring 228 cannot overcome the force needed to lift the lift 156 and release the carriage 150 (described in more detail below). Nonetheless, the latching sled 132 may overcome the spring 224 (through assistance of the door 102 causing actuation of the door catch 114) thereby allowing the hook cam 144 to rotate such that the first stop 256 again engages the first flange 252; however, as soon as the user lets go of the lever 104, the lever 104 will quickly open causing the hook cam 144 to quickly retract the latching sled 132 again because of charge of the second shaft spring 248.

FIG. 32 shows a cross-sectional view of the peristaltic pump 100 of FIG. 1. The gears 212 can actuate the carriage 150 by actuation of the main shaft 118. That is, the gears 212 couple the main shaft 118 to the carriage 150 (see FIGS. 34-36) so that the carriage 150 (see FIGS. 34-36) can rotate. Rotation of the carriage 150 causes the tube 216 to either be in an occluding position or a non-occluding position within the flow stop 152. FIGS. 32, 34, 35 correspond to the carriage 150 being in a position that positions the tube 216 to be occluded within the flow stop 152, while FIG. 36 corresponds to the carriage 150 being in a position that positions the tube 216 to be non-occluded within the flow stop 152. FIG. 33 shows the lifter pin 226 in the position that can correspond to either FIG. 35 or FIG. 36.

FIG. 32 shows the lifter pin 226 in a position that prevents the carriage 150 from rotating when a user attempts to shut the lever 104 with the door 102 open. FIG. 33 shows the lifter pin 226 in a position that allows the carriage 150 to rotate in response to a user closing the lever 104 when the door 102 is closed.

When the door 102 is open as shown in FIG. 32, the lifter pin 226 sticks out of a hole (see FIGS. 2-4 for a clear view of the end of the lifter pin 226) to ensure that the carriage 150 is locked and is prevented from rotation in direction 608 as shown in FIG. 34. As shown in FIG. 34, the pawl 154 is located in a groove of the notches 268 which prevents the carriage 150 from rotating to the position shown in FIG. 36. That is, the pawl 154 has locked the carriage 150. When the door 102 is open as shown in FIG. 32, the pawl 154 is engaged with the notches 268 as shown in FIG. 34. Because the door 102 is open, the lifter pin 226 is not pushing on the lift 156 through the lifter spring 228. This prevents the lever 104 from being actuated toward the closed position because the carriage 150 is coupled to the gears 212, which in turn is mechanically coupled to the main shaft 118. This feature prevents the user from actuating the lever 104 closed while the door 102 is open. Closing the door 102 actuates the pawl 154 out of the notches 268 (via the lifter pin 226).

FIG. 33 shows the same cross-sectional view as in FIG. 32 but with the door 102 closed which thereby actuates the lifter pin 226 away from the door 102 to compress the lifter spring 228 which actuates the lift 156. That is, as shown in FIG. 33, when the door 102 is shut, the door 102 presses on an end of the lifter pin 226 (see FIGS. 2-4) which actuates the lifter pin 226 in a direction that is illustrated by an arrow 604 in FIG. 33. The lifter-pin collar 230 is rigidly coupled to the lifter pin 226 and thus both the lifter-pin collar 230 and the lifter pin 226 move in the direction of the arrow 604 when the door 102 is shut to the position shown in FIG. 33.

As previously mentioned, the door 102 impinges on the end (see FIGS. 2-4) of the lifter pin 226 when the door 102 is shut thereby actuating the lifter pin 226 in the direction of arrow 604 as shown in FIGS. 32-33. As the lifter pin 226 actuates away from the door 102, the lifter-pin collar 230 also moves away from the door 102 to thereby compress a lifter spring 228 against the lift 156. Compression of the lifter spring 228 applies a force against the lifter pin 226 which actuates the lift 156 away from the door 102 because the lift 156 is coupled to a pawl 154 as shown in FIGS. 34-36. The pawl 154 is pivotably coupled to the carriage assembly 160 via a pawl pivot 606.

When the door 102 is open as shown in FIG. 32, the lifter pin 226 is actuated away from the lift 156 such that the pawl 154 engages with the notches 268 as is shown in FIG. 34. FIG. 34 shows a cross-sectional view of the peristaltic pump 100 of FIG. 1 to show a cross-sectional view of the carriage assembly 160 with the door 102 open and the lever 104 open. As shown in FIG. 33, when the lift 156 is actuated away from the carriage 150 by closing the door 102, the pawl 154 is also actuated away from the carriage 150 as is shown in FIG. 35 by compression of the lifter spring 228 against the lift 156 coupled to the pawl 154. FIG. 35 shows the same cross-sectional view as in FIG. 34 but the door 102 is closed which actuates the pawl 154 out of the notches 268.

That is, actuation of the lift 156 away from the carriage 150 actuates the pawl 154 such that the carriage 150 can freely rotate. When the pawl 154 is lifted by the lift 156, the pawl 154 cannot engage with the notches 268 of the carriage 150 as shown in FIG. 35 and therefore the carriage 150 can freely rotate. When the pawl 154 is engaged with the notches 268 as shown in FIG. 34 of the carriage 150, the carriage 150 cannot rotate to the position shown in FIG. 36. The carriage 150 can rotate in the direction 608 shown as the clockwise arrow in FIGS. 34 and 35 into the position shown in FIG. 36 when the lever 104 is closed. FIG. 36 shows the same cross-sectional view as in FIG. 35 but with the carriage 150 in a rotated position which is caused by closure of the lever 104.

As shown in FIG. 34, the flow-stop retainer 170 includes a retainer hook 286 and a spring body 288. The flow-stop retainer 170 allows the flow stop 152 to be snap-fitted in the carriage 150 and also provides resistance when pulling the flow stop 152 out of the carriage 150.

FIG. 37 shows the carriage assembly 160 of the peristaltic pump 100 of FIG. 1 from a bottom side of the carriage 150, and FIG. 38 shows the carriage assembly 160 of the peristaltic pump 100 of FIG. 1 from a top side of the carriage 150. FIG. 37 shows the gear connector 290 that mechanically couples the carriage 150 to the main shaft 118. The carriage assembly 160 includes a carriage housing 148, a pawl 154, a pawl spring 180, the gear connector 290, and a window 264. The window 264 allows light (e.g., generated by an LED) to shine through the window 264. A sensor on the other side of the window 264 can sense which portions of the window 264 are blocked and/or which positions of the window 264 has light shining therethrough. Flow-stop ID holes 294 on a flow stop 152 can indicate a binary number which can be used to identify the flow stop 152 and/or the set the flow stop 152 is attached to. As shown in FIG. 37, when the carriage 150 is in the closed position, a cover 266 blocks the entrance to the carriage assembly 160 (also see FIG. 37).

FIG. 39 shows the carriage assembly 160 of the peristaltic pump 100 of FIG. 1 from a bottom side of the carriage assembly 160 with the bottom portion of the carriage housing 148 removed for clarity. As shown, the cover 266 can be easily seen as blocking the entrance of the carriage assembly 160, which in turn prevents insertion of anything into the carriage 150 while the carriage 150 is rotated to the closed position. The flowing portion 270 of the flow stop 152 is over the carriage-assembly hole 292 which allows fluid to flow through the tube 216. When the carriage 150 is in the open position, the carriage-assembly hole 292 holds the tube 216 such that the tube 216 is positioned within the occluding portion 272 of the flow stop 152. This requires the flow stop 152 to be loaded and unloaded into the carriage 150 by the user only when the flow stop 152 is occluding the tube 216.

After the flow stop 152 is secured within the carriage 150 and the door 102 is shut, actuation of the lever 104 to the closed position rotates the carriage 150 such that the carriage-assembly hole 292 holds the tube 216 such that the tube 216 can reside within the flowing portion 270 of the flow stop 152. When the tube 216 is positioned within the flowing portion 270, fluid may easily flow through the tube 216. FIGS. 40 and 41 show views of the carriage 150 of the peristaltic pump 100 of FIG. 1. The notches 268 are easily seen as is the cover 266.

FIG. 42 shows the carriage 150 with the top portion removed to illustrate a guide surface 149 of the carriage 150. The guide surface 149 is configured to allow the stabilizer 278 of the flow stop 152 to translate insertion force applied to the thumb rest 280 into sliding of the tube 216 within an arcuate slot 151 of the flow stop 152 which is described in greater detail below.

FIGS. 43-48 show several views of the flow stop 152 that can be inserted into the carriage 150 of the peristaltic pump 100 of FIG. 1. The flow stop 152 includes a body 296 defining an arcuate slot 151 that receives a tube 216 therein. The arcuate slot 151 includes an occluding portion 272 and a flowing portion 270. The flow stop 152 also includes a stabilizer 278. The stabilizer 278 facilitates insertion of the flow stop 152 into the carriage 150. A thumb rest 280 is shown that provides a frictional area for a person to press the flow stop 152 into the carriage 150. As is easily seen in FIG. 43, the thumb rest 280 includes an extension 274. Within the extension 274 are the flow-stop ID holes 294 for the light to identify the flow stop 152. The flow-stop ID holes 294 are easily seen in FIG. 43. The back 276 is easily seen in FIG. 45.

FIGS. 49-53 show a sequence of events to illustrate the flow stop 152 of FIGS. 43-48 being inserted in the carriage assembly 160 of the peristaltic pump 100 of FIG. 1. The carriage 150 as shown in FIGS. 49-53 is shown with the top removed for easy viewing of the interaction of the stabilizer 278 and the guide surface 149. The stabilizer 278 and the guide surface 149 interact with each other in order to prevent the flow stop 152 from being inserted into the carriage at an angle that would pinch the tube 216.

Initially, prior to insertion of a flow stop 152 of an administration set, a user may place the tube 216 anywhere within the arcuate slot 151. If the user places the tube 216 within the end of the occluding portion 272 of the arcuate slot 151, the carriage 150 can receive the flow stop 152 with the tube 216 being occluded without moving or repositioning the tube 216 within the arcuate slot 151.

However, if the user has the tube 216 positioned in the flowing portion 270 or partially between the flowing portion 270 and the end of the occluding portion 272, the carriage assembly 160 will reposition the tube 216 to the end of the occluding portion 272 as the flow stop 152 is inserted into the carriage 150.

FIG. 49 shows the initial insertion of the flow stop 152 where the tube 216 is in the flowing portion 270. As can be seen in the sequence of events from FIG. 49 to FIG. 53, as the flow stop 152 is inserted, the tube 216 slides into the end of the occluding portion 272 as shown in FIG. 34. During this process, the stabilizer 278 and the guide surface 149 interact with each other to prevent the tube 216 from getting pinched or damaged from forces orthogonal to the center line of the arcuate slot 151.

That is, as a user pushes on the thumb rest 280, the guide surface 149 causes the flow stop 152 to be guided to the fully-inserted position in the carriage 150 as shown in FIG. 53 while adjusting the angle of the flow stop 152 to translate forces on the thumb rest 280 to the tube 216 so that the tube 216 experiences a force substantiality parallel with the center line of the arcuate slot 151. The stabilizer 278 is guided by the guide surface 149 because the stabilizer 278 will abut the guide surface 149 if the user attempts to rotate the flow stop 152 counterclockwise (from the perspective shown in FIGS. 49-53) while attempting to insert the flow stop 152. Thus, the stabilizer 278 of the flow stop 152 prevents the tube 216 from becoming pinched or damaged by the interface between the carriage 150 and the flow stop 152. The stabilizer 278 and the guide surface 149 mitigate the force of the user pressing on the flow stop 152 from being translated on the tube 216 to push the tube orthogonal with the center line of the arcuate slot 151 which would cause the tube 216 to become pinched because the tube 216 would be trapped within the channel defined by the hole 106 (see FIG. 2) if the tube 216 was forced to move orthogonally to the center line of the arcuate slot 151.

FIG. 54 shows the carriage assembly 160 from the top side with a sensor board 161 coupled thereto of the peristaltic pump 100 of FIG. 1. FIG. 55 shows the same view as FIG. 54 but with the sensor board 161 shown as being transparent to show a group of LEDs 165 which are part of a flow-stop ID sensor 163. The flow-stop ID sensor 163 includes the LEDs 165 that are used to generate light, which may be visible light, non-visible light, infrared light, near infrared light, ultraviolet light, narrow-band light, wide-band light, within an optical portion of the electromagnetic spectrum, or some suitable combination thereof. The flow-stop ID sensor 163 also includes an optical sensor 153, which may be a linear array of light sensitive elements, e.g., 128 grayscale detectors. Also, as is easily seen in FIG. 56, the flow-stop ID sensor 163 includes a light pipe 155.

The LEDs 165 emit light that is transmitted within the light pipe 155 to route light to the side of the carriage assembly 160 opposite to the side that the sensor board 161 is coupled to. FIG. 57 shows the light pipe 155 including a receiver aperture 167 that receives light from the LEDs 165 (see FIG. 56) and a transmission aperture 157 that transmits light through a window 264 of the carriage assembly 160 on the bottom side of the carriage assembly 160 (see FIG. 37 for the window 264 on the bottom side). The light is transmitted through any of the flow-stop ID holes 294 of the extension 274 (see FIG. 43) when the flow stop 152 is in the carriage 150 and the carriage 150 is positioned in the lever-closed position (as shown in FIG. 39) when the lever 104 is closed.

Referring again to FIGS. 55 and 56, as is easily seen, using the light pipe 155 allows a single sensor board 161 to house the LEDs 165 and the optical sensor 153. The sensor board 161 also includes a rotation sensor 169 that may be a rotary encoder coupled to an end of the upper shaft 298 (see FIG. 11).

FIG. 58 shows a flow-chart diagram to illustrate a method 400 of using the peristaltic pump 100 of FIG. 1. The method 400 may include acts 401-415. Act 401 actuates the lever 104 to the open position by a user. That is, if the lever 104 was previously closed, the user can actuate the lever 104 open, which will open the door 102 as described above and is illustrated as Act 402 in the method. Act 402 opens the door 102 and rotates the carriage 150 to a position to receive a flow stop 152 in response to actuation of the lever 104 to the open position (see FIG. 34). In this position, if the carriage 150 already includes a flow stop 152 (e.g., from a previous therapy), the user can remove the flow stop 152 and replace it with a new flow stop 152 because in Act 402, the carriage 150 was rotated to a position where a user can remove or insert the flow stop 152. Act 403 actuates the spring-biased plunger 116, the inlet valve 198, and the outlet valve 200 into a retracted position in response to actuation of the lever 104 to the open position. This facilitates easy insertion of the tube 216 into the platen 168 without being impeded by one or more of the spring-biased plunger 116, the inlet valve 198, and/or the outlet valve 200.

Act 404 moves the flow stop 152 to an occluding position on the tube 216 by the user. Act 404 is optional because during Act 405 the user will insert the flow stop 152 into the carriage 150 and, as described above with reference to FIGS. 49-53, the tube 216 may be moved to the occluding position within the arcuate slot 151 automatically during flow stop 152 insertion into the carriage 150.

Act 406 prevents actuation of the lever 104 to the closed position if attempted by the user while the door 102 remains open. Act 407 closes the door 102 by the user. Act 408 unlocks the carriage 150 in response to closing the door 102. Act 409 actuates the lever 104 to the closed position by the user. Act 410 rotates the carriage 150 to position the tube 216 within the flow stop 152 to a non-occluding position in response to actuation of the lever 104 to the closed position. Act 411 releases the spring-biased plunger 116, inlet valve 198, and the outlet valve 200 from the retracted position in response to actuation of the lever 104 to the closed position. That is, the lift cam 120 or (or 302) will no longer interact with the spring-biased plunger 116, inlet valve 198, and outlet valve 200. Act 412 illuminates a plurality of LEDs 165 onto a plurality of predetermined locations on the flow stop 152, such as on the flow-stop ID holes 294. Act 413 determine whether each of the plurality of predetermined locations on the flow stop 152 is optically blocked or unblocked by sensing the illuminations from the plurality of LEDs 165. Act 414 generates a binary number based upon of the predetermined locations on the flow stop 152. Act 415 authorizes or denies the pump 100 to permit infusion therapy based upon the binary number.

FIG. 59 shows a driver circuit 338 of the peristaltic pump 100 of FIG. 1 for driving the LEDs 165 of the flow-stop ID sensor 163. The driver circuit includes an op-amp U15 which is arranged in a negative feedback loop to a drive transistor Q3. The op-amp U15 drives its output such that a target voltage is achieved. This target voltage controls the base of the transistor Q3 which in turn causes the transistor Q3 to control for a constant current through resistor R163. This causes the current flowing from terminal 3 to terminal 2 of the transistor Q3 is be substantially constant. The schematic shows signal LED_SETID_ADC that is a voltage that directly correlates to the amount of current traveling through the LEDs. This voltage may be measured to verify that the LEDs' current consumption matches the commanded value. Using this measurement, a processor may detect some cases of shorted or open LEDs.

FIG. 60 shows an LED circuit 339 of the peristaltic pump 100 of FIG. 1 showing the arrangement of the LEDs 165 of the flow-stop ID sensor 163. The LED_SETID_SINK_F_INT pin is coupled to the output of the circuit of FIG. 59 which includes the same label. The constant current causes the LEDs D1, D2, D3 to generate visible light which is directed through the light pipe 155. The LEDs D1, D2, D3 may be the LEDs 165 shown in FIG. 55-56. In other embodiments, more LEDs (e.g., four) or fewer LEDs may be used as would be known to one of ordinary skill in the relevant art.

FIG. 61 shows an optical-sensor circuit 340 of the peristaltic pump 100 of FIG. 1 for sensing light received after light from the LEDs 165 has passed through the flow-stop ID holes 294 of the extension 274 of the flow stop 152. The optical sensor circuit of FIG. 61 uses a linear detector shown as IC U3. In some embodiments of the present disclosure, the IC U3 may be part number TSL1401CCS manufactured by ams AG of Tobelbader Strasse 308141, Premstaetten, Austria. In other embodiments, the sensor used may be part number LF1401 manufacturer by iC-Haus GmbH of Am Kuemmerling 18, 55294 Bodenheim, Germany. However, any suitable optical sensor 153 may be used including, but not limited to, other linear optical sensors. The IC U3 may be the optical sensor 153 shown in FIG. 55. Output of the IC U3 is sent to a processor via pin 6 of the IC U3 after being processed, e.g., by an analog-to-digital converter (not shown) that in some specific embodiments, is integrated into the processor. However, the analog-to-digital converter may be a separate integrated circuit from the processor.

FIG. 62 shows a flow chart diagram 1000 illustrating a method of using data from the light sensor shown in FIG. 61 to identify a flow stop 152. The holes or absence of holes on the flow stop 152 may include 10 locations that correspond to 10 bits such that 10 different codes can be identified each of which corresponds to an infusion-set model number connected to the flow stop 152. The 10 codes can have a hamming distance of four relative to each other. And if any code is shifted to the left or right, the shifted code will have a hamming distance of three when the shift is less than 3 and a hamming distance of two when the shift is greater than or equal to 3. The codes may have an even number of ones and zeroes, e.g., 6/4 or 8/2. The codes may have at least six transitions from 1 to 0 or from 0 to 1.

The method includes Acts 1001-1014. Act 1001 performs a self-test on the optical sensor when the door 102 is opened. The optical sensor may be 128 pixels wide and each bit may be no less than 11 pixels wide. Act 1002 generates a dust map while the door 102 remains open. Act 1003 calibrates the optical sensor. Act 1004 inserts a flow stop. Act 1005 shuts the door 102. Act 1006 closes the lever. Act 1007 illuminates the LEDs. Act 1008 reads an image from the optical sensor. A PI controller may control the exposure so that a mean image intensity is at or close to a mid-range value or other predetermined value. Act 1009 downsamples the image. For example, each grayscale pixel in the image may be downsampled from 12 bits to 8 bits. Act 1010 validates the image. For example, variance and mean values must be within predetermined ranges to be validated. Act 1011 performs edge detection of the image to generate an edged-detected image. The edge detection may be performed using a modified Prewitt kernel with a kernel function of {−1, −2, −3, 0, 3, 2, 1}. Act 1012 convolves the edge-detected image with a correlated template to generate a convolved image. Act 1013 identifies edge transitions using the convolved image. An area of the highest intensity may be considered to be a center of a bit. Thereafter, a location is based upon fixed distances to the left and/or right where values are expected to be. That is, bit indices are used to sample the original image with a threshold value to determine whether a location is a ‘1’ or ‘0’. Each value is an average of five pixels centered on the sample point, in some specific embodiments. Act 1014 identifies the flow stop. A lookup table may be used to correspond values with infusion set part numbers.

Open spaces may be correlated with a binary 1 and the correlation of the closed spaces with a binary 0. For example, there may be 13 binary digits, and an optical sensor may recognize the outer boundaries of the encoded binary number. The binary-number encoded may correspond to a model number (or product number) of the flow stop or the infusion set having the flow stop. For example, there may be 10, 9, or 5 unique codes. These code values may be selected to be resistant to partial occlusions, misalignment, unexpected LED illumination brightness, etc. For example, a Monte Carlo simulation may be run to maximize the Hamming distance of the binary numbers used. The Hamming distance is a measure of how many bits have to be changed to turn one sequence of valid bits into another valid binary number. There may be a minimum Hamming distance of three between any two codes considered valid by the peristaltic pump. That is, three single-bit errors will accumulate before one barcode looks identical to another when the minimum Hamming distance is three. Monte Carlo simulation indicates that 3 is a sufficient number for this application. There may be a minimum of 2 contiguous bits of the same digit (e.g., 11 or 00) and there may be a maximum of five contiguous bits of the same digit.

Referring generally to the drawings, FIGS. 63-96 show an alternative embodiment of the peristaltic pump 100 of FIG. 1 where an alternative lift cam 121, an alternative mechanical linkage between the shaft and carriage 150, and an alternative door catch 308 are used and is labeled generally as peristaltic pump 300.

FIG. 63 shows a rear view of the peristaltic pump 300 with the rear cover removed. A lift cam 302 is shown and includes a flange 304. The flange 304 limits the movement of the lift cam 302 toward the spring-biased plunger 116. FIG. 64 shows another view of the peristaltic pump 300 of FIG. 63 to illustrate the operation of the lift cam 120 by showing the lever 104 in the open position. The lift cam 302 is rotated into a lifting position, but, as is shown in FIG. 64, the flange 304 prevents the lift cam 302 from slipping under the spring-biased plunger 116. FIG. 65 shows a cross-sectional view of the lift cam 120 of the peristaltic pump 300 of FIG. 63 when the lever 104 is in the open position. As shown in FIG. 65, the flange 304 prevents the lift cam 302 from slipping beyond a predetermined rotational angle. The lift cam 302 is biased by a cam-lifter torsion spring 126 in the direction of arrow 311. FIGS. 66-72 show the lift cam 120 of the peristaltic pump 300 of FIG. 63 from various viewing angles.

FIG. 73 shows the peristaltic pump 300 of FIG. 63 from a back view to show a door-catch linkage bar 306 between the door catch 308 and a linear ratchet 309. A door-catch spring 310 is coupled to the door-catch linkage bar 306 and the linear ratchet 309. The door-catch linkage bar 306 can rock back-and-forth because it is pivotally coupled to a frame 312. The door-catch spring 310 operates using an over-center action as described above which makes the door catch 308 bi-stable. FIG. 74 shows the peristaltic pump 300 of FIG. 63 to provide another view of the door-catch linkage bar 306 between the door catch 114 and a linear ratchet 309. As is shown in FIG. 74, a central span of the door-catch linkage bar 306 is rotatably coupled to the frame 312 so that actuation of the door catch 308 causes the linear ratchet 309 to change states. The linear ratchet 309 can be in a ratcheting state or in a non-ratcheting state. In the ratcheting state, the linear ratchet 309 can Act as a lock to prevent rotation of the carriage 150. That is, the linear ratchet 309 in the peristaltic pump 300 performs the locking action that is performed by the pawl 154 in the peristaltic pump 100 of FIG. 1. The linear ratchet 309 also includes a pawl 318 that locks the main shaft 118 via a carriage linkage bar 335 rather than directly acting on the carriage 150.

FIG. 75 shows a close-up view of the interface of the door-catch spring 310 and the door catch 308. FIG. 75 also shows the door-catch linkage bar 306 of the peristaltic pump 300 of FIG. 63 with a door catch 308 in the door 102 open position and the lever 104 in the open position. As can be seen, the door-catch spring 310 includes a ball 314 that interfaces with a socket 315 to form a ball-and-socket joint 316. When the door 102 is open, the door catch 308 can be in a position such that the door-catch linkage bar 306 has actuated the linear ratchet 309 to a ratcheting state. In the ratcheting state, the linear ratchet 309 prevents the main shaft 118 from rotating when a user attempts to close the lever 104 thereby preventing the user from closing the lever 104 while the door 102 remains open. FIG. 76 shows the same close-up view of FIG. 75 but with the door catch 308 in the door-shut position and the lever 104 in the open position. When a user shuts the door 102, it actuates the door catch 308, which actuates the door-catch spring 310, which actuates the door-catch linkage bar 306 which places the linear ratchet 309 in the non-ratcheting state. That is, the lever 104 can now be shut by the user because the door 102 is closed. FIG. 77 shows the same close-up view of FIG. 76 but after the lever 104 was actuated to the closed position. The lever 104 could be shut because the linear ratchet 309 was in the non-locking position when the lever 104 was actuated closed as described above.

FIG. 78-84 show several views of the door catch 308 including the socket 315 that receives the ball 314 from the door-catch spring 310. The door catch 308 of FIGS. 78-84 operates in the same manner as the door catch 308 shown in FIG. 25; however, the door catch 308 has a socket 315 to connect to an the door-catch spring 310 rather than a door-catch anchor 232 as shown in FIG. 25. The door catch 308 includes a door catch 114, and the door catch 308 includes a pin catch 166, a door-catch hold 234, and a channel 236 to allow the door catch 308 to pivot.

FIG. 85 shows a close-up view of the linear ratchet 309 when the door 102 is open and the lever 104 is open. The linear ratchet 309 includes a toothed linkage bar 317 and a pawl 318 that can be rotated along pivots 319, 320 so that the pawl 318 can engage or disengage with the toothed linkage bar 317. The pawl 318 is coupled to the linkage bar 325 through a pawl hole 321. The linkage bar 325 may slide through the pawl hole 321.

The pawl 318 includes a pivotable end that is coupled to the pivots 319, 320 and is configured to so that the an engagement end, such as a tooth 341 (see FIGS. 90-92) can pivot to engage the toothed linkage bar 317 or disengage the toothed linkage bar 317. The door-catch linkage bar 306 can rotate around an axis 329. Because the door-catch linkage bar 306 is in sliding engagement with the pawl hole 321 movement of the door-catch linkage bar 306 around an axis 329 can raise or lower the tooth 341 of the pawl 318 to engage or disengage the toothed linkage bar 317.

As shown in FIG. 85, the door catch 114 is in the open door 102 position which actuates the door-catch spring 310 to pivot along spring pivots 322. Because the door-catch spring 310 is coupled to the door-catch linkage bar 306 via a door-catch spring hole 323 (see FIG. 77), when the door-catch spring 310 is actuated to the door-open position, the linkage bar 325 is rotated along arrow 324 which in turn actuates the linkage bar 325 coupled to the pawl 318. The tooth 341 of the pawl 318 is actuated in direction of the arrow 326. This latching state of the pawl 318 means the pawl 318 is pivoted such that a tooth 341 of the pawl 318 engages with the toothed linkage bar 317 to prevent the user from closing the lever. That is, when the tooth 341 engages with the toothed linkage bar 317, the linear ratchet 309 is in the locking state.

FIG. 86 shows a close-up view of the linear ratchet 309 when the door 102 is closed and the lever 104 is open. The tooth 341 of the pawl 318 has actuated in the direction of arrow 330 by rotation of the door-catch linkage bar 306 in the direction indicated by arrow 331. Actuation of the tooth 341 of the pawl 318 away from the toothed linkage bar 317 thereby disengages the pawl 318 from the toothed linkage bar 317 thereby making the linear ratchet 309 to be in the non-latching state. A user can close the lever 104 when the linear ratchet 309 is in the non-latching state as shown in FIG. 87. That is, FIG. 87 shows a close-up view of the linear ratchet 309 when the door 102 is closed and the lever 104 is also closed.

FIGS. 88-89 show the peristaltic pump 300 of FIG. 63 with some parts removed to illustrate the mechanical linkage between the main shaft and the carriage 150 where the door catch 308, the door 102, and the lever 104 are in the open position. The mechanical linkage includes the toothed linkage bar 317 that is coupled to the main shaft 118 via a first pin pivot 332. The toothed linkage bar 317 is only connected at one end (i.e., via the first pin pivot 332). The mechanical linkage also includes a carriage linkage bar 335 where one end is connected to the main shaft 118 via a second pin pivot 333 and to a carriage-shaft collar 336 via a third pin pivot 334.

As previously mentioned, when the door catch 114 is in the door-open position, the tooth 341 of the pawl 318 engages with the toothed linkage bar 317. As can be seen in FIG. 88, in this position, the toothed linkage bar 317 cannot be actuated toward the main shaft 118 when a user attempts to close the lever 104 because the main shaft 118 is prevented from being rotated in direction 337 because the toothed linkage bar 317 is locked by the pawl 318. This prevents the user from closing the lever 104 prior to the door 102 being closed.

FIGS. 90-91 show the peristaltic pump 300 of FIG. 63 with some parts removed to illustrate the mechanical linkage between the shaft and the carriage 150 where the door 102 and the door catch 308 are in the closed position and the lever 104 is in open position. As can be seen, the tooth 341 of the pawl 318 has been actuated away from the toothed linkage bar 317 thereby allowing the toothed linkage bar 317 to retract toward the main shaft 118. Thus, a user can now actuate the lever 104 to the closed position.

FIG. 92 shows the peristaltic pump 300 of FIG. 63 with some parts removed to illustrate the mechanical linkage between the shaft and the carriage 150 where the door 102 and the door catch 114 are in the closed position while the lever 104 is between the open and closed position. Because the main shaft 118 has partially rotated in direction 337 by actuation of the lever 104, the carriage linkage bar 335 has pulled on the carriage-shaft collar 336 such that it is rotated along with the carriage 150 attached thereto. FIG. 93 shows the peristaltic pump 300 of FIG. 63 when the lever 104 has been closed. As can be seen, the carriage-shaft collar 336 has been fully rotated such that the carriage 150 is now in the position as shown in FIG. 36.

FIG. 94-96 show the pawl 318 of the peristaltic pump 300 of FIG. 63 from several views. FIG. 96 shows a cross-sectional view of the pawl 318 along the view indicated in FIG. 94. In FIG. 96, a tooth 341 is shown that engages with the teeth of the toothed linkage bar 317 shown above in FIGS. 73-93.

FIGS. 97-98 shows an alternative embodiment of the peristaltic pump 10200 where an alternative mechanical assembly 1021 between the lever 104 and the main shaft 118 is used. FIGS. 97-98 also shows an embodiment of the peristaltic pump 10200 where an alternative carriage 1036 is used. The peristaltic pump 10200 provides resilience between the lever 104 and a main shaft 118 via a spring 1026. The spring 1026 is a torsion spring, in some specific embodiments.

As shown in FIG. 97, when in operation the spring 1026 provides resilience such that the spring 1026, via its ends, urges the first linkage 1022 and the second linkage 1024 outward toward the ends of the track 1028. When the ends of the spring 1026 remain at the ends of the track 1028, the track 1028 moves when the lever 104 is actuated which moves the second linkage 1024. That is, the first linkage 1022 and the second linkage 1024 remain at a predetermined distance from each other at a respective end of the track 1028 when the spring 1026 maintains the first linkage 1022 and the second linkage 1024 at a maximal distance between each other in the track 1028. However, if the door 102 is open, the main shaft 118 (see FIG. 98) cannot be rotated because it is effectively locked. Therefore, the spring 1026 can become compressed as described below. The guides 1034 are configured to guide the linkages 1022, 1024 along the track 1028. Each of the linkages 1022, 1024 includes guides 1034 to keep the linkages 1022, 1024 disposed on a predetermined position on the track 1028.

Referring now to FIG. 99, when the lever 104 is actuated, the first linkage 1022 applies a force to the spring 1026; but, when the second linkage 1024 is locked (because, for example, the carriage is locked because the door 102 is open), the first linkage 1022 approaches the second linkage 1024 as guided by the track 1028 as the spring 1026 compresses. Eventually, the first linkage 1022 will engage with the second linkage 1024, in which case, the lever 104 will be stopped by a hard stop.

The lever 104 can pivot to actuate a first linkage 1022. When the main shaft 118 is not locked, this actuation also actuates the second linkage 1024. As shown in FIGS. 100-101, actuating the second linkage 1024 rotates the first bevel gear 1030, which in turn rotates a second bevel gear 1032. The second bevel gear 1032 is attached to the main shaft 118. The lower portion of the shaft may extend from the second bevel gear 1032 by being attached to the second bevel gear 1032 thereto (not shown in FIGS. 100-101). The first linkage 1022 slides along the track 1028 when the main shaft 118 is unable to rotate thereby compressing the spring 1026. Additionally or alternatively, the second linkage 1024 slides along the track when the main shaft 118 is unable to rotate.

FIGS. 102-105 show several views a flow-stop assembly 1038 in accordance with an embodiment of the present disclosure. The flow-stop assembly 1038 includes a top housing 1040 and a bottom housing 1042. A tube 1046 is coupled to the flow-stop assembly 1038 via a tube coupling 1044. The flow-stop assembly 1038 can occlude fluid flow through the tube 1046 or can allow fluid to flow freely therethrough.

Non-occluded and occluded fluid flow may be effected through the tube 1046 via actuation of a first link 1052 and a second link 1050. FIGS. 102-103 show the flow-stop assembly 1038 in the occluding position and FIGS. 104-105 show the flow-stop assembly 1038 in the non-occluding position. When the flow-stop assembly 1038 is in the occluding position, as shown in FIGS. 102-103, a user can press on the first link 1052 via a finger groove 1062 to actuate the second link 1050 and first link 1052 to the non-occluding position as shown in FIGS. 104-105. Likewise, when the flow-stop assembly 1038 is in the non-occluding position as shown in FIGS. 104-105, a user can press on a flange 1058 to actuate the second link 1050 and first link 1052 to the occluding position as shown in FIGS. 102-103.

The flow-stop assembly 1038 also includes a housing aperture 1048 which can be used to sense the configuration of an identification aperture 1060, can be used to determine if the flow-stop assembly 1038 is loaded properly or improperly, and can be used to determine the configuration of the flow-stop assembly 1038 (e.g., the occluding vs. non-occluding position, etc.). The identification may take place as is described herein using optical recognition of a pattern of the identification aperture 1060. FIG. 106 shows a cross-sectional view of the flow-stop assembly 1038, which shows a pivot post 1054 about which the second link 1050 can pivot. When the first link 1052 and the second link 1050 are in the occluding position, as shown in FIG. 106, a plunger 1064 occludes a tube 1046 by wedging the tube 1046 between the plunger 1064 and a backstop 1066. A shutter aperture 1056 is shown which blocks or allow light to pass through depending whether or not the flow-stop assembly 1038 is in the occluding position or the non-occluding position.

The second link 1050 pivots around the pivot post 1054. The first link 1052 is coupled to the second link 1050 via a ball-and-socket joint 1068 (see FIG. 107). As the first link 1052 is actuated, it is guided within a track 1072 by guides 1070. FIG. 108A shows the flow-stop assembly 1038 with the top housing 1040 removed while in the occluding position and FIG. 108B shows the flow-stop assembly 1038 with the top housing 1040 while in the non-occluding position. As is shown in FIG. 108A, when the first link 1052 is in the occluding position, the plunger 1064 is closer to the backstop 1066 and when the second link 1050 is in the non-occluding position, the plunger 1064 is a predetermined distance from the backstop 1066. The second link 1050 and the first link 1052 are coupled together via a ball-and-socket joint 1068. The guides 1070 positions the first link 1052 such that rotation of the second link 1050 along the pivot post 1054 translates to linear motion of the guides 1070 along the track 1072 as shown in FIGS. 110-114. FIGS. 108A-108B also illustrate how the position of the shutter aperture 1056 is positioned in different locations based upon the position of the second link 1050. FIGS. 110-114 show several views of the bottom housing 1042 of the flow-stop assembly 1038 including the track 1072. Please note the identification aperture 1060 where identification of the flow-stop assembly 1038 can be made, as described herein.

The first link 1052 includes a first contacting surface 1114 and a third contacting surface 1118. The second link 1050 includes a second contacting surface 1116 and a fourth contacting surface 1120. As shown in FIG. 108A, when the flow-stop assembly 1038 is in the occluding position, the first contacting surface 1114 contacts the second contacting surface 1116. As shown in FIG. 108B, when the flow-stop assembly 1038 is in the non-occluding position, the third contacting surface 1118 contacts the fourth contacting surface 1120. In some embodiments, a secondary guide 1074 can limit the movement of the first link 1052 via limiting the range of motion the secondary guide 1074 can travel within a secondary track 1076 (see FIG. 119). Referring again to FIGS. 108A-108B, in some specific embodiments of the present disclosure, the compliance of the tube 1046 may make the flow-stop assembly 1038 bi-stable, with one stable configuration being the occluding position as shown in FIG. 108A and the other stable configuration being the non-occluding configuration as shown in FIG. 108B (a hole 1122 is shown in FIGS. 108A and 108B where the tube 1046 is positioned (see FIG. 107). In alternative embodiments, a spring or springs may be used to urge the second link 1050 and first link 1052 into the two bi-stable configurations.

FIG. 109A shows an alternative embodiment of the flow-stop assembly 1051 of FIG. 108B with knife-edge pivots in accordance with an embodiment of the present disclosure. The knife-edge pivots may each includes a v-shaped member 1055, 1057 that interface with a v-shaped pivot surface 1053, 1059, respectively. The v-shaped pivot surfaces 1053, 1059 may be configured to limit movement of the v-shaped members 1055, 1057 to thereby limit their actuation to between two positions. The materials of the v-shaped pivot surfaces 1053, 1059 and/or the v-shaped members 1055, 1057 should have sufficient hardness and modulus characteristics.

FIG. 109B shows an alternative embodiment of the flow-stop assembly 1051 of FIG. 108B with a flexure 1069 that includes arms 1067 in place of the ball-and-socket joint 1068. The flexure 1069 would allow the links 1050 and 1052 to be molded by a single piece. The flexure in FIG. 109B is known as an X flexure, where two arms 1067 from each of link 1052 and 1050 connect at a joint 1069a. Other flexures may be used in place of 1067, 1069 such as a simple living hinge.

FIGS. 115-119 show several views of the top housing 1040 of the flow-stop assembly 1038. A track 1072 is shown which can guide the movement of first link 1052 via a secondary guide 1074 (see FIGS. 108A and 108B in conjunction with FIGS. 115-119). FIGS. 120-124 show several views of a first link 1052 of the flow-stop assembly 1038 having the plunger 1064 and FIGS. 125-129 show several views of a second link 1050 of the flow-stop assembly 1038. FIGS. 130-133 show several views of a tube coupling 1044 of the flow-stop assembly 1038.

FIGS. 134-138 show the flow-stop assembly 1038 being inserted into a carriage 1036. In FIG. 134, the flow-stop assembly 1038 is in the non-occluding configuration. As the flow-stop assembly 1038 is inserted into the alternative carriage 1036, a cooperating surface 1094 interacts with the second link 1050 to actuate both the second link 1050 and the first link 1052 to place the flow-stop assembly 1038 in the occluding position illustrated in FIGS. 135-136. As shown in FIG. 136, the flow-stop assembly 1038 will be actuated into the occluding position prior to insertion into the alternative carriage 1036. Thus, in some embodiments of the present disclosure, the peristaltic pump 10200 is configured to only receive a flow-stop assembly 1038 in the occluding position; And, if the flow-stop assembly 1038 is not in the occluding position prior to insertion, the peristaltic pump 10200 will actuate the flow-stop assembly 1038 into the occluding position prior to being received (in some specific embodiments, before being partially received and in others, before or during being fully received).

FIG. 137 shows the flow-stop assembly 1038 fully inserted where the gripper finger 1086 engages with the flange 1058. Also shown, is a tube shutter 1078 that actuates when the flow-stop assembly 1038 engages with it. A shaft coupler 1080 is coupled to a shaft of the peristaltic pump 10200. The shaft coupler 1080 may be coupled directly to the main shaft 118, to the main shaft 118 via one or more gears or linkages, through another shaft, or through any other mechanical mechanism known to one of ordinary skill in the relevant art. In yet additional embodiments, the shaft coupler 1080 may be coupled to an upper end of the upper shaft 298.

When the flow-stop assembly 1038 is fully inserted into the carriage 1036, a user can actuate the lever 104 thereby causing the shaft coupler 1080 to rotate along with a pin 1082. An interlock arm 1084 includes a second finger 1088 and a first finger 1090 such that actuation of the pin 1082 into a catch well 1124 causes actuation of the interlock arm 1084, which actuates a gripper finger 1086. Because the gripper finger 1086 engages with the flange 1058, actuation of the gripper finger 1086 actuates the first link 1052 and the second link 1050 into the non-occluding position by pulling the flange 1058 in a direction away from the flow-stop assembly 1038. FIG. 139 shows a perspective view of the internal mechanism of the carriage 1036 when the end effector 1092 is engaged with a flange 1058 of the flow-stop assembly 1038 and FIG. 140 shows a perspective view of the internal mechanism of the carriage 1036 when the end effector 1092 is engaged with a flange 1058 of the flow-stop assembly 1038 in the non-occluding position. The end effector 1092 may apply a force on the flange 1058 to actuate the flow-stop assembly 1038 to the non-occluding position as shown in FIG. 140. In some embodiment of the present disclosure, the first finger 1090 and the second finger 1088 may be integrated together as a single structure, e.g., and may form a loop around the pin 1082. The pin 1082 may be a protraction, a roller wheel, a roller bearing, a cam, a rolling cam, a wheel, a slidable protrusion, or any suitable device known to one of ordinary skill in the relevant art.

FIGS. 134-138 also show the actuation of the tube shutter 1078. FIG. 141 shows the front of the carriage orifice with a cooperating surface 1094 and a tube shutter 1078. FIG. 142 shows the front of the carriage orifice when the flow-stop assembly 1038 has been inserted and the tube shutter 1078 has been opened. In some embodiments of the present disclosure, a small magnet may be coupled to the interlock arm 1084 such that an adjacent hall-effect sensor is configured to measure the position (e.g., angular or rotational position) of the interlock arm 1084 for use by the processor.

In some embodiments of the present disclosure, the tube shutter 1078 can detect “dark loading” to detect certain user actions that occur when (1) the user mis-loads the administration set having the flow-stop assembly 1038 into the pump, and/or 2) the device is powered off (i.e., dark). For example, if a user loads an administration set having the flow-stop assembly 1038, but neglects to stick the flow-stop assembly 1038 into the device, the tube shutter 1078 can detect that the flow-stop assembly 1038 was not inserted and/or can be configured to pinch any tube within the platen 168 to prevent a free-flow condition.

In some embodiments of the present disclosure, the shaft coupler 1080 can rotate in FIG. 138 (clockwise as seen in FIG. 138) to actuate the flow-stop assembly 1038 to the occluding position when the end effector 1092 of the gripper finger is suitably shaped and configured. In yet an additional embodiment of the present disclosure, when a user pulls the flow-stop assembly 1038 out of the carriage in FIG. 138, the walls of the carriage actuate the first and second links 1050, 1052 to the occluding position.

FIGS. 143-146 show several views of another embodiment of the flow-stop assembly 1038. The flow-stop assembly 1038 of FIGS. 143-146 is similar to the flow-stop assembly 1038 of FIGS. 102-105 described supra; however, alternative features are described herein or are readily apparent to one of ordinary skill in the relevant art.

As shown in FIG. 143, the identification aperture 1060 is on the top housing 1040. FIG. 144 shows the housing aperture 1048 on the bottom housing 1042. Non-occluded and occluded fluid flow may be effected through the tube 1046 via actuation of a first link 1052 and second link 1050. FIGS. 143-144 shows the flow-stop assembly 1038 in an occluding position, and FIGS. 145-146 show the flow-stop assembly 1038 in the non-occluding position. When the flow-stop assembly 1038 is in the occluding position, as shown in FIGS. 143-144, a user can press on the first link 1052 via a finger groove 1062 to actuate the second link 1050 and first link 1052 to the non-occluding position as shown in FIGS. 145 and 146. Likewise, when the flow-stop assembly 1038 is in the non-occluding position as shown in FIGS. 145-146, a user can press on a flange 1058 to actuate the second link 1050 and first link 1052 to the occluding position as shown in FIGS. 143-144.

The flow-stop assembly 1038 also includes a housing aperture 1048 which can be used to sense the configuration of an identification aperture 1060, can be used to determine if the flow-stop assembly 1038 is loaded properly or improperly, and can be used to determine the configuration of the flow-stop assembly 1038 (e.g., the occluding vs. non-occluding position, etc.) using an optical sensor as described herein. FIG. 147 shows a cross-sectional view of the flow-stop assembly 1038, which shows a pivot post 1054 about which the second link 1050 can pivot. When the first link 1052 and the second link 1050 are in the occluding position, as shown in FIG. 166, a plunger 1064 occludes the tube 1046 by wedging the tube 1046 between the plunger 1064 and a backstop 1066.

The second link 1050 pivots around the pivot post 1054. The first link 1052 is coupled to the second link 1050 via a hinge 1126. As the first link 1052 is actuated, it is guided within a track 1072 by guides 1070. FIG. 148 shows the flow-stop assembly 1038 with the top housing 1040 removed while in the occluding position and FIG. 149 shows the flow-stop assembly 1038 with the top housing 1040 while in the non-occluding position. As is shown in FIG. 150, when the first link 1052 is in the occluding position, the plunger 1064 is closer to the backstop 1066 and when the second link 1050 is in the non-occluding position as shown in FIG. 149, the plunger 1064 is a predetermined distance from the backstop 1066. The second link 1050 and the first link 1052 are coupled together via a ball-and-socket joint 1068. The guides 1070 positions the first link 1052 such that rotation of the second link 1050 along the pivot post 1054 translates to linear motion of the guides 1070 along the track 1072.

In some embodiments, the flow-stop assembly 1038 includes a notch 1096 configured to use optical recognition to determine when the flow-stop assembly 1038 is in the occluding or non-occluding position. As shown in FIG. 150, the notch 1096 aligns with the housing aperture 1048 such that the optical recognition determines that the flow-stop assembly 1038 is inserted into the carriage 1036 and is in the occluding position.

FIGS. 151-155 show several views of the top housing 1040 of the flow-stop assembly 1038. The tube coupling 1044 is integrated with top housing 1040 in the embodiment shown in FIGS. 151-155. In some embodiments, a tube 1046 may include a snap-fit adapter 1130 (see FIG. 148) configured to interface with the tube coupling 1044 of FIGS. 151-155. FIGS. 156-160 show several views of the bottom housing 1042 of the flow-stop assembly 1038 of FIGS. 143-146. As shown in FIGS. 157-158, the bottom housing 1042 includes a secondary track 1076. The secondary track 1076 is configured such that a flange 1128 of a second link 1050 serves a guide and stops movement of the second link 1050 in one (or both) directions of actuation as is readily apparent by one of ordinary skill in the art. FIGS. 161-165 show several views of a first link 1052 of the flow-stop assembly 1038 having a plunger 1064 and FIGS. 166-170 show several views of the second link 1050 of the flow-stop assembly 1038 of FIGS. 143-146.

FIGS. 171-174 shows several views of a pinching flow-stop assembly 1100 having a flow stop 1104 with an arcuate slot 1110 FIGS. 171-172 show the pinching flow-stop assembly 1100 when the flow stop 1104 is in the occluding position. The flow stop 1104 can pivot around a pivot post 1054. FIGS. 173-174 show the pinching flow-stop assembly 1100 in the non-occluding position. A user can actuate the flow stop 1104 to transition the pinching flow-stop assembly 1100 to one of the occluding position or the non-occluding position.

FIGS. 175-178 show several views of the flow stop 1104 of the pinching flow-stop assembly 1100. A tube 1046 can be placed within an arcuate slot 1110 between a narrow portion or a wider portion based upon a pivot of the flow stop 1104 relative to a housing 1102 via the pivot hole 1108. As shown in FIGS. 175-178, the flow stop 1104 includes a notch 1096 that can be engaged by an end effector 1092 of a gripper finger 1086. FIGS. 179-181 show several views of the housing 1102 of the pinching flow-stop assembly 1100. The housing 1102 may be on a top side of the flow stop 1104, a bottom side of flow stop 1104, surrounding both, and in some embodiments, integrated together in a single piece that partially surrounds the flow stop 1104. The pivot hole 1108 of the flow stop 1104 engages with the pivot post 1106 to pivot relative to each other.

FIGS. 182-184 show the pinching flow-stop assembly 1100 being inserted into a carriage 1036. As shown in FIG. 182, when the pinching flow-stop assembly 1100 is inserted, a notch 1112 approaches and can engage with the end effector 1092 of the gripper finger 1086. As shown in FIG. 182, the alternative carriage 1036 also includes an optical sensor 1132. FIG. 183 shows the pinching flow-stop assembly 1100 fully inserted, but with the flow stop 1104 in the occluding position. FIG. 184 shows the gripper finger 1086 actuating the flow stop 1104 into the non-occluding position to treat a patient. The identification aperture 1060 is now aligned such that the pinching flow-stop assembly 1100 can be identified. A shutter can also be used as part of the alternative carriage 1036 with the pinching flow-stop assembly 1100.

In some embodiments of the present disclosure, the shaft coupler 1080 can rotate in FIG. 184 (clockwise as seen in FIG. 184) to actuate the pinching flow-stop assembly 1100 to the occluding position when the end effector 1092 of the gripper finger is suitably shaped and configured. In yet an additional embodiment of the present disclosure, when a user pulls the pinching flow-stop assembly 1100 out of the carriage in FIG. 184, the walls of the carriage actuate the flow stop 1104 to the occluding position.

### Modular Pump System

FIG. 187 shows a block diagram of a modular pump system 500 having a central unit 502 and a plurality of medical-device assemblies 504 coupled together. One or more of the medical-device assemblies 504 may be the peristaltic pump 100 or 300 shown and described herein. Additionally or alternatively, the medical-device assemblies 504 may include syringe pumps, battery packs, micropumps, or other medical devices.

The central unit 502 provides power to the medical-device assemblies 504. The central unit 502 includes a left central-unit electrical modular interconnector (MIC) 506 and a right central-unit electrical MIC 508. The left central-unit electrical MIC 506 and the right central-unit electrical MIC 508 each may include a power pin, a communications pin, and one or more ground pins. The central unit 502 provides power to the connected medical-device assemblies 504 through the left central-unit electrical MIC 506 when activated and/or the right central-unit electrical MIC 508 when activated.

The central unit 502 further includes a left mechanical modular interconnector (MIC) 510A and a right mechanical modular interconnector (MIC) 512A. The left mechanical MIC 510A on the center unit provides an attachment interface for a right mechanical MIC 512A on module #1. The right mechanical MIC 512A on the center unit provides an attachment interface for a left mechanical MIC 510A on module #2. The left and right mechanical MICs 510A, 512A provide a detachable mechanical interface between the central unit and medical device assemblies or modules 504 that support the weight of the modules.

All of the medical-device assemblies 504 includes a left mechanical medical interface connector 510 and a right medical-device connector 512 which allow the medical-device assemblies 504 to be connected to the modular pump system 500 from the left side or the right side to receive power and communicate using a common bus. Additionally, the connected medical-device assemblies 504 may be configured to connect the power from the central unit 502 to power a connected medical-device assembly 504 downstream. For example, a medical-device assembly 504 connected just to the right of the central unit 502 may be configured to subsequently power another medical-device assembly 504 connected to it on the right.

FIG. 188 shows a block diagram of a modular pump system 500 to illustrate the power circuitry of the modular pump system 500. The modular pump system 500 includes a central unit 502 and one or more medical-device assemblies 504. Although one medical-device assembly 504 is shown in FIG. 188, one or more medical-device assemblies 504 may be attached to the right of the medical-device assembly 504 shown in FIG. 188 and/or to the left of the central unit 502. Also, medical-device assemblies 504 may be serially coupled together such as is shown in FIG. 187 to the left or right side of the central unit 502.

The central unit 502 includes primary electronics 583 including a CPU 585. The primary electronics 583 includes addition functions beyond the power circuitry illustrated in FIG. 188. The medical-device assembly 504 includes module electronics 579 that includes a CPU 581. The module electronics 579 includes an electric motor for pumping fluid, power circuits, and other electronics.

The modular pump system 500 is configured such that each of the medical-device assemblies 504 can be coupled to either a right central-unit connector 508 of the central unit 502, a left central-unit connector 506 of the central unit 502, a left medical-device connector 510 or a right medical-device connector 512 of another medical-device assembly 504 (not shown in FIG. 188) to establish communication prior to receiving power through a power pin. For example, the right power pin 578 is not powered until after the medical-device assembly 504 is connected to the right central-unit connector 508 via left medical-device connector 510. Initially, the medical device assembly 504 can power itself sufficiently using the signal received via the communications pin 584. The medical device assembly 504 can request power by using the signal received via a communications pin 584 to power the medical device assembly suitably to passively request power from the device (e.g., the central unit or a medical-device assembly 504) through the communications pin 584. Power can thereafter be received via the left power pin 582 by the medical-device assembly 504 from the central unit 502 when using the system as shown in FIG. 188.

When the central unit 502 is powered up, the central-unit controller 526 may turn on a left signal switch 556 to apply a signal generated by the left signal generating circuit 530 to a left communications pin 576 of a left medical-device connector 510. Also after power up, the central-unit controller 526 may switch the right signal switch 562 into the on position to apply a signal from the right signal generating circuit 536 to the right communications pin 580 of the right central-unit connector 508. In additional embodiments of the present disclosure, the left signal generating circuit 530 and the right signal generating circuit 536 may be combined into a single circuit that generates a single signal for application to the left communications pin 576 and to the right communications pin 580. Additionally or alternatively, enable/disable circuits may be used in place of switches 556, 562, respectively, where the central-unit controller 526 can signal to enable or disable the signal generating circuits 530, 536.

The central-unit controller 526 is coupled to a left load-detect circuit 546 and a right load-detect circuit 548. The left load-detect circuit 546 is configured to detect a passive indication of a request for power of a left connected medical-device assembly 504 (none is shown in FIG. 188). The right load-detect circuit 548 is configured to detect a passive indication of a request for power of a right connected medical-device assembly 504 (one is shown in FIG. 188). The central-unit controller 526 keeps the left power switch 558 open until a request for power by a left connected medical-device assembly 504 has been received and likewise keeps the right power switch 560 open until a request for power by a right connected medical-device assembly 504 has been received. The left load-detect circuit 546 and the right load-detect circuit 548 may be current sense circuits in some embodiments. However, any circuit known to one of ordinary skill in the art may be used to detect a passive indication of a request for power. In some embodiments of the present disclosure, the passive indication of a request for power may be a change in impedance, e.g., a coupling of a resistor to the communications pin 584. Load detection may be done by monitoring current, voltage, frequency response, decay rate, an RC constant, the like, or some combination thereof.

As previously mentioned, the right load-detect circuit 548 may in some embodiments be a current sensor. Thus, if the signal from the right signal generating circuit 536 is a voltage waveform (e.g., a square waveform), the current of the right signal generating circuit 536 may be monitored by the right load-detect circuit 548 to determine if an impedance change (e.g., a decreased resistance) has occurred on the load impedance as detected by the right load-detect circuit 548.

As previously mentioned, after power up, the central-unit controller 526 switches the right signal switch 562 into the on position to apply a signal from the right signal generating circuit 536 to the right communications pin 580 of the right central-unit connector 508. When the medical-device assembly 504 is initially coupled to the central unit 502, a signal is received from the right signal generating circuit 536 through the right communications pin 580 of the right central-unit connector 508 via the left communications pin 584 of the left medical-device connector 510. The signal is used by the power receiver circuit 554 to initially power the power receiver circuit 554. That is, energy harvesting, such as a rectifier, a charge pump, etc., may be used by the power receiver circuit 554 to power itself.

The power receiver circuit 554 powers the module-detect controller 528. Upon determination by the module-detect controller 528 that a signal is present on the left communications pin 584, the module-detect controller 528 signals the left load switch 566 to close so that the left resistor 540 is now coupled to the left communications pin 584. That is, the left load switch 566 is closed thereby connecting the left resistor 540 to the left communications pin 584. This change in impedance is detected by the right load-detect circuit 548 of the central unit 502 which is communicated to the central-unit controller 526. The central-unit controller 526 takes this change in impedance to be a passive request for power. Therefore, the central-unit controller 526 switches the right power switch 560 ON so that the right power circuit 534 supplies power to the right power pin 578 through the right central-unit connector 508 via the left power pin 582 of the left medical-device connector 510. Then a switch 573 can be closed to provide power to the cross-bar bus 571 which is receivable by the power receiver circuit 554. The power is received by the power receiver circuit 554 which is then used to power the module electronics 579 by closing the switch 577. The power receiver circuit 554 can use its power to power the module-detect controller 528. In some embodiments, the switch 577 may be replaced by a diode or other circuitry to allow power to flow to the module electronics 579 anytime power is supplied to the crossbar bus 571.

After the module-detect controller 528 determines that power is being supplied from the left power pin 582, the module-detect controller 528 can configure the right side of the medical-device assembly 504 to accept another medical-device assembly 504 on its right as seen from FIG. 188 and in this example. The module-detect controller 528 may set the frequency of the right signal generating circuit 536 to half of the frequency it receives via the right signal generating circuit 536 of the central unit 502. Thereafter, the module-detect controller 528 closes the right signal switch 570 and monitors the right communications pin 588 load by monitoring the right load-detect circuit 552. Please note that load-detect circuit 550 performs the same function, but on the other side of the medical-device assembly 504. If or when the module-detect controller 528 detects a passive request for power, the module-detect controller 528 may close a right cross-bar switch 575 of a crossbar 572 so that power is supplied downstream, i.e., to the right from the view of FIG. 188. Also a right resistor 542 is coupled to a right load switch 568 that are used to passively request power, e.g., when the medical-device assembly 504 is connected to the other side of the central unit 502 from what is shown in FIG. 188.

Because the central-unit controller 526 generates a fixed frequency by the signal generating circuits 530, 536, and each medical-device assembly 504 reduces the frequency sent downstream by half, each of the medical-device assemblies 504 coupled to the modular pump system 500 can determine its position relative to the central unit 502 by monitoring the frequency of the signal coming in on respective communications pin 584, 588 because the frequency of the signals generated by 530 and 536 are predetermined and known by all of the medical-device assemblies 504. For example, the frequency values of the signals generated by 530 and 536 may be stored in non-volatile memory within the module electronics 579. Also, the side on which the medical-device assembly 504 initially receives the signal via a communication pin 584, 588 may be used by the module-detect controller 528 to know on which side of the central unit 502 it resides and by monitoring the frequency of the signal initially incoming, the medical-device assembly 504 will know how many other medical-device assemblies 504 (if any) reside between it and the central unit 502. Thus, a medical-device assembly's 504 position may be used as a bus-communications address to communicate with other medical-device assemblies and/or with the central unit 502, e.g., using on-off keying modulated signal carrying a Controller Area Network (“CAN”)-protocol signal.

FIG. 189 shows a power-on state diagram 590 of the central unit 502 power circuitry shown in FIGS. 97-98. A state 592, a state 594, and a state 596 illustrate the left power circuitry of the central unit 502 which can provide power to an attached medical-device assembly 504 through the left central-unit connector 506. A state 598, a state 600, and a state 602 illustrate the right power circuitry of the central unit 502 which can power an attached medical-device assembly 504 through the right central-unit connector 508. Please note that the two sides of power-on state diagram 590 can occur in parallel, and, in some embodiments, out of sync with each other.

In state 592, designated as POWER UP, the circuitry of the central unit 502 is powered up, for example, when a user turns on a power switch and/or plugs the central unit 502 into an A/C outlet. Thereafter, state 594 is entered into, which is designated as LEFT DETECT. In state 594, a left reference clock (e.g., signal generating circuit 530 of FIG. 188) will be turned on (e.g., the switch 556 is closed) and a left bus power (e.g., the left power circuit 532) will remain off (e.g., switch 558 remains open). The left reference clock may be created and/or controlled by a signal generating circuit 530 that is coupled to a left communications pin 576 of the left central-unit connector 506. The left bus power is a left power circuit 532 that can send power to a left power pin 574 of the left central-unit connector 506. As described in greater detail below, the left reference clock signal is monitored via left load-detect circuit 546 to sense if an impedance change indicates a passive indication of a request for power of a left connected medical-device assembly 504. For example, a left connected medical-device assembly 504 can change a resistance, e.g., by grounding (e.g., sinking) a resistor, to the communications pin 588 of the right medical-device connector 512 that is coupled to the left communications pin 576 of the left central-unit connector 506 to indicate a request for power.

As shown in FIG. 189, state 594 will continue to transition to itself as long as the passive request for power is not detected as indicated by the LEFT LOAD DETECT NOT ASSERTED transition. In state 594, if the left signal detects a load for 100 milliseconds, it is interpreted as a passive request for power, after which, the state 594 transitions to the state 596. This transition is indicated by the “LEFT LOAD DETECT ASSERTED FOR 100 ms” transition in the state diagram 590. In state 596, the central unit 502 switches to a left power-on mode and applies power to the left power pin 574 of the left central-unit connector 506 (indicated as LEFT BUS POWER=ON). The central unit 502 will continue to apply power as long as the passive request for power is detected; this is illustrated as “LEFT LOAD DETECT ASSERTED” transition in the state diagram 590. The LEFT BUS POWER=ON may signify that the left power switch 558 is closed to connect the left power circuit 532 to the left power pin 574 of the left central-unit connector 506.

The right side of the power-on state diagram 590 operates in a similar manner as the left side of the power-on state diagram 590. The two sides of the power-on state diagram 590 may operate independently and/or in parallel. As shown in FIG. 189, state 598, state 600, and state 602 illustrate the right power circuitry of the central unit 502 which can provide power to an attached medical-device assembly 504 through the right central-unit connector 508.

In state 598, designated as POWER UP, the circuitry is powered up, for example, when a user turns on a power switch and/or plugs the central unit 502 into an A/C outlet. Thereafter, the state 600 is entered into, which is designated as RIGHT DETECT. In the state 600, a right reference clock (e.g., signal generating circuit 536 of FIG. 188) will be turned on and a right bus power (e.g., the right power circuit 534) will remain off or unconnected via the right power switch 560. The right reference clock may be created and/or controlled by a signal generating circuit 536 that is coupled to a communications pin 588 of the right central-unit connector 508. The right bus power is a right power circuit 534 that can send power to a right power pin 578 of the right connector 508. The right reference clock signal is monitored via right load-detect circuit 548 to sense if an impedance change indicates a passive indication of a request for power of a right connected medical-device assembly 504. For example, a right connected medical-device assembly 504 can apply a resistance, e.g., by grounding a resistor, to the communications pin 580 of the left medical-device connector 510 that is coupled to the right communications pin 580 of the right central-unit connector 508 to indicate a passive request for power.

As shown in FIG. 189, the state 600 will continue to transition to itself as long as the passive request for power is not detected and is indicated by the “RIGHT LOAD DETECT NOT ASSERTED” transition. In the state 600, if the right signal detects a load for 100 milliseconds, it is interpreted as a passive request for power, after which, the state 600 transitions to the state 602. This transition is indicated by the “RIGHT LOAD DETECT ASSERTED FOR 100 ms” transition in the state diagram 590. In the state 602, a central-unit switchable power circuit switches to a power-on mode and applies power to a power pin of the right central unit connector 508 (indicated as RIGHT BUS POWER=ON). The right power circuit 534 will continue to apply power as long as the passive request for power is detected and is designated as RIGHT LOAD DETECT ASSERTED in the state diagram 590. The RIGHT BUS POWER=ON may signify that the right power switch 560 is closed to connect the right power circuit 534 to the right power pin 578 of the right central-unit connector 508.

FIG. 190 shows a state diagram 612 of the medical device assembly 504 power circuitry. The state diagram 612 includes states 614, 616, 618, 620, 622, 624, and 626. Within each state of the state diagram 612, Table 1 defines the output values as follows:

TABLE 3

| | | | Correspondence |
|---|---|---|---|
| Label | Description | Possible Values | to FIG. 188 |
| L LOAD | Controls | HiZ (high impedance) | Signal from |
| En | whether a | or 1 (resistor | Module-detect |
| | resistive load is | connected) | controller 528 |
| | coupled to a left | | to left load |
| | communications | | switch 566. |
| | pin. | | |
| R LOAD | Controls whether | HiZ (high impedance) | Signal from |
| En | a resistor is | or 1 (resistor | Module-detect |
| | coupled to a right | connected) | controller 528 |
| | communications | | to right load |
| | pin. | | switch 568. |
| L Ref | Controls a left | HiZ (high | Signal from |
| CLOCK | clock | impedance) | Module-detect |
| OUT | signal to a left | Clkin/2 (outputs a | controller 528 |
| | communications | signal one-half of the | to the left signal |
| | pin. | frequency received | switch 564 and |
| | | via a communications | frequency |
| | | pin | selection |
| | | | of the |
| | | | right power pin |
| | | | 578 by the |
| | | | module-detect |
| | | | controller 528. |
| R Ref | Controls a right | HiZ (high | Signal from |
| CLOCK | clock signal | impedance) | Module-detect |
| OUT | to a right | Clkin/2 (outputs a | controller 528 |
| | communications | signal one-half of the | to right signal |
| | pin. | frequency received | switch 570 |
| | | via a | and frequency |
| | | communications | selection |
| | | pin | of the signal |
| | | | generator |
| | | | 544 by the |
| | | | module-detect |
| | | | controller 528. |
| BUS | Controls | Off (both power pins | Signal from |
| POWER | whether both | are not coupled to the | Module-detect |
| Xbar | power pins are | cross-bar bus). | controller 528 |
| SWITCH | coupled to the | On (both power pins | to crossbar |
| | cross-bar bus. | are coupled to the | 572 closes |
| | | cross-bar bus). | switches 573, 575 |
| L BUS | Controls | Off (power not | Signal from |
| POWER | whether the | applied from the left | Module-detect |
| En | left cross- | power pin to the | controller 528 |
| | bar switch | cross-bar bus) | to the left |
| | couples the | On (power is applied | cross-bar |
| | left power | from the left power | switch 573. |
| | pin to the | pin to the cross-bar | |
| | cross-bar | bus) | |
| | bus. | Turns on power to | |
| | | device electronics in | |
| | | some embodiments. | |
| R BUS | Controls | Off (power not | Signal from |
| POWER | whether the | applied from the right | Module-detect |
| En | right cross-bar | power pin to the | controller 528 |
| | switch couples | cross-bar bus) | to the right |
| | the right | On (power is applied | cross-bar |
| | power pin to the | from the right power | switch 575. |
| | cross-bar bus. | pin to the cross-bar | |
| | | bus) | |
| | | Turns on power to | |
| | | device electronics in | |
| | | some embodiments. | |
| PULSE | A signal to the | ClkIn (signals to the | Signal from |
| TO uP | processor to | processor that a clock | Module-detect |
| | indicate | has been received). | controller 528 |
| | a presence | 0 (signals to the | to CPU. |
| | of a received | processor that a clock | |
| | communications | has not been received. | |
| | signal. | | |
| Dir | A signal to the | 0 (Clock signal | Signal from |
| TO uP | processor to | received from module | Module-detect |
| | indicate | coupled to the left | controller 528 |
| | the direction, | connector) | to CPU. |
| | e.g., left | 1 (Clock signal | |
| | or right, the | received from module | |
| | communications | coupled to the right | |
| | signal comes | connector) | |
| | from. | May be ignored if no | |
| | | pulse signal is | |
| | | present. | |

Initially, state 614 is entered into. In state 614, the medical device assembly 504 is a state where it is detached from all power sources, such as when it is resting within a cabinet. States 616, 618, and 620 correspond to the left side of the medical device assembly 504 being connected to a central unit 502 or another medical-device assembly 504 on its left side. Likewise, states 622, 624, and 626 correspond to the medical device assembly 504 being connected to a central unit 502 or another medical-device assembly 504 on its right side.

The transition “LEF REF CLOCK IS PRESENT IMMEDIATELY” from state 614 to state 616 occurs when the left connector 510 detects a signal from the left communications pin 584. In state 616, the “L LOAD En” is set to “1”, which means that the left resistor 540 is coupled to the left communications pin 584 (e.g., by closing the switch 566). The state 616 will continue to transition back to itself if no power is detected from either the left side from the left power pin 582 or the right side from the right power pin 586 after 4 ms, as indicated by the “LEFT AND RIGHT POWER ARE NOT PRESENT FOR 4 ms” transition. However, if the left clock signal is not detected via the left communications pin 584 for at least 4 ms, the medical-device assembly 504 transitions from state 616 to 614 by the transition labeled as “LEFT REF CLOCK IS NOT PRESENT FOR 4 ms”.

When the power is received from the left power pin for at least 32 ms, state 616 transitions to state 618 as indicated by the “LEFT OR RIGHT BUS POWER IS PRESENT FOR 32 ms”. In state 618, the “L BUS POWER En” is set to ON, which would close the left cross-bar switch 573 thereby sending power to the common bus 571. In some embodiments, the switch 577 is closed at state 618 to send power to the module electronics 579. Also in state 618, the “R Ref Clock Out” turns on the right clock at half the frequency received via the left communications pin 584. That is, the switch 570 is closed while signal generator 544 generates a square wave that is one-half the frequency received via the left communications pin 584. Also, the “Pulse to uP” CkIn signal is sent to the CPU 581 (connection not explicitly shown in FIG. 188, but it may be a wired connection) so that the CPU 581 knows that a clock signal has been received via the left communications pin 584. The “Dir To uP” signal is set to 0, which is sent to the CPU 581 so that the CPU 581 can determine which direction the signal is received from. In this exemplary embodiment, the 0 value indicates that the signal is coming from the left communications pin 584; however, the particular logic values used may be changed.

If the left clock is not present for 4 ms, the medical-device assemblies 504 transitions from state 618 to state 614 via transition labeled “LEFT REF CLOCK IS NOT PRESENT FOR 4 ms”. If neither the left power pin nor the right power pin is powered up for 4 ms, the medical-device assemblies 504 transition from state 618 to state 616. If a passive request for power is detected via the right communications pin of the medical-device assemblies 504, the medical-device assemblies 504 transitions from state 618 to state 620 when the load is detected for 100 ms via the right clock output. The transition is labeled “RIGHT LOAD DETECTED FOR 100 ms OF RIGHT CLOCK OUT”, which corresponds to the case in which the BUS POWER XbarSWITH is turned ON, which means that both of switches 573 and 575 are closed thereby allowing power to flow from the left power pin to the right power pin.

At state 620, if the left and right power pins are ever not receiving power for 4 ms, then the medical-device assembly 504 transition from state 620 to state 616 via the transition labeled “LEFT AND RIGHT POWER ARE NOT PRESENT FOR 4 ms”. If, at state 620, the left reference clock is not present for 4 ms, the medical-device assemblies 504 transitions from state 620 to state 614 via the transition labeled “LEFT REF CLOCK IS NOT PRESENT FOR 4 ms”.

Referring again to FIG. 190, the right branch from state 614 will now be described. The transition “RIGHT REF CLOCK IS PRESENT IMMEDIATELY” from state 614 to state 622 occurs when the right connector 512 detects a signal from the right communications pin 588. In state 622, the “R LOAD En” is set to “1”, which means that the right resistor 542 is coupled to the right communications pin 588 (e.g., by closing the switch 568). The state 622 will continue to transition back to itself if no power is detected from either the left side from the left power pin 582 or the right side from the right power pin 586 after 4 ms, as indicated by the “LEFT AND RIGHT POWER ARE NOT PRESENT FOR 4 ms” transition. However, if the right clock signal is not detected via the right communications pin 588 for at least 4 ms, the medical-device assembly 504 transitions from state 622 to 614 by the transition labeled as “RIGHT REF CLOCK IS NOT PRESENT FOR 4 ms”.

When the power is received from the right power pin for at least 32 ms, state 616 transitions to state 624 as indicated by the transition label “LEFT OR RIGHT BUS POWER IS PRESENT FOR 32 ms”. In state 624, the “R BUS POWER En” is set to ON, which would close the right cross-bar switch 575 thereby sending power to the common bus 571. In some embodiments, the switch 577 is closed at state 624 to send power to the module electronics 579. Also in state 624, the “L Ref Clock Out” turns on the left clock at half the frequency received via the right communications pin 588. That is, the switch 564 is closed while signal generator 569 generates a square wave that is one-half the frequency received via the right communications pin 588. Also, the “Pulse to uP” CkIn signal is sent to the CPU 581 (connection not explicitly shown in FIG. 188, but it may be a wired connection) so that the CPU 581 knows the clock signal has been received via the left communications pin 584. The “Dir To uP” signal is set to 1, which is sent to the CPU 581 so that the CPU 581 can determine which direction the signal is received from. In this exemplary embodiment, the 1 value indicate that the signal is coming from the right communications pin 588; however, the particular logic values used may be changed.

If the left clock is not present for 4 ms, the medical-device assemblies 504 transitions from state 624 to state 614 via transition labeled “RIGHT REF CLOCK IS NOT PRESENT FOR 4 ms”. If neither the left power pin nor the right power pin is powered up for 4 ms, the medical-device assemblies 504 transition from state 624 to state 622. If a passive request for power is detected via the left communications pin of the medical-device assemblies 504, the medical-device assemblies 504 transitions from state 624 to state 626 when the load is detected for 100 ms via the left communication pin. The transition is labeled “LEFT LOAD DETECTED FOR 100 ms OF RIGHT CLOCK OUT”, which corresponds to the case in which the BUS POWER XbarSWITH is turned ON, which means the both of switches 573 and 575 are closed thereby allowing power to flow from the left power pin to the right power pin. At state 626, if the left and right power pins are ever not receiving power for 4 ms, then medical-device assemblies 504 transition from state 626 to state 622 via the transition labeled “LEFT AND RIGHT POWER ARE NOT PRESENT FOR 4 ms”. If, at state 626, the right reference clock is not present for 4 ms, the medical-device assemblies 504 transitions from state 626 to state 614 via the transition labeled “RIGHT REF CLOCK IS NOT PRESENT FOR 4 ms”.

FIGS. 191A-191B show a timing diagram 700 of the modular pump system 500 as two medical device assemblies 504 are coupled to the central unit 502 to illustrate the powering-up sequence of the system. The timing diagram 700 shows a central unit 722, which may be the same as the central unit 502 described herein, and the timing diagram 700 shows two medical device assemblies 723, 724 which may be the same as the medical device assembly 504 described herein.

At 701, the central unit 722 has an initial power up. At 702 the reference clock generates a square wave and couples it to a communication pin of the module 723 after the module is attached at 708. At 703, a passible indication of a request for power is determined by using an operational amplifier to detect impedance on the communications pin. If a load is not detected at 100 ms at 705, then at 704 the power applied to a right power pin is turned off (if already on). If it is detected, then at 706, a detected load is communication to the microprocessor and the right power bus is turned on as to supply power to the right power pin at 707.

The timing diagram 700 also shows the operation of the medical-device assembly 723 when it is coupled to the central unit 722. The attachment is shown as 708. At 709, the medical-device assembly 723 uses the signal received from the central unit 722 and harvests it using a charge pump. If the clock is validated 710 (e.g., a predetermined number of signals determines it is a clock having the proper characteristics), then 710 transitions through 711 to 713. Otherwise, 710 transitions to 711 and back to 710. For example, the first few samples of a square wave may be ignored so that transient signals generated by a users' touch does not cause a false positive for a passive request for power. Additionally or alternatively, a clock may start on the rising edge of a waveform and a predetermined amount of time may be required to pass where the clock is above a predetermined threshold until the square wave is considered valid. One of ordinary skill in the art will appreciate variations including the use of positive logic, negative logic, or inverted logic to implement this touch-detect feature. In some specific embodiments, a predetermined number of valid pulses must be detected until the signal is considered to be valid. At 712, a copy of the reference signal clock and incoming side of the signal is sent to the processor so that it can determine its position within the system 500.

At 713, a load is applied to the communications pin and then, the assembly 723 transitions to 714 where it waits for power via the power pin. That is, 714 transitions from 716 to 715 until power is received after which the assembly 723 transitions to 717. At 717, the module is powered from the power bus.

At 718, a signal is turned on the opposite side connected to the central unit 722 for application to the communications pin that is opposite to the central unit 722. At 719, an op-amp monitors the load on the communications pin and if a load is not detected for 100 ms continuously, then it will turn off the power bus at 720 and transitions back to 719. Otherwise, 721 transitions from 722 to turn on the crossbar to dower downstream to the assembly 724. The assembly 724 operates in the same way as the assembly 723 and as indicated by the timing diagram 700. Please note that the assembly 723, 724 operate the same way regardless as to whether a central unit 722 is applying the signal or another assembly 504 is applying the power (however the frequency changes of the clock to indicate relative position is used).

FIGS. 192A-192C show a block diagram of a modular pump system 500 including a central unit 800, and medical-device assemblies 801. The central unit 800 includes a dual hot swap controller 822 to apply power to a power pin, which is controlled by a controller 802 via a driver 804. The controller generates a clock signal via driver 805 which then uses the current sensor 803 to determine changes in impedance as described above. An analog comparator 806 communicates the output of the current sensor 803 (op-amp design) to the control logic 807. The controller 802 uses the state diagrams described above and/or the timing diagrams described above.

The assembly 801 (shown in FIG. 192B and another one is shown in FIG. 192C) includes a controller 808. The controller 808 controls the cross-bar switch 817 via a driver 818. The controller 808 can be powered via left charge pump diodes 809 or right charge pump diodes 810. A clock may be generated to apply to the left communications pin via driver 813 or a right communications pin via driver 814. A left current sense 811 detects changes in impedances of the left communications pin and the right current sense 812 detects changes in impedance as the clock is applied to the right communications pin.

A driver 815 controls whether or not a load 819 is coupled to the left communications pin while a driver 816 controls whether or not a load 820 is applied to the right communications pin. A dual hot swap controller 822 allows power to be applied to the module electronics 821 via a left power pin or right power pin.

FIGS. 103A-103J shows circuitry of the modular pump system 500 to illustrate the assembly ID circuitry, e.g., that may be used with the modular pump system described herein.

FIG. 193A shows a buffer circuit to buffer the output signal as applied to a communications pin. U3 may be a part number SN74LVC2G17DBVR manufactured by Texas Instruments of 12500 TI Blvd., Dallas, Tex. 75243.

FIG. 193B shows the controller U5. The controller U5 may be part number SLG46721V of Dialog Semiconductor of 100 Longwater Avenue, Green Park, Reading RG2 6GP, United Kingdom. FIG. 193C shows a debugging header. FIG. 193D shows the voltage regulator for the central unit or modular assembly. FIG. 193E shows a power conditioning circuit. FIGS. 193F and 103G shows power conditioning circuits. FIG. 193H shows another debugging header. FIG. 193I shows the dual hot-swap controller. The device U4, may be part number LTC4226IMS-2#PBF made by Analog Devices of One Technology Way, P. O. Box 9106, Norwood, Mass. 02062-9106, United States of America. FIG. 193J shows the cross-bar switch.

FIG. 194 shows a block diagram of the communication circuitry of the modular pump system. The communication module 900, 901 and 902 are shown. The modules 900, 901, and 902 may each be part of a central unit or an assembly. The module 901 includes a RF stripline 906 which forms a communications bus. The communications bus may be dual use with the start-up sequence described above. One end of the bus includes a transceiver coil 903. On the other end is another transceiver coil 904 coupled to a resonator 905. The resonator communication with another module via an air gap as shown in FIG. 194. A top is coupled to the resonator 905 to interface with the bus via transceiver 907.

FIG. 195 shows a diagram of the circuitry for interfacing into the communications bus of the modular pump system. A CAN peripheral 918 is coupled to a buffer 916 for transmitting and another buffer 917 for receiving a signal.

The transceiver module 908 modules the CAN values on an On-Off keying carrier signal. For transmission, the carrier frequency is generated using a spread-spectrum clock generator 914, which is on-off modulated with a clock buffer 912. A band pass filter 910 isolates the circuitry and the splitter 909 allows the signal to interface with the bus. The on-off carrier signal is also received by the splitter 909, which goes through a band pass filter 911 and is demodulated by a power detector 913. A comparator 915 translates the broadband signal to CAN on-off signals for being received by the buffer 917. FIG. 196 shows a PCB diagram of the resonator 905.

In alternative embodiments, the central unit generates the broad-spectrum signal and each of the assemblies grounds the signal to on-off keying modulation to communicate the on-off values needed for CAN communications.

FIG. 197 shows another embodiment of the peristaltic pump 10200 where a relief mechanism 1200 is used. The relief mechanism 1200 may also be referred to as, but is not limited to, a mechanical fuse. The relief mechanism 1200 between the lever 104 and a main shaft 118 includes a first linkage 1202, a second linkage 1204, a first rigid member 1206, and a second rigid member 1208. The relief mechanism 1200 also includes a first spring 1210, a second spring 1212, and a hold 1214. The relief mechanism 1200 operates to relieve high torsion forces. These high torsion forces may be applied to the main shaft 118 by excessive actuation force being applied to the lever 104. The relief mechanism 1200 includes the springs 1210, 1212 and the hold 1214 that form a general triangle shape. The springs 1210, 1212 have enough force to hold the relief mechanism 1200 in place up to a specific load, at which point the relief mechanism 1200 buckles. After buckling, raising the lever 104 causes the relief mechanism 1200 to reset automatically. For example, if the door 102 is open, the main shaft 118 (see FIG. 98) cannot be rotated because of an interlock that prevents a user from closing the lever 104 shut. The force applied to the lever 104, when the door 102 is open, is therefore translated to the relief mechanism 1200 when a user attempts to close the lever 104 with the door 102 open. The relief mechanism 1200 will buckle if a predetermined threshold amount of force is applied to the lever 104 with the door 102 open.

The springs 1210, 1212 bias the first rigid member 1206 and the second rigid member 1208 to rotate via a linkage pivot 1216 toward the hold 1214. The springs 1210, 1212 are coupled together via the hold 1214. Because the spring 1210 is attached to the first rigid member 1206 at a first protrusion 1222, the springs 1210, 1212 rotationally bias the first rigid member 1206 to rotate via the pivot 1218. Likewise, the springs 1210, 1212 rotationally bias the second rigid member 1208 to rotate via the pivot 1220 because the second spring 1212 is attached to the second rigid member 1208 at a second protrusion 1224. However, rotation of the first and second protrusions 1222, 1224 toward the hold 1214 is limited by interactions between a stop 1226 and a surface 1228 (see FIGS. 198A-198C) that occur between the first rigid member 1206 and the second rigid member 1208. The relief mechanism 1200 is in a hold state when the first protrusion 1222 and the second protrusion 1224 are rotated maximally toward each other such that the stop 1226 and surface 1228 are engaged together.

Refer now to FIGS. 198A-198C for an illustration of the transition from a hold state to a triggered state of the relief mechanism 1200. FIG. 198A shows the relief mechanism 1200 in the hold state, FIG. 198B shows the relief mechanism 1200 in an intermediate state, and FIG. 198C shows the relief mechanism 1200 in the triggered state. In FIG. 198A, the stop 1226 and the surface 1228 are interacting with each other because the springs 1210, 1212 apply a rotational force on the pivots 1218, 1220 thereby keeping a fixed distance between the two pivots 1218, 1220. This fixed distance between the pivot 1218 of the first linkage 1202 and the pivot 1220 of the second linkage 1204 correlates the rotations of the first linkage 1202 and the second linkage 1204 via their respective pivots 1230, 1232.

However, if the door 102 is open, the second linkage 1204 cannot fully rotate in the same direction as the first linkage 1202. FIG. 198B shows the effect of a non-moving second linkage 1204, where the first linkage 1202 continues to rotate thereby causing the surface 1228 and the stop 1226 to separate from each other. This separation is due to the rotation of the first rigid member 1206 and limited or no rotation of the second linkage 1204. As the first linkage 1202 rotates towards the second linkage 1204, the first rigid member 1206 and the second rigid member 1208 rotate along the linkage pivot 1216. Rotation along the linkage pivot 1216 in this manner causes the springs 1210, 1212 to stretch and increase their pulling force. FIG. 198C shows the relief mechanism 1200 when rotation along the linkage pivot 1216 has reached its maximum amount of rotation and the distance between pivots 1218, 1220 have reached their minimum distance. In some specific embodiments, if a user releases the lever 104 while the relief mechanism 1200 is in the triggered state, the relief mechanism 1200 will snap back into the hold 1214 state.

However, in some embodiments of the present disclosure, a predetermined amount of rotation of the linkage pivot 1216 may cause the relief mechanism 1200 to quickly actuate into the triggered state. For example, one or ordinary skill in the relevant art would know how to implement an over-center action of the relief mechanism. Additionally, alternatively, or optionally, the relief mechanism 1200 may be bi-stable where the stable states are the hold state and the triggered state.

Referring now to FIGS. 199A-199C, which shows the first rigid member 1206. The first rigid member 1206 engages with the second rigid member 1208 (shown in FIGS. 200A-200C). The first rigid member 1206 also includes a fastening point 1236 configured to fasten to an end of a spring (e.g., spring 1210). As is easily seen in FIG. 199A, the first rigid member 1206 includes the surface 1228 that disengages with the stop 1226 of the second rigid member 1208. The surface 1228 is disengaged from the stop 1226 when the relief mechanism 1200 is in the triggered state.

FIGS. 200A-200C shows several views of the second rigid member 1208. The second rigid member 1208 includes the linkage pivot 1216 that engages with the first rigid member 1206 (not shown in FIGS. 200A-200C). The second rigid member 1208 also includes a fastening point 1234 configured to fasten to an end of a spring (e.g., spring 1212). As is easily seen in FIG. 199A, the second rigid member 1208 includes the stop 1226, which is some embodiments is a surface. The stop 1226 is engaged by the first rigid member 1206 when the relief mechanism 1200 is in the hold state.

FIG. 201 shows a keyed end effector 1240 that is part of a plunger 1242 in accordance with an embodiment of the present disclosure. The end effector 1240 includes a key 1246 that cooperates with a notch 1244 of the plunger 1242. The end effector 1240 is mounted to the plunger 1242 at a right angle thus the end effector 1240 is coupled to the plunger 1242 at a surface opposite to the surface of the end effector 1240 that faces the platen 1238. That is, the end effector 1240 is attached to the side of the end effector 1240 that faces away from the platen 1238.

FIGS. 202A-202D show another embodiment of an adjustable end effector 1248 of the plunger 1250. The adjustable end effector 1248 is attached to the plunger 1250 at a side of the adjustable end effector 1248. In some embodiments of the present disclosure, the adjustable end effector 1248 may be made out of a thermal insulator, such as a plastic, polymer, rubber, etc. The thermal insulation may reduce or eliminate the transfer of heat to a fluid in an IV line thereby decreasing the occurrence of outgassing. The adjustable end effector 1248 attaches to the plunger 1250 at two attachment points 1252. Shown in FIG. 202B, the adjustable end effector 1248 is part of the plunger 1250 that pivots around the pivot shaft 202. And any change of angle, orientation, or position of the pivot shaft 202 in relation to the platen 1238 will change/affect the position of the end effector 1248 with respect to the platen 1238. That is, the pivot shaft 202 can be adjusted to thereby adjust the adjustable end effector 1248 during manufacturer and/or in field use.

FIGS. 202C-202D show a first shaft adjuster 1254 and a second shaft adjuster 1256 that move the end of pivot shaft 202 in a plane approximately aligned with axis of the pivot shaft 202. The first shaft adjuster 1254 is disposed adjacent to the pivot shaft 202 and a first ramp 1258. Likewise, the second shaft adjuster 1256 is disposed between the pivot shaft 202 and the second ramp 1260. The first shaft adjuster 1254 and the second shaft adjuster 1256 may be adjusted by a first adjustment screw 1262 and a second adjustment screw 1264, respectively.

As is shown in FIG. 202C, the first shaft adjuster 1254 is positioned closer to the first adjustment screw 1262 thereby moving the pivot shaft 202 away from the first shaft adjuster 1254 as it engages with the first ramp 1258. Also, the second adjustment screw 1264 has been set to position the second shaft adjuster 1256 away from the second adjustment screw 1264. Because the first shaft adjuster 1254 interfaces with the first ramp 1258 as it is actuated toward the first adjustment screw 1262, the pivot shaft 202 is moved away from the first shaft adjuster 1254 and toward the second shaft adjuster 1256.

Likewise, as is shown in FIG. 202D, the second shaft adjuster 1256 is positioned closer to the second adjustment screw 1264 thereby moving the pivot shaft 202 away from the second shaft adjuster 1256 as it engages with the second ramp 1260. Also, the first adjustment screw 1262 has been set to position the first shaft adjuster 1254 away from the first adjustment screw 1262. Because the second shaft adjuster 1256 interfaces with the second ramp 1260 as it is actuated toward the second adjustment screw 1264, the pivot shaft 202 is moved away from the second shaft adjuster 1256 and toward the first shaft adjuster 1254.

FIGS. 203A-203G illustrate an adjustable platen 1266 in accordance with an embodiment of the present disclosure. The adjustable platen 1266 may optionally, alternatively, or additionally be used in one or more embodiments described herein, or in no embodiments. Any platen described or disclosed herein, such as the adjustable platen 1266, may be made out of a thermal insulator, such as a plastic, polymer, or rubber. The thermal insulation may prevent heat from being transferred to a fluid in an IV line thereby decreasing the occurrence of outgassing. The thermal insulation (e.g., use of non-thermally conductive materials such as plastic wedges and/or washers between components and/or plastic plunger) may prevent heat from being transferred to a fluid in an IV line thereby decreasing the occurrence of outgas sing.

FIG. 203A shows the adjustable platen 1266 that is mountable by a first mount 1300 and a second mount 1302. A first mount screw 1268 secures a first end of the adjustable platen 1266 to the first mount 1300. A second mount screw 1270 secures a second end of the adjustable platen 1266 to the second mount 1302. The first mount screw 1268 engages with a first threaded hole 1288 while the second mount screw 1270 engages with a second threaded hole 1290.

The first adjuster 1276 includes a threaded hole 1294, a first side 1280, and a second side 1282. The second side 1282 of the first adjuster 1276 engages with a first engagement surface 1296 of the first mount 1300. The first adjustment screw 1272 fastens to the first adjuster 1276 to control its distance relative to the first mount 1300. As the first adjuster 1276 is actuated away from the first mount 1300 by the first adjustment screw 1272, the distance between the first side 1284 and the second side 1282 increases along the thickness of the first adjuster 1276 that engages with the first engagement surface 1296. That is, the second side 1282 and the first engagement surface 1296 act as a ramp to actuate the adjustable platen 1266 away from the first mount 1300 as the adjuster is actuated away from the first mount 1300.

Similarly, the second adjuster 1278 includes a threaded hole 1292, a first side 1284, and a second side 1286. A second engagement surface 1298 of the second mount 1302 engages with the second side 1282 of the second adjuster 1278. The second adjustment screw 1274 fastens to the second adjuster 1278 to control its distance relative to the second mount 1302. As the second adjuster 1278 is actuated away from the second mount 1302 by the second adjustment screw 1274, the distance between the first side 1284 and the second side 1282 increases along the thickness of the second adjuster 1278 that engages with the second engagement surface 1298. That is, the second side 1286 and the second engagement surface 1298 Act as a ramp to actuate the adjustable platen 1266 away from the second mount 1302 as the second adjuster 1278 is actuated away from the second mount 1302.

In some embodiments, any fastener may be used for one or more of the first adjustment screw 1272, the second adjustment screw 1274, the first mount screw 1268, or the second mount screw 1270, such as bolts, latches, glues, epoxies, etc.

FIGS. 203B-203D show how an adjustment to the platen1266 may be made. In FIG. 203B, the first adjuster 1276 is positioned adjacent to the first mount 1300 and the second adjuster 1278 is positioned adjacent to the second mount 1302. The first adjuster 1276 has an optional first planar portion 1304 that can be positioned between the adjustable platen 1266 and the pump, when assembled. The second adjuster 1278 also has an optional second planar portion 1306 that can be positioned between the adjustable platen 1266 and the pump.

As is easily seen in FIG. 203B, actuating the first adjustment screw 1272 into the first adjuster 1276 actuates the first adjuster 1276 toward the plunger 1312 (FIG. 203C shows the plunger 1312 mounted). In FIG. 203B, the plunger mount screws 1310, 1308 are shown. FIG. 203C shows the adjustable platen 1266 mounted and FIG. 203D shows the plunger 1312 mounted. Referring now to FIGS. 203B-203D, it can be easily seen that movement of the adjustable platen 1266 relative to the plunger 1312 adjusts how the plunger 1312 will interact with a tube placed within the adjustable platen 1266. The plunger 1312, in some embodiments, may have a repeatable motion that is stopped (e.g., it can be seen in FIG. 14 how an end effector 128 has actuation limited by contact with a fixed stop).

FIGS. 203E-203G show a cross-sectional view between the pump and the adjustable platen 1266. As can be seen, the adjustable platen 1238 includes a space 1314 that has a surface 1316 configured for engagement with the first side 1280 of the first adjuster 1276 and also for engagement with the first side 1284 of the second adjuster 1278. In accordance with one embodiment of the present disclosure, a head of the second adjustment screw 1274 and a head of the first adjustment screw 1272 may be in a fixed position relative to the adjustable platen 1266 such that actuation of the adjustment screws 1272, 1274 actuate the first and second adjusters 1276, 1278, respectively. As can be seen, FIG. 203E shows the first and second adjusters 1276, 1278 at a position to maximally raise the adjustable platen 1266, FIG. 203F shows an intermediate position, and FIG. 203G shows the first and second adjusters 1276, 1278 at a position to keep the adjustable platen 1266 maximally away from the plunger 1312.

FIGS. 204A-204B show a multi-stage, spring-biased plunger 1318 of a peristaltic pump in accordance with an embodiment of the present disclosure. The plunger 1318 includes a follower 1324 that can follow a cam to actuate an end effector (not shown in FIGS. 204A-204B) coupled to an attachment point 1334. The plunger 1318 includes a roller 1322 that pivots around a pivot pin 1320. The roller 1322 is coupled to the rest of the plunger 1318 via a leaf spring 1332. The leaf spring 1332 allows the roller 1322 to actuate between two positions by interaction with a stop 1326. FIG. 204A shows a first position of the roller 1322 and FIG. 204B shows a second position of the roller 1322, both of which are based upon the position of the leaf spring 1332.

In operation, when the roller 1322 is in contact with a cam and the spring-biased plunger 1318 starts to interact with the tube, the stop 1326 will have contact with the first contact 1330. Initially, as the spring-biased plunger 1318 actuates the end effector toward the tube, the cam holds the follower 1324 against the stop 1326 at the first contact 1330 as shown in FIG. 204A. Also, as the spring-biased plunger 1318 actuates an end effector against the tube, the resilience of the tube will, after a threshold amount of force is applied to the end effector by the tube, cause the leaf spring 1332 to transition such that the follower 1324 contacts against the second contact 1328 as shown in FIG. 204B.

The amount of force needed to transition the leaf spring 1332 is predetermined and may be a function of the amount of force applied to the tube. For example, the leaf spring 1332 may transition based upon the amount of force applied to the tube when the inlet and outlet valve are closed.

FIG. 205 shows a flow chart diagram of a method 1336 for actuating the spring-biased plunger 1319 that is biased by a multi-stage spring. Act 1338 closes the inlet valve. Act 1340 closes the outlet value. Act 1342 actuates the plunger cam, e.g., to lower the end effector onto the tube such as in 1344. Act 1346 contacts the tube with the end effector. Air may be in the tube. Thus, in Act 1348, the spring-biased plunger 1318 is actuated a first amount, during which time, the air, e.g., a bubble, will compress more readily than any liquid contained therein. In Act 1350, the leaf spring 1332 of the spring-biased plunger 1318 transitions from a first position to a second position, e.g., when a predetermined force is applied to the tube. This force may be determined such that, any air within the tube is substantially (e.g., 95% less volume) is compressed to a small volume. Thus, the position of the plunger and/or end effector during this transition can be used to determine how much liquid is within the tube. Thereafter, the spring-biased plunger 1318 may continue to be actuated until the cam follower disengages the plunger cam, such as in Act 1354. Act 1356 determines a second position of the spring-biased plunger 116. The first and second positions may be used to estimate the amount of air in the tube in Act 1358. The resulting amount of fluid calculated to remain in the tube thus can be used to estimate the amount of fluid delivered downstream when the outlet value is opened and/or the fluid is ejected past the outlet value. The two positions of the plunger 1318 may be linearly related to the amount of air in the tube, or any suitable model may be used to estimate the air in the tube, such as linear or polynomial regression from experimentally derived data. The first measurement position is a point where the leave spring is positioned such that 1326 is positioned between 1328 and 1330, not making contact with either of them. The measurement happens in that middle region where the force should be approximately constant.

### Heat Transfer Design

FIG. 206 shows a back side of the peristaltic pump 1020 having a thermal dissipation assembly 1360 including a heatsink 1362 in accordance with an embodiment of the present disclosure. FIG. 206 shows the overall positioning of the thermal dissipation assembly 1360 relative to the body of the peristaltic pump 1020.

FIGS. 207A-207G show several views of the thermal dissipation assembly 1360. As is shown in FIG. 207A, the thermal dissipation assembly 1360 includes the heatsink 1362, a planar-thermal connector 1364, and a heat-strap bracket 1366. In FIG. 207B, it can be seen that the planar-thermal connector 1364 includes a first arm 1372 and a second arm 1374 both extending to connect to a motor (not shown) to absorb thermal energy therefrom and transfer the thermal energy to the heatsink 1362. The first arm 1372 and the second arm 1374 may be soldered onto the motor. The planar-thermal connector 1364 may be made of any metal, alloy, or thermally-conductive material, including, but not limited to, copper or aluminum. In some embodiments, a braided wire may be used in place of the planar metal portions of the planar-thermal connector 1364. In yet additional embodiments, the planar portion could be replaced by one or more heat pipes.

FIG. 207C shows a side-view of the thermal dissipation assembly 1360. The thermal dissipation assembly 1360 includes a first interface plate 1376 that can interface into a powerbar 1430 (see FIGS. 210-2011) to absorb heat therefrom. The powerbar 1430 may serve to absorb heat from high-heat electronics, such as power MOSFET's or other power semiconductors. A powerbar 1430 may be, for example, an EMI shield surrounding power electronics that is also being used to dissipate thermal energy. In some embodiments, thermal paste may be used.

FIG. 207D shows the thermal pad 1368 of the thermal dissipation assembly 1360. The thermal dissipation assembly 1360 is attached to the first interface plate 1376, which can be seen in FIG. 207E in which the thermal pad 1368 is removed thereby exposing the first interface plate 1376. FIGS. 207F-207G show the thermal dissipation assembly 1360 with the heatsink 1362 removed. A second thermal pad 1370 is shown and it is disposed between a second interface plate 1378 and the heatsink 1362 to facilitate thermal flow between the heatsink 1362 and the rest of the planar-thermal connector 1364. In some embodiments, thermal paste may be used. Additionally, a heat-strap bracket 1366 may be disposed between the first interface plate 1376 and the second interface plate 1378 to ensure a spaced-relationship between the first and second interface plates 1376, 1378. The heat-strap bracket 1366 may be made of any insulating material, such as an insulating plastic or polymer. Screws 1382, 1384 can be used to secure the heatsink 1362 to the heat-strap bracket 1366.

Refer now to both of FIGS. 208A-208B, which show additional views of the planar-thermal connector 1364. As is easily seen in FIG. 208A, a spring 1380 is used to resiliently secure the first and second plates 1376, 1378 thereby facilitating contact of the first and second thermal pads 1368, 1370. The spring 1380 of the planar-thermal connector 1364 facilitates the accommodation of varying manufacturing tolerances. That is, the spring 1380 can expand the distance between the first interface plate 1376 and the second interface plate 1378 to ensure a good fit within the peristaltic pump 1020. The holes 1386, 1388 allow for the screws 1382, 1384, respectively, to traverse through the first interface plate 1376 to the heat-strap bracket 1366 (see FIG. 207G).

FIGS. 209A-209E show several views of the heat-strap bracket 1366. The heat-strap bracket 1366 includes threaded holes 1390, 1392, to receive the screws 1382, 1384, respectively. The heat-strap bracket 1366 also includes springs 1394, 1396, to provide resilience so that the heat-strap bracket 1366 can accommodate various manufacturing tolerances. Additionally, alternatively, or optionally, the springs 1394, 1396 can provide resilience to press the thermal pads 1368, 1370 against the powerbar 1430 and heatsink 1362, respectively.

FIG. 210A shows another embodiment of a planar thermal connector 1434 in accordance with an embodiment of the present disclosure. The planar thermal connector 1434 is thermally coupled to a motor 1432 and to the heatsink 1362. The planar thermal connector 1434 includes a first arm 1434a and a second arm 1434b, both thermally connected to the motor 1432 to dissipated heat toward the heatsink 1362. The planar thermal connector 1434 includes a first end 1438 thermally coupled to the powerbar 1430 and a second end 1440 thermally coupled to the heatsink 1362. The planar thermal connector 1434 can absorb heat from the motor 1432 and the powerbar 1430 to thereby dissipate heat energy into the surround ambient air through the heatsink 1362. The powerbar 1430 may be an EMI shield configured to shield power electronics that is also used to dissipate heat from the power electronics, for example.

FIG. 210B shows an embodiment of a heatsink 1362 having a braided-wire 1442 to transfer heat to the heatsink 1362 from the motor 1432 and the powerbar 1430. The braided-wire 1442 includes a first end 1450 coupled to the powerbar 1430 and a second end 1448 coupled to the heatsink 1362. The braided-wire 1443 is also coupled to a heatsink 1362 of the motor 1432 via third ends 1444, 1446. The third ends 1444, 1446 may also be referred to as arms.

In some embodiments, the third ends 1444, 1446, are coupled directly to the heatsink 1362 to provide a direct connection between the heatsink 1362 and the motor 1432. Additionally or alternatively, the first end 1450 and the second end 1448 may be connected directly together. The braided-wire 1443 may be soldered onto the powerbar 1430, the heatsink 1362, the motor 1432, and/or each other. Additionally or alternatively, clips may be used to secure the braided-wire 1443 onto the powerbar 1430, the heatsink 1362, the motor 1432, and/or each other. It will be appreciated by one of ordinary skill in the relevant art, that in place of the braided-wire, any combination of braided-wire and/or heat pipes may be used to transfer heat to the heatsink 1362 or to any other part of the peristaltic pump 1020.

In a further embodiment, described with reference to FIGS. 211A-211D, a thermal dissipation assembly 1361 may include a first portion 1361A (FIGS. 211A and 211C) and a second portion 1361B (FIGS. 211B and 211C).

The first portion 1361A may include the heatsink 1362 and a heatsink thermal pad 1371a that is thermally coupled to the heatsink 1362. The thermal pad 1371a may be substantially similar to the thermal pads 1368, previously described. A heatstrap bracket 1391 (FIG. 211D) may be thermally coupled and/or physically coupled to the heatsink 1362 with the thermal pad 1371a therebetween. The heatstrap bracket 1391 may have a unitary construction and may define a generally U-shaped configuration such that it defines first and second leaves 1391a that define surfaces that generally oppose one another and have a spring-like property capable of small deflections such that the leaves 1391a may be tensioned farther apart from one another and will be biased in a direction toward one another. The heatstrap bracket 1391 may include an upper portion 1391b that may be secured in a thermal connection and/or in physical contact with the thermal pad 1371a.

The thermal dissipation assembly 1361 may also include a second portion 1361B that may include a heatstrap lower 1393 (FIG. 211E) that defines a generally U-shaped configuration such that generally opposing sides of the U-shape define first and second leaves 1393A that have a spring-like property. A bracket 1393b may secure the heatstrap lower to be thermally coupled to a thermal pad 1371b that may provide thermal shielding for a Module Control Unit (MCU) 1395a. Additionally, radio frequency (RF) shielding may be provided by a shield 1395b for the MCU 1395a.

The first and second portions 1361A and 1361B are configured to be coupled together, as shown in FIG. 211C such that an overall length L is adjustable as the spring-like leaves of the heatstraps 1391a and 1393a frictionally interact with one another such that the two are thermally connected to one another. Since the length L is adjustable as the heatstraps 1391a, 1393a slide relative to one another, advantageously, the assembly tolerances may be looser allowing for easier assembly.

### Pumping Actions

FIG. 212 shows a flow chart diagram of a method 1398 for dislodging bubbles in an IV line in accordance with an embodiment of the present disclosure. The method 1398 includes acts 1399-1406. Act 1399 infuses fluid into a patient, e.g., using a peristaltic pump 1020. Act 1400 determines if a predetermined amount of time has passed. In other embodiments, a predetermined amount of fluid volume, a number of peristaltic pumping cycle, and/or an amount of air pumped past a downstream air sensor may be used in place of a predetermined amount of time. If act 1400 determined a predetermined amount of time has passed, then Act 1401 closes a downstream valve. Act 1403 disengages the actuator from the spring-biased plunger 116. Act 1404 interrupts the actuation (e.g., rotation) of the actuator. Act 1405 rapidly reverses actuation (e.g., rotation) of the actuator (e.g., a cam shaft) to eject bubbles upstream and passed an upstream valve. Act 1406 may reverse the fluid flow by a predetermined amount. The predetermined amount may be a predetermined amount of time, fluid volume, and/or a number of peristaltic pumping cycles, including fractions thereof. In some embodiments, the peristaltic pump includes an inlet valve, an outlet valve, and a spring-biased plunger 116 where the spring-biased plunger 116 includes a spring configured to bias the plunger again the tube and the cam is configured to actuate the plunger away from the tube. The method 1398 may reverse fluid flow when the outlet valve is closed and the inlet valve in open, e.g., fluid flow may be reversed as the plunger is being actuated toward the tube. In yet additional embodiments, the test at Act 1400 may be done at other locations, e.g., somewhere between 1401 and 1404. In yet additional embodiments, Act 1403 may be removed.

FIG. 213 shows a flow chart diagram of a method 1408 for detecting a bubble in accordance with an embodiment of the present disclosure. The method 1408 may be used on a pump, such as a peristaltic pump 1020 or syringe pump. The pump may have an air sensor, such as an ultrasonic air sensor. The method 1408 uses two thresholds to determine whether or not a bubble has been detected and/or should be accounted for using an ultrasonic signal, e.g., signal strength, a drop in signal strength, a gain in signal strength, etc. The method includes acts 1410-1440. Act 1410 sets a trigger threshold to a first bubble threshold. Act 1412 determines if the ultrasonic signal is above the first threshold. If the ultrasonic signal is above the first threshold, then the method continues return to Act 1410. Otherwise, the method continues to Act 1414 to determine if the ultrasonic signal is below the first threshold and above a second threshold. If it is, the method transitions to Act 1422. Otherwise, the method 1408 transitions to Act 1416. Act 1416 determines if the ultrasonic signal is below a second threshold and above the trigger threshold. If it is true, the method 1408 transitions to Act 1428. If Act 1416 has a false determination, then the method 1408 transitions to Act 1418. Act 1418 determines if the ultrasonic signal is below the trigger threshold. If not, then the method 1408 continues to Act 1420 in which case it is determined that no bubble is detected, and the method 1408 proceeds back to Act 1412.

At Act 1422, a first timer is started. At Act 1424, if the ultrasonic signal is below a second threshold, then the method transitions to Act 1416. Otherwise, the method continues to Act 1426. If the ultrasonic signal is above the first threshold at Act 1426, then the method continues at Act 1410. Otherwise, the method continues to Act 1427 to determine if a predetermined amount of time has elapsed for the first timer (see Act 1422). If a predetermined amount of time has elapsed for the first timer, the method continues to Act 1441. Otherwise, the method continues to Act 1426.

After a first predetermined amount of time has elapsed for the first timer at Act 1427, Act 1441 sets the trigger threshold to a second bubble threshold that is lower than the first bubble threshold and the method continues to prior to Act 1412.

As previously mentioned, a second timer is started at Act 1428. At Act 1431, it is determined if the ultrasonic signal is below the trigger threshold. If it is, then a bubble is detected at Act 1437. Or, if the ultrasonic signal is not below the trigger threshold at Act 1431, then Act 1435 determines if the ultrasonic signal is above the second threshold. If it is, then the method 1408 proceeds to before Act 1412. Otherwise, if the ultrasonic sign is below the second threshold, Act 1436 determines if a second predetermined amount of time elapsed for the second timer. If the second predetermined amount of time has elapsed for the second timer, Act 1435 sets the trigger threshold to a third bubble threshold that is lower than the first bubble threshold and the second bubble threshold, after which, the method continues to before Act 1412. If a second predetermined amount of time has not elapsed for the second timer in Act 1436, then the method returns to Act 1431 as is shown in FIG. 213.

FIGS. 214-217 show various ultrasonic-based bubble sensors in accordance with several embodiments of the present disclosure. Each of these embodiments shown in FIGS. 214-217 illustrate a sensor with multiple acoustic paths, which provide additional means for detecting air in the tubing. FIG. 214 shows an ultrasonic-based bubble sensor 1452 that includes a single piezoelectric transmitter 1466 and two piezoelectric receiver 1470, 1472. FIG. 215 shows an ultrasonic-based bubble sensor 1454 that includes two piezoelectric transmitters 1466, 1468 and a piezoelectric receiver 1470. FIG. 216 shows an ultrasonic-based bubble sensor 1456 having two piezoelectric transmitters 1466, 1468 and two piezoelectric receivers 1470, 1472. FIG. 216 is shows as being configured in an “aligned”-configuration. FIG. 217 shows an ultrasonic-based bubble sensor 1453 that is an “anti-aligned” configuration.

FIG. 214 shows an ultrasonic-based bubble sensor 1452 having a transmit module 1462 and a receive module 1464. The transmit module 1462 includes a first piezoelectric transmitter 1466. The receive module 1464 includes a first piezoelectric receiver 1470 and a second piezoelectric receiver 1472. The first piezoelectric receiver 1470 is separated from second piezoelectric receiver 1472 by a gap 1471. The gap 1471 may be arbitrarily small or large, and filled with any medium such as, but not limited to, air, foam, plastic, epoxy, etc. The single piezoelectric transmitter 1466 may transmit an ultrasonic signal across the tube 1458 to be received by the receive module 1464. The two piezoelectric receivers 1470, 1472 each can receive the ultrasonic signal, which is then converted to an electronic signal to be analyzed by a processor. The two piezoelectric receivers 1470, 1472 can be used together to determine the presence, position, size, velocity, or travel direction (e.g., upstream or downstream) of the bubble 1460. Additionally or alternatively, the two piezoelectric receivers 1470, 1472 can be used in a redundant fashion to ensure the bubble 1460 is moving or to determine the size or velocity of the bubble.

FIG. 215 shows an ultrasonic-based bubble sensor 1454 having a transmit module 1462 and a receive module 1464. The transmit module 1462 includes a first piezoelectric transmitter 1466 and a second piezoelectric transmitter 1468 which can both transmit ultrasonic energy across the tube 1458. The ultrasonic energy may be received by the module 1464 that includes a piezoelectric receiver 1470. The first piezoelectric transmitter 1466 and the second piezoelectric transmitter 1468 may be utilized such that only one is transmitting ultrasonic energy across the tube 1458 at any given time interval, in some specific embodiments. By periodically alternating between the first piezoelectric transmitter 1466 and the second piezoelectric transmitter 1468, a bubble 1460 may be tracked to determine its direction of movement and/or size. For example, if fluid is being pumped through the tube 1458 at a known flow rate, the amount of time the bubble 1460 is sensed by the piezoelectric receiver 1470 can be used in conjunction with the flow rate to estimate the size of the bubble 1460.

FIG. 216 shows an ultrasonic-based bubble sensor 1456 also having a transmit module 1462 and a receive module 1464. The transmit module 1462 includes a first piezoelectric transmitter 1466 and a second piezoelectric transmitter 1468. The receive module 1464 includes a first piezoelectric receiver 1470 and a second piezoelectric receiver 1472. The two piezoelectric transmitters 1466, 1468 can be modulated and/or multiplexed in various configurations.

In some embodiments, both piezoelectric transmitters 1466, 1468 are active at the same time and the two piezoelectric receivers 1470, 1472 each receive a respective ultrasonic signal simultaneously. Each of the piezoelectric transmitters 1466, 1468 may be coded and/or modulated so that each of the two piezoelectric receivers 1470, 1472 can distinguish which piezoelectric transmitter of the transmitters 1466, 1468 sent a respective signal (e.g., through demodulation or decoding). Additionally, alternatively, or optionally, there may be sufficient distance between the piezoelectric transmitters 1466, 1468 and/or sufficient distance between the piezoelectric receivers 1470, 1472 to prevent or mitigate cross-talk. In some embodiments, there may be a physical shield or barrier to shape and/or block ultrasonic energy from crossing paths.

In yet additional embodiments, the first piezoelectric transmitter 1466 and second piezoelectric transmitter 1468 are modulated with different frequencies relative to each other. For example, the first piezoelectric transmitter 1466 may transmit ultrasonic energy at a first frequency and the second piezoelectric transmitters 1468 may transmit ultrasonic energy at a second frequency. The first piezoelectric receiver 1470 may include filtering to filter the signal to receive the ultrasonic energy generated by the first piezoelectric transmitter 1466 while filtering out ultrasonic energy generated by the second piezoelectric transmitter 1468. Likewise, the second piezoelectric receiver 1472 may include filtering to filter the signal to receive the ultrasonic energy generated by the second piezoelectric transmitter 1468 while filtering out the ultrasonic energy generated by the first piezoelectric transmitter 1466. In some embodiments, the first piezoelectric transmitter 1466 and second piezoelectric transmitter 1468 may be active simultaneously, asynchronously, or may employ time-division multiplexing to avoid transmitting at the same time. Additionally, alternatively, or optionally, coded-division multiplexing may be used.

The two piezoelectric receivers 1470, 1472 may be used to estimate the position of the bubble 1460. The first piezoelectric transmitter 1466 and second piezoelectric transmitter 1468 may alternate the generation of the ultrasonic energy such that the first piezoelectric receiver 1470 and the second piezoelectric receiver 1472 are used to determine the direction and/or position of the bubble 1460 using triangulation known to one of ordinary skill in the relevant art.

FIG. 217 shows an ultrasonic-based bubble sensor 1453 that is in an “anti-aligned” configuration. That is, the ultrasonic-based bubble sensor includes a first module 1455 and a second module 1457. The first module 1455 includes a piezoelectric receiver 1466 and a piezoelectric transmitter 1472. The second module 1457 includes a piezoelectric receiver 1470 and a piezoelectric transmitter 1468. The piezoelectric transmitter 1468 transmits to the piezoelectric receiver 1472. The piezoelectric transmitter 1466 transmits to the piezoelectric receiver 1470. The ultrasonic-based bubble sensor 1453 may utilizing may of the same techniques described above with regard to one or more of FIGS. 214-216. The ultrasonic-based bubble sensor 1453 is anti-aligned because each side has a piezoelectric receiver 1466 and a piezoelectric transmitter 1468 configured to cooperate with a piezoelectric receiver 1470 and piezoelectric transmitter 1468 on an opposite side thereof.

Referring to the drawings, FIGS. 217-219 show a method 1474 for estimating air pumped downstream toward a patient by a peristaltic pump 1020 in accordance with an embodiment of the present disclosure. The method 1474 includes acts 1476-1512. The method 1474 may be implemented by utilizing a processor associated with a peristaltic pump, e.g., a peristaltic pump 1020 as described here. The method 1474 may be coupled to one or more bubble sensors as previously mentioned. The method 1474 may utilize the ultrasonic bubble sensor of FIG. 214 or 216 where the receiving module 1464 includes two piezoelectric receivers or by FIG. 215 where the first and second transmitters 1466, 1468 are modulated to have two different air sensor readings using a single receiver 1470.

For the method 1474 described in FIGS. 217-219, please refer to the terminology described in Table 2 as follows:

TABLE 4

| Term | Description |
|---|---|
| Bubble Event | A single Bubble Event includes any series of bubbles |
| | observed by both sensors that are not separated by |
| | sufficient volume of fluid (see Fluid Clear Volume). |
| Fluid Clear | Volume of consecutive fluid seen by one or more |
| Volume | sensors to declare the end of a Bubble Event |
| Hold-up | Volume within the tube between the upstream and |
| | downstream sensing paths. Calculated by using the |
| Volume (V) | cross-sectional area of the tube and the distance |
| | between the centerline of both sensing paths. |
| Overall Bubble | Final air bubble volume estimate, comprised of the |
| Estimate | individual raw air volume estimates from both |
| | sensing elements. |
| Raw | The raw air volume estimate from the downstream |
| Downstream | sensor during one Bubble Event. Calculated by |
| Estimate | summing the volume delivered while air is reported |
| | by the downstream sensing element. |
| Raw Upstream | The raw air volume estimate from the upstream sensor |
| Estimate | during one Bubble Event. Calculated by summing |
| | the volume delivered while air is reported by the |
| | upstream sensing element. |
| Single Sensor | Volume of consecutive fluid seen by a single sensor |
| Fluid Clear | required to declare the end of a Bubble Event. |
| Volume | This is used in the case where a bubble becomes |
| | stuck in front of one of the sensing elements. |
| | This volume is larger than the Fluid Clear Volume |

Act 1476 is the start of the method 1474. Act 1476 may be a function call made by other software being executed by a processor, for example. A function call may also be called a subroutine call. A subroutine (also known as a procedure, function, method or routine) includes the operation where one piece of code causes another piece of code to execute, inter alia, and also may be a system call or a function call. After Act 1476, Act 1480 clears both raw air estimates, which may include setting an upstream raw estimate and a downstream raw air estimate to zero. These estimates may be stored in a memory location and/or may be a calculated variable. At Act 1482, the method 1474 monitors for upstream air. For example, Act 1482 may monitor the signal received by the first piezoelectric receiver 1470 that is generated by the first piezoelectric transmitter 1466 of FIG. 216 of the ultrasonic-based bubble sensor 1456.

Act 1482 may be a subroutine of processor executable code that implements method 1474. Act 1482 monitors for upstream air and may include acts 1484-1490. Act 1484 reads the sensor data (e.g., ultrasonic air sensor data). Act 1486 updates the raw air estimates. Act 1488 determines if the upstream sensor reports air (e.g., if the received ultrasonic signal drops by a predetermined amount). If no air is reported by the upstream sensor, Act 1490 clears (sets to zero) the raw downstream estimate and Act 1484 is repeated. If air is reported by the upstream sensor at Act 1488, the method executes Act 1478, e.g., by a method call, execution of software, etc.

Act 1478 monitors for downstream air. Act 1478 may include acts 1492-1500. Act 1492 reads the sensor data. Act 1494 updates the raw air estimates. Act 1496 determines if air is reported by the downstream sensor (e.g., second piezoelectric transmitter 1468 and second piezoelectric receiver 1472). If Act 1496 determines that air is reported by the downstream sensor, the method continues to Act 1502, otherwise, the method continues to Act 1498. Act 1498 determines if persistent fluid is determined at the upstream sensor. If not, Act 1500 limits the raw upstream estimate and returns to Act 1492. Otherwise, Act 1498 returns to Act 1480. Act 1502 is entered into if air is reported by the downstream sensor in Act 1496.

Act 1502 may include acts 1504-1508. Act 1504 reads the sensor data, e.g., the upstream ultrasonic sensor data. Act 1506 updates the raw air estimates. Act 1508 determines if there is persistent fluid reported. If no persistent fluid is reported in Act 1508, the method continues to Act 1504. Otherwise, the method continues to Act 1510 to complete a bubble estimate and at Act 1512, the final air bubble volume is reported, e.g., to the control system or operating system.

Referring now to FIGS. 217-218, Acts 1486, 1494, and/or 1506 may be Act 1514 as shown in FIG. 218. Act 1514 updates the raw air estimates and includes Acts 1516-1530. Act 1516 is the start, which may be a function call, for example. Act 1518 determines if the air is reported by the upstream ultrasonic air sensor. This may be a parameter passed as part of the function call (e.g., a subroutine). If no air is reported by the upstream sensor, Act 1518 continues to Act 1520. If air is reported by the upstream sensor, the method continues to Act 1522.

Act 1520 adds the delta volume of fluid to the upstream fluid volume total (e.g., USFluidVol_uL+=Δvol_uL). That is, Act 1520 adds the amount of fluid pumped downstream to a total value. If air was reported by the upstream sensor in Act 1518, Act 1522 adds the amount of air pump downstream by the upstream ultrasonic sensor to a raw air estimate (e.g., USRawAirEstimate_ul+=Δvol_uL) and sets the volume of fluid of the upstream sensor to 0 (e.g., USFluidVol_uL=0).

After either Act 1520 or Act 1522, Act 1524 determines if air has been reported by the downstream ultrasonic sensor. The downstream ultrasonic sensor may be the downstream one of 1470, 1472, the downstream one of 1466, 1468, or the downstream one of 1462 and 1472 or 1466 and 1470 of FIGS. 214, 215, and 216, respectively.

If air has been reported, Act 1514 continues to Act 1526 where the reported delta volume is added to the downstream raw air estimate (e.g., DSRawAirEstimate_uL+=Δvol_uL) and the downstream fluid volume is set to zero (e.g., DSFluidVol_uL=0). Otherwise, if no air is reported by the downstream sensor, then Act 1524 transitions to Act 1528 where the delta volume pumped downstream is added to the downstream fluid volume (e.g., DSFluidVol_uL+=Δvol_uL).

Referring now to FIG. 219, a method 1532 for completing a bubble estimate is shown. The method 1532 may be Act 1510 of FIG. 217. The method 1532 includes Act 1534 which may be the initial start of the method 1532. Act 1534 may be a function call, an API call, a subroutine call, a system call, etc. Act 1536 determines an air volume ratio and calculates a bubble estimate. The air volume ratio may be the upstream raw air estimate divided by the downstream raw air estimate (e.g., AirVolRatio=USRawAirEstimate_uL/DSRawAirEstimate_uL). Act 1536 also calculates an initial bubble estimate by taking an average of the upstream and downstream air estimates (e.g., (USRawAirEstimate_uL+DSRawAirEstimate_uL)/2). Act 1538 and Act 1540 work together to determine if the air volume ratio is outside of a predetermined range. If it is not, then the bubble estimate is reported by the method 1532 as is calculated by Act 1536.

Act 1538 determines if the air volume ratio is greater than 2, in which case Act 1544 sets the bubble estimate to the downstream raw air estimate. However, if the air volume ratio is less than 0.5, then Act 1542 sets the bubble estimate to the upstream raw air estimate. As previously mentioned, if the air volume ratio is less than 2 but greater than 0.5, the value set in 1536 is return by act 1546, which is the end. Act 1546 may be a return act, such as providing return values from a function call made by another routine, code, subroutine, etc.

FIGS. 220A-220C show several views of an in-line pressure sensor 1548 and FIGS. 202D-202F show the in-line pressure sensor of FIGS. 220A-220C with a clip 1550 in accordance with an embodiment of the present disclosure.

Referring now to FIG. 220A, the in-line pressure sensor 1548 is shown and may be configured to measure a pressure inside an intravenous fluid line, or other fluid line. The in-line pressure sensor 1548 may be disposed between a first portion 1552 of an IV line and a second portion 1554 of the IV line.

The in-line pressure sensor 1548 includes a first port 1558 and a second port 1556. The second port 1556 includes a first arm 1564 and a second arm 1566. The distal end 1560 of the first arm 1564 arcs toward a central axis 1572 of the second portion 1554 of the IV line. Similarly, the distal end 1562 of the second arm 1566 arcs toward the central axis 1572. The second port 1556 is inserted into the second portion 1554 of the IV line to stretch the second portion 1554 out along length 1570. This stretching creates a first flat side 1574. The second port 1556 is inserted into the second portion 1554 until the second portion 1554 engages with a raised flange 1568. The second portion 1554 may be attached, glued, welded, ultrasonically welded, etc. to the raised flange 1568. The first portion 1552 is attached to a first port 1558 which may be attached, glued, welded, ultrasonically welded, etc. to the raised flange 1568.

Referring now to FIG. 220B, the in-line pressure sensor 1548 is shown assembled. Please note the first flat side 1574. Because fluid contained within the IV can cause deformation of the second portion 1554 of the IV tube, those deformations would be enlarged by the first flat side 1574 and the second flat side 1576, which are both easily seen in FIG. 220C.

FIG. 220D shows the in-line pressure sensor 1548 being coupled by a clip 1550 in accordance with an embodiment of the present disclosure. The clip 1550 includes a first elongated member 1578 and a second elongated member 1580. The first and second elongated members 1578, 1580 pivot along a living hinge 1582. As is easily seen in FIG. 220E, as the first and second elongated members 1578, 1580 expand due to increased pressure within the IV tube, the living hinge 1582. A sensor 1584 can be utilized to measure the pivot amount of the clip 1550. The sensor 1584 may be any sensor to measure a pivot, movement, displacement, etc., such as a beam-breaking sensor, a hall sensor, a potentiometer, any sensor known to one of ordinary skill in the relevant art, etc. FIG. 220F shows another view of the clip 1550 to illustrate a stop member 1586, which is optional. The stop member 1586 may prevent the first and second elongated members 1578, 1580 from contacting each other.

FIG. 221 shows another in-line pressure sensor 1594 in accordance with yet another embodiment of the present disclosure. The in-line pressure sensor 1594 includes an insert or tube 1596. Fluid between the insert 1596 and the IV tube 1598 will cause the deflection of the material needed for pressure measurement by the clip 1550.

FIG. 222 shows a downstream bladder or an in-line pressure sensor 1588 in accordance with yet another embodiment of the present disclosure. The in-line pressure senor 1588 of FIG. 222 includes a narrow region 1590 adjacent to the flange 1592. In some embodiments, the item shown in FIG. 222 can be utilized as a downstream bladder 1588 in accordance with an embodiment of the present disclosure. The downstream bladder 1588 includes a narrow region 1590 adjacent to a flange 1592. The flange 1592 may be utilized as an attachment point by the tube 1594. The tube 1594 of the passageway in the flange 1592 connecting the first portion of the tube 1594 to a second portion of the tube 1596 may be of a reduced diameter relative to the tube 1594, 1596 to dislodge bubbles. For example, the tube portion 1599 of FIG. 223 may be part of the IV tube 1598 or may be embedded within the flange 1592. The tube portion 1599 may use the impeders 1600 to dislodge bubbles. The first portion of the tube 1594 may be coupled to an output of a peristaltic pump 1020, e.g., a peristaltic pump disclosed herein. The downstream bladder 1588 may smooth out irregular fluid flow caused by the pulsating process of a peristaltic pump. In some embodiments of the present disclosure, a very compliant tube may be used in place of a downstream bladder 1588. The impeders 1600 of the tube 1599 of FIG. 223 may be used to slightly increase pressure downstream of a highly compliant tube. In some embodiments of the present disclosure, the impeders 1600 may be configured to be a nucleation site for air bubbles. The nucleation site may be formed by roughing, scoring, or etching the impeders 1600. A heater 1601 may be utilized to increase the fluid temperature prior to having the fluid come into contact with a nucleation site.

FIG. 224 shows another embodiment of a downstream bladder 1602 in accordance with another embodiment of the present disclosure. The downstream bladder 1602 includes a tube 1608. The tube 1610 includes a compliant section 1604 that allows for expansion to absorb irregular fluid flow. The rest of the tube 1608 may have standard IV compliance, while the compliant section 1604 may be substantially more compliant than the bulk of the tube 1608.

FIG. 225 shows a section of tubing 1606 that includes nucleation sites 1616 with an air trap 1612 in accordance with an embodiment of the present disclosure. Liquid fluid can flow downstream in a direction 1614. As the liquid fluid flows downstream, the liquid fluid passes an air trap 1612 and passes by nucleation sites 1616. The nucleation sites 1616 may be configured to encourage gasses within the liquid fluid to form bubbles at microscopic locations along the nucleation sites 1616. The bubbles may start to flow upstream, which is opposite to the direction 1614. The air trap 1612 may trap the bubbles as they flow upstream along a wall of the tube 1610. The air trap 1612 may be formed by interfacing together two tube sections 1610 such that one tube section folds back on itself thereby taking a general toroid shape for the air trap 1612. In yet an additional embodiment of the present disclosure, the liquid fluid may be heated upstream of the nucleation sites 1616 to help facilitate outgassing via bubble formation on the nucleation sites 1616.

FIG. 226 shows a captive screw 1618 in accordance with an embodiment of the present disclosure. The captive screw 1618 includes a head 1632, a narrow neck 1620, an enlarged mid-body 1626, an unthreaded end 1628, and a threaded end 1630. The enlarged mid-body 1626 may be captured within an enlarged cavity 1636 of a first section of material 1622 (e.g., a metal panel). The narrow neck 1620 of the captive screw 1618 may be disposed within a narrow cavity 1634. The optional second section of material 1624 may surround the unthreaded end 1628. In some embodiments the threaded end 1630 is coupled directly to the enlarged mid-body 1626. The captive screw 1618 may be pressed into the narrow cavity 1634 and the enlarged cavity 1636 as part of an access panel to a peristaltic pump, e.g., a back or side panel of a peristaltic pump described here.

FIGS. 227A-227D illustrate a pole clamp 1638 in accordance with an embodiment of the present disclosure. The pole clamp 1638 is configured to allow for coarse and fine adjustments to allow a user to quickly attach, for example, a medical device to a pole.

The pole clamp 1638 includes a fixed jaw member 1642 and a moveable jaw member 1644. The pole clamp 1638 includes a body having a lower-half of the body 1640 and an upper-half of the body 1640 (see FIGS. 227C-227D). The pole clamp 1638 also includes a knob 1648 coupled to a threaded screw 1650. The threaded screw 1650 engages with a collar 1656. The collar 1656 includes cams 1652. The cams 1652 engage with the cam trenches 1654. The threaded screw 1650 can engage with internal threads of the collar 1656 to actuate the moveable jaw member 1644 as the knob 1648 is turned.

Prior to securing the pole clamp 1638 to a medical pole, a slight counter-clockwise turn of the knob 1648, the cams 1652 of the collar 1656 will rotate such that the cams 1652 are in the trench 1658 of FIG. 227D or the trench 1660 of FIG. 227C. In this position, the knob 1648 can easily be moved toward or away from the fixed jaw member 1642 thereby actuating the moveable jaw member 1644. When a coarse position has been made by the user, a clockwise turn of the knob 1648 will rotate the collar 1656 such that the cams 1652 actuate into the cam trenches 1654. Because the cam trenches 1654 are only long enough so that the collar 1656 is actuated about a quarter of a turn, the cams 1652 will stop rotating and the threaded screw 1650 will instead rotate. The rotation of threaded screw 1650 engages with internal threads of the collar 1656 such that the moveable jaw member 1644 is pushed toward the fixed jaw member 1642 thereby providing a fine adjustment mechanism. FIG. 227B shows the top view of the pole clamp 1638 with the upper-half of the body 1640 (see FIG. 227D) removed. FIG. 227C shows a close-up view of the lower-half of the body 1640 with the threaded screw 1650 removed to illustrate the trench 1660 while FIG. 227D shows a close-up view of the upper-half of the body 1643 to also illustrate the trench 1658 and the cam trenches 1662.

FIG. 228 shows a block diagram that illustrates a system 1664 for pumping fluid from a primary IV bag 1670 and a secondary IV bag 1668 in accordance with an embodiment of the present disclosure. The system 1664 includes the primary IV bag 1670 coupled to a check valve 1672. The check value 1672 may be a one-way valve, such as a duck-bill valve, configured to allow fluid to flow from the primary IV bag 1670 and a Y-connector 1674. The secondary IV bag 1668 is also connected to the Y-connector 1674 such that fluid may be pumped downstream by action for a pump 1676. The pump 1676 may be a spring-biased peristaltic pump 1020.

The pump 1676 also includes a GUI 1678, which may be a touch screen, for example. A user can interact with the GUI 1678 to set the pump 1676 into a secondary-infusion mode. Alternatively, the user can interact with the GUI on the Main Processing Control Unit (PCU) (see FIG. 187) to achieve the same effect. The secondary-infusion mode may be a state of a control system implemented by a processor executing a plurality of executable instructions (e.g., a discrete PID control loop implemented in software).

After fluid has been discharged downstream, the outlet valve of the pump 1676 may be closed and the upstream inlet valve opened. The processor may limit the rise of the plunger to limit the amount of fluid that flows from the Y-connector 1674 to thereby control the pressure of the fluid within the upstream fluid lines. The processor may be configured to limit sympathetic flow by limiting the actuation of the plunger by keeping the upstream fluid pressure above the predetermined threshold. The predetermined threshold may be a pressure above a crack pressure of the check valve 1672, such as pressure between 1.5 to 5 lbs-per-square inch (or about 6,900 Pascals to about 34,500 Pascals).

The actuation limit may be a plunger speed limit or a rotation speed limit of a cam shaft. The processor may not limit the actuation when in the pump 1676 is in the secondary-infusion mode unless the fluid flow rate is above a predetermined fluid flow rate, e.g., 250 milliliters per hour, 500 milliliters per hour, or any rate in between. The flow rate may be limited when in the secondary-infusion mode, e.g., to 500 milliliters/hr or less. Is some embodiments, the pump 1676 may limit the actuation to ensure sympathetic flow is less than 5% of a volume to be infused (e.g., then volume to be infused may be between 50 milliliters/hr to 1000 milliliters/hr.

FIG. 229 shows a block diagram that illustrates a system 1708 for pumping fluid in conjunction with a check valve 1714 to prevent outgas sing in accordance with an embodiment of the present disclosure. The check valve 1714 may be a duck-billed valve. The system 1708 includes an IV bag 1710 with a fluid line coupled to a pump 1712 (e.g., the peristaltic pump 1020). A check valve 1714 may be configured to increase the fluid pressure inside the IV tube in the pump 1712. That is, the check valve 1714 may be configured to provide a nadir pressure within the tube. The crack pressure of the check valve 1714 may be configured to raise the nadir pressure above a predetermined threshold. The predetermined threshold may be above an outgassing pressure of the fluid within the IV line (e.g., between the valve 1714 and the pump 1712 and/or within the pump 1712 adjacent to a spring-biased plunger). In some embodiments the crack pressure is adjustable in the check valve 1714 and may be adjusted to account for temperature changes. The pump 1712 may electronically adjust the crack pressure of the check valve in some embodiments. Additionally, alternatively, or optionally, the check valve 1714 may be adjusted in accordance with measured ambient pressure, measured pressure of the fluid or the expected or measured amount of dissolved gas. In some embodiments, a pump 1712 may control the spring-biased plunger to keep the nadir pressure above the outgas pressure.

FIG. 230A shows a system 1724 for pumping fluid having a retainer 1726 configured to secure the retainer 1726 to the pump body. FIG. 230B shows a close up view of the retainer 1726. The retainer 1726 includes a first tube 1728 and a second tube 1732. The first and second tubes 1728, 1732 are coupled to a plastic retainer that may be concentrically surround the tube or may be rectangular shape for being secured within the peristaltic pump 1020.

FIG. 231 shows a flowchart diagram of a method 1680 for pumping fluid and adjusting fluid delivery estimates to account for air or bubbles in accordance with an embodiment of the present disclosure. The method 1680 includes Acts 1682-1706. The method 1680 may be performed by a peristaltic pump 1020 having a spring-biased plunger where an actuator lift the plunger thereby charging the spring and the actuator and the plunger can disengage from each other.

Act 1682 opens an upstream valve. For example, after fluid has been discharged downstream by the plunger, the peristaltic pump needs to fill the tube adjacent of the plunger with fluid. Thus, in Act 1684, the method 1680 actuates (e.g., using a cam shaft) the spring-biased plunger away from the tube. Once fluid is filled within the tube adjacent to the spring-biased plunger, Act 1686 closes the upstream valve thereby fluidly sealing the filled tube adjacent to the spring-biased plunger. Act 1688 actuates the spring-biased plunger toward the tube and Act 1690 disengages the actuator from the spring-biased plunger. The spring-biased plunger is held against the tubing adjacent to the spring-biased plunger by a force of the spring after act 1690. Thus, Act 1692 determines a first position of the spring-biased plunger, which may deviate from the relative position of the actuator. Any air adjacent to the spring-biased plunger may be substantially compressed when the first position of the plunger is determined. Act 1694 actuates the spring-biased plunger away from the tube.

Act 1696 opens a downstream valve and act 1698 actuates the spring-biased plunger toward the tube to thereby discharge fluid downstream. However, Act 1700 closes the downstream outlet valve prior to full discharge of the fluid by the spring-biased plunger. Act 1702 disengages the actuator from the spring-biased plunger. As before, fluid is sealed between the inlet and outlet valves and the position of the spring-biased plunger now corresponds to the amount of fluid in the tubing adjacent to the spring-biased plunger. Also, any air or bubbles are substantially compressed during Act 1702. Act 1704 determines a second position of the spring-biased plunger.

Act 1706 uses the two positions to determine the amount of fluid that has been discharged downstream. Because both of the two positions were made with the spring applying pressure to the tube (via the spring-biased plunger) and thereby substantially compressing any air within the tube, the positions should “filter out” any air from the fluid downstream discharge calculations. Act 1700 may close the downstream outlet valve to ensure that a predetermined amount of fluid remains adjacent to the spring-biased plunger such that the spring-biased plunger does not engage a mechanical stop (that is, the spring-biased plunger could move further toward the tube if less fluid was in the tube).

Various alternatives and modifications can be devised by those skilled in the art without departing from the disclosure. Accordingly, the present disclosure is intended to embrace all such alternatives, modifications and variances. Additionally, while several embodiments of the present disclosure have been shown in the drawings and/or discussed herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. And, those skilled in the art will envision other modifications within the scope and spirit of the claims appended hereto. Other elements, steps, methods and techniques that are insubstantially different from those described above and/or in the appended claims are also intended to be within the scope of the disclosure.

The embodiments shown in the drawings are presented only to demonstrate certain examples of the disclosure. And, the drawings described are only illustrative and are non-limiting. In the drawings, for illustrative purposes, the size of some of the elements may be exaggerated and not drawn to a particular scale. Additionally, elements shown within the drawings that have the same numbers may be identical elements or may be similar elements, depending on the context.

Where the term “comprising” is used in the present description and claims, it does not exclude other elements or steps. Where an indefinite or definite article is used when referring to a singular noun, e.g., “a,” “an,” or “the,” this includes a plural of that noun unless something otherwise is specifically stated. Hence, the term “comprising” should not be interpreted as being restricted to the items listed thereafter; it does not exclude other elements or steps, and so the scope of the expression “a device comprising items A and B” should not be limited to devices consisting only of components A and B. This expression signifies that, with respect to the present disclosure, the only relevant components of the device are A and B.

Furthermore, the terms “first,” “second,” “third,” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein.

1. A medical pump comprising:
a plunger transitionable between a first position and a second position, wherein when a tube is loaded within the pump and the plunger is in the second position, the plunger applies a force against the tube;
a cam shaft configured to actuate the plunger to transition the plunger from the first position to the second position;
a motor coupled to the cam shaft;
a heatsink to dissipate heat from the motor, the heatsink including a first part and a second part; and
a housing, the housing containing at least a portion of each of the plunger, the cam shaft, the motor and the heatsink,
wherein the first part of the heatsink extends through a surface of the housing, the second part is thermally in contact with the motor, and the first and second parts of the heatsink are movable relative to one another along a longitudinally extending axis such that the first and second parts define an overall length that is adjustable.

2. The pump according to claim 1, wherein:
the first and second parts of the heatsink are slidable relative to one another while frictionally fit together.

3. The pump according to claim 1, wherein the heatsink further comprises:
a thermally conductive braided wire having a first end and a second end, the first end being coupled to the motor to dissipate heat from the motor.

4. The pump according to claim 3, wherein the second end is coupled to the heatsink.

5. The pump according to claim 4, wherein the second end is soldered onto the heatsink.

6. The pump according to claim 4, further comprising a clip, wherein the second end is clipped onto the heatsink using the clip.

7. The pump according to claim 1, wherein the first end is soldered onto the motor.

8. The pump according to claim 1, further comprising a clip, wherein the first end is clipped onto the heatsink using the clip.

9. The pump according to claim 3, wherein the thermally conductive, braided wire comprises a metal.

10. The pump according to claim 9, wherein the metal is copper.

11. The pump according to claim 9, wherein the metal is aluminum.

12. The pump according to claim 1, wherein the heatsink is disposed on a back of the pump.

13. The pump according to claim 1, wherein the heatsink is positioned to dissipate heat into a surrounding ambient air.

14. The pump according to claim 1, further comprising a heat pipe coupled to the heatsink to dissipate heat therebetween.

15. The pump according to claim 9, wherein the thermally conductive, braided wire comprising a non-metallic thermally conductive material.

16. The pump according to claim 3, further comprising a powerbar heat dissipater, wherein the first end of thermally-conductive, braided wire is thermally coupled to the heatsink and the second end of the thermally-conductive, braided wire is thermally coupled to the powerbar heat dissipater.

17. The pump according to claim 16, further comprising a heat pipe coupled to the powerbar heat dissipater and the thermally-conductive, braided wire to dissipate heat therebetween.

18. The pump according to claim 16, further comprising a heat pipe coupled to the powerbar heat dissipater and the heatsink to dissipate heat therebetween.

19. A pump for treating a patient, the pump comprising:
a plunger configured for actuation against a tube;
a cam shaft configured to actuate the plunger;
a motor coupled to the cam shaft;
a heatsink; and
a planar-thermal connector having a first end and a second end, wherein the planar-thermal connector is configured to thermally conduct heat from the motor.

20. The pump according to claim 19, the planar-thermal connector having at least one arm extending to thermally contact the motor.

21. The pump according to claim 19, the planar-thermal connector having two arms extending to contact two metal connectors, the two metal connectors being in thermal contact with the motor.

22. The pump according to claim 19, further comprising a powerbar heat dissipater, wherein the first end of the planar-thermal connector is thermally coupled to the heatsink and the second end of the planar-thermal connector is thermally coupled to the powerbar heat dissipater.

23. The pump according to claim 22, further comprising a heat pipe coupled to the powerbar heat dissipater and the planar-thermal connector to dissipate heat therebetween.

24. The pump according to claim 22, further comprising a heat pipe coupled to the powerbar heat dissipater and the heatsink to dissipate heat therebetween.

25. The pump according to claim 19, wherein the planar-thermal connector includes two arms extending therefrom, wherein the two arms are in thermal contact with the motor.

26. The pump according to claim 19, wherein the heatsink is disposed on a back of the pump.

27. The pump according to claim 19, the planar-thermal connector further comprising:
a powerbar heat dissipater;
two arms extending to contact two metal connectors, the two metal connectors being in thermal contact with the motor; and
a heat-strap bracket configured to be disposed between the powerbar heat dissipater and the heatsink, the heat-strap bracket being configured to provide compliance between the powerbar heat dissipater and the heatsink.

28. The pump according to claim 27, wherein the heat-strap bracket is electrically insulating.

29. The pump according to claim 27, wherein the heat-strap bracket includes a bottom side disposed away from the heatsink and toward a powerbar heat dissipater, wherein the first end of the planar-thermal connector is disposed adjacent to the bottom side of the heat-strap bracket.

30. The pump according to claim 29, further comprising a thermal pad disposed between the first end of the planar-thermal connector and the powerbar heat dissipater.

31. The pump according to claim 30, the heat-strap bracket further comprising at least one protrusion extending toward the powerbar heat dissipater, the thermal pad having at least one hole configured to receive a corresponding one of the at least one protrusion, wherein the at least one protrusion is configured to limit a compression of the thermal pad.

32. The pump according to claim 27, wherein the heat-strap bracket includes at least one latch receiver configure to receive at least one latch, wherein each of the at least one latch is configured to secure the planar-thermal connector to the heatsink.

33. The pump according to claim 32, wherein the planar-thermal connector includes at least one hole to receive a respective one of the at least one latch.

34. The pump according to claim 19, wherein the heatsink is positioned to dissipate heat into a surrounding ambient air.

35. The pump according to claim 19, further comprising a heat pipe coupled to the planar-thermal connector and the heatsink to dissipate heat therebetween.

36. The pump according to claim 19, further comprising a heat pipe coupled to the planar-thermal connector and the motor to dissipate heat therebetween.

37.-240. (canceled)
